(19) United States
(12) Patent Application Publication
(10) Pub. No.: US 2025/0214976 A1
(43) Pub. Date: Jul. 3, 2025

(54) GPR35 MODULATORS AND USES THEREOF

(71) Applicant: Crossignal Therapeutics, Inc., La Jolla, CA (US)

(72) Inventors: Sangdon Han, La Jolla, CA (US); Zhi Liang Chu, La Jolla, CA (US); Hongmei He, La Jolla, CA (US)

(21) Appl. No.: 18/851,390

(22) PCT Filed: Apr. 6, 2023
(86) PCT No.: PCT/US2023/017794

§ 371 Date: Sep. 26, 2024

Related U.S. Application Data
(60) Provisional application No. 63/328,415, filed on Apr. 7, 2022.

(51) Int. Cl. C07D 405/04; A61K 31/41; A61K 31/436; A61K 31/437; A61K 31/4439; C07D 307/84; C07D 403/04; C07D 405/12; C07D 405/14; C07D 409/04; C07D 409/14; C07D 471/04; C07D 491/048

(52) U.S. Cl. CPC C07D 405/04; A61K 31/41; A61K 31/436; A61K 31/437; A61K 31/4439; C07D 307/84; C07D 403/04; C07D 405/12; C07D 405/14; C07D 409/04; C07D 409/14; C07D 471/04; C07D 491/048

(57) ABSTRACT

Substituted benzofuran, benzothiophene, and indole carboxamide compounds, conjugates, and pharmaceutical compositions for use as immunotherapeutics in the treatment of cancer, such as solid tumors, are disclosed herein. The disclosed compounds are useful, among other things, in the treatment of a solid tumor originating in the breast, brain, kidney, ovary, cervix, pancreas, thyroid, colon, bladder, prostate, liver, stomach, or lung.

### CROSS-REFERENCE

This application claims the benefit of U.S. Provisional Application Ser. No. 63/328,415 filed Apr. 7, 2022, which is hereby incorporated by reference in its entirety.

### BACKGROUND OF THE INVENTION

A need exists in the medicinal arts for the effective treatment of solid tumors with small molecule therapeutics.

### SUMMARY OF THE INVENTION

The present disclosure generally relates to substituted benzofuran, benzothiophene and indole carboxamide compounds, conjugates,compounds or salts of Formula (I) or (II) and pharmaceutical compositions thereof. In certain aspects, the disclosure provides a compound represented by Formula (I):

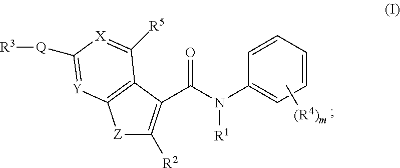

or a pharmaceutically acceptable salt thereof, wherein:
X is selected from CR⁶ and N;
Y is selected from CR¹² and N;
Z is selected from —O—, —S—, and —NR⁸—;
Q is absent or selected from:
   —W—, —O—, —O—W—, —WO—, —S—, —S—W—, —WS—, —NR¹⁰—W—, —W—NR¹⁰—, —C(O)—, —C(O)N(R¹⁰)—, —N(R¹⁰)C(O)—, —C(O)O—, —OC(O)—, —N(R¹⁰)C(O)N(R¹⁰)—, —OC(O)N(R¹⁰)—, —N(R¹⁰)C(O)O—, —S(O)—, —S(O)₂—, and —P(O)₂O—;
m is selected from 1, 2, 3, 4, and 5;
R¹ is selected from:
   hydrogen;
   C₁₋₆ alkyl, C₂₋₆ alkenyl, and C₂₋₆ alkynyl, each of which is optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —C(O)OR¹⁰, —OC(O)R¹⁰, —N(R¹⁰)C(O)N(R¹⁰)₂, —OC(O)N(R¹⁰)₂, —N(R¹⁰)C(O)OR¹⁰, —S(O)R¹⁰, —S(O)₂R¹⁰, —NO₂, ═O, ═S, ═N(R¹⁰), —CN, C₃₋₁₀ carbocycle and 3- to 10-membered heterocycle, wherein the C₃₋₁₀ carbocycle and 3- to 10-membered heterocycle are each optionally substituted with one or more R⁹; and
   C₃₋₁₀ carbocycle and 3- to 10-membered heterocycle, each of which is optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —N(R¹⁰)C(O)N(R¹⁰)₂, —OC(O)N(R¹⁰)₂, —N(R¹⁰)C(O)OR¹⁰, —C(O)OR¹⁰, —OC(O)R¹⁰, —S(O)R¹⁰, —S(O)₂R¹⁰, —NO₂, ═O, ═S, ═N(R¹⁰), —CN, C₁₋₆ alkyl, C₂₋₆ alkenyl, and C₂₋₆ alkynyl, wherein C₁₋₆ alkyl, C₂₋₆ alkenyl, and C₂₋₆ alkynyl are each optionally substituted with one or more R⁹
R² is selected from:
   hydrogen;
   halogen, —OR¹⁰, —SR¹⁰, —N(R¹⁰)₂, —C(O)R¹⁰, —C(O)N(R¹⁰)₂, —N(R¹⁰)C(O)R¹⁰, —N(R¹⁰)C(O)N(R¹⁰)₂, —OC(O)N(R¹⁰)₂, —N(R¹⁰)C(O)OR¹⁰, —C(O)OR¹⁰, —OC(O)R¹⁰, —S(O)R¹⁰, —S(O)₂R¹⁰, —NO₂, and —CN;
   C₁₋₆ alkyl, C₂₋₆ alkenyl, and C₂₋₆ alkynyl, each of which is optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —C(O)OR¹⁰, —OC(O)R¹⁰, —N(R¹⁰)C(O)N(R¹⁰)₂, —OC(O)N(R¹⁰)₂, —N(R¹⁰)C(O)OR¹⁰, —S(O)R¹⁰, —S(O)₂R¹⁰, —NO₂, ═O, ═S, ═N(R¹⁰), —CN, C₃₋₁₀ carbocycle and 3- to 10-membered heterocycle, wherein the C₃₋₁₀ carbocycle and 3- to 10-membered heterocycle are each optionally substituted with one or more R⁹; and
   C₃₋₆ carbocycle and 3- to 6-membered heterocycle, each of which is optionally substituted with one or more substituents independently selected from halogen, —OH, —SR¹⁰, —N(R¹⁰)₂, —C(O)R¹⁰, —C(O)N(R¹⁰)₂, —N(R¹⁰)C(O)R¹⁰, —N(R¹⁰)C(O)N(R¹⁰)₂, —OC(O)N(R¹⁰)₂, —N(R¹⁰)C(O)OR¹⁰, —C(O)OR¹⁰, —OC(O)R¹⁰, —S(O)R¹⁰, —S(O)₂R¹⁰, —NO₂, ═O, ═S, ═N(R¹⁰), —CN, C₁₋₆ alkyl, C₂₋₆ alkenyl, and C₂₋₆ alkynyl, wherein C₁₋₆ alkyl, C₂₋₆ alkenyl, and C₂₋₆ alkynyl are each optionally substituted with one or more R⁹
R³ is selected from:
   C₃₋₆ carbocycle and 3- to 6-membered heterocycle, each of which is optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —N(R¹⁰)C(O)N(R¹⁰)₂, —OC(O)N(R¹⁰)₂, —N(R¹⁰)C(O)OR¹⁰, —C(O)OR¹⁰, —OC(O)R¹⁰, —S(O)R¹⁰, —S(O)₂R¹⁰, —NO₂, ═O, ═S, ═N(R¹⁰), —CN, C₁₋₆ alkyl, C₂₋₆ alkenyl, and C₂₋₆ alkynyl, wherein C₁₋₆ alkyl, C₂₋₆ alkenyl, and C₂₋₆ alkynyl are each optionally substituted with one or more R⁹;
   wherein if Q is —W—, then R³ is additionally selected from halogen, —OR¹⁰, —SR¹⁰, —N(R¹⁰)₂, —C(O)R¹⁰, —C(O)N(R¹⁰)₂, —N(R¹⁰)C(O)R¹⁰, —N(R¹⁰)C(O)N(R¹⁰)₂, —OC(O)N(R¹⁰)₂, —N(R¹⁰)C(O)OR¹⁰, —C(O)OR¹⁰, —OC(O)R¹⁰, —S(O)R¹⁰, —S(O)₂R¹⁰, —NO₂, and —CN; and
   wherein R³ is not pyrimidine;
each R⁴ is independently selected from:
   halogen, —OR⁷, —SR⁷, —N(R⁷)₂, —C(O)R⁷, —C(O)N(R⁷)₂, —N(R⁷)C(O)R⁷, —N(R⁷)C(O)N(R⁷)₂, —OC(O)N(R⁷)₂, —N(R⁷)C(O)OR⁷, —C(O)OR⁷, —OC(O)R⁷, —S(O)R⁷, —S(O)₂R⁷, —NO₂, and —CN;
   C₁₋₆ alkyl, C₂₋₆ alkenyl, and C₂₋₆ alkynyl, each of which is optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —C(O)OR¹⁰, —OC(O)R¹⁰, —N(R¹⁰)C(O)N(R¹⁰)₂, —OC(O)N(R¹⁰)₂, —N(R¹⁰)C(O)OR¹⁰, —S(O)R¹⁰, —S(O)₂R¹⁰, —NO₂, ═O, ═S, ═N(R¹⁰), —CN, C₃₋₁₀ carbocycle and 3- to 10-membered heterocycle, wherein the C₃₋₁₀ carbocycle and 3- to 10-membered heterocycle are each optionally substituted with one or more R⁹; and
   C₃₋₁₀ carbocycle and 3- to 10-membered heterocycle, each of which is optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —N(R¹⁰)C(O)N(R¹⁰)₂, —OC(O)N(R¹⁰)₂, —N(R¹⁰)C(O)OR¹⁰, —C(O)OR¹⁰, —OC(O)R¹⁰, —S(O)R¹⁰, —S(O)₂R¹⁰, —NO₂, ═O, ═S, ═N(R¹⁰), —CN, C₁₋₆ alkyl, C₂₋₆ alkenyl, and C₂₋₆ alkynyl, wherein C₁₋₆ alkyl, C₂₋₆ alkenyl, and C₂₋₆ alkynyl are each optionally substituted with one or more R⁹
R⁵ is selected from:
   hydrogen, halogen, —OR¹⁰, —SR¹⁰, —N(R¹⁰)₂, —NO₂, and —CN; and
   C₁₋₆ alkyl optionally substituted with one or more substituents independently selected from halogen, —OR¹⁰, —SR¹⁰, —N(R¹⁰)₂, —NO₂, and —CN;
R⁶ is selected from:
   hydrogen, halogen, —OR¹⁰, —SR¹⁰, —N(R¹⁰)₂, —NO₂, and —CN; and
   C₁₋₆ alkyl optionally substituted with one or more substituents independently selected from halogen, —OR¹⁰, —SR¹⁰, —N(R¹⁰)₂, —NO₂, and —CN;
each R⁷ is independently selected from:
   hydrogen;
   C₁₋₆ alkyl, C₂₋₆ alkenyl, and C₂₋₆ alkynyl, each of which is optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —C(O)OR¹⁰, —OC(O)R¹⁰, —N(R¹⁰)C(O)N(R¹⁰)₂, —OC(O)N(R¹⁰)₂, —N(R¹⁰)C(O)OR¹⁰, —S(O)R¹⁰, —S(O)₂R¹⁰, —NO₂, ═O, ═S, ═N(R¹⁰), —CN, C₃₋₁₀ carbocycle and 3- to 10-membered heterocycle, wherein the C³⁻¹⁰ carbocycle and 3- to 10-membered heterocycle are each optionally substituted with one or more R⁹; and
   C₃₋₁₀ carbocycle and 3- to 10-membered heterocycle, each of which is optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —N(R¹⁰)C(O)N(R¹⁰)₂, —OC(O)N(R¹⁰)₂, —N(R¹⁰)C(O)OR¹⁰, —C(O)OR¹⁰, —OC(O)R¹⁰, —S(O)R¹⁰, —S(O)₂R¹⁰, —NO₂, ═O, ═S, ═N(R¹⁰), —CN, C₁₋₆ alkyl, C₂₋₆ alkenyl, and C₂₋₆ alkynyl, wherein C₁₋₆ alkyl, C₂₋₆ alkenyl, and C₂₋₆ alkynyl are each optionally substituted with one or more R⁹;
R⁸ is selected from:
   hydrogen;
   C₁₋₆ alkyl, C₂₋₆ alkenyl, and C₂₋₆ alkynyl, each of which is optionally substituted with one or more substituents independently selected from halogen, —SR¹⁰, —N(R¹⁰)₂, —C(O)R¹⁰, —C(O)N(R¹⁰)₂, —N(R¹⁰)C(O)R¹⁰, —C(O)OR¹⁰, —OC(O)R¹⁰, —N(R¹⁰)C(O)N(R¹⁰)₂, —OC(O)N(R¹⁰)₂, —N(R¹⁰)C(O)OR¹⁰, —S(O)R¹⁰, —S(O)₂R¹⁰, —NO₂, ═O, ═S, ═N(R¹⁰), —CN, C₃₋₁₀ carbocycle and 3- to 10-membered heterocycle, wherein the C₃₋₁₀ carbocycle and 3- to 10-membered heterocycle are each optionally substituted with one or more R⁹; and
   C₃₋₁₀ carbocycle and 3- to 10-membered heterocycle, each of which is optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —N(R¹⁰)C(O)N(R¹⁰)₂, —OC(O)N(R¹⁰)₂, —N(R¹⁰)C(O)OR¹⁰, —C(O)OR¹⁰, —OC(O)R¹⁰, —S(O)R¹⁰, —S(O)₂R¹⁰, —NO₂, ═O, ═S, ═N(R¹⁰), —CN, C₁₋₆ alkyl, C₂₋₆ alkenyl, and C₂₋₆ alkynyl, wherein C₁₋₆ alkyl, C₂₋₆ alkenyl, and C₂₋₆ alkynyl are each optionally substituted with one or more R⁹;
each R⁹ is independently selected from:
   halogen, —R¹⁰, —OR¹⁰, —SR¹⁰, —N(R¹⁰)₂, —C(O)R¹⁰, —C(O)N(R¹⁰)₂, —N(R¹⁰)C(O)R¹⁰, —N(R¹⁰)C(O)N(R¹⁰)₂, —OC(O)N(R¹⁰)₂, —N(R¹⁰)C(O)OR¹⁰, —C(O)OR¹⁰, —OC(O)R¹⁰, —OC(O)₂R¹⁰, —S(O)R¹⁰, —S(O)₂R¹⁰, —NO₂, ═O, ═S, ═N(R¹⁰), and —CN; and
   C₁₋₃ alkyl, C₂₋₃ alkenyl, and C₂₋₃ alkynyl, each of which is optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —N(R¹⁰)C(O)N(R¹⁰)₂, —OC(O)N(R¹⁰)₂, —N(R¹⁰)C(O)OR¹⁰, —C(O)OR¹⁰, —OC(O)R¹⁰, —S(O)R¹⁰, —S(O)₂R¹⁰, —NO₂, ═O, ═S, ═N(R¹⁰), and —CN; and
   C₃₋₁₀ carbocycle and 3- to 10-membered heterocycle, each of which is optionally substituted with one or more substituents independently selected from halogen, —R¹⁰, —OR¹⁰, —SR¹⁰, —N(R¹⁰)₂, —C(O)R¹⁰, —C(O)N(R¹⁰)₂, —N(R¹⁰)C(O)R¹⁰, —N(R¹⁰)C(O)N(R¹⁰)₂, —OC(O)N(R¹⁰)₂, —N(R¹⁰)C(O)OR¹⁰, —C(O)OR¹⁰, —OC(O)R¹⁰, —S(O)R¹⁰, —S(O)₂R¹⁰, —NO₂, ═O, ═S, ═N(R¹⁰), and —CN; and

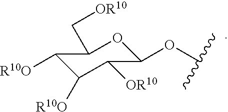

each R¹⁰ is independently selected from:
   hydrogen; and
   C₁₋₆ alkyl, C₂₋₆ alkenyl, and C₂₋₆ alkynyl, each of which is optionally substituted with one or more substituents independently selected from halogen, —CN, —OH, —SH, —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₃₋₁₀ carbocycle, 3- to 10-membered heterocycle; and
   C₃₋₁₀ carbocycle, and 3- to 10-membered heterocycle, each of which is optionally substituted with one or more substituents independently selected from halogen, —CN, —OH, —SH, —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₁₋₆ alkyl, C₂₋₆ alkenyl, C₂₋₆ alkynyl, C₃₋₁₀ carbocycle, 3- to 10-membered heterocycle, and C₁₋₆ haloalkyl;
W is selected from:
   C₁₋₆ alkylene, C₂₋₆ alkenylene, and C₂₋₆ alkynylene, each of which is optionally substituted with one or more substituents independently selected from halogen, —CN, —OH, —SH, —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₃₋₁₀ carbocycle, and 3- to 10-membered heterocycle;
R¹² is selected from:
   hydrogen, halogen, —OR¹⁰, —SR¹⁰, —N(R¹⁰)₂, —NO₂, and —CN; and
   C₁₋₆ alkyl optionally substituted with one or more substituents independently selected from halogen, —OR¹⁰, —SR¹⁰, —N(R¹⁰)₂, —NO₂, and —CN;
wherein if Z is NH then R⁴ is not NH₂, and
wherein if Z is O or S, R⁴ is —OR³, and R⁵ is H, then W—R³ is additionally selected from C₁₋₆ alkyl, C₂₋₆ alkenyl, and C₂₋₆ alkynyl.

In certain aspects, the disclosure provides a compound represented by Formula (II):

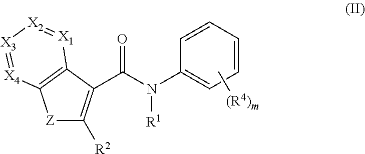

or a pharmaceutically acceptable salt, wherein:
X₁, X₂, X₃, and X₄ are each independently selected from C-Q-R³, CR⁶ and N;
Z is selected from —O—, —S—, and —NR⁸—;
Q is absent or selected from:
   —W—, —O—, —O—W—, —W—O—, —S—, —S—W—, —W—S—, —NR¹⁰—W—, —W—NR¹⁰—, —C(O)—, —C(O)N(R¹⁰)—, —N(R¹⁰)C(O)—, —C(O)O—, —OC(O)—, —N(R¹⁰)C(O)N(R¹⁰)—, —OC(O)N(R¹⁰)—, —N(R¹⁰)C(O)O—, —S(O)—, —S(O)₂—, and —P(O)₂O—;
m is selected from 1, 2, 3, 4, and 5;
R¹ is selected from:
   hydrogen;
   C₁₋₆ alkyl, C₂₋₆ alkenyl, and C₂₋₆ alkynyl, each of which is optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —C(O)OR¹⁰, —OC(O)R¹⁰, —N(R¹⁰)C(O)N(R¹⁰)₂, —OC(O)N(R¹⁰)₂, —N(R¹⁰)C(O)OR¹⁰, —S(O)R¹⁰, —S(O)₂R¹⁰, —NO₂, ═O, ═S, ═N(R¹⁰), —CN, C₃₋₁₀ carbocycle and 3- to 10-membered heterocycle, wherein the C₃₋₁₀ carbocycle and 3- to 10-membered heterocycle are each optionally substituted with one or more R⁹; and
   C₃₋₁₀ carbocycle and 3- to 10-membered heterocycle, each of which is optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —N(R¹⁰)C(O)N(R¹⁰)₂, —OC(O)N(R¹⁰)₂, —N(R¹⁰)C(O)OR¹⁰, —C(O)OR¹⁰, —OC(O)R¹⁰, —S(O)R¹⁰, —S(O)₂R¹⁰, —NO₂, ═O, ═S, ═N(R¹⁰), —CN, C₁₋₆ alkyl, C₂₋₆ alkenyl, and C₂₋₆ alkynyl, wherein C₁₋₆ alkyl, C₂₋₆ alkenyl, and C₂₋₆ alkynyl are each optionally substituted with one or more R⁹;
R² is selected from:
   hydrogen;
   halogen, —OR¹⁰, —SR¹⁰, —N(R¹⁰)₂, —C(O)R¹⁰, —C(O)N(R¹⁰)₂, —N(R¹⁰)C(O)R¹⁰, —N(R¹⁰)C(O)N(R¹⁰)₂, —OC(O)N(R¹⁰)₂, —N(R¹⁰)C(O)OR¹⁰, —C(O)OR¹⁰, —OC(O)R¹⁰, —S(O)R¹⁰, —S(O)₂R¹⁰, —NO₂, ═O, ═S, ═N(R¹⁰), and —CN;
   C₁₋₆ alkyl, C₂₋₆ alkenyl, and C₂₋₆ alkynyl, each of which is optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —C(O)OR¹⁰, —OC(O)R¹⁰, —N(R¹⁰)C(O)N(R¹⁰)₂, —OC(O)N(R¹⁰)₂, —N(R¹⁰)C(O)OR¹⁰, —S(O)R¹⁰, —S(O)₂R¹⁰, —NO₂, ═O, ═S, ═N(R¹⁰), —CN, C₃₋₁₀ carbocycle and 3- to 10-membered heterocycle, wherein the C₃₋₁₀ carbocycle and 3- to 10-membered heterocycle are each optionally substituted with one or more R⁹; and
   C₃₋₁₀ carbocycle and 3- to 10-membered heterocycle, each of which is optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —N(R¹⁰)C(O)N(R¹⁰)₂, —OC(O)N(R¹⁰)₂, —N(R¹⁰)C(O)OR¹⁰, —C(O)OR¹⁰, —OC(O)R¹⁰, —S(O)R¹⁰, —S(O)₂R¹⁰, —NO₂, ═O, ═S, ═N(R¹⁰), —CN, C₁₋₆ alkyl, C₂₋₆ alkenyl, and C₂₋₆ alkynyl, wherein C₁₋₆ alkyl, C₂₋₆ alkenyl, and C₂₋₆ alkynyl are each optionally substituted with one or more R⁹
R³ is selected from:
   hydrogen;
   halogen, —OR¹⁰, —SR¹⁰, —N(R¹⁰)₂, —C(O)R¹⁰, —C(O)N(R¹⁰)₂, —N(R¹⁰)C(O)R¹⁰, —N(R¹⁰)C(O)N(R¹⁰)₂, —OC(O)N(R¹⁰)₂, —N(R¹⁰)C(O)OR¹⁰, —C(O)OR¹⁰, —OC(O)R¹⁰, —S(O)R¹⁰, —S(O)₂R¹⁰, —NO₂, ═O, ═S, ═N(R¹⁰), and —CN;
   C₁₋₆ alkyl, C₂₋₆ alkenyl, and C₂₋₆ alkynyl, each of which is optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —C(O)OR¹⁰, —OC(O)R¹⁰, —N(R¹⁰)C(O)N(R¹⁰)₂, —OC(O)N(R¹⁰)₂, —N(R¹⁰)C(O)OR¹⁰, —S(O)R¹⁰, —S(O)₂R¹⁰, —NO₂, ═O, ═S, ═N(R¹⁰), —CN, C₃₋₁₀ carbocycle and 3- to 10-membered heterocycle, wherein the C₃₋₁₀ carbocycle and 3- to 10-membered heterocycle are each optionally substituted with one or more R⁹;
   C₃₋₁₀ carbocycle and 3- to 10-membered heterocycle, each of which is optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —N(R¹⁰)C(O)N(R¹⁰)₂, —OC(O)N(R¹⁰)₂, —N(R¹⁰)C(O)OR¹⁰, —C(O)OR¹⁰, —OC(O)R¹⁰, —S(O)R¹⁰, —S(O)₂R¹⁰, —NO₂, ═O, ═S, ═N(R¹⁰), —CN, C₁₋₆ alkyl, C₂₋₆ alkenyl, and C₂₋₆ alkynyl, wherein C₁₋₆ alkyl, C₂₋₆ alkenyl, and C₂₋₆ alkynyl are each optionally substituted with one or more R⁹; and
   wherein R³ is not pyrimidine;
each R⁴ is independently selected from:
   halogen, —OR⁷, —SR⁷, —N(R⁷)₂, —C(O)R⁷, —C(O)N(R⁷)₂, —N(R⁷)C(O)R⁷, —N(R⁷)C(O)N(R⁷)₂, —OC(O)N(R⁷)₂, —N(R⁷)C(O)OR⁷, —C(O)OR⁷, —OC(O)R⁷, —S(O)R⁷, —S(O)₂R⁷, —NO₂, and —CN;
   C₁₋₆ alkyl, C₂₋₆ alkenyl, and C₂₋₆ alkynyl, each of which is optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —C(O)OR¹⁰, —OC(O)R¹⁰, —N(R¹⁰)C(O)N(R¹⁰)₂, —OC(O)N(R¹⁰)₂, —N(R¹⁰)C(O)OR¹⁰, —S(O)R¹⁰, —S(O)₂R¹⁰, —NO₂, ═O, ═S, ═N(R¹⁰), —CN, C₃₋₁₀ carbocycle and 3- to 10-membered heterocycle, wherein the C₃₋₁₀ carbocycle and 3- to 10-membered heterocycle are each optionally substituted with one or more R⁹; and
   C₃₋₁₀ carbocycle and 3- to 10-membered heterocycle, each of which is optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —N(R¹⁰)C(O)N(R¹⁰)₂, —OC(O)N(R¹⁰)₂, —N(R¹⁰)C(O)OR¹⁰, —C(O)OR¹⁰, —OC(O)R¹⁰, —S(O)R¹⁰, —S(O)₂R¹⁰, —NO₂, ═O, ═S, ═N(R¹⁰), —CN, C₁₋₆ alkyl, C₂₋₆ alkenyl, and C₂₋₆ alkynyl, wherein C₁₋₆ alkyl, C₂₋₆ alkenyl, and C₂₋₆ alkynyl are each optionally substituted with one or more R⁹ each R⁶ is independently selected from:
   hydrogen, halogen, —OR¹⁰, —SR¹⁰, —N(R¹⁰)₂, —NO₂, and —CN; and
   C₁₋₆ alkyl optionally substituted with one or more substituents independently selected from halogen, —OR¹⁰, —SR¹⁰, —N(R¹⁰)₂, —NO₂, and —CN;
each R⁷ is independently selected from:
   hydrogen;
   C₁₋₆ alkyl, C₂₋₆ alkenyl, and C₂₋₆ alkynyl, each of which is optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —C(O)OR¹⁰, —OC(O)R¹⁰, —N(R¹⁰)C(O)N(R¹⁰)₂, —OC(O)N(R¹⁰)₂, —N(R¹⁰)C(O)OR¹⁰, —S(O)R¹⁰, —S(O)₂R¹⁰, —NO₂, ═O, ═S, ═N(R¹⁰), —CN, C₃₋₁₀ carbocycle and 3- to 10-membered heterocycle, wherein the C₃₋₁₀ carbocycle and 3- to 10-membered heterocycle are each optionally substituted with one or more R⁹; and
   C₃₋₁₀ carbocycle and 3- to 10-membered heterocycle, each of which is optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —N(R¹⁰)C(O)N(R¹⁰)₂, —OC(O)N(R¹⁰)₂, —N(R¹⁰)C(O)OR¹⁰, —C(O)OR¹⁰, —OC(O)R¹⁰, —S(O)R¹⁰, —S(O)₂R¹⁰, —NO₂, ═O, ═S, ═N(R¹⁰), —CN, C₁₋₆ alkyl, C₂₋₆ alkenyl, and C₂₋₆ alkynyl, wherein C₁₋₆ alkyl, C₂₋₆ alkenyl, and C₂₋₆ alkynyl are each optionally substituted with one or more R⁹
R⁸ is selected from:
   hydrogen;
   C₁₋₆ alkyl, C₂₋₆ alkenyl, and C₂₋₆ alkynyl, each of which is optionally substituted with one or more substituents independently selected from halogen, —SR¹⁰, —N(R¹⁰)₂, —C(O)R¹⁰, —C(O)N(R¹⁰)₂, —N(R¹⁰)C(O)R¹⁰, —C(O)OR¹⁰, —OC(O)R¹⁰, —N(R¹⁰)C(O)N(R¹⁰)₂, —OC(O)N(R¹⁰)₂, —N(R¹⁰)C(O)OR¹⁰, —S(O)R¹⁰, —S(O)₂R¹⁰, —NO₂, ═O, ═S, ═N(R¹⁰), —CN, C₃₋₁₀ carbocycle and 3- to 10-membered heterocycle, wherein the C₃₋₁₀ carbocycle and 3- to 10-membered heterocycle are each optionally substituted with one or more R⁹; and
   C₃₋₁₀ carbocycle and 3- to 10-membered heterocycle, each of which is optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —N(R¹⁰)C(O)N(R¹⁰)₂, —OC(O)N(R¹⁰)₂, —N(R¹⁰)C(O)OR¹⁰, —C(O)OR¹⁰, —OC(O)R¹⁰, —S(O)R¹⁰, —S(O)₂R¹⁰, —NO₂, ═O, ═S, ═N(R¹⁰), —CN, C₁₋₆ alkyl, C₂₋₆ alkenyl, and C₂₋₆ alkynyl, wherein C₁₋₆ alkyl, C₂₋₆ alkenyl, and C₂₋₆ alkynyl are each optionally substituted with one or more R⁹;
each R⁹ is independently selected from:
   halogen, —R¹⁰, —OR¹⁰, —SR¹⁰, —N(R¹⁰)₂, —C(O)R¹⁰, —C(O)N(R¹⁰)₂, —N(R¹⁰)C(O)R¹⁰, —N(R¹⁰)C(O)N(R¹⁰)₂, —OC(O)N(R¹⁰)₂, —N(R¹⁰)C(O)OR¹⁰, —C(O)OR¹⁰, —OC(O)R¹⁰, —OC(O)₂R¹⁰, —S(O)R¹⁰, —S(O)₂R¹⁰, —NO₂, ═O, ═S, ═N(R¹⁰), and —CN; and
   C₁₋₃ alkyl, C₂₋₃ alkenyl, and C₂₋₃ alkynyl, each of which is optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —N(R¹⁰)C(O)N(R¹⁰)₂, —OC(O)N(R¹⁰)₂, —N(R¹⁰)C(O)OR¹⁰, —C(O)OR¹⁰, —OC(O)R¹⁰, —S(O)R¹⁰, —S(O)₂R¹⁰, —NO₂, ═O, ═S, ═N(R¹⁰), and —CN; and
   C₃₋₁₀ carbocycle and 3- to 10-membered heterocycle, each of which is optionally substituted with one or more substituents independently selected from halogen, —R¹⁰, —OR¹⁰, —SR¹⁰, —N(R¹⁰)₂, —C(O)R¹⁰, —C(O)N(R¹⁰)₂, —N(R¹⁰)C(O)R¹⁰, —N(R¹⁰)C(O)N(R¹⁰)₂, —OC(O)N(R¹⁰)₂, —N(R¹⁰)C(O)OR¹⁰, —C(O)OR¹⁰, —OC(O)R¹⁰, —S(O)R¹⁰, —S(O)₂R¹⁰, —NO₂, ═O, ═S, ═N(R¹⁰), and —CN; and

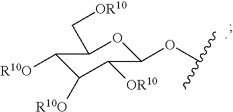

each R¹⁰ is independently selected from:
   hydrogen; and
   C₁₋₆ alkyl, C₂₋₆ alkenyl, and C₂₋₆ alkynyl, each of which is optionally substituted with one or more substituents independently selected from halogen, —CN, —OH, —SH, —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₃₋₁₀ carbocycle, 3- to 10-membered heterocycle; and
   C₃₋₁₀ carbocycle, and 3- to 10-membered heterocycle, each of which is optionally substituted with one or more substituents independently selected from halogen, —CN, —OH, —SH, —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₁₋₆ alkyl, C₂₋₆ alkenyl, C₂₋₆ alkynyl, C₃₋₁₀ carbocycle, 3- to 10-membered heterocycle, and C₁₋₆ haloalkyl;
W is selected from:
   C₁₋₆ alkylene, C₂₋₆ alkenylene, and C₂₋₆ alkynylene, each of which is optionally substituted with one or more substituents independently selected from halogen, —CN, —OH, —SH, —NO₂, —NH₂, ═O, ═S, —O—C₁₋₆ alkyl, —S—C₁₋₆ alkyl, —N(C₁₋₆ alkyl)₂, and —NH(C₁₋₆ alkyl), C₃₋₁₀ carbocycle, and 3- to 10-membered heterocycle.

In certain aspects, the disclosure provides a pharmaceutical composition comprising a compound or salt of any one Formulas (I) and (II), or a pharmaceutically acceptable excipient.

### DETAILED DESCRIPTION OF THE INVENTION

Disclosed herein are substituted benzofuran, benzothiophene and indole carboxamide compounds, conjugates, and pharmaceutical compositions for use in the treatment of cancer, such as solid tumors. In some embodiments, the compounds disclosed herein are modulators of G protein couple receptor 35 (GPR35). In some embodiments, the compounds disclosed inhibit GPR35 expressed in immune cells and promote anti-tumor immunity.

### Definitions

Unless defined otherwise, all technical and scientific terms used herein have the same meaning as is commonly understood by one of skill in the art to which this invention belongs.

As used in the specification and claims, the singular form “a”, “an” and “the” includes plural references unless the context clearly dictates otherwise.

The term “C_{x-y}” or “Cₓ-C_{y}” when used in conjunction with a chemical moiety, such as alkyl, alkenyl, or alkynyl is meant to include groups that contain from x to y carbons in the chain. For example, the term “C₁₋₆alkyl” refers to substituted or unsubstituted saturated hydrocarbon groups, including straight-chain alkyl and branched-chain alkyl groups that contain from 1 to 6 carbons. The term —C_{x-y}alkylene- refers to a substituted or unsubstituted alkylene chain with from x to y carbons in the alkylene chain. For example —C₁₋₆alkylene- may be selected from methylene, ethylene, propylene, butylene, pentylene, and hexylene, any one of which is optionally substituted.

The terms “C_{x-y}alkenyl” and “C_{x-y}alkynyl” refer to substituted or unsubstituted unsaturated aliphatic groups analogous in length and possible substitution to the alkyls described above, but that contain at least one double or triple bond, respectively. The term —C_{x-y}alkenylene- refers to a substituted or unsubstituted alkenylene chain with from x to y carbons in the alkenylene chain. For example, —C₂₋₆alkenylene- may be selected from ethenylene, propenylene, butenylene, pentenylene, and hexenylene, any one of which is optionally substituted. An alkenylene chain may have one double bond or more than one double bond in the alkenylene chain. The term —C_{x-y}alkynylene- refers to a substituted or unsubstituted alkynylene chain with from x to y carbons in the alkenylene chain. For example, —C₂₋₆alkynylene- may be selected from ethynylene, propynylene, butynylene, pentynylene, and hexynylene, any one of which is optionally substituted. An alkynylene chain may have one triple bond or more than one triple bond in the alkynylene chain.

“Alkylene” refers to a straight divalent hydrocarbon chain linking the rest of the molecule to a radical group, consisting solely of carbon and hydrogen, containing no unsaturation, and preferably having from one to twelve carbon atoms, for example, methylene, ethylene, propylene, butylene, and the like. The alkylene chain is attached to the rest of the molecule through a single bond and to the radical group through a single bond. The points of attachment of the alkylene chain to the rest of the molecule and to the radical group are through the terminal carbons respectively. In other embodiments, an alkylene comprises one to five carbon atoms (i.e., C₁-C₅ alkylene). In other embodiments, an alkylene comprises one to four carbon atoms (i.e., C₁-C₄ alkylene). In other embodiments, an alkylene comprises one to three carbon atoms (i.e., C₁-C₃ alkylene). In other embodiments, an alkylene comprises one to two carbon atoms (i.e., C₁-C₂ alkylene). In other embodiments, an alkylene comprises one carbon atom (i.e., C₁ alkylene). In other embodiments, an alkylene comprises five to eight carbon atoms (i.e., C₅-C₈ alkylene). In other embodiments, an alkylene comprises two to five carbon atoms (i.e., C₂-C₅ alkylene). In other embodiments, an alkylene comprises three to five carbon atoms (i.e., C₃-C₅ alkylene). Unless stated otherwise specifically in the specification, an alkylene chain is optionally substituted by one or more substituents such as those substituents described herein.

“Alkenylene” refers to a straight divalent hydrocarbon chain linking the rest of the molecule to a radical group, consisting solely of carbon and hydrogen, containing at least one carbon-carbon double bond, and preferably having from two to twelve carbon atoms. The alkenylene chain is attached to the rest of the molecule through a single bond and to the radical group through a single bond. The points of attachment of the alkenylene chain to the rest of the molecule and to the radical group are through the terminal carbons, respectively. In other embodiments, an alkenylene comprises two to five carbon atoms (i.e., C₂-C₅ alkenylene). In other embodiments, an alkenylene comprises two to four carbon atoms (i.e., C₂-C₄ alkenylene). In other embodiments, an alkenylene comprises two to three carbon atoms (i.e., C₂-C₃ alkenylene). In other embodiments, an alkenylene comprises two carbon atoms (i.e., C₂ alkenylene). In other embodiments, an alkenylene comprises five to eight carbon atoms (i.e., C₅-C₈ alkenylene). In other embodiments, an alkenylene comprises three to five carbon atoms (i.e., C₃-C₅ alkenylene). Unless stated otherwise specifically in the specification, an alkenylene chain is optionally substituted by one or more substituents such as those substituents described herein.

“Alkynylene” refers to a straight divalent hydrocarbon chain linking the rest of the molecule to a radical group, consisting solely of carbon and hydrogen, containing at least one carbon-carbon triple bond, and preferably having from two to twelve carbon atoms. The alkynylene chain is attached to the rest of the molecule through a single bond and to the radical group through a single bond. The points of attachment of the alkynylene chain to the rest of the molecule and to the radical group are through the terminal carbons respectively. In other embodiments, an alkynylene comprises two to five carbon atoms (i.e., C₂-C₅ alkynylene). In other embodiments, an alkynylene comprises two to four carbon atoms (i.e., C₂-C₄ alkynylene). In other embodiments, an alkynylene comprises two to three carbon atoms (i.e., C₂-C₃ alkynylene). In other embodiments, an alkynylene comprises two carbon atoms (i.e., C₂ alkynylene). In other embodiments, an alkynylene comprises five to eight carbon atoms (i.e., C₅-C₈ alkynylene). In other embodiments, an alkynylene comprises three to five carbon atoms (i.e., C₃-C₅ alkynylene). Unless stated otherwise specifically in the specification, an alkynylene chain is optionally substituted by one or more substituents such as those substituents described herein.

The term “carbocycle” as used herein refers to a saturated, unsaturated or aromatic ring in which each atom of the ring is carbon. Carbocycle includes 3- to 10-membered monocyclic rings, 5- to 12-membered bicyclic rings, spiro bicycles, and 5- to 12-membered bridged rings. Each ring of a bicyclic carbocycle may be selected from saturated, unsaturated, and aromatic rings. In an exemplary embodiment, an aromatic ring, e.g., phenyl, may be fused to a saturated or unsaturated ring, e.g., cyclohexane, cyclopentane, or cyclohexene. A bicyclic carbocycle includes any combination of saturated, unsaturated and aromatic bicyclic rings, as valence permits. A bicyclic carbocycle further includes spiro bicylic rings such as spiropentane. A bicyclic carbocycle includes any combination of ring sizes such as 3-3 spiro ring systems, 4-5 fused ring systems, 5-5 fused ring systems, 5-6 fused ring systems, 6-6 fused ring systems, 5-7 fused ring systems, 6-7 fused ring systems, 5-8 fused ring systems, and 6-8 fused ring systems. Exemplary carbocycles include cyclopentyl, cyclohexyl, cyclohexenyl, adamantyl, phenyl, indanyl, naphthyl, and bicyclo[1.1.1]pentanyl.

The term “aryl” refers to an aromatic monocyclic or aromatic multicyclic hydrocarbon ring system. The aromatic monocyclic or aromatic multicyclic hydrocarbon ring system contains only hydrogen and carbon and from five to eighteen carbon atoms, where at least one of the rings in the ring system is aromatic, i.e., it contains a cyclic, delocalized (4n+2) π-electron system in accordance with the Hückel theory. The ring system from which aryl groups are derived include, but are not limited to, groups such as benzene, fluorene, indane, indene, tetralin and naphthalene. Unless stated otherwise specifically in the specification, the term “aryl” or the prefix “ar” (such as in “aralkyl”) is meant to include aryl radicals optionally substituted by one or more substituents such as those substituents described herein.

The term “cycloalkyl” refers to a saturated ring in which each atom of the ring is carbon. Cycloalkyl may include monocyclic and polycyclic rings such as 3- to 10-membered monocyclic rings, 5- to 12-membered bicyclic rings, spiro bicycles, and 5- to 12-membered bridged rings. In certain embodiments, a cycloalkyl comprises three to ten carbon atoms. In other embodiments, a cycloalkyl comprises five to seven carbon atoms. The cycloalkyl may be attached to the rest of the molecule by a single bond. Examples of monocyclic cycloalkyls include, e.g., cyclopropyl, cyclobutyl, cyclopentyl, cyclohexyl, cycloheptyl, and cyclooctyl. Polycyclic cycloalkyl radicals include, for example, adamantyl, spiropentane, norbornyl (i.e., bicyclo[2.2.1]heptanyl), decalinyl, 7,7 dimethyl bicyclo[2.2.1]heptanyl, bicyclo[1.1.1]pentanyl, and the like. Unless otherwise stated specifically in the specification, the term “cycloalkyl” is meant to include cycloalkyl radicals that are optionally substituted by one or more substituents such as those substituents described herein.

The term “cycloalkenyl” refers to a saturated ring in which each atom of the ring is carbon and there is at least one double bond between two ring carbons. Cycloalkenyl may include monocyclic and polycyclic rings such as 3- to 10-membered monocyclic rings, 6- to 12-membered bicyclic rings, and 5- to 12-membered bridged rings. In other embodiments, a cycloalkenyl comprises five to seven carbon atoms. The cycloalkenyl may be attached to the rest of the molecule by a single bond. Examples of monocyclic cycloalkenyls include, e.g., cyclopentenyl, cyclohexenyl, cycloheptenyl, and cyclooctenyl. Unless otherwise stated specifically in the specification, the term “cycloalkenyl” is meant to include cycloalkenyl radicals that are optionally substituted by one or more substituents such as those substituents described herein.

The term “halo” or, alternatively, “halogen” or “halide,” means fluoro, chloro, bromo or iodo. In some embodiments, halo is fluoro, chloro, or bromo.

The term “haloalkyl” refers to an alkyl radical, as defined above, that is substituted by one or more halo radicals, for example, trifluoromethyl, dichloromethyl, bromomethyl, 2,2,2-trifluoroethyl, 1-chloromethyl-2-fluoroethyl, and the like. In some embodiments, the alkyl part of the haloalkyl radical is optionally further substituted as described herein.

The term “heterocycle” as used herein refers to a saturated, unsaturated or aromatic ring comprising one or more heteroatoms. Exemplary heteroatoms include N, O, Si, P, B, and S atoms. Heterocycles include 3- to 10-membered monocyclic rings, 6- to 12-membered bicyclic rings, and 5- to 12-membered bridged rings. A bicyclic heterocycle includes any combination of saturated, unsaturated and aromatic bicyclic rings, as valence permits. In an exemplary embodiment, an aromatic ring, e.g., pyridyl, may be fused to a saturated or unsaturated ring, e.g., cyclohexane, cyclopentane, morpholine, piperidine or cyclohexene. A bicyclic heterocycle includes any combination of ring sizes such as 4-5 fused ring systems, 5-5 fused ring systems, 5-6 fused ring systems, 6-6 fused ring systems, 5-7 fused ring systems, 6-7 fused ring systems, 5-8 fused ring systems, and 6-8 fused ring systems. A bicyclic heterocycle further includes spiro bicylic rings such as 2-oxa-6-azaspiro[3.3]heptane. The term “heterocycle” is meant to encompass “heteroaryl,” “heterocycloalkyl” and “heterocycloalkenyl.”

The term “heteroaryl” refers to a radical derived from a 5 to 18 membered aromatic ring radical that comprises two to seventeen carbon atoms and from one to six heteroatoms selected from nitrogen, oxygen and sulfur. As used herein, the heteroaryl radical is a monocyclic, bicyclic, tricyclic or tetracyclic ring system, wherein at least one of the rings in the ring system is aromatic, i.e., it contains a cyclic, delocalized (4n+2) π-electron system in accordance with the Hückel theory. Heteroaryl includes fused or bridged ring systems. The heteroatom(s) in the heteroaryl radical is optionally oxidized. One or more nitrogen atoms, if present, are optionally quaternized. The heteroaryl is attached to the rest of the molecule through any atom of the ring(s). Examples of heteroaryls include, but are not limited to, azepinyl, acridinyl, benzimidazolyl, benzindolyl, 1,3-benzodioxolyl, benzofuranyl, benzoxazolyl, benzo[d]thiazolyl, benzothiadiazolyl, benzo[b][1,4]dioxepinyl, benzo[b][1,4]oxazinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H-benzo[6,7]cyclohepta[1,2-c]pyridazinyl, dibenzofuranyl, dibenzothiophenyl, furanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl, isothiazolyl, imidazolyl, indazolyl, indolyl, indazolyl, isoindolyl, indolinyl, isoindolinyl, isoquinolyl, indolizinyl, isoxazolyl, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h]quinazolinyl, 1-phenyl-1H-pyrrolyl, phenazinyl, phenothiazinyl, phenoxazinyl, phthalazinyl, pteridinyl, purinyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, thiazolyl, thiadiazolyl, triazolyl, tetrazolyl, triazinyl, thieno[2,3-d]pyrimidinyl, thieno[3,2-d]pyrimidinyl, thieno[2,3-c]pyridinyl, and thiophenyl (i.e. thienyl).

The term “heterocycloalkyl” refers to a saturated ring with carbon atoms and at least one heteroatom. Exemplary heteroatoms include N, O, Si, P, B, and S atoms. Heterocycloalkyl may include monocyclic and polycyclic rings such as 3- to 10-membered monocyclic rings, 6- to 12-membered bicyclic rings, spiro bicycles, and 5- to 12-membered bridged rings. The heteroatoms in the heterocycloalkyl radical are optionally oxidized. One or more nitrogen atoms, if present, are optionally quaternized. The heterocycloalkyl is attached to the rest of the molecule through any atom of the heterocycloalkyl, valence permitting, such as any carbon or nitrogen atoms of the heterocycloalkyl. Examples of heterocycloalkyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 2-oxa-6-azaspiro[3.3]heptane, and 1,1-dioxo-thiomorpholinyl. Unless stated otherwise specifically in the specification, the term “heterocycloalkyl” is meant to include heterocycloalkyl radicals as defined above that are optionally substituted by one or more substituents such as those substituents described herein.

The term “heterocycloalkenyl” refers to an unsaturated ring with carbon atoms and at least one heteroatom and there is at least one double bond between two ring carbons. Heterocycloalkenyl does not include heteroaryl rings. Exemplary heteroatoms include N, O, Si, P, B, and S atoms. Heterocycloalkenyl may include monocyclic and polycyclic rings such as 3- to 10-membered monocyclic rings, 6- to 12-membered bicyclic rings, and 5- to 12-membered bridged rings. In other embodiments, a heterocycloalkenyl comprises five to seven ring atoms. The heterocycloalkenyl may be attached to the rest of the molecule by a single bond. Examples of monocyclic cycloalkenyls include, e.g., pyrroline (dihydropyrrole), pyrazoline (dihydropyrazole), imidazoline (dihydroimidazole), triazoline (dihydrotriazole), dihydrofuran, dihydrothiophene, oxazoline (dihydrooxazole), isoxazoline (dihydroisoxazole), thiazoline (dihydrothiazole), isothiazoline (dihydroisothiazole), oxadiazoline (dihydrooxadiazole), thiadiazoline (dihydrothiadiazole), dihydropyridine, tetrahydropyridine, dihydropyridazine, tetrahydropyridazine, dihydropyrimidine, tetrahydropyrimidine, dihydropyrazine, tetrahydropyrazine, pyran, dihydropyran, thiopyran, dihydrothiopyran, dioxine, dihydrodioxine, oxazine, dihydrooxazine, thiazine, and dihydrothiazine. Unless otherwise stated specifically in the specification, the term “heterocycloalkenyl” is meant to include heterocycloalkenyl radicals that are optionally substituted by one or more substituents such as those substituents described herein.

The term “substituted” refers to moieties having substituents replacing a hydrogen on one or more carbons or substitutable heteroatoms, e.g., an NH or NH₂ of a compound. It will be understood that “substitution” or “substituted with” includes the implicit proviso that such substitution is in accordance with permitted valence of the substituted atom and the substituent, and that the substitution results in a stable compound, i.e., a compound which does not spontaneously undergo transformation such as by rearrangement, cyclization, elimination, etc. In certain embodiments, substituted refers to moieties having substituents replacing two hydrogen atoms on the same carbon atom, such as substituting the two hydrogen atoms on a single carbon with an oxo, imino or thioxo group. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds.

In some embodiments, substituents may include any substituents described herein, for example: halogen, hydroxy, oxo (═O), thioxo (═S), cyano (—CN), nitro (—NO₂), imino (═N—H), oximo (═N—OH), hydrazino (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2), and —R^{b}—S(O)ₜN(R^{a})₂ (where t is 1 or 2); and alkyl, alkenyl, alkynyl, aryl, aralkyl, aralkenyl, aralkynyl, cycloalkyl, cycloalkylalkyl, heterocycloalkyl, heterocycloalkylalkyl, heteroaryl, and heteroarylalkyl, any of which may be optionally substituted by alkyl, alkenyl, alkynyl, halogen, haloalkyl, haloalkenyl, haloalkynyl, oxo (═O), thioxo (═S), cyano (—CN), nitro (—NO₂), imino (═N—H), oximo (═N—OH), hydrazine (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2); wherein each R^{a} is independently selected from hydrogen, alkyl, cycloalkyl, cycloalkylalkyl, aryl, aralkyl, heterocycloalkyl, heterocycloalkylalkyl, heteroaryl, or heteroarylalkyl, wherein each R^{a}, valence permitting, may be optionally substituted with alkyl, alkenyl, alkynyl, halogen, haloalkyl, haloalkenyl, haloalkynyl, oxo (═O), thioxo (═S), cyano (—CN), nitro (—NO₂), imino (═N—H), oximo (═N—OH), hydrazine (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2); and wherein each R^{b} is independently selected from a direct bond or a straight or branched alkylene, alkenylene, or alkynylene chain, and each R^{c} is a straight or branched alkylene, alkenylene or alkynylene chain.

Double bonds to oxygen atoms, such as oxo groups, are represented herein as both “═O” and “(O)”. Double bonds to nitrogen atoms are represented as both “═NR” and “(NR)”. Double bonds to sulfur atoms are represented as both “═S” and “(S)”.

The phrases “parenteral administration” and “administered parenterally” as used herein means modes of administration other than enteral and topical administration, usually by injection, and includes, without limitation, intravenous, intramuscular, intra-arterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable excipient” or “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The term “salt” or “pharmaceutically acceptable salt” refers to salts derived from a variety of organic and inorganic counter ions well known in the art. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. In some embodiments, the pharmaceutically acceptable base addition salt is chosen from ammonium, potassium, sodium, calcium, and magnesium salts.

### Compounds

The following is a discussion of compounds and salts thereof that may be used in the methods of the disclosure. In certain embodiments, the compounds and salts are described in Formulas (I)

In one aspect, disclosed herein is a compound represented by Formula (I):

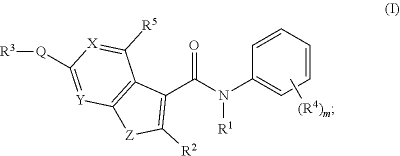

or a pharmaceutically acceptable salt thereof, wherein:
X is selected from CR⁶ and N;
Y is selected from CR¹² and N;
Z is selected from —O—, —S—, and —NR⁸—;
Q is absent or selected from:
   —W—, —O—, —OW—, —WO—, —S—, —SW—, —WS—, —NR¹⁰—W—, —W—NR¹⁰—, C(O)—, —C(O)N(R¹⁰)—, —N(R¹⁰)C(O)—, —C(O)O—, —OC(O)—, —N(R¹⁰)C(O)N(R¹⁰)0-, —OC(O)N(R¹⁰)—, —N(R¹⁰)C(O)O—, —S(O)—, —S(O)₂—, and —P(O)₂O—;
m is selected from 1, 2, 3, 4, and 5;
R¹ is selected from hydrogen, C₁₋₆ alkyl, C₂₋₆ alkenyl, C₂₋₆ alkynyl, C₃₋₁₀ carbocycle and 3- to 10-membered heterocycle, each of which is optionally substituted;
R² is selected from:
   hydrogen, halogen, —OR¹⁰, —SR¹⁰, —N(R¹⁰)₂, —C(O)R¹⁰, —C(O)N(R¹⁰)₂, —N(R¹⁰)C(O)R¹⁰, —N(R¹⁰)C(O)N(R¹⁰)₂, —OC(O)N(R¹⁰)₂, —N(R¹⁰)C(O)OR¹⁰, —C(O)OR¹⁰, —OC(O)R¹⁰, —S(O)R¹⁰, —S(O)₂R¹⁰, —NO₂, and —CN; C₁₋₆ alkyl, C₂₋₆ alkenyl, C₂₋₆ alkynyl, C₃₋₆ carbocycle and 3- to 6-membered heterocycle, each of which is optionally substituted;
R³ is selected from C₃₋₆ carbocycle and 3- to 6-membered heterocycle, each of which is optionally substituted;
each R⁴ is independently selected from:
   halogen, —OR⁷, —SR⁷, —N(R⁷)₂, —C(O)R⁷, —C(O)N(R⁷)₂, —N(R⁷)C(O)R⁷, —N(R⁷)C(O)N(R⁷)₂, —OC(O)N(R⁷)₂, —N(R⁷)C(O)OR⁷, —C(O)OR⁷, —OC(O)R⁷, —S(O)R⁷, —S(O)₂R⁷, —NO₂, and —CN;
   C₁₋₆ alkyl, C₂₋₆ alkenyl, C₂₋₆ alkynyl, C₃₋₁₀ carbocycle and 3- to 10-membered heterocycle, each of which is optionally substituted;
each of R⁵ and R⁶ is independently selected from hydrogen, halogen, —OR¹⁰, —SR¹⁰, —N(R¹⁰)₂, —NO₂, —CN and optionally substituted C₁₋₆ alkyl;
each R⁷ is independently selected from hydrogen, C₁₋₆ alkyl, C₂₋₆ alkenyl, C₂₋₆ alkynyl, C₃₋₁₀ carbocycle and 3- to 10-membered heterocycle, each of which is optionally substituted;
R⁸ is selected from hydrogen, C₁₋₆ alkyl, C₂₋₆ alkenyl, C₂₋₆ alkynyl, C₃₋₁₀ carbocycle and 3- to 10-membered heterocycle, each of which is optionally substituted;
each R¹⁰ is independently selected hydrogen, C₁₋₆ alkyl, C₂₋₆ alkenyl, C₂₋₆ alkynyl, C₃₋₁₀ carbocycle and 3- to 10-membered heterocycle, each of which is optionally substituted;
W is selected from C₁₋₆ alkylene, C₂₋₆ alkenylene, and C₂₋₆ alkynylene, each of which is optionally substituted;
R¹² is selected from hydrogen, halogen, —OR¹⁰, —SR¹⁰, —N(R¹⁰)₂, —NO₂, —CN, and optionally substituted C₁₋₆ alkyl with one or more substituents independently selected from halogen, —OR¹⁰, —SR¹⁰, —N(R¹⁰)₂, —NO₂, and —CN;
wherein if Z is NH then R⁴ is not NH₂, and
wherein if Z is O or S, R⁴ is —OR³, and R⁵ is H, then W—R³ is additionally selected from C₁₋₆ alkyl, C₂₋₆ alkenyl, and C₂₋₆ alkynyl.

In one aspect, disclosed herein is a compound represented by Formula (I):

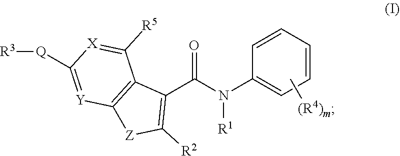

or a pharmaceutically acceptable salt thereof, wherein:
X is selected from CR⁶ and N;
Y is selected from CR¹² and N;
Z is selected from —O—, —S—, and —NR⁸—;
Q is absent or selected from:
   W—, —O—, —OW—, —WO—, —S—, —SW—, —WS—, —NR¹⁰—W—, —W—NR¹⁰—, C(O)—, —C(O)N(R¹⁰)—, —N(R¹⁰)C(O)—, —C(O)O—, —OC(O)—, —N(R¹⁰)C(O)N(R¹⁰)0-, —OC(O)N(R¹⁰)—, —N(R¹⁰)C(O)O—, —S(O)—, —S(O)₂—, and —P(O)₂O—;
m is selected from 1, 2, 3, 4, and 5;
R¹ is selected from:
   hydrogen;
   C₁₋₆ alkyl, C₂₋₆ alkenyl, and C₂₋₆ alkynyl, each of which is optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —C(O)OR¹⁰, —OC(O)R¹⁰, —N(R¹⁰)C(O)N(R¹⁰)₂, —OC(O)N(R¹⁰)₂, —N(R¹⁰)C(O)OR¹⁰, —S(O)R¹⁰, —S(O)₂R¹⁰, —NO₂, ═O, ═S, ═N(R¹⁰), —CN, C₃₋₁₀ carbocycle and 3- to 10-membered heterocycle, wherein the C₃₋₁₀ carbocycle and 3- to 10-membered heterocycle are each optionally substituted with one or more R⁹; and
   C₃₋₁₀ carbocycle and 3- to 10-membered heterocycle, each of which is optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —N(R¹⁰)C(O)N(R¹⁰)₂, —OC(O)N(R¹⁰)₂, —N(R¹⁰)C(O)OR¹⁰, —C(O)OR¹⁰, —OC(O)R¹⁰, —S(O)R¹⁰, —S(O)₂R¹⁰, —NO₂, ═O, ═S, ═N(R¹⁰), —CN, C₁₋₆ alkyl, C₂₋₆ alkenyl, and C₂₋₆ alkynyl, wherein C₁₋₆ alkyl, C₂₋₆ alkenyl, and C₂₋₆ alkynyl are each optionally substituted with one or more R⁹;
R² is selected from:
   hydrogen;
   halogen, —OR¹⁰, —SR¹⁰, —N(R¹⁰)₂, —C(O)R¹⁰, —C(O)N(R¹⁰)₂, —N(R¹⁰)C(O)R¹⁰, —N(R¹⁰)C(O)N(R¹⁰)₂, —OC(O)N(R¹⁰)₂, —N(R¹⁰)C(O)OR¹⁰, —C(O)OR¹⁰, —OC(O)R¹⁰, —S(O)R¹⁰, —S(O)₂R¹⁰, —NO₂, and —CN;
   C₁₋₆ alkyl, C₂₋₆ alkenyl, and C₂₋₆ alkynyl, each of which is optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —C(O)OR¹⁰, —OC(O)R¹⁰, —N(R¹⁰)C(O)N(R¹⁰)₂, —OC(O)N(R¹⁰)₂, —N(R¹⁰)C(O)OR¹⁰, —S(O)R¹⁰, —S(O)₂R¹⁰, —NO₂, ═O, ═S, ═N(R¹⁰), —CN, C₃₋₁₀ carbocycle and 3- to 10-membered heterocycle, wherein the C₃₋₁₀ carbocycle and 3- to 10-membered heterocycle are each optionally substituted with one or more R⁹; and
   C₃₋₆ carbocycle and 3- to 6-membered heterocycle, each of which is optionally substituted with one or more substituents independently selected from halogen, —OH, —SR¹⁰, —N(R¹⁰)₂, —C(O)R¹⁰, —C(O)N(R¹⁰)₂, —N(R¹⁰)C(O)R¹⁰, —N(R¹⁰)C(O)N(R¹⁰)₂, —OC(O)N(R¹⁰)₂, —N(R¹⁰)C(O)OR¹⁰, —C(O)OR¹⁰, —OC(O)R¹⁰, —S(O)R¹⁰, —S(O)₂R¹⁰, —NO₂, ═O, ═S, ═N(R¹⁰), —CN, C₁₋₆ alkyl, C₂₋₆ alkenyl, and C₂₋₆ alkynyl, wherein C₁₋₆ alkyl, C₂₋₆ alkenyl, and C₂₋₆ alkynyl are each optionally substituted with one or more R⁹
R³ is selected from:
   C₃₋₆ carbocycle and 3- to 6-membered heterocycle, each of which is optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —N(R¹⁰)C(O)N(R¹⁰)₂, —OC(O)N(R¹⁰)₂, —N(R¹⁰)C(O)OR¹⁰, —C(O)OR¹⁰, —OC(O)R¹⁰, —S(O)R¹⁰, —S(O)₂R¹⁰, —NO₂, ═O, ═S, ═N(R¹⁰), —CN, C₁₋₆ alkyl, C₂₋₆ alkenyl, and C₂₋₆ alkynyl, wherein C₁₋₆ alkyl, C₂₋₆ alkenyl, and C₂₋₆ alkynyl are each optionally substituted with one or more R⁹; and
wherein if Q is —W—, then R³ is additionally selected from halogen, —OR¹⁰, —SR¹⁰, —N(R¹⁰)₂, —C(O)R¹⁰, —C(O)N(R¹⁰)₂, —N(R¹⁰)C(O)R¹⁰, —N(R¹⁰)C(O)N(R¹⁰)₂, —OC(O)N(R¹⁰)₂, —N(R¹⁰)C(O)OR¹⁰, —C(O)OR¹⁰, —OC(O)R¹⁰, —S(O)R¹⁰, —S(O)₂R¹⁰, —NO₂, and —CN; each R⁴ is independently selected from:
   halogen, —OR⁷, —SR⁷, —N(R⁷)₂, —C(O)R⁷, —C(O)N(R⁷)₂, —N(R⁷)C(O)R⁷, —N(R⁷)C(O)N(R⁷)₂, —OC(O)N(R⁷)₂, —N(R⁷)C(O)OR⁷, —C(O)OR⁷, —OC(O)R⁷, —S(O)R⁷, —S(O)₂R⁷, —NO₂, and —CN;
   C₁₋₆ alkyl, C₂₋₆ alkenyl, and C₂₋₆ alkynyl, each of which is optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —C(O)OR¹⁰, —OC(O)R¹⁰, —N(R¹⁰)C(O)N(R¹⁰)₂, —OC(O)N(R¹⁰)₂, —N(R¹⁰)C(O)OR¹⁰, —S(O)R¹⁰, —S(O)₂R¹⁰, —NO₂, ═O, ═S, ═N(R¹⁰), —CN, C₃₋₁₀ carbocycle and 3- to 10-membered heterocycle, wherein the C₃₋₁₀ carbocycle and 3- to 10-membered heterocycle are each optionally substituted with one or more R⁹; and
   C₃₋₁₀ carbocycle and 3- to 10-membered heterocycle, each of which is optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —N(R¹⁰)C(O)N(R¹⁰)₂, —OC(O)N(R¹⁰)₂, —N(R¹⁰)C(O)OR¹⁰, —C(O)OR¹⁰, —OC(O)R¹⁰, —S(O)R¹⁰, —S(O)₂R¹⁰, —NO₂, ═O, ═S, ═N(R¹⁰), —CN, C₁₋₆ alkyl, C₂₋₆ alkenyl, and C₂₋₆ alkynyl, wherein C₁₋₆ alkyl, C₂₋₆ alkenyl, and C₂₋₆ alkynyl are each optionally substituted with one or more R⁹
R⁵ is selected from:
   hydrogen, halogen, —OR¹⁰, —SR¹⁰, —N(R¹⁰)₂, —NO₂, and —CN; and
   C₁₋₆ alkyl optionally substituted with one or more substituents independently selected from halogen, —OR¹⁰, —SR¹⁰, —N(R¹⁰)₂, —NO₂, and —CN;
R⁶ is selected from:
   hydrogen, halogen, —OR¹⁰, —SR¹⁰, —N(R¹⁰)₂, —NO₂, and —CN; and
   C₁₋₆ alkyl optionally substituted with one or more substituents independently selected from halogen, —OR¹⁰, —SR¹⁰, —N(R¹⁰)₂, —NO₂, and —CN;
each R⁷ is independently selected from:
   hydrogen;
   C₁₋₆ alkyl, C₂₋₆ alkenyl, and C₂₋₆ alkynyl, each of which is optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —C(O)OR¹⁰, —OC(O)R¹⁰, —N(R¹⁰)C(O)N(R¹⁰)₂, —OC(O)N(R¹⁰)₂, —N(R¹⁰)C(O)OR¹⁰, —S(O)R¹⁰, —S(O)₂R¹⁰, —NO₂, ═O, ═S, ═N(R¹⁰), —CN, C₃₋₁₀ carbocycle and 3- to 10-membered heterocycle, wherein the C₃₋₁₀ carbocycle and 3- to 10-membered heterocycle are each optionally substituted with one or more R⁹; and
   C₃₋₁₀ carbocycle and 3- to 10-membered heterocycle, each of which is optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —N(R¹⁰)C(O)N(R¹⁰)₂, —OC(O)N(R¹⁰)₂, —N(R¹⁰)C(O)OR¹⁰, —C(O)OR¹⁰, —OC(O)R¹⁰, —S(O)R¹⁰, —S(O)₂R¹⁰, —NO₂, ═O, ═S, ═N(R¹⁰), —CN, C₁₋₆ alkyl, C₂₋₆ alkenyl, and C₂₋₆ alkynyl, wherein C₁₋₆ alkyl, C₂₋₆ alkenyl, and C₂₋₆ alkynyl are each optionally substituted with one or more R⁹
R⁸ is selected from:
   hydrogen;
   C₁₋₆ alkyl, C₂₋₆ alkenyl, and C₂₋₆ alkynyl, each of which is optionally substituted with one or more substituents independently selected from halogen, —SR¹⁰, —N(R¹⁰)₂, —C(O)R¹⁰, —C(O)N(R¹⁰)₂, —N(R¹⁰)C(O)R¹⁰, —C(O)OR¹⁰, —OC(O)R¹⁰, —N(R¹⁰)C(O)N(R¹⁰)₂, —OC(O)N(R¹⁰)₂, —N(R¹⁰)C(O)OR¹⁰, —S(O)R¹⁰, —S(O)₂R¹⁰, —NO₂, ═O, ═S, ═N(R¹⁰), —CN, C₃₋₁₀ carbocycle and 3- to 10-membered heterocycle, wherein the C₃₋₁₀ carbocycle and 3- to 10-membered heterocycle are each optionally substituted with one or more R⁹; and
   C₃₋₁₀ carbocycle and 3- to 10-membered heterocycle, each of which is optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —N(R¹⁰)C(O)N(R¹⁰)₂, —OC(O)N(R¹⁰)₂, —N(R¹⁰)C(O)OR¹⁰, —C(O)OR¹⁰, —OC(O)R¹⁰, —S(O)R¹⁰, —S(O)₂R¹⁰, —NO₂, ═O, ═S, ═N(R¹⁰), —CN, C₁₋₆ alkyl, C₂₋₆ alkenyl, and C₂₋₆ alkynyl, wherein C₁₋₆ alkyl, C₂₋₆ alkenyl, and C₂₋₆ alkynyl are each optionally substituted with one or more R⁹
each R⁹ is independently selected from:
   halogen, —R¹⁰, —OR¹⁰, —SR¹⁰, —N(R¹⁰)₂, —C(O)R¹⁰, —C(O)N(R¹⁰)₂, —N(R¹⁰)C(O)R¹⁰, —N(R¹⁰)C(O)N(R¹⁰)₂, —OC(O)N(R¹⁰)₂, —N(R¹⁰)C(O)OR¹⁰, —C(O)OR¹⁰, —OC(O)R¹⁰, —OC(O)₂R¹⁰, —S(O)R¹⁰, —S(O)₂R¹⁰, —NO₂, ═O, ═S, ═N(R¹⁰), and —CN;
   C₁₋₃ alkyl, C₂₋₃ alkenyl, and C₂₋₃ alkynyl, each of which is optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —N(R¹⁰)C(O)N(R¹⁰)₂, —OC(O)N(R¹⁰)₂, —N(R¹⁰)C(O)OR¹⁰, —C(O)OR¹⁰, —OC(O)R¹⁰, —S(O)R¹⁰, —S(O)₂R¹⁰, —NO₂, ═O, ═S, ═N(R¹⁰), and —CN;
   C₃₋₁₀ carbocycle and 3- to 10-membered heterocycle, each of which is optionally substituted with one or more substituents independently selected from halogen, —R¹⁰, —OR¹⁰, —SR¹⁰, —N(R¹⁰)₂, —C(O)R¹⁰, —C(O)N(R¹⁰)₂, —N(R¹⁰)C(O)R¹⁰, —N(R¹⁰)C(O)N(R¹⁰)₂, —OC(O)N(R¹⁰)₂, —N(R¹⁰)C(O)OR¹⁰, —C(O)OR¹⁰, —OC(O)R¹⁰, —S(O)R¹⁰, —S(O)₂R¹⁰, —NO₂, ═O, ═S, ═N(R¹⁰), and —CN; and

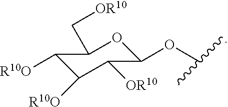

each R¹⁰ is independently selected from:
   hydrogen;
   C₁₋₆ alkyl, C₂₋₆ alkenyl, and C₂₋₆ alkynyl, each of which is optionally substituted with one or more substituents independently selected from halogen, —CN, —OH, —SH, —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₃₋₁₀ carbocycle, 3- to 10-membered heterocycle; and
   C₃₋₁₀ carbocycle, and 3- to 10-membered heterocycle, each of which is optionally substituted with one or more substituents independently selected from halogen, —CN, —OH, —SH, —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₁₋₆ alkyl, C₂₋₆ alkenyl, C₂₋₆ alkynyl, C₃₋₁₀ carbocycle, 3- to 10-membered heterocycle, and C₁₋₆ haloalkyl;
W is selected from:
   C₁₋₆ alkylene, C₂₋₆ alkenylene, and C₂₋₆ alkynylene, each of which is optionally substituted with one or more substituents independently selected from halogen, —CN, —OH, —SH, —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₃₋₁₀ carbocycle, and 3- to 10-membered heterocycle;
R¹² is selected from:
   hydrogen, halogen, —OR¹⁰, —SR¹⁰, —N(R¹⁰)₂, —NO₂, and —CN; and
   C₁₋₆ alkyl optionally substituted with one or more substituents independently selected from halogen, —OR¹⁰, —SR¹⁰, —N(R¹⁰)₂, —NO₂, and —CN;
wherein if Z is NH then R⁴ is not NH₂, and
wherein if Z is O or S, R⁴ is —OR³, and R⁵ is H, then W—R³ is additionally selected from C₁₋₆ alkyl, C₂₋₆ alkenyl, and C₂₋₆ alkynyl.

In some embodiments, for a compound or salt of Formula (I), X is selected from CR⁶ and N. In some embodiments, X is selected from CR⁶. In some embodiments, X is selected from N. In some embodiments, X is CR⁶.

In some embodiments, for a compound or salt of Formula (I), Y is selected from CR¹² and N. In some embodiments, Y is selected from CR¹². In some embodiments, Y is selected from N. In some embodiments, Y is CR¹².

In some embodiments, for a compound or salt of Formula (I), Z is selected from —O—, —S—, and —NR⁸—. In some embodiments, Z is selected from —O— and —S—. In some embodiments, Z is —O—.

In some embodiments, for a compound or salt of Formula (I), Q is absent or selected from —W—, —O—, —OW—, —WO—, —S—, —SW—, —WS—, —NR¹⁰W—, —WNR¹⁰—, C(O)—, —C(O)N(R¹⁰)—, —N(R¹⁰)C(O)—, —C(O)O—, —OC(O)—, —N(R¹⁰)C(O)N(R¹⁰)0-, —OC(O)N(R¹⁰)—, —N(R¹⁰)C(O)O—, —S(O)—, —S(O)₂—, and —P(O)₂O—;In some embodiments, Q is absent or selected from —W—, —O—, —OW—, and —WO—. In some embodiments, Q is absent or —O—. In some embodiments, Q is absent.

In some embodiments, for a compound or salt of Formula (I), m is selected from 1, 2, 3, 4, and 5. In some embodiments, m is selected from 1 and 2. In some embodiments, m is 1.

In some embodiments, for a compound or salt of Formula (I), R¹ is selected from hydrogen, C₁₋₆ alkyl, C₂₋₆ alkenyl, and C₂₋₆ alkynyl, each of which is optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —C(O)OR¹⁰, —OC(O)R¹⁰, —N(R¹⁰)C(O)N(R¹⁰)₂, —OC(O)N(R¹⁰)₂, —N(R¹⁰)C(O)OR¹⁰, —S(O)R¹⁰, —S(O)₂R¹⁰, —NO₂, ═O, ═S, ═N(R¹⁰), —CN, C₃₋₁₀ carbocycle and 3- to 10-membered heterocycle, wherein the C₃₋₁₀ carbocycle and 3- to 10-membered heterocycle are each optionally substituted with one or more R⁹; and C₃₋₁₀ carbocycle and 3- to 10-membered heterocycle, each of which is optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —N(R¹⁰)C(O)N(R¹⁰)₂, —OC(O)N(R¹⁰)₂, —N(R¹⁰)C(O)OR¹⁰, —C(O)OR¹⁰, —OC(O)R¹⁰, —S(O)R¹⁰, —S(O)₂R¹⁰, —NO₂, ═O, ═S, ═N(R¹⁰), —CN, C₁₋₆ alkyl, C₂₋₆ alkenyl, and C₂₋₆ alkynyl, wherein C₁₋₆ alkyl, C₂₋₆ alkenyl, and C₂₋₆ alkynyl are each optionally substituted with one or more R⁹. In some embodiments, R¹ is selected from hydrogen, C₁₋₆ alkyl, C₂₋₆ alkenyl, and C₂₋₆ alkynyl, each of which is optionally substituted with one or more substituents independently selected from halogen, —CN, —OH, —SH, —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₃₋₁₀ carbocycle, 3- to 10-membered heterocycle; and C₃₋₆ carbocycle, and 3- to 6-membered heterocycle, each of which is optionally substituted with one or more substituents independently selected from halogen, —CN, —OH, —SH, —NO₂, —NH₂, ═O, ═S, —O—C₁₋₆ alkyl, —S—C₁₋₆ alkyl, —N(C₁₋₆ alkyl)₂, and —NH(C₁₋₆ alkyl). In some embodiments, R¹ is selected from hydrogen and C₁₋₃ alkyl.

In some embodiments, for a compound or salt of Formula (I), R² is selected from hydrogen, halogen, —OR¹⁰, —SR¹⁰, —N(R¹⁰)₂, —C(O)R¹⁰, —C(O)N(R¹⁰)₂, —N(R¹⁰)C(O)R¹⁰, —N(R¹⁰)C(O)N(R¹⁰)₂, —OC(O)N(R¹⁰)₂, —N(R¹⁰)C(O)OR¹⁰, —C(O)OR¹⁰, —OC(O)R¹⁰, —S(O)R¹⁰, —S(O)₂R¹⁰, —NO₂, and —CN; C₁₋₆ alkyl, C₂₋₆ alkenyl, and C₂₋₆ alkynyl, each of which is optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —C(O)OR¹⁰, —OC(O)R¹⁰, —N(R¹⁰)C(O)N(R¹⁰)₂, —OC(O)N(R¹⁰)₂, —N(R¹⁰)C(O)OR¹⁰, —S(O)R¹⁰, —S(O)₂R¹⁰, —NO₂, ═O, ═S, ═N(R¹⁰), —CN, C₃₋₁₀ carbocycle and 3- to 10-membered heterocycle, wherein the C₃₋₁₀ carbocycle and 3- to 10-membered heterocycle are each optionally substituted with one or more R⁹; and C₃₋₆ carbocycle and 3- to 6-membered heterocycle, each of which is optionally substituted with one or more substituents independently selected from halogen, —OH, —SR¹⁰, —N(R¹⁰)₂, —C(O)R¹⁰, —C(O)N(R¹⁰)₂, —N(R¹⁰)C(O)R¹⁰, —N(R¹⁰)C(O)N(R¹⁰)₂, —OC(O)N(R¹⁰)₂, —N(R¹⁰)C(O)OR¹⁰, —C(O)OR¹⁰, —OC(O)R¹⁰, —S(O)R¹⁰, —S(O)₂R¹⁰, —NO₂, ═O, ═S, ═N(R¹⁰), —CN, C₁₋₆ alkyl, C₂₋₆ alkenyl, and C₂₋₆ alkynyl, wherein C₁₋₆ alkyl, C₂₋₆ alkenyl, and C₂₋₆ alkynyl are each optionally substituted with one or more R⁹. In some embodiments, R² is selected from hydrogen, halogen, —CN, —OH, —SH, —NO₂, —NH₂, ═O, ═S, —O—C₁₋₆ alkyl, —S—C₁₋₆ alkyl, —N(C₁₋₆ alkyl)₂, —NH(C₁₋₆ alkyl); C₁₋₆ alkyl, C₂₋₆ alkenyl, and C₂₋₆ alkynyl, each of which is optionally substituted with one or more substituents independently selected from halogen, —CN, —OH, —SH, —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₃₋₁₀ carbocycle, 3- to 10-membered heterocycle; and C₃₋₆ carbocycle, and 3- to 6-membered heterocycle, each of which is optionally substituted with one or more substituents independently selected from halogen, —CN, —OH, —SH, —NO₂, —NH₂, =0, ═S, —S—C₁₋₆ alkyl, —N(C₁₋₆ alkyl)₂, and —NH(C₁₋₆ alkyl). In some embodiments, R² is selected from: hydrogen; halogen, —CN, —OH, —SH, —NO₂, —NH₂, ═O, ═S, —O—C₁₋₆ alkyl, —S—C₁₋₆ alkyl, —N(C₁₋₆ alkyl)₂, —NH(C₁₋₆ alkyl); C₁₋₆ alkyl optionally substituted with one or more substituents independently selected from halogen, —CN, —OH, —SH, —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₃₋₁₀ carbocycle, 3- to 10-membered heterocycle; and C₆ carbocycle, and 5- to 6-membered heterocycle, each of which is optionally substituted with one or more substituents independently selected from halogen. In some embodiments, R² is selected from: hydrogen; halogen, —CN, —OH, —SH, —NO₂, —NH₂, ═O, ═S, —O—C₁₋₆ alkyl, —S—C₁₋₆ alkyl, —N(C₁₋₆ alkyl)₂, —NH(C₁₋₆ alkyl); C₁₋₆ alkyl optionally substituted with one or more substituents independently selected from halogen, —CN, —OH, —SH, —NO₂, —NH₂, =0, ═S, —O—C₁₋₆ alkyl, —S—C₁₋₆ alkyl, —N(C₁₋₆ alkyl)₂, —NH(C₁₋₆ alkyl), C₃₋₁₀ carbocycle, and 3- to 10-membered heterocycle. In some embodiments, R² is selected from hydrogen, halogen, and C₁₋₃ alkyl.

In some embodiments, for a compound or salt of Formula (I), R³ is C₃₋₆ carbocycle and 3- to 6-membered heterocycle, each of which is optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —N(R¹⁰)C(O)N(R¹⁰)₂, —OC(O)N(R¹⁰)₂, —N(R¹⁰)C(O)OR¹⁰, —C(O)OR¹⁰, —OC(O)R¹⁰, —S(O)R¹⁰, —S(O)₂R¹⁰, —NO₂, ═O, ═S, ═N(R¹⁰), —CN, C₁₋₆ alkyl, C₂₋₆ alkenyl, and C₂₋₆ alkynyl, wherein C₁₋₆ alkyl, C₂₋₆ alkenyl, and C₂₋₆ alkynyl are each optionally substituted with one or more R⁹; and wherein if Q is —W—, then R³ is additionally selected from halogen, —OR¹⁰, —SR¹⁰, —N(R¹⁰)₂, —C(O)R¹⁰, —C(O)N(R¹⁰)₂, —N(R¹⁰)C(O)R¹⁰, —N(R¹⁰)C(O)N(R¹⁰)₂, —OC(O)N(R¹⁰)₂, —N(R¹⁰)C(O)OR¹⁰, —C(O)OR¹⁰, —OC(O)R¹⁰, —S(O)R¹⁰, —S(O)₂R¹⁰, —NO₂, and —CN. In some embodiments, R³ is a C₃₋₆ carbocycle optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —N(R¹⁰)C(O)N(R¹⁰)₂, —OC(O)N(R¹⁰)₂, —N(R¹⁰)C(O)OR¹⁰, —C(O)OR¹⁰, —OC(O)R¹⁰, —S(O)R¹⁰, —S(O)₂R¹⁰, —NO₂, ═O, ═S, ═N(R¹⁰), —CN, C₁₋₆ alkyl, C₂₋₆ alkenyl, and C₂₋₆ alkynyl, wherein C₁₋₆ alkyl, C₂₋₆ alkenyl, and C₂₋₆ alkynyl are each optionally substituted with one or more R⁹. In some embodiments, R³ is a C₆ carbocycle optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —N(R¹⁰)C(O)N(R¹⁰)₂, —OC(O)N(R¹⁰)₂, —N(R¹⁰)C(O)OR¹⁰, —C(O)OR¹⁰, —OC(O)R¹⁰, —S(O)R¹⁰, —S(O)₂R¹⁰, —NO₂, ═O, ═S, ═N(R¹⁰), —CN, C₁₋₆ alkyl, C₂₋₆ alkenyl, and C₂₋₆ alkynyl, wherein C₁₋₆ alkyl, C₂₋₆ alkenyl, and C₂₋₆ alkynyl are each optionally substituted with one or more R⁹. In some embodiments, R³ is a phenyl optionally substituted with one or more substituents independently selected from halogen, —CN, —OH, —SH, —NO₂, —NH₂, —O—C₁₋₆ alkyl, —S—C₁₋₆ alkyl, —N(C₁₋₆ alkyl)₂, and —NH(C₁₋₆ alkyl).

In some embodiments, for a compound or salt of Formula (I), R³ is a 3- to 6-membered heterocycle optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —N(R¹⁰)C(O)N(R¹⁰)₂, —OC(O)N(R¹⁰)₂, —N(R¹⁰)C(O)OR¹⁰, —C(O)OR¹⁰, —OC(O)R¹⁰, —S(O)R¹⁰, —S(O)₂R¹⁰, —NO₂, ═O, ═S, ═N(R¹⁰), —CN, C₁₋₆ alkyl, C₂₋₆ alkenyl, and C₂₋₆ alkynyl, wherein C₁₋₆ alkyl, C₂₋₆ alkenyl, and C₂₋₆ alkynyl are each optionally substituted with one or more R⁹. In some embodiments, R³ is a 5- to 6-membered heterocycle optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —N(R¹⁰)C(O)N(R¹⁰)₂, —OC(O)N(R¹⁰)₂, —N(R¹⁰)C(O)OR¹⁰, —C(O)OR¹⁰, —OC(O)R¹⁰, —S(O)R¹⁰, —S(O)₂R¹⁰, —NO₂, ═O, ═S, ═N(R¹⁰), —CN, C₁₋₆ alkyl, C₂₋₆ alkenyl, and C₂₋₆ alkynyl, wherein C₁₋₆ alkyl, C₂₋₆ alkenyl, and C₂₋₆ alkynyl are each optionally substituted with one or more R⁹. In some embodiments, R³ is a 6-membered heterocycle optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —N(R¹⁰)C(O)N(R¹⁰)₂, —OC(O)N(R¹⁰)₂, —N(R¹⁰)C(O)OR¹⁰, —C(O)OR¹⁰, —OC(O)R¹⁰, —S(O)R¹⁰, —S(O)₂R¹⁰, —NO₂, ═O, ═S, ═N(R¹⁰), —CN, C₁₋₆ alkyl, C₂₋₆ alkenyl, and C₂₋₆ alkynyl, wherein C₁₋₆ alkyl, C₂₋₆ alkenyl, and C₂₋₆ alkynyl are each optionally substituted with one or more R⁹. In some embodiments, R³ is a 6-membered heterocycle optionally substituted with one or more substituents independently selected from halogen, —CN, —OH, —SH, —NO₂, —NH₂, —O—C₁₋₆ alkyl, —S—C₁₋₆ alkyl, —N(C₁₋₆ alkyl)₂, and —NH(C₁₋₆ alkyl). In some embodiments, for a compound or salt of Formula (I), R³ is a 5-membered heterocycle optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —N(R¹⁰)C(O)N(R¹⁰)₂, —OC(O)N(R¹⁰)₂, —N(R¹⁰)C(O)OR¹⁰, —C(O)OR¹⁰, —OC(O)R¹⁰, —S(O)R¹⁰, —S(O)₂R¹⁰, —NO₂, ═O, ═S, ═N(R¹⁰), —CN, C₁₋₆ alkyl, C₂₋₆ alkenyl, and C₂₋₆ alkynyl, wherein C₁₋₆ alkyl, C₂₋₆ alkenyl, and C₂₋₆ alkynyl are each optionally substituted with one or more R⁹. In some embodiments, R³ is a 5-membered heterocycle optionally substituted with one or more substituents independently selected from halogen, —CN, —OH, ═O, ═S, ═NH, —SH, —NO₂, —NH₂, —O—C₁₋₆ alkyl, —S—C₁₋₆ alkyl, —N(C₁₋₆ alkyl)₂, —NH(C₁₋₆ alkyl), and C₁₋₆ alkyl. In some embodiments, R³ is a 5-membered heterocycle is selected from isoxazole, oxazole, thiadiazole, oxadiazole, pyrazole, tetrazole, and thiazole, any of which is optionally substituted with one or more R⁹. In some embodiments, R³ is a 5-membered heterocycle is selected tetrazole and oxadiazole. In some embodiments, R³ is a 5-membered heterocycle selected from isothiazol-3-ol, isoxazole-3-ol, squaric acid, thiazolidinedione, oxazolidinedione, tetrazole, 1,2,4-oxadiazol-5(4H)-one, 1,2,4-thiadiazol-5(4H)-one, 1,2,4-oxadiazole-5(4H)-thione, 2-methyl-2,4-dihydro-3H-1,2,4-triazole-3-thione, 3H-1,2,3,5-oxathiadiazole 2-oxide, 3-(methylsulfonyl)-4H-1,2,4-triazole, or 1,4-dihydro-5H-tetrazol-5-one, 3-hydroxyquinolin-2-one, and tetramic acid, any of which is optionally substituted with one or more R⁹. In some embodiments, R³ is a 5-membered heterocycle is selected from

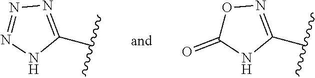 [SMILES: *c1nnn[nH]1 ; *c1noc(=O)[nH]1]

optionally substituted with one or more R⁹. In some embodiments, R³ is a 5-membered heterocycle is selected from

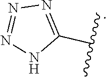 [SMILES: *c1nnn[nH]1]

In some embodiments, R³ is selected from,

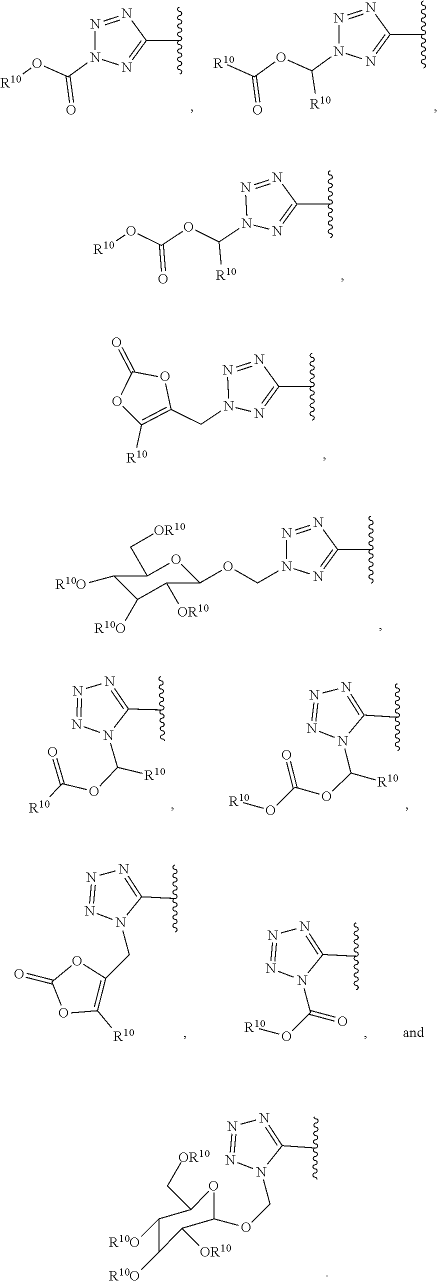

In some embodiments, Q is —O— and R³ is tetrazole.

In some embodiments, for a compound or salt of Formula (I), Q is —W—; R³ is additionally selected from halogen, —OR¹⁰, —SR¹⁰, —N(R¹⁰)₂, —C(O)R¹⁰, —C(O)N(R¹⁰)₂, —N(R¹⁰)C(O)R¹⁰, —N(R¹⁰)C(O)N(R¹⁰)₂, —OC(O)N(R¹⁰)₂, —N(R¹⁰)C(O)OR¹⁰, —C(O)OR¹⁰, —OC(O)R¹⁰, —S(O)R¹⁰, —S(O)₂R¹⁰, —NO₂, —CN; and —W— is C₁₋₆ alkylene optionally substituted with one or more substituents independently selected from halogen, —CN, —OH, —SH, —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₃₋₁₀ carbocycle, and 3- to 10-membered heterocycle. In some embodiments, Q is —W—; and R³ is additionally selected from halogen, —CN, —OH, —SH, —NO₂, —NH₂, —O—C₁₋₆ alkyl, —S—C₁₋₆ alkyl, —N(C₁₋₆ alkyl)₂, and —NH(C₁₋₆ alkyl), and —W— is C₁₋₆ alkylene optionally substituted with one or more substituents independently selected from halogen, —CN, —OH, —SH, —NO₂, —NH₂, =0, ═S, —O—C₁₋₆ alkyl, —S—C₁₋₆ alkyl, —N(C₁₋₆ alkyl)₂, —NH(C₁₋₆ alkyl), C₃₋₁₀ carbocycle, and 3- to 10-membered heterocycle. In some embodiments, Q is —W—; and R³ is additionally selected from —OH, —SH, —NH₂, —O—C₁₋₆ alkyl, —S—C₁₋₆ alkyl, —N(C₁₋₆ alkyl)₂, and —NH(C₁₋₆ alkyl); and —W— is C₁ alkylene optionally substituted with one or more substituents independently selected from halogen, —CN, —OH, —SH, —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₃₋₁₀ carbocycle, and 3- to 10-membered heterocycle. In some embodiments, Q is —W—; and R³ is additionally selected from —OH, —SH, —NH₂, —O—C₁₋₆ alkyl, —S—C₁₋₆ alkyl, —N(C₁₋₆ alkyl)₂, and —NH(C₁₋₆ alkyl); and —W— is —C(═O)—.

In some embodiments, R³ is not phenyl. In some embodiments, R³ is not pyrimidine. In some embodiments, R³ is not pyridine. In some embodiments, R³ is not.

In some embodiments, for a compound or salt of Formula (I), R⁴ is selected from halogen, —OR⁷, —SR⁷, —N(R⁷)₂, —C(O)R⁷, —C(O)N(R⁷)₂, —N(R⁷)C(O)R⁷, —N(R⁷)C(O)N(R⁷)₂, —OC(O)N(R⁷)₂, —N(R⁷)C(O)OR⁷, —C(O)OR⁷, —OC(O)R⁷, —S(O)R⁷, —S(O)₂R⁷, —NO₂, and —CN; C₁₋₆ alkyl, C₂₋₆ alkenyl, and C₂₋₆ alkynyl, each of which is optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —C(O)OR¹⁰, —OC(O)R¹⁰, —N(R¹⁰)C(O)N(R¹⁰)₂, —OC(O)N(R¹⁰)₂, —N(R¹⁰)C(O)OR¹⁰, —S(O)R¹⁰, —S(O)₂R¹⁰, —NO₂, ═O, ═S, ═N(R¹⁰), —CN, C₃₋₁₀ carbocycle and 3- to 10-membered heterocycle, wherein the C₃₋₁₀ carbocycle and 3- to 10-membered heterocycle are each optionally substituted with one or more R⁹; and C₃₋₁₀ carbocycle and 3- to 10-membered heterocycle, each of which is optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —N(R¹⁰)C(O)N(R¹⁰)₂, —OC(O)N(R¹⁰)₂, —N(R¹⁰)C(O)OR¹⁰, —C(O)OR¹⁰, —OC(O)R¹⁰, —S(O)R¹⁰, —S(O)₂R¹⁰, —NO₂, ═O, ═S, ═N(R¹⁰), —CN, C₁₋₆ alkyl, C₂₋₆ alkenyl, and C₂₋₆ alkynyl, wherein C₁₋₆ alkyl, C₂₋₆ alkenyl, and C₂₋₆ alkynyl are each optionally substituted with one or more R⁹. In some embodiments, R⁴ is selected from: halogen, —OR⁷, —SR⁷, —N(R⁷)₂, —NO₂, and —CN; C₁₋₆ alkyl, C₂₋₆ alkenyl, and C₂₋₆ alkynyl, each of which is optionally substituted with one or more substituents independently selected from halogen, —CN, —OH, —SH, —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₃₋₁₀ carbocycle, 3- to 10-membered heterocycle; and C₃₋₆ carbocycle, and 3- to 6-membered heterocycle, each of which is optionally substituted with one or more substituents independently selected from halogen, —CN, —OH, —SH, —NO₂, —NH₂, ═O, ═S, —O—C₁₋₆ alkyl, —S—C₁₋₆ alkyl, —N(C₁₋₆ alkyl)₂, and —NH(C₁₋₆ alkyl). In some embodiments, each R⁴ is independently selected from —OR⁷, —SR⁷, and —N(R⁷)₂. In some embodiments, m is 1 and R⁴ is —OR⁷.

In some embodiments, for a compound or salt of Formula (I), each R⁷ is independently selected from hydrogen, C₁₋₆ alkyl, C₂₋₆ alkenyl, and C₂₋₆ alkynyl, each of which is optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —C(O)OR¹⁰, —OC(O)R¹⁰, —N(R¹⁰)C(O)N(R¹⁰)₂, —OC(O)N(R¹⁰)₂, —N(R¹⁰)C(O)OR¹⁰, —S(O)R¹⁰, —S(O)₂R¹⁰, —NO₂, ═O, ═S, ═N(R¹⁰), —CN, C₃₋₁₀ carbocycle and 3- to 10-membered heterocycle, wherein the C₃₋₁₀ carbocycle and 3- to 10-membered heterocycle are each optionally substituted with one or more R⁹; and C₃₋₁₀ carbocycle and 3- to 10-membered heterocycle, each of which is optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —N(R¹⁰)C(O)N(R¹⁰)₂, —OC(O)N(R¹⁰)₂, —N(R¹⁰)C(O)OR¹⁰, —C(O)OR¹⁰, —OC(O)R¹⁰, —S(O)R¹⁰, —S(O)₂R¹⁰, —NO₂, ═O, ═S, ═N(R¹⁰), —CN, C₁₋₆ alkyl, C₂₋₆ alkenyl, and C₂₋₆ alkynyl, wherein C₁₋₆ alkyl, C₂₋₆ alkenyl, and C₂₋₆ alkynyl are each optionally substituted with one or more R⁹. In some embodiments, each R⁷ is selected from C₁₋₆ alkyl, C₂₋₆ alkenyl, and C₂₋₆ alkynyl, each of which is optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —C(O)OR¹⁰, —OC(O)R¹⁰, —N(R¹⁰)C(O)N(R¹⁰)₂, —OC(O)N(R¹⁰)₂, —N(R¹⁰)C(O)OR¹⁰, —S(O)R¹⁰, —S(O)₂R¹⁰, —NO₂, ═O, ═S, ═N(R¹⁰), —CN, C₃₋₁₀ carbocycle and 3- to 10-membered heterocycle, wherein the C₃₋₁₀ carbocycle and 3- to 10-membered heterocycle are each optionally substituted with one or more R⁹; and C₃₋₁₀ carbocycle and 3- to 10-membered heterocycle, each of which is optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —N(R¹⁰)C(O)N(R¹⁰)₂, —OC(O)N(R¹⁰)₂, —N(R¹⁰)C(O)OR¹⁰, —C(O)OR¹⁰, —OC(O)R¹⁰, —S(O)R¹⁰, —S(O)₂R¹⁰, —NO₂, ═O, ═S, ═N(R¹⁰), —CN, C₁₋₆ alkyl, C₂₋₆ alkenyl, and C₂₋₆ alkynyl, wherein C₁₋₆ alkyl, C₂₋₆ alkenyl, and C₂₋₆ alkynyl are each optionally substituted with one or more R⁹. In some embodiments, each R⁷ is selected from: C₁₋₆ alkyl optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —C(O)OR¹⁰, —OC(O)R¹⁰, —N(R¹⁰)C(O)N(R¹⁰)₂, —OC(O)N(R¹⁰)₂, —N(R¹⁰)C(O)OR¹⁰, —S(O)R¹⁰, —S(O)₂R¹⁰, —NO₂, ═O, ═S, ═N(R¹⁰), —CN, C₃₋₁₀ carbocycle and 3- to 10-membered heterocycle, wherein the C₃₋₁₀ carbocycle and 3- to 10-membered heterocycle are each optionally substituted with one or more R⁹; and C₃₋₁₀ carbocycle and 3- to 10-membered heterocycle, each of which is optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —N(R¹⁰)C(O)N(R¹⁰)₂, —OC(O)N(R¹⁰)₂, —N(R¹⁰)C(O)OR¹⁰, —C(O)OR¹⁰, —OC(O)R¹⁰, —S(O)R¹⁰, —S(O)₂R¹⁰, —NO₂, ═O, ═S, ═N(R¹⁰), —CN, C₁₋₆ alkyl, C₂₋₆ alkenyl, and C₂₋₆ alkynyl, wherein C₁₋₆ alkyl, C₂₋₆ alkenyl, and C₂₋₆ alkynyl are each optionally substituted with one or more R⁹. In some embodiments, each R⁷ is selected from: C₁₋₆ alkyl optionally substituted with one or more substituents independently selected from C₃₋₁₀ carbocycle and 3- to 10-membered heterocycle, wherein the C₃₋₁₀ carbocycle and 3- to 10-membered heterocycle are each optionally substituted with one or more R⁹; and C₃₋₁₀ carbocycle and 3- to 10-membered heterocycle, each of which is optionally substituted with one or more substituents independently selected from halogen, —OR¹⁰, —SR¹⁰, —N(R¹⁰)₂, —CN, and C₁₋₆ alkyl optionally substituted with one or more R⁹. In some embodiments, R⁷ is C₁₋₆ alkyl substituted with one or more substituents independently selected from C₃₋₁₀ carbocycle and 3- to 10-membered heterocycle, wherein the C₃₋₁₀ carbocycle and 3- to 10-membered heterocycle are each optionally substituted with one or more substituents independently selected from halogen, —CN, —OH, —SH, —NO₂, —NH₂, —O—C₁₋₆ alkyl, —S—C₁₋₆ alkyl, —N(C₁₋₆ alkyl)₂, and —NH(C₁₋₆ alkyl). In some embodiments, R⁷ is C₁₋₆ alkyl substituted with one or more substituents independently selected from C₃₋₆ carbocycle and 3- to 6-membered heterocycle, wherein the C₃₋₆ carbocycle and 3- to 6-membered heterocycle are each optionally substituted with one or more substituents independently selected from halogen, —CN, —OH, —SH, —NO₂, —NH₂, —O—C₁₋₆ alkyl, —S—C₁₋₆ alkyl, —N(C₁₋₆ alkyl)₂, and —NH(C₁₋₆ alkyl). In some embodiments, R⁷ is selected from C₃₋₁₀ carbocycle and 3- to 10-membered heterocycle, each of which is optionally substituted with one or more substituents independently selected from halogen, —CN, —OH, —SH, —NO₂, —NH₂, —O—C₁₋₆ alkyl, —S—C₁₋₆ alkyl, —N(C₁₋₆ alkyl)₂, and —NH(C₁₋₆ alkyl).

In some embodiments, for a compound or salt of Formula (I), R⁵ is selected from hydrogen, halogen, —OR¹⁰, —SR¹⁰, —N(R¹⁰)₂, —NO₂, and —CN; and C₁₋₆ alkyl optionally substituted with one or more substituents independently selected from halogen, —OR¹⁰, —SR¹⁰, —N(R¹⁰)₂, —NO₂, and —CN. In some embodiments, R⁵ is selected from hydrogen, halogen, —CN, —OH, —SH, —NO₂, —NH₂, —O—C₁₋₆ alkyl, —S—C₁₋₆ alkyl, —N(C₁₋₆ alkyl)₂, and —NH(C₁₋₆ alkyl). In some embodiments, R⁵ is hydrogen.

In some embodiments, for a compound or salt of Formula (I), R⁶ is selected from hydrogen, halogen, —CN, —OH, —SH, —NO₂, —NH₂, —O—C₁₋₆ alkyl, —S—C₁₋₆ alkyl, —N(C₁₋₆ alkyl)₂, and —NH(C₁₋₆ alkyl). In some embodiments, R⁶ is selected from hydrogen and halogen.

In some embodiments, for a compound or salt of Formula (I), Z is —NR⁸—. In some embodiments, hydrogen, C₁₋₆ alkyl, C₂₋₆ alkenyl, and C₂₋₆ alkynyl, each of which is optionally substituted with one or more substituents independently selected from halogen, —SR¹⁰, —N(R¹⁰)₂, —C(O)R¹⁰, —C(O)N(R¹⁰)₂, —N(R¹⁰)C(O)R¹⁰, —C(O)OR¹⁰, —OC(O)R¹⁰, —N(R¹⁰)C(O)N(R¹⁰)₂, —OC(O)N(R¹⁰)₂, —N(R¹⁰)C(O)OR¹⁰, —S(O)R¹⁰, —S(O)₂R¹⁰, —NO₂, ═O, ═S, ═N(R¹⁰), —CN, C₃₋₁₀ carbocycle and 3- to 10-membered heterocycle, wherein the C₃₋₁₀ carbocycle and 3- to 10-membered heterocycle are each optionally substituted with one or more R⁹; and C₃₋₁₀ carbocycle and 3- to 10-membered heterocycle, each of which is optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —N(R¹⁰)C(O)N(R¹⁰)₂, —OC(O)N(R¹⁰)₂, —N(R¹⁰)C(O)OR¹⁰, —C(O)OR¹⁰, —OC(O)R¹⁰, —S(O)R¹⁰, —S(O)₂R¹⁰, —NO₂, ═O, ═S, ═N(R¹⁰), —CN, C₁₋₆ alkyl, C₂₋₆ alkenyl, and C₂₋₆ alkynyl, wherein C₁₋₆ alkyl, C₂₋₆ alkenyl, and C₂₋₆ alkynyl are each optionally substituted with one or more R⁹. In some embodiments, R⁸ is selected from hydrogen. In some embodiments, R⁸ is selected from C₁₋₆ alkyl, C₂₋₆ alkenyl, and C₂₋₆ alkynyl, each of which is optionally substituted with one or more substituents independently selected from halogen, —SR¹⁰, —N(R¹⁰)₂, —C(O)R¹⁰, —C(O)N(R¹⁰)₂, —N(R¹⁰)C(O)R¹⁰, —C(O)OR¹⁰, —OC(O)R¹⁰, —N(R¹⁰)C(O)N(R¹⁰)₂, —OC(O)N(R¹⁰)₂, —N(R¹⁰)C(O)OR¹⁰, —S(O)R¹⁰, —S(O)₂R¹⁰, —NO₂, ═O, ═S, ═N(R¹⁰), —CN, C₃₋₁₀ carbocycle and 3- to 10-membered heterocycle, wherein the C₃₋₁₀ carbocycle and 3- to 10-membered heterocycle are each optionally substituted with one or more R⁹. In some embodiments, R⁸ is selected from C₁₋₆ alkyl, C₂₋₆ alkenyl, and C₂₋₆ alkynyl, each of which is optionally substituted with one or more substituents independently selected from halogen, —CN, —SH, —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₃₋₁₀ carbocycle, 3- to 10-membered heterocycle. In some embodiments, R⁸ is selected from C₁₋₆ alkyl optionally substituted with one or more substituents independently selected from halogen, —CN, —SH, —NO₂, —NH₂, —O—C₁₋₆ alkyl, —S—C₁₋₆ alkyl, —N(C₁₋₆ alkyl)₂, and —NH(C₁₋₆ alkyl). In some embodiments, R⁸ is selected from C₁₋₆ alkyl. In some embodiments, R⁸ is selected from C₃₋₁₀ optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —N(R¹⁰)C(O)N(R¹⁰)₂, —OC(O)N(R¹⁰)₂, —N(R¹⁰)C(O)OR¹⁰, —C(O)OR¹⁰, —OC(O)R¹⁰, —S(O)R¹⁰, —S(O)₂R¹⁰, —NO₂, ═O, ═S, ═N(R¹⁰), —CN, C₁₋₆ alkyl, C₂₋₆ alkenyl, and C₂₋₆ alkynyl, wherein C₁₋₆ alkyl, C₂₋₆ alkenyl, and C₂₋₆ alkynyl are each optionally substituted with one or more R⁹. In some embodiments, R⁸ is selected from C₃₋₁₀ carbocycle optionally substituted with one or more substituents independently selected from halogen, —CN, —OH, —SH, —NO₂, —NH₂, —O—C₁₋₆ alkyl, —S—C₁₋₆ alkyl, —N(C₁₋₆ alkyl)₂, and —NH(C₁₋₆ alkyl). In some embodiments, R⁸ is selected from 3- to 10-membered heterocycle optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —N(R¹⁰)C(O)N(R¹⁰)₂, —OC(O)N(R¹⁰)₂, —N(R¹⁰)C(O)OR¹⁰, —C(O)OR¹⁰, —OC(O)R¹⁰, —S(O)R¹⁰, —S(O)₂R¹⁰, —NO₂, ═O, ═S, ═N(R¹⁰), —CN, C₁₋₆ alkyl, C₂₋₆ alkenyl, and C₂₋₆ alkynyl, wherein C₁₋₆ alkyl, C₂₋₆ alkenyl, and C₂₋₆ alkynyl are each optionally substituted with one or more R⁹. In some embodiments, R⁸ is selected from 3- to 10-membered heterocycle optionally substituted with one or more substituents independently selected from halogen, —CN, —OH, —SH, —NO₂, —NH₂, —O—C₁₋₆ alkyl, —S—C₁₋₆ alkyl, —N(C₁₋₆ alkyl)₂, and —NH(C₁₋₆ alkyl). In some embodiments, R⁸ is hydrogen.

In some embodiments, for a compound or salt of Formula (I), each R⁹ is selected from halogen, —R¹⁰, —OR¹⁰, —SR¹⁰, —N(R¹⁰)₂, —C(O)R¹⁰, —C(O)N(R¹⁰)₂, —N(R¹⁰)C(O)R¹⁰, —N(R¹⁰)C(O)N(R¹⁰)₂, —OC(O)N(R¹⁰)₂, —N(R¹⁰)C(O)OR¹⁰, —C(O)OR¹⁰, —OC(O)R¹⁰, —OC(O)₂R¹⁰, —S(O)R¹⁰, —S(O)₂R¹⁰, —NO₂, ═O, ═S, ═N(R¹⁰), and —CN; C₁₋₃ alkyl, C₂₋₃ alkenyl, and C₂₋₃ alkynyl, each of which is optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —N(R¹⁰)C(O)N(R¹⁰)₂, —OC(O)N(R¹⁰)₂, —N(R¹⁰)C(O)OR¹⁰, —C(O)OR¹⁰, —OC(O)R¹⁰, —S(O)R¹⁰, —S(O)₂R¹⁰, —NO₂, ═O, ═S, ═N(R¹⁰), and —CN; C₃₋₁₀ carbocycle and 3- to 10-membered heterocycle, each of which is optionally substituted with one or more substituents independently selected from halogen, —R¹⁰, —OR¹⁰, —SR¹⁰, —N(R¹⁰)₂, —C(O)R¹⁰, —C(O)N(R¹⁰)₂, —N(R¹⁰)C(O)R¹⁰, —N(R¹⁰)C(O)N(R¹⁰)₂, —OC(O)N(R¹⁰)₂, —N(R¹⁰)C(O)OR¹⁰, —C(O)OR¹⁰, —OC(O)R¹⁰, —S(O)R¹⁰, —S(O)₂R¹⁰, —NO₂, ═O, ═S, ═N(R¹⁰), and —CN; and

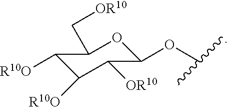

In some embodiments, each R⁹ is selected from halogen, —CN, —OH, —SH, —NO₂, —NH₂, —O—C₁₋₆ alkyl, —S—C₁₋₆ alkyl, —N(C₁₋₆ alkyl)₂, and —NH(C₁₋₆ alkyl); and C₁₋₃ alkyl optionally substituted with one or more substituents independently selected from halogen, —CN, —OH, —SH, —NO₂, —NH₂, —O—C₁₋₆ alkyl, —S—C₁₋₆ alkyl, —N(C₁₋₆ alkyl)₂, and —NH(C₁₋₆ alkyl).

In some embodiments, for a compound or salt of Formula (I), each R¹⁰ hydrogen; C₁₋₆ alkyl, C₂₋₆ alkenyl, and C₂₋₆ alkynyl, each of which is optionally substituted with one or more substituents independently selected from halogen, —CN, —OH, —SH, —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₃₋₁₀ carbocycle, 3- to 10-membered heterocycle; and C₃₋₁₀ carbocycle, and 3- to 10-membered heterocycle, each of which is optionally substituted with one or more substituents independently selected from halogen, —CN, —OH, —SH, —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₁₋₆ alkyl, C₂₋₆ alkenyl, C₂₋₆ alkynyl, C₃₋₁₀ carbocycle, 3- to 10-membered heterocycle, and C₁₋₆ haloalkyl. In some embodiments, each is R¹⁰ hydrogen.

In some embodiments, for a compound or salt of Formula (I), W is C₁₋₆ alkylene optionally substituted with one or more substituents independently selected from halogen, —CN, —OH, —SH, —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₃₋₁₀ carbocycle, and 3- to 10-membered heterocycle. In some embodiments, W is C₁₋₆ alkylene optionally substituted with one or more substituents independently selected from halogen, —CN, —OH, —SH, —NO₂, —NH₂, —O—C₁₋₆ alkyl, —S—C₁₋₆ alkyl, —N(C₁₋₆ alkyl)₂, and —NH(C₁₋₆ alkyl). In some embodiments, W is C₁₋₆ alkylene optionally substituted with one or more substituents independently selected from C₃₋₁₀ carbocycle, and 3- to 10-membered heterocycle. In some embodiments, W is C₁₋₆ alkylene substituted with one or more substituents independently selected from C₃₋₆ carbocycle, and 3- to 5-membered heterocycle. In some embodiments, W is C₁₋₆ alkylene substituted with one or more substituents independently selected from C₃₋₆ carbocycle. In some embodiments, for a compound or salt of Formula (I), wherein W is selected from

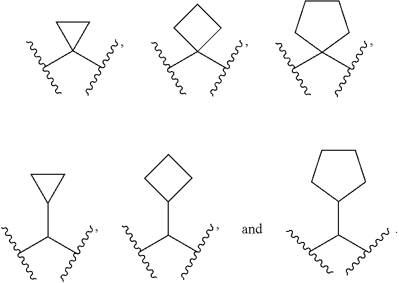 [SMILES: *C1(*)CC1 ; *C1(*)CCC1 ; *C1(*)CCCC1 ; *C(*)C1CC1 ; *C(*)C1CCC1 ; *C(*)C1CCCC1]

In some embodiments, W is C₁₋₆ alkylene.

In some embodiments, for a compound or salt of Formula (I), R¹² is selected from hydrogen, halogen, —OR¹⁰, —SR¹⁰, —N(R¹⁰)₂, —NO₂, and —CN; and C₁₋₆ alkyl optionally substituted with one or more substituents independently selected from halogen, —OR¹⁰, —SR¹⁰, —N(R¹⁰)₂, —NO₂, and —CN. In some embodiments, R¹² is selected from hydrogen and halogen.

In some embodiments, the compound or salt of Formula (I) is selected from:

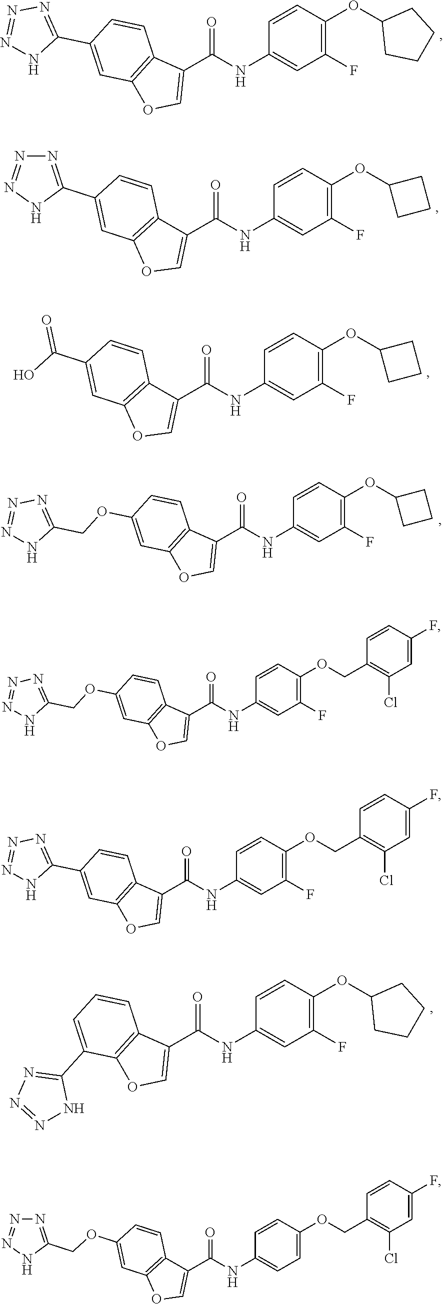

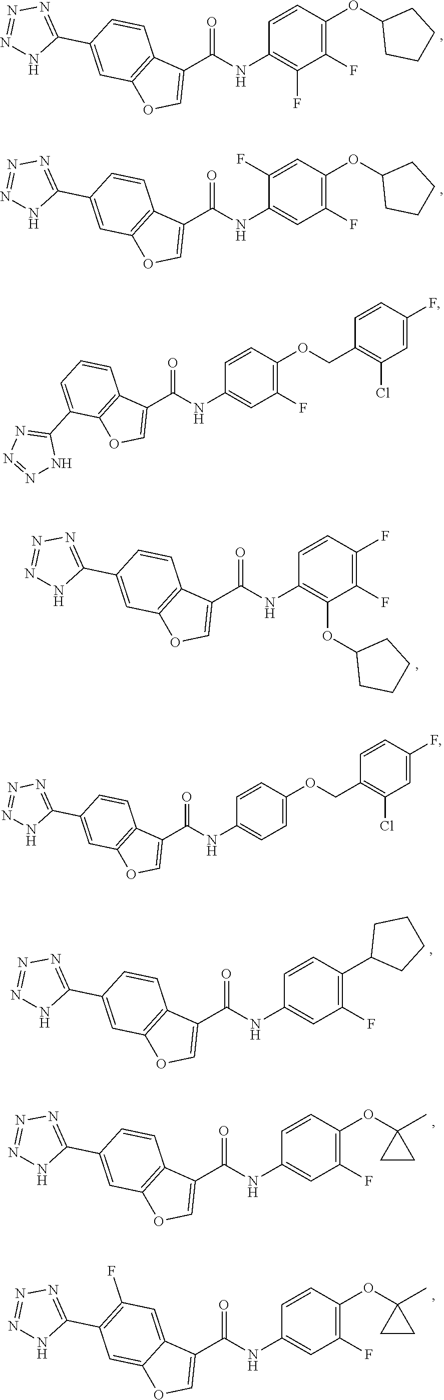

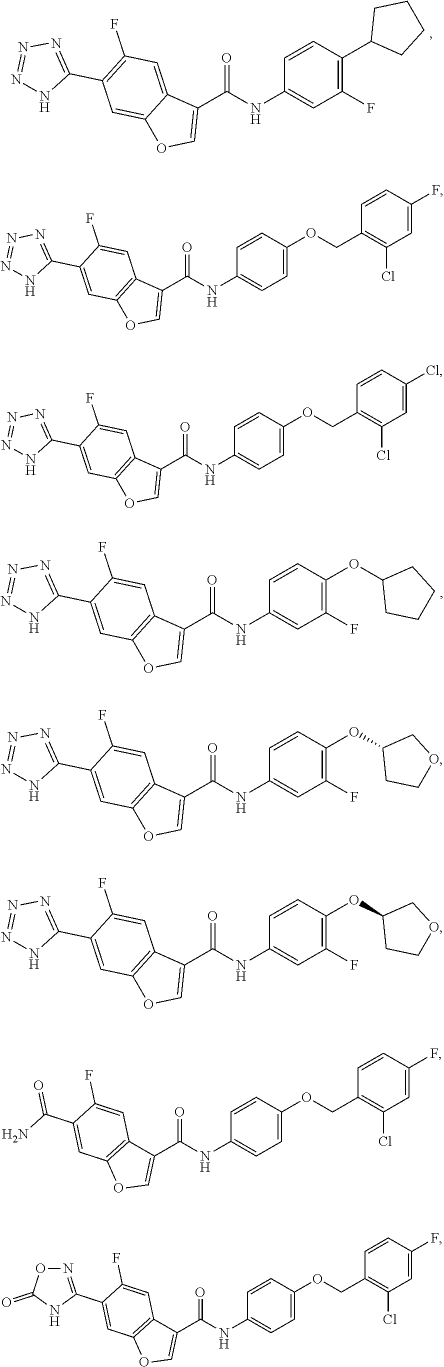

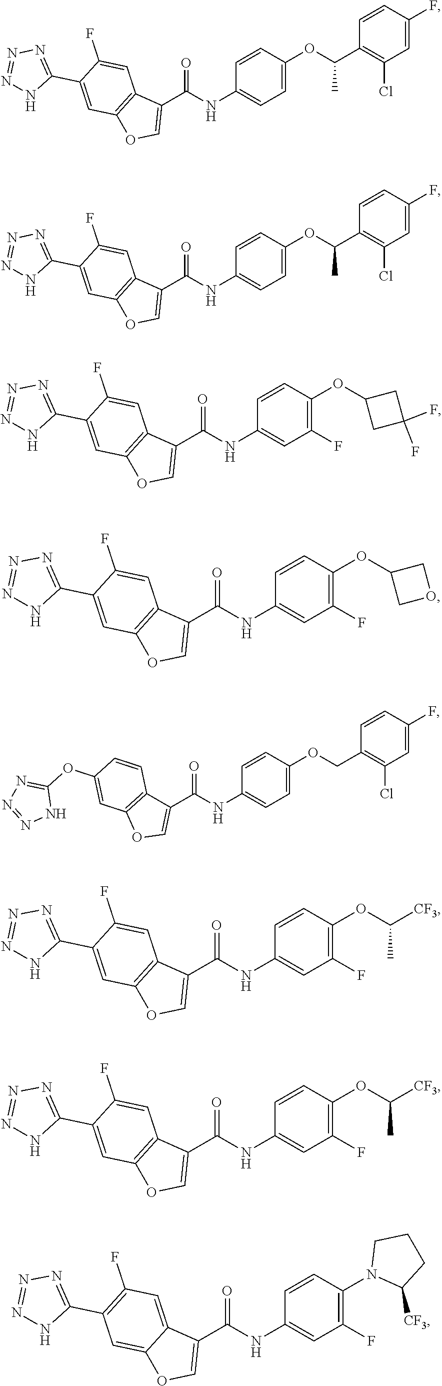

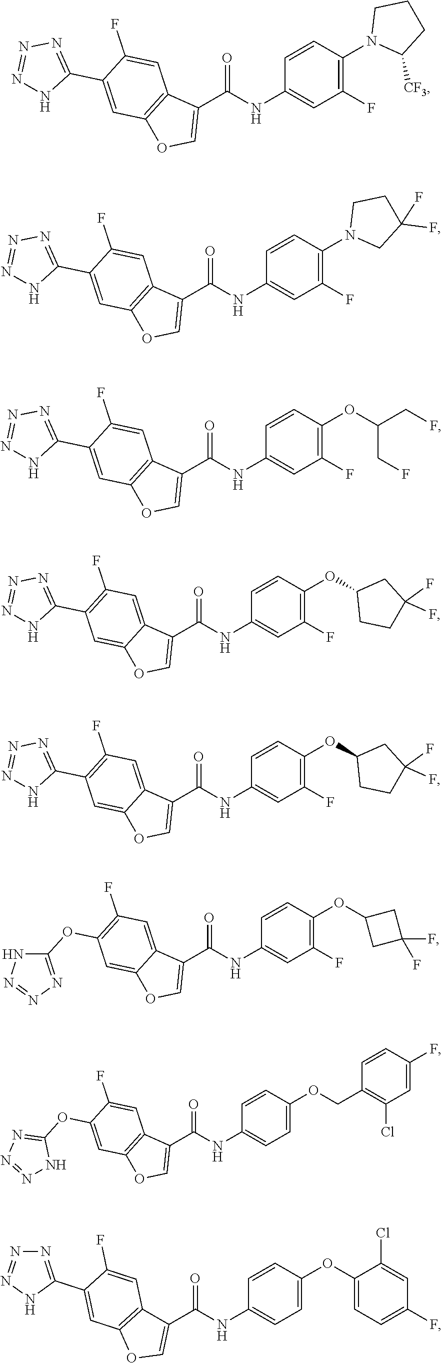

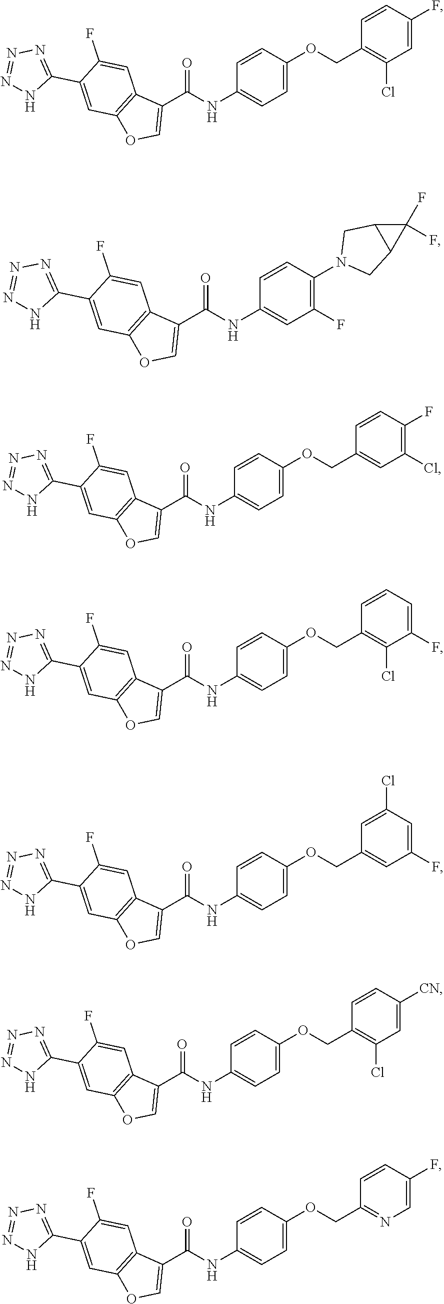

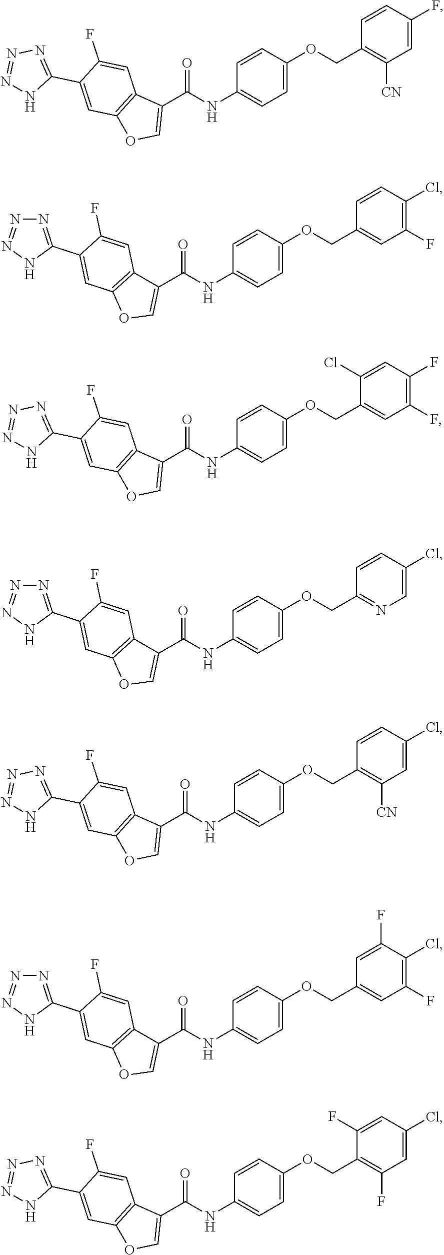

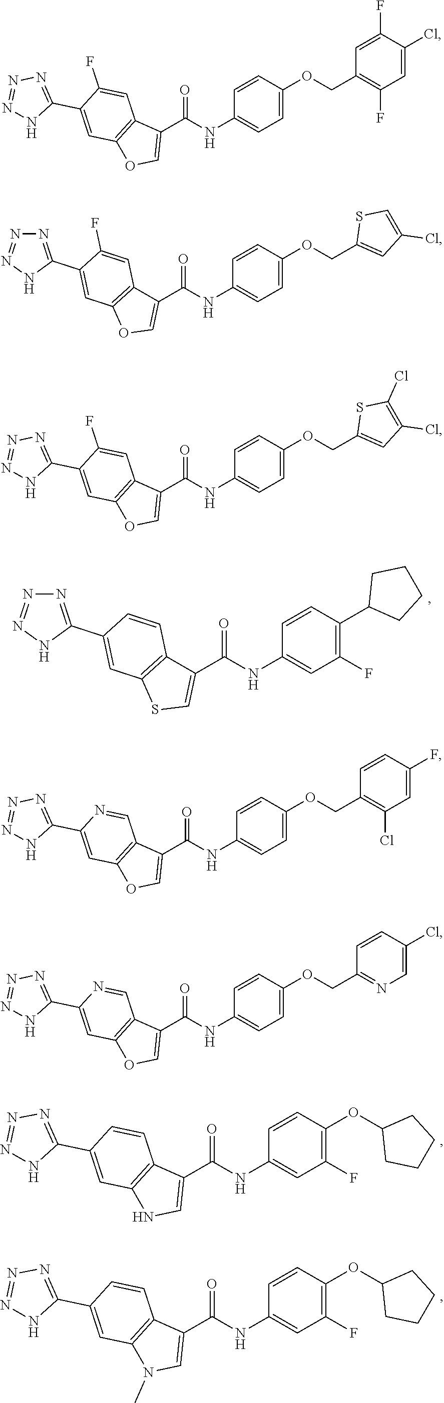

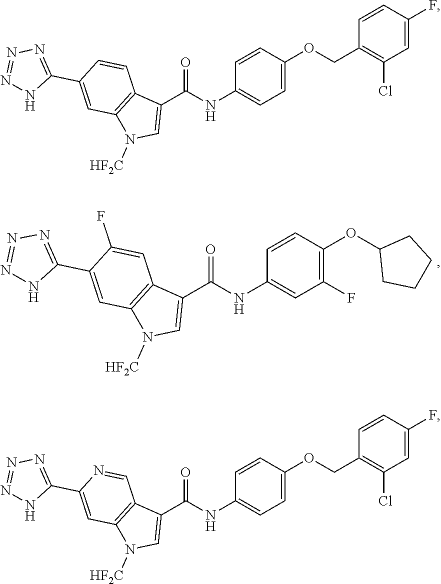

and combinations thereof.

In some embodiments, the compound or salt of Formula (I) is selected from:

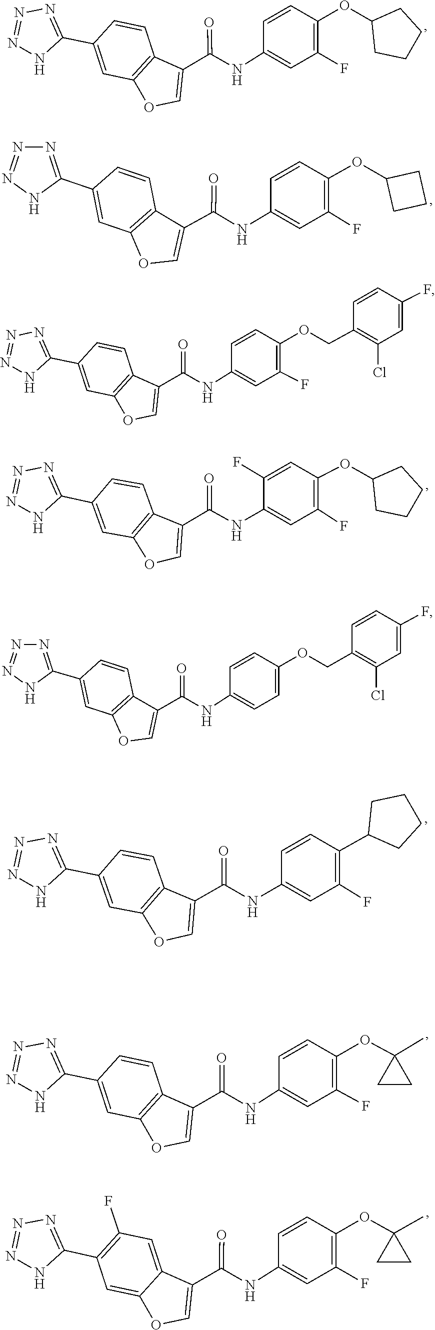

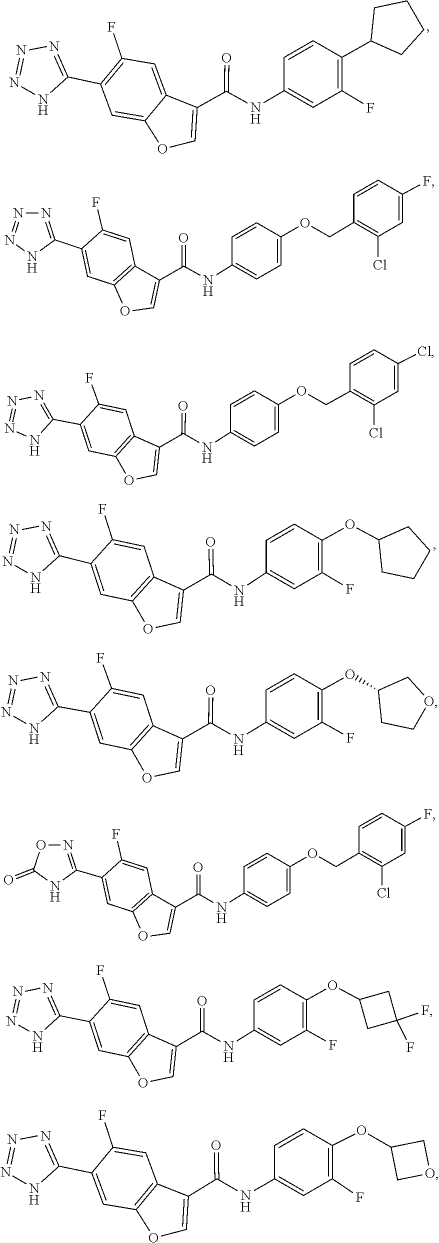

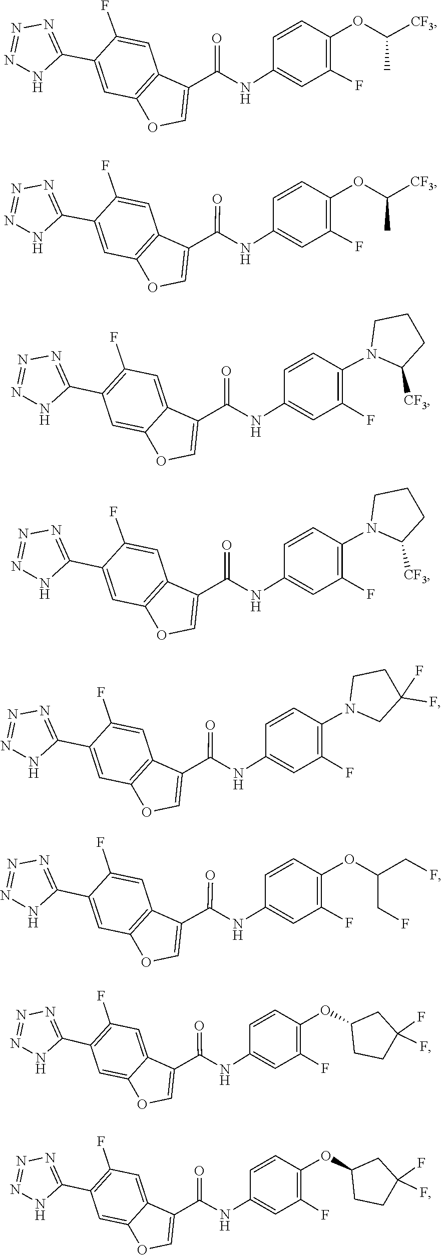

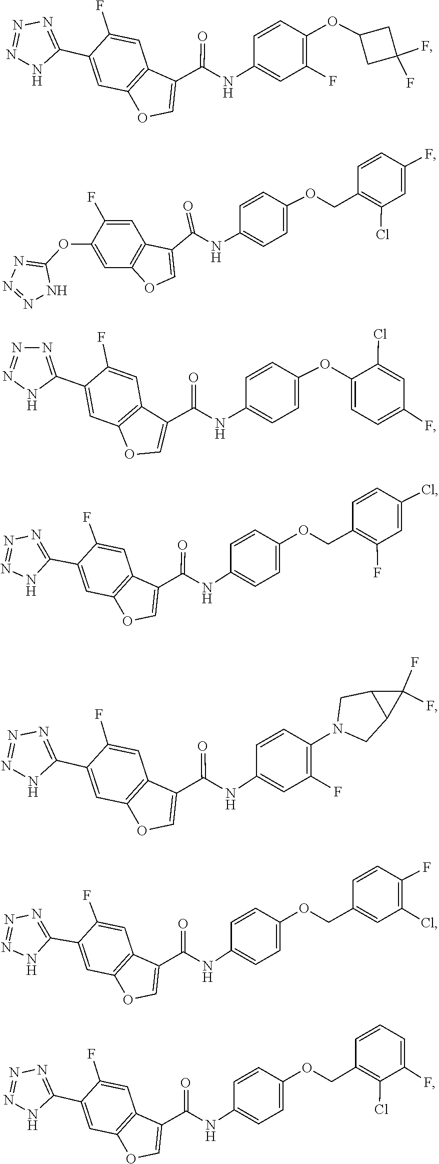

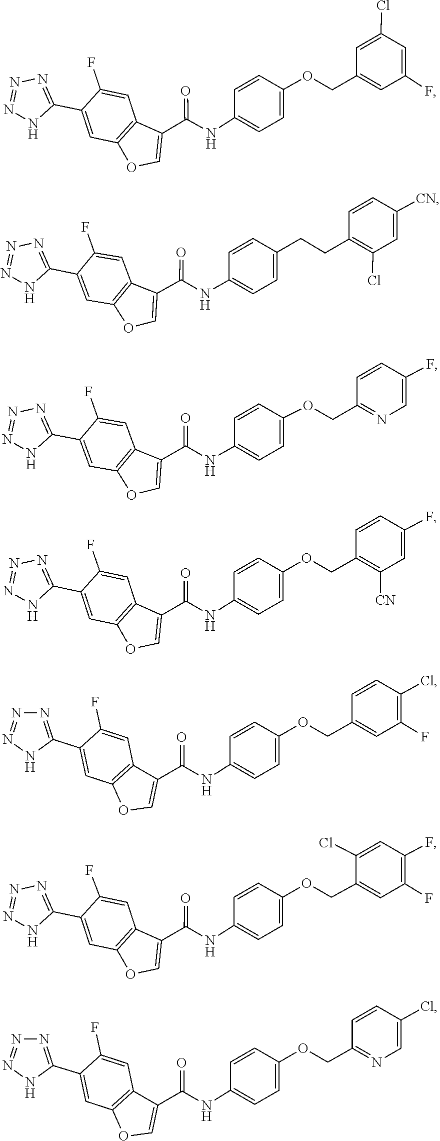

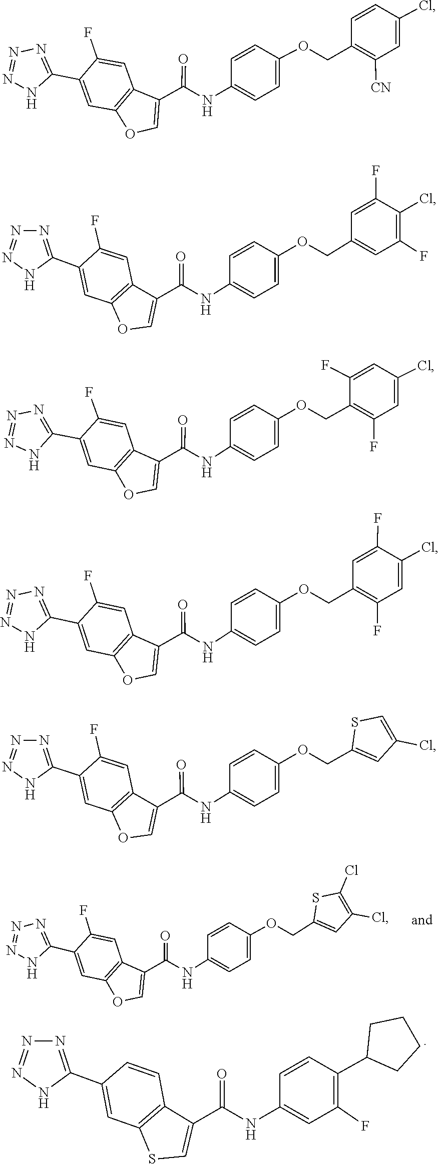

In some embodiments, the compound or salt of Formula (I) is selected from:

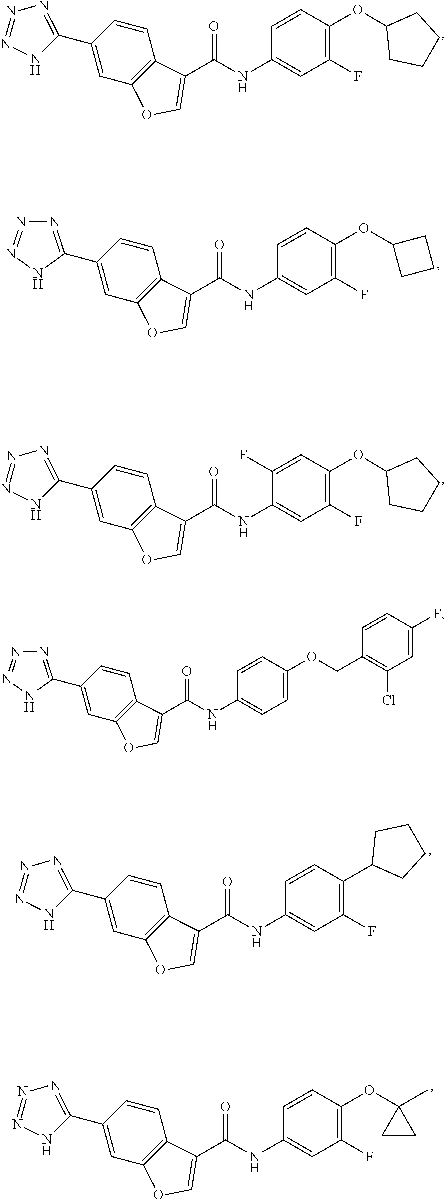 [SMILES: O=C(Nc1ccc(OC2CCCC2)c(F)c1)c1coc2cc(-c3nnn[nH]3)ccc12 ; O=C(Nc1ccc(OC2CCC2)c(F)c1)c1coc2cc(-c3nnn[nH]3)ccc12 ; O=C(Nc1cc(F)c(OC2CCCC2)cc1F)c1coc2cc(-c3nnn[nH]3)ccc12 ; O=C(Nc1ccc(OCc2ccc(F)cc2Cl)cc1)c1coc2cc(-c3nnn[nH]3)ccc12 ; O=C(Nc1ccc(C2CCCC2)c(F)c1)c1coc2cc(-c3nnn[nH]3)ccc12 ; CC1(Oc2ccc(NC(=O)c3coc4cc(-c5nnn[nH]5)ccc34)cc2F)CC1]

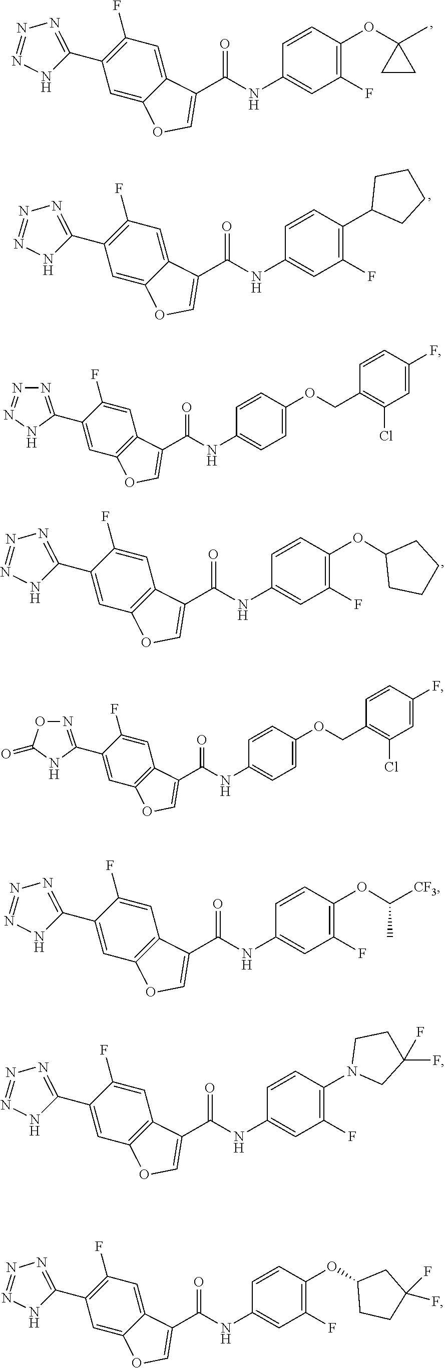

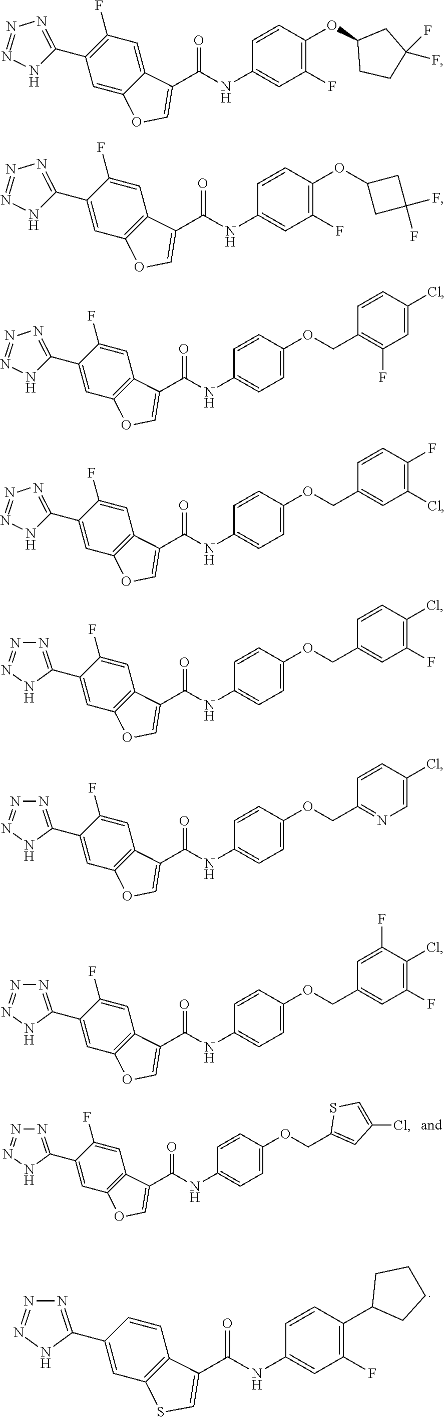

Compounds of the present invention also include crystalline and amorphous forms of those compounds, pharmaceutically acceptable salts, and active metabolites of these compounds having the same type of activity, including, for example, polymorphs, pseudopolymorphs, solvates, hydrates, unsolvated polymorphs (including anhydrates), conformational polymorphs, and amorphous forms of the compounds, as well as mixtures thereof.

Included in the present disclosure are salts, particularly pharmaceutically acceptable salts, of the compounds described herein. The compounds of the present disclosure that possess a sufficiently acidic, a sufficiently basic, or both functional groups, can react with any of a number of inorganic bases, and inorganic and organic acids, to form a salt. Alternatively, compounds that are inherently charged, such as those with a quaternary nitrogen, can form a salt with an appropriate counterion, e.g., a halide such as bromide, chloride, or fluoride, particularly bromide.

The compounds described herein may in some cases exist as diastereomers, enantiomers, or other stereoisomeric forms. The compounds presented herein include all diastereomeric, enantiomeric, and epimeric forms as well as the appropriate mixtures thereof. Separation of stereoisomers may be performed by chromatography or by forming diastereomers and separating by recrystallization, or chromatography, or any combination thereof (Jean Jacques, Andre Collet, Samuel H. Wilen, “Enantiomers, Racemates and Resolutions”, John Wiley And Sons, Inc., 1981, herein incorporated by reference for this disclosure). Stereoisomers may also be obtained by stereoselective synthesis.

The methods and compositions described herein include the use of amorphous forms as well as crystalline forms (also known as polymorphs). The compounds described herein may be in the form of pharmaceutically acceptable salts. As well, in some embodiments, active metabolites of these compounds having the same type of activity are included in the scope of the present disclosure. In addition, the compounds described herein can exist in unsolvated as well as solvated forms with pharmaceutically acceptable solvents such as water, ethanol, and the like. The solvated forms of the compounds presented herein are also considered to be disclosed herein.

In certain embodiments, compounds or salts of the compounds may be prodrugs, e.g., wherein a hydroxyl in the parent compound is presented as an ester or a carbonate, or carboxylic acid present in the parent compound is presented as an ester. The term “prodrug” is intended to encompass compounds which, under physiologic conditions, are converted into pharmaceutical agents of the present disclosure. One method for making a prodrug is to include one or more selected moieties which are hydrolyzed under physiologic conditions to reveal the desired molecule. In other embodiments, the prodrug is converted by an enzymatic activity of the host animal such as specific target cells in the host animal. For example, esters or carbonates (e.g., esters or carbonates of alcohols or carboxylic acids and esters of phosphonic acids) are preferred prodrugs of the present disclosure.

Prodrug forms of the herein described compounds, wherein the prodrug is metabolized in vivo to produce a compound as set forth herein are included within the scope of the claims. In some cases, some of the herein-described compounds may be a prodrug for another derivative or active compound.

Prodrugs are often useful because, in some situations, they may be easier to administer than the parent drug. They may, for instance, be bioavailable by oral administration whereas the parent is not. Prodrugs may help enhance the cell permeability of a compound relative to the parent drug. The prodrug may also have improved solubility in pharmaceutical compositions over the parent drug. Prodrugs may be designed as reversible drug derivatives, for use as modifiers to enhance drug transport to site-specific tissues or to increase drug residence inside of a cell.

In some embodiments, the design of a prodrug increases the lipophilicity of the pharmaceutical agent. In some embodiments, the design of a prodrug increases the effective water solubility. See, e.g., Fedorak et al., Am. J. Physiol., 269:G210-218 (1995); McLoed et al., Gastroenterol, 106:405-413 (1994); Hochhaus et al., Biomed. Chrom., 6:283-286 (1992); J. Larsen and H. Bundgaard, Int. J. Pharmaceutics, 37, 87 (1987); J. Larsen et al., Int. J. Pharmaceutics, 47, 103 (1988); Sinkula et al., J. Pharm. Sci., 64:181-210 (1975); T. Higuchi and V. Stella, Pro-drugs as Novel Delivery Systems, Vol. 14 of the A.C.S. Symposium Series; and Edward B. Roche, Bioreversible Carriers in Drug Design, American Pharmaceutical Association and Pergamon Press, 1987, all incorporated herein for such disclosure). According to another embodiment, the present disclosure provides methods of producing the above-defined compounds. The compounds may be synthesized using conventional techniques. Advantageously, these compounds are conveniently synthesized from readily available starting materials.

Synthetic chemistry transformations and methodologies useful in synthesizing the compounds described herein are known in the art and include, for example, those described in R. Larock, Comprehensive Organic Transformations (1989); T. W. Greene and P. G. M. Wuts, Protective Groups in Organic Synthesis, 2d. Ed. (1991); L. Fieser and M. Fieser, Fieser and Fieser's Reagentsfor Organic Synthesis (1994); and L. Paquette, ed., Encyclopedia of Reagents for Organic Synthesis (1995).

### Therapeutic Applications

Methods of administration of a compound or salt of Formula (I) or (II) discussed herein may be used for the treatment of cancer.

In one aspect, disclosed herein is a compound represented by Formula (II):

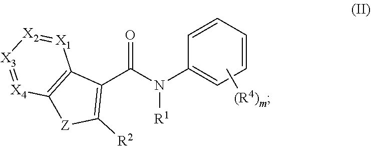

or a pharmaceutically acceptable salt thereof, wherein:
X₁, X₂, X₃, and X₄ are each independently selected from C-Q-R³, CR⁶ and N;
Z is selected from —O—, —S—, and —NR⁸—;
Q is absent or selected from:
   W—, —O—, —OW—, —WO—, —S—, —SW—, —WS—, —NR¹⁰—W—, —W—NR¹⁰—, C(O)—, —C(O)N(R¹⁰)—, —N(R¹⁰)C(O)—, —C(O)O—, —OC(O)—, —N(R¹⁰)C(O)N(R¹⁰)0-, —OC(O)N(R¹⁰)—, —N(R¹⁰)C(O)O—, —S(O)—, —S(O)₂—, and —P(O)₂O—;
m is selected from 1, 2, 3, 4, and 5;
R¹ is selected from:
   hydrogen, C₁₋₆ alkyl, C₂₋₆ alkenyl, and C₂₋₆ alkynyl, C₃₋₁₀ carbocycle and 3- to 10-membered heterocycle, each of which is optionally substituted;
R² is selected from:
   hydrogen, halogen, —OR¹⁰, —SR¹⁰, —N(R¹⁰)₂, —C(O)R¹⁰, —C(O)N(R¹⁰)₂, —N(R¹⁰)C(O)R¹⁰, —N(R¹⁰)C(O)N(R¹⁰)₂, —OC(O)N(R¹⁰)₂, —N(R¹⁰)C(O)OR¹⁰, —C(O)OR¹⁰, —OC(O)R¹⁰, —S(O)R¹⁰, —S(O)₂R¹⁰, —NO₂, ═O, ═S, ═N(R¹⁰), and —CN;
   C₁₋₆ alkyl, C₂₋₆ alkenyl, and C₂₋₆ alkynyl, C₃₋₁₀ carbocycle and 3- to 10-membered heterocycle, each of which is optionally substituted;
R³ is selected from C₃₋₁₀ carbocycle and 3- to 10-membered heterocycle, each of which is optionally substituted;
each R⁴ is independently selected from:
   halogen, —OR⁷, —SR⁷, —N(R⁷)₂, —C(O)R⁷, —C(O)N(R⁷)₂, —N(R⁷)C(O)R⁷, —N(R⁷)C(O)N(R⁷)₂, —OC(O)N(R⁷)₂, —N(R⁷)C(O)OR⁷, —C(O)OR⁷, —OC(O)R⁷, —S(O)R⁷, —S(O)₂R⁷, —NO₂, and —CN;
   C₁₋₆ alkyl, C₂₋₆ alkenyl, and C₂₋₆ alkynyl, C₃₋₁₀ carbocycle and 3- to 10-membered heterocycle, each of which is optionally substituted;
each R⁶ is independently selected from:
   hydrogen, halogen, —OR¹⁰, —SR¹⁰, —N(R¹⁰)₂, —NO₂, and —CN; and optionally substituted C₁₋₆ alkyl;
each R⁷ is independently selected from hydrogen, C₁₋₆ alkyl, C₂₋₆ alkenyl, and C₂₋₆ alkynyl, C₃₋₁₀ carbocycle and 3- to 10-membered heterocycle, each of which is optionally substituted;
R⁸ is selected from hydrogen, C₁₋₆ alkyl, C₂₋₆ alkenyl, and C₂₋₆ alkynyl, C₃₋₁₀ carbocycle and 3- to 10-membered heterocycle, each of which is optionally substituted;
each R¹⁰ is independently selected from hydrogen, C₁₋₆ alkyl, C₂₋₆ alkenyl, and C₂₋₆ alkynyl, C₃₋₁₀ carbocycle, and 3- to 10-membered heterocycle, each of which is optionally substituted;
W is selected from C₁₋₆ alkylene, C₂₋₆ alkenylene, and C₂₋₆ alkynylene, each of which is optionally substituted.

In one aspect, disclosed herein is a compound represented by Formula (II):

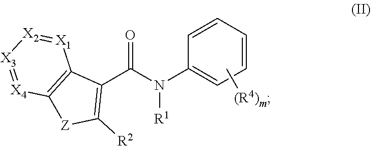

or a pharmaceutically acceptable salt thereof, wherein:
X₁, X₂, X₃, and X₄ are each independently selected from C-Q-R³, CR⁶ and N;
Z is selected from —O—, —S—, and —NR⁸—;
Q is absent or selected from:
   —W—, —O—, —OW—, —WO—, —S—, —SW—, —WS—, —NR¹⁰—W—, —W—NR¹⁰—, C(O)—, —C(O)N(R¹⁰)—, —N(R¹⁰)C(O)—, —C(O)O—, —OC(O)—, —N(R¹⁰)C(O)N(R¹⁰)0-, —OC(O)N(R¹⁰)—, —N(R¹⁰)C(O)O—, —S(O)—, —S(O)₂—, and —P(O)₂O—;
m is selected from 1, 2, 3, 4, and 5;
R¹ is selected from:
   hydrogen;
   C₁₋₆ alkyl, C₂₋₆ alkenyl, and C₂₋₆ alkynyl, each of which is optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —C(O)OR¹⁰, —OC(O)R¹⁰, —N(R¹⁰)C(O)N(R¹⁰)₂, —OC(O)N(R¹⁰)₂, —N(R¹⁰)C(O)OR¹⁰, —S(O)R¹⁰, —S(O)₂R¹⁰, —NO₂, ═O, ═S, ═N(R¹⁰), —CN, C₃₋₁₀ carbocycle and 3- to 10-membered heterocycle, wherein the C₃₋₁₀ carbocycle and 3- to 10-membered heterocycle are each optionally substituted with one or more R⁹; and
   C₃₋₁₀ carbocycle and 3- to 10-membered heterocycle, each of which is optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —N(R¹⁰)C(O)N(R¹⁰)₂, —OC(O)N(R¹⁰)₂, —N(R¹⁰)C(O)OR¹⁰, —C(O)OR¹⁰, —OC(O)R¹⁰, —S(O)R¹⁰, —S(O)₂R¹⁰, —NO₂, ═O, ═S, ═N(R¹⁰), —CN, C₁₋₆ alkyl, C₂₋₆ alkenyl, and C₂₋₆ alkynyl, wherein C₁₋₆ alkyl, C₂₋₆ alkenyl, and C₂₋₆ alkynyl are each optionally substituted with one or more R⁹
R² is selected from:
   hydrogen;
   halogen, —OR¹⁰, —SR¹⁰, —N(R¹⁰)₂, —C(O)R¹⁰, —C(O)N(R¹⁰)₂, —N(R¹⁰)C(O)R¹⁰, —N(R¹⁰)C(O)N(R¹⁰)₂, —OC(O)N(R¹⁰)₂, —N(R¹⁰)C(O)OR¹⁰, —C(O)OR¹⁰, —OC(O)R¹⁰, —S(O)R¹⁰, —S(O)₂R¹⁰, —NO₂, ═O, ═S, ═N(R¹⁰), and —CN;
   C₁₋₆ alkyl, C₂₋₆ alkenyl, and C₂₋₆ alkynyl, each of which is optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —C(O)OR¹⁰, —OC(O)R¹⁰, —N(R¹⁰)C(O)N(R¹⁰)₂, —OC(O)N(R¹⁰)₂, —N(R¹⁰)C(O)OR¹⁰, —S(O)R¹⁰, —S(O)₂R¹⁰, —NO₂, ═O, ═S, ═N(R¹⁰), —CN, C₃₋₁₀ carbocycle and 3- to 10-membered heterocycle, wherein the C₃₋₁₀ carbocycle and 3- to 10-membered heterocycle are each optionally substituted with one or more R⁹; and
   C₃₋₁₀ carbocycle and 3- to 10-membered heterocycle, each of which is optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —N(R¹⁰)C(O)N(R¹⁰)₂, —OC(O)N(R¹⁰)₂, —N(R¹⁰)C(O)OR¹⁰, —C(O)OR¹⁰, —OC(O)R¹⁰, —S(O)R¹⁰, —S(O)₂R¹⁰, —NO₂, ═O, ═S, ═N(R¹⁰), —CN, C₁₋₆ alkyl, C₂₋₆ alkenyl, and C₂₋₆ alkynyl, wherein C₁₋₆ alkyl, C₂₋₆ alkenyl, and C₂₋₆ alkynyl are each optionally substituted with one or more R⁹
R³ is selected from:
   hydrogen;
   halogen, —OR¹⁰, —SR¹⁰, —N(R¹⁰)₂, —C(O)R¹⁰, —C(O)N(R¹⁰)₂, —N(R¹⁰)C(O)R¹⁰, —N(R¹⁰)C(O)N(R¹⁰)₂, —OC(O)N(R¹⁰)₂, —N(R¹⁰)C(O)OR¹⁰, —C(O)OR¹⁰, —OC(O)R¹⁰, —S(O)R¹⁰, —S(O)₂R¹⁰, —NO₂, ═O, ═S, ═N(R¹⁰), and —CN;
   C₁₋₆ alkyl, C₂₋₆ alkenyl, and C₂₋₆ alkynyl, each of which is optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —C(O)OR¹⁰, —OC(O)R¹⁰, —N(R¹⁰)C(O)N(R¹⁰)₂, —OC(O)N(R¹⁰)₂, —N(R¹⁰)C(O)OR¹⁰, —S(O)R¹⁰, —S(O)₂R¹⁰, —NO₂, ═O, ═S, ═N(R¹⁰), —CN, C₃₋₁₀ carbocycle and 3- to 10-membered heterocycle, wherein the C₃₋₁₀ carbocycle and 3- to 10-membered heterocycle are each optionally substituted with one or more R⁹; and
   C₃₋₁₀ carbocycle and 3- to 10-membered heterocycle, each of which is optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —N(R¹⁰)C(O)N(R¹⁰)₂, —OC(O)N(R¹⁰)₂, —N(R¹⁰)C(O)OR¹⁰, —C(O)OR¹⁰, —OC(O)R¹⁰, —S(O)R¹⁰, —S(O)₂R¹⁰, —NO₂, ═O, ═S, ═N(R¹⁰), —CN, C₁₋₆ alkyl, C₂₋₆ alkenyl, and C₂₋₆ alkynyl, wherein C₁₋₆ alkyl, C₂₋₆ alkenyl, and C₂₋₆ alkynyl are each optionally substituted with one or more R⁹;
each R⁴ is independently selected from:
   halogen, —OR⁷, —SR⁷, —N(R⁷)₂, —C(O)R⁷, —C(O)N(R⁷)₂, —N(R⁷)C(O)R⁷, —N(R⁷)C(O)N(R⁷)₂, —OC(O)N(R⁷)₂, —N(R⁷)C(O)OR⁷, —C(O)OR⁷, —OC(O)R⁷, —S(O)R⁷, —S(O)₂R⁷, —NO₂, and —CN;
   C₁₋₆ alkyl, C₂₋₆ alkenyl, and C₂₋₆ alkynyl, each of which is optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —C(O)OR¹⁰, —OC(O)R¹⁰, —N(R¹⁰)C(O)N(R¹⁰)₂, —OC(O)N(R¹⁰)₂, —N(R¹⁰)C(O)OR¹⁰, —S(O)R¹⁰, —S(O)₂R¹⁰, —NO₂, ═O, ═S, ═N(R¹⁰), —CN, C₃₋₁₀ carbocycle and 3- to 10-membered heterocycle, wherein the C₃₋₁₀ carbocycle and 3- to 10-membered heterocycle are each optionally substituted with one or more R⁹; and
   C₃₋₁₀ carbocycle and 3- to 10-membered heterocycle, each of which is optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —N(R¹⁰)C(O)N(R¹⁰)₂, —OC(O)N(R¹⁰)₂, —N(R¹⁰)C(O)OR¹⁰, —C(O)OR¹⁰, —OC(O)R¹⁰, —S(O)R¹⁰, —S(O)₂R¹⁰, —NO₂, ═O, ═S, ═N(R¹⁰), —CN, C₁₋₆ alkyl, C₂₋₆ alkenyl, and C₂₋₆ alkynyl, wherein C₁₋₆ alkyl, C₂₋₆ alkenyl, and C₂₋₆ alkynyl are each optionally substituted with one or more R⁹
each R⁶ is independently selected from:
   hydrogen, halogen, —OR¹⁰, —SR¹⁰, —N(R¹⁰)₂, —NO₂, and —CN; and
   C₁₋₆ alkyl optionally substituted with one or more substituents independently selected from halogen, —OR¹⁰, —SR¹⁰, —N(R¹⁰)₂, —NO₂, and —CN;
each R⁷ is independently selected from:
   hydrogen;
   C₁₋₆ alkyl, C₂₋₆ alkenyl, and C₂₋₆ alkynyl, each of which is optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —C(O)OR¹⁰, —OC(O)R¹⁰, —N(R¹⁰)C(O)N(R¹⁰)₂, —OC(O)N(R¹⁰)₂, —N(R¹⁰)C(O)OR¹⁰, —S(O)R¹⁰, —S(O)₂R¹⁰, —NO₂, ═O, ═S, ═N(R¹⁰), —CN, C₃₋₁₀ carbocycle and 3- to 10-membered heterocycle, wherein the C₃₋₁₀ carbocycle and 3- to 10-membered heterocycle are each optionally substituted with one or more R⁹; and
   C₃₋₁₀ carbocycle and 3- to 10-membered heterocycle, each of which is optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —N(R¹⁰)C(O)N(R¹⁰)₂, —OC(O)N(R¹⁰)₂, —N(R¹⁰)C(O)OR¹⁰, —C(O)OR¹⁰, —OC(O)R¹⁰, —S(O)R¹⁰, —S(O)₂R¹⁰, —NO₂, ═O, ═S, ═N(R¹⁰), —CN, C₁₋₆ alkyl, C₂₋₆ alkenyl, and C₂₋₆ alkynyl, wherein C₁₋₆ alkyl, C₂₋₆ alkenyl, and C₂₋₆ alkynyl are each optionally substituted with one or more R⁹;
R⁸ is selected from:
   hydrogen;
   C₁₋₆ alkyl, C₂₋₆ alkenyl, and C₂₋₆ alkynyl, each of which is optionally substituted with one or more substituents independently selected from halogen, —SR¹⁰, —N(R¹⁰)₂, —C(O)R¹⁰, —C(O)N(R¹⁰)₂, —N(R¹⁰)C(O)R¹⁰, —C(O)OR¹⁰, —OC(O)R¹⁰, —N(R¹⁰)C(O)N(R¹⁰)₂, —OC(O)N(R¹⁰)₂, —N(R¹⁰)C(O)OR¹⁰, —S(O)R¹⁰, —S(O)₂R¹⁰, —NO₂, ═O, ═S, ═N(R¹⁰), —CN, C₃₋₁₀ carbocycle and 3- to 10-membered heterocycle, wherein the C₃₋₁₀ carbocycle and 3- to 10-membered heterocycle are each optionally substituted with one or more R⁹; and
   C₃₋₁₀ carbocycle and 3- to 10-membered heterocycle, each of which is optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —N(R¹⁰)C(O)N(R¹⁰)₂, —OC(O)N(R¹⁰)₂, —N(R¹⁰)C(O)OR¹⁰, —C(O)OR¹⁰, —OC(O)R¹⁰, —S(O)R¹⁰, —S(O)₂R¹⁰, —NO₂, ═O, ═S, ═N(R¹⁰), —CN, C₁₋₆ alkyl, C₂₋₆ alkenyl, and C₂₋₆ alkynyl, wherein C₁₋₆ alkyl, C₂₋₆ alkenyl, and C₂₋₆ alkynyl are each optionally substituted with one or more R⁹;
each R⁹ is independently selected from:
   halogen, —R¹⁰, —OR¹⁰, —SR¹⁰, —N(R¹⁰)₂, —C(O)R¹⁰, —C(O)N(R¹⁰)₂, —N(R¹⁰)C(O)R¹⁰, —N(R¹⁰)C(O)N(R¹⁰)₂, —OC(O)N(R¹⁰)₂, —N(R¹⁰)C(O)OR¹⁰, —C(O)OR¹⁰, —OC(O)R¹⁰, —OC(O)₂R¹⁰, —S(O)R¹⁰, —S(O)₂R¹⁰, —NO₂, ═O, ═S, ═N(R¹⁰), and —CN; and
   C₁₋₃ alkyl, C₂₋₃ alkenyl, and C₂₋₃ alkynyl, each of which is optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —N(R¹⁰)C(O)N(R¹⁰)₂, —OC(O)N(R¹⁰)₂, —N(R¹⁰)C(O)OR¹⁰, —C(O)OR¹⁰, —OC(O)R¹⁰, —S(O)R¹⁰, —S(O)₂R¹⁰, —NO₂, ═O, ═S, ═N(R¹⁰), and —CN; and
   C₃₋₁₀ carbocycle and 3- to 10-membered heterocycle, each of which is optionally substituted with one or more substituents independently selected from halogen, —R¹⁰, —OR¹⁰, —SR¹⁰, —N(R¹⁰)₂, —C(O)R¹⁰, —C(O)N(R¹⁰)₂, —N(R¹⁰)C(O)R¹⁰, —N(R¹⁰)C(O)N(R¹⁰)₂, —OC(O)N(R¹⁰)₂, —N(R¹⁰)C(O)OR¹⁰, —C(O)OR¹⁰, —OC(O)R¹⁰, —S(O)R¹⁰, —S(O)₂R¹⁰, —NO₂, ═O, ═S, ═N(R¹⁰), and —CN; and

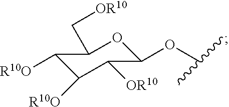

each R¹⁰ is independently selected from:
   hydrogen; and
   C₁₋₆ alkyl, C₂₋₆ alkenyl, and C₂₋₆ alkynyl, each of which is optionally substituted with one or more substituents independently selected from halogen, —CN, —OH, —SH, —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₃₋₁₀ carbocycle, 3- to 10-membered heterocycle; and
   C₃₋₁₀ carbocycle, and 3- to 10-membered heterocycle, each of which is optionally substituted with one or more substituents independently selected from halogen, —CN, —OH, —SH, —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₁₋₆ alkyl, C₂₋₆ alkenyl, C₂₋₆ alkynyl, C₃₋₁₀ carbocycle, 3- to 10-membered heterocycle, and C₁₋₆ haloalkyl;
W is selected from:
   C₁₋₆ alkylene, C₂₋₆ alkenylene, and C₂₋₆ alkynylene, each of which is optionally substituted with one or more substituents independently selected from halogen, —CN, —OH, —SH, —NO₂, —NH₂, ═O, ═S, —O—C₁₋₆ alkyl, —S—C₁₋₆ alkyl, —N(C₁₋₆ alkyl)₂, and —NH(C₁₋₆ alkyl), C₃₋₁₀ carbocycle, and 3- to 10-membered heterocycle.

In some embodiments, for a compound or salt of Formula (II), X₁, X₂, X₃, and X₄ are each independently selected from C-Q-R³, CR⁶ and N. In some embodiments, X₁, X₂, and X₃ are each independently CR⁶, and X₄ is C-Q-R³. In some embodiments, X₁, X₂, and X₄ are each independently CR⁶, and X₃ is C-Q-R³. In some embodiments, one of X₁, X₂, X₃, or X₄ is N. In some embodiments, none of X₁, X₂, X₃, or X₄ is N.

In some embodiments, for a compound or salt of Formula (II), Z is selected from —O—, —S—, and —NR⁸—. In some embodiments, if Z is NH, then R⁴ is not NH₂. In some embodiments, Z is selected from —O— and —S—. In some embodiments, Z is —O—. In some embodiments, if Z is O or S, R⁴ is —OR³, and R⁵ is H, then W—R³ is additionally selected from C₁₋₆ alkyl, C₂₋₆ alkenyl, and C₂₋₆ alkynyl.

In some embodiments, for a compound or salt of Formula (II), Q is absent or selected from —W—, —O—, —OW—, —WO—, —S—, —SW—, —WS—, —NR¹⁰W—, —WNR¹⁰—, C(O)—, —C(O)N(R¹⁰)—, —N(R¹⁰)C(O)—, —C(O)O—, —OC(O)—, —N(R¹⁰)C(O)N(R¹⁰)0-, —OC(O)N(R¹⁰)—, —N(R¹⁰)C(O)O—, —S(O)—, —S(O)₂—, and —P(O)₂O—. In some embodiments, Q is absent or selected from —W—, —O—, —OW—, and —WO—. In some embodiments, Q is absent or —O—. In some embodiments, Q is absent.

In some embodiments, for a compound or salt of Formula (II), m is selected from 1, 2, 3, 4, and 5. In some embodiments, m is selected from 1 and 2. In some embodiments, m is 1.

In certain embodiments, for a compound or salt of Formula (II), R¹ is selected from hydrogen, C₁₋₆ alkyl, C₂₋₆ alkenyl, and C₂₋₆ alkynyl, each of which is optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —C(O)OR¹⁰, —OC(O)R¹⁰, —N(R¹⁰)C(O)N(R¹⁰)₂, —OC(O)N(R¹⁰)₂, —N(R¹⁰)C(O)OR¹⁰, —S(O)R¹⁰, —S(O)₂R¹⁰, —NO₂, ═O, ═S, ═N(R¹⁰), —CN, C₃₋₁₀ carbocycle and 3- to 10-membered heterocycle, wherein the C₃₋₁₀ carbocycle and 3- to 10-membered heterocycle are each optionally substituted with one or more R⁹; and C₃₋₁₀ carbocycle and 3- to 10-membered heterocycle, each of which is optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —N(R¹⁰)C(O)N(R¹⁰)₂, —OC(O)N(R¹⁰)₂, —N(R¹⁰)C(O)OR¹⁰, —C(O)OR¹⁰, —OC(O)R¹⁰, —S(O)R¹⁰, —S(O)₂R¹⁰, —NO₂, ═O, ═S, ═N(R¹⁰), —CN, C₁₋₆ alkyl, C₂₋₆ alkenyl, and C₂₋₆ alkynyl, wherein C₁₋₆ alkyl, C₂₋₆ alkenyl, and C₂₋₆ alkynyl are each optionally substituted with one or more R⁹. In some embodiments, R¹ is selected from: hydrogen; C₁₋₆ alkyl, C₂₋₆ alkenyl, and C₂₋₆ alkynyl, each of which is optionally substituted with one or more substituents independently selected from halogen, —CN, —OH, —SH, —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₃₋₁₀ carbocycle, 3- to 10-membered heterocycle; and C₃₋₆ carbocycle, and 3- to 6-membered heterocycle, each of which is optionally substituted with one or more substituents independently selected from halogen, —CN, —OH, —SH, —NO₂, —NH₂, ═O, ═S, —O—C₁₋₆ alkyl, —S—C₁₋₆ alkyl, —N(C₁₋₆ alkyl)₂, and —NH(C₁₋₆ alkyl). In some embodiments, R¹ is selected from hydrogen and C₁₋₃ alkyl.

In certain embodiments, for a compound or salt of Formula (II), R² is selected from hydrogen, halogen, —OR¹⁰, —SR¹⁰, —N(R¹⁰)₂, —C(O)R¹⁰, —C(O)N(R¹⁰)₂, —N(R¹⁰)C(O)R¹⁰, —N(R¹⁰)C(O)N(R¹⁰)₂, —OC(O)N(R¹⁰)₂, —N(R¹⁰)C(O)OR¹⁰, —C(O)OR¹⁰, —OC(O)R¹⁰, —S(O)R¹⁰, —S(O)₂R¹⁰, —NO₂, and —CN; C₁₋₆ alkyl, C₂₋₆ alkenyl, and C₂₋₆ alkynyl, each of which is optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —C(O)OR¹⁰, —OC(O)R¹⁰, —N(R¹⁰)C(O)N(R¹⁰)₂, —OC(O)N(R¹⁰)₂, —N(R¹⁰)C(O)OR¹⁰, —S(O)R¹⁰, —S(O)₂R¹⁰, —NO₂, ═O, ═S, ═N(R¹⁰), —CN, C₃₋₁₀ carbocycle and 3- to 10-membered heterocycle, wherein the C₃₋₁₀ carbocycle and 3- to 10-membered heterocycle are each optionally substituted with one or more R⁹; and C₃₋₆ carbocycle and 3- to 6-membered heterocycle, each of which is optionally substituted with one or more substituents independently selected from halogen, —OH, —SR¹⁰, —N(R¹⁰)₂, —C(O)R¹⁰, —C(O)N(R¹⁰)₂, —N(R¹⁰)C(O)R¹⁰, —N(R¹⁰)C(O)N(R¹⁰)₂, —OC(O)N(R¹⁰)₂, —N(R¹⁰)C(O)OR¹⁰, —C(O)OR¹⁰, —OC(O)R¹⁰, —S(O)R¹⁰, —S(O)₂R¹⁰, —NO₂, ═O, ═S, ═N(R¹⁰), —CN, C₁₋₆ alkyl, C₂₋₆ alkenyl, and C₂₋₆ alkynyl, wherein C₁₋₆ alkyl, C₂₋₆ alkenyl, and C₂₋₆ alkynyl are each optionally substituted with one or more R⁹. In some embodiments, R² is selected from: hydrogen; halogen, —CN, —OH, —SH, —NO₂, —NH₂, ═O, ═S, —O—C₁₋₆ alkyl, —S—C₁₋₆ alkyl, —N(C₁₋₆ alkyl)₂, —NH(C₁₋₆ alkyl); C₁₋₆ alkyl, C₂₋₆ alkenyl, and C₂₋₆ alkynyl, each of which is optionally substituted with one or more substituents independently selected from halogen, —CN, —OH, —SH, —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₃₋₁₀ carbocycle, 3- to 10-membered heterocycle; and C₃₋₆ carbocycle, and 3- to 6-membered heterocycle, each of which is optionally substituted with one or more substituents independently selected from halogen, —CN, —OH, —SH, —NO₂, —NH₂, ═O, ═S, —S—C₁₋₆ alkyl, —N(C₁₋₆ alkyl)₂, and —NH(C₁₋₆ alkyl). In some embodiments, R² is selected from: hydrogen; halogen, —CN, —OH, —SH, —NO₂, —NH₂, ═O, ═S, —O—C₁₋₆ alkyl, —S—C₁₋₆ alkyl, —N(C₁₋₆ alkyl)₂, —NH(C₁₋₆ alkyl); C₁₋₆ alkyl optionally substituted with one or more substituents independently selected from halogen, —CN, —OH, —SH, —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₃₋₁₀ carbocycle, 3- to 10-membered heterocycle; and C₆ carbocycle, and 5- to 6-membered heterocycle, each of which is optionally substituted with one or more substituents independently selected from halogen. In some embodiments, R² is selected from hydrogen; halogen, —CN, —OH, —SH, —NO₂, —NH₂, ═O, ═S, —O—C₁₋₆ alkyl, —S—C₁₋₆ alkyl, —N(C₁₋₆ alkyl)₂, —NH(C₁₋₆ alkyl); C₁₋₆ alkyl optionally substituted with one or more substituents independently selected from halogen, —CN, —OH, —SH, —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₃₋₁₀ carbocycle, and 3- to 10-membered heterocycle. In some embodiments, R² is selected from hydrogen, halogen, and C₁₋₃ alkyl.

In certain embodiments, for a compound or salt of Formula (II), hydrogen; halogen, —OR¹⁰, —SR¹⁰, —N(R¹⁰)₂, —C(O)R¹⁰, —C(O)N(R¹⁰)₂, —N(R¹⁰)C(O)R¹⁰, —N(R¹⁰)C(O)N(R¹⁰)₂, —OC(O)N(R¹⁰)₂, —N(R¹⁰)C(O)OR¹⁰, —C(O)OR¹⁰, —OC(O)R¹⁰, —S(O)R¹⁰, —S(O)₂R¹⁰, —NO₂, ═O, ═S, ═N(R¹⁰), and —CN; C₁₋₆ alkyl, C₂₋₆ alkenyl, and C₂₋₆ alkynyl, each of which is optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —C(O)OR¹⁰, —OC(O)R¹⁰, —N(R¹⁰)C(O)N(R¹⁰)₂, —OC(O)N(R¹⁰)₂, —N(R¹⁰)C(O)OR¹⁰, —S(O)R¹⁰, —S(O)₂R¹⁰, —NO₂, ═O, ═S, ═N(R¹⁰), —CN, C₃₋₁₀ carbocycle and 3- to 10-membered heterocycle, wherein the C₃₋₁₀ carbocycle and 3- to 10-membered heterocycle are each optionally substituted with one or more R⁹; and C₃₋₁₀ carbocycle and 3- to 10-membered heterocycle, each of which is optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —N(R¹⁰)C(O)N(R¹⁰)₂, —OC(O)N(R¹⁰)₂, —N(R¹⁰)C(O)OR¹⁰, —C(O)OR¹⁰, —OC(O)R¹⁰, —S(O)R¹⁰, —S(O)₂R¹⁰, —NO₂, ═O, ═S, ═N(R¹⁰), —CN, C₁₋₆ alkyl, C₂₋₆ alkenyl, and C₂₋₆ alkynyl, wherein C₁₋₆ alkyl, C₂₋₆ alkenyl, and C₂₋₆ alkynyl are each optionally substituted with one or more R⁹.

In certain embodiments, for a compound or salt of Formula (II), R³ is a C₃₋₆ carbocycle optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —N(R¹⁰)C(O)N(R¹⁰)₂, —OC(O)N(R¹⁰)₂, —N(R¹⁰)C(O)OR¹⁰, —C(O)OR¹⁰, —OC(O)R¹⁰, —S(O)R¹⁰, —S(O)₂R¹⁰, —NO₂, ═O, ═S, ═N(R¹⁰), —CN, C₁₋₆ alkyl, C₂₋₆ alkenyl, and C₂₋₆ alkynyl, wherein C₁₋₆ alkyl, C₂₋₆ alkenyl, and C₂₋₆ alkynyl are each optionally substituted with one or more R⁹. In some embodiments, R³ is a C₆ carbocycle optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —N(R¹⁰)C(O)N(R¹⁰)₂, —OC(O)N(R¹⁰)₂, —N(R¹⁰)C(O)OR¹⁰, —C(O)OR¹⁰, —OC(O)R¹⁰, —S(O)R¹⁰, —S(O)₂R¹⁰, —NO₂, ═O, ═S, ═N(R¹⁰), —CN, C₁₋₆ alkyl, C₂₋₆ alkenyl, and C₂₋₆ alkynyl, wherein C₁₋₆ alkyl, C₂₋₆ alkenyl, and C₂₋₆ alkynyl are each optionally substituted with one or more R⁹. In some embodiments, R³ is phenyl optionally substituted with one or more substituents independently selected from halogen, —CN, —OH, —SH, —NO₂, —NH₂, —O—C₁₋₆ alkyl, —S—C₁₋₆ alkyl, —N(C₁₋₆ alkyl)₂, and —NH(C₁₋₆ alkyl).

In certain embodiments, for a compound or salt of Formula (II), R³ is a 3- to 6-membered heterocycle optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —N(R¹⁰)C(O)N(R¹⁰)₂, —OC(O)N(R¹⁰)₂, —N(R¹⁰)C(O)OR¹⁰, —C(O)OR¹⁰, —OC(O)R¹⁰, —S(O)R¹⁰, —S(O)₂R¹⁰, —NO₂, ═O, ═S, ═N(R¹⁰), —CN, C₁₋₆ alkyl, C₂₋₆ alkenyl, and C₂₋₆ alkynyl, wherein C₁₋₆ alkyl, C₂₋₆ alkenyl, and C₂₋₆ alkynyl are each optionally substituted with one or more R⁹. In some embodiments, R³ is a 5- to 6-membered heterocycle optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —N(R¹⁰)C(O)N(R¹⁰)₂, —OC(O)N(R¹⁰)₂, —N(R¹⁰)C(O)OR¹⁰, —C(O)OR¹⁰, —OC(O)R¹⁰, —S(O)R¹⁰, —S(O)₂R¹⁰, —NO₂, ═O, ═S, ═N(R¹⁰), —CN, C₁₋₆ alkyl, C₂₋₆ alkenyl, and C₂₋₆ alkynyl, wherein C₁₋₆ alkyl, C₂₋₆ alkenyl, and C₂₋₆ alkynyl are each optionally substituted with one or more R⁹. In some embodiments, R³ is a 6-membered heterocycle optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —N(R¹⁰)C(O)N(R¹⁰)₂, —OC(O)N(R¹⁰)₂, —N(R¹⁰)C(O)OR¹⁰, —C(O)OR¹⁰, —OC(O)R¹⁰, —S(O)R¹⁰, —S(O)₂R¹⁰, —NO₂, ═O, ═S, ═N(R¹⁰), —CN, C₁₋₆ alkyl, C₂₋₆ alkenyl, and C₂₋₆ alkynyl, wherein C₁₋₆ alkyl, C₂₋₆ alkenyl, and C₂₋₆ alkynyl are each optionally substituted with one or more R⁹. In some embodiments, R³ is 6-membered heterocycle optionally substituted with one or more substituents independently selected from halogen, —CN, —OH, —SH, —NO₂, —NH₂, —O—C₁₋₆ alkyl, —S—C₁₋₆ alkyl, —N(C₁₋₆ alkyl)₂, and —NH(C₁₋₆ alkyl).

In certain embodiments, for a compound or salt of Formula (II), R³ is a 5-membered heterocycle optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —N(R¹⁰)C(O)N(R¹⁰)₂, —OC(O)N(R¹⁰)₂, —N(R¹⁰)C(O)OR¹⁰, —C(O)OR¹⁰, —OC(O)R¹⁰, —S(O)R¹⁰, —S(O)₂R¹⁰, —NO₂, ═O, ═S, ═N(R¹⁰), —CN, C₁₋₆ alkyl, C₂₋₆ alkenyl, and C₂₋₆ alkynyl, wherein C₁₋₆ alkyl, C₂₋₆ alkenyl, and C₂₋₆ alkynyl are each optionally substituted with one or more R⁹. In some embodiments, R³ is a 5-membered heterocycle optionally substituted with one or more substituents independently selected from halogen, —CN, —OH, ═O, ═S, ═NH, —SH, —NO₂, —NH₂, —O—C₁₋₆ alkyl, —S—C₁₋₆ alkyl, —N(C₁₋₆ alkyl)₂, —NH(C₁₋₆ alkyl), and C₁₋₆ alkyl. In some embodiments, R³ is a 5-membered heterocycle is selected from isoxazole, oxazole, thiadiazole, oxadiazole, pyrazole, tetrazole, and thiazole, any of which is optionally substituted with one or more R⁹. In some embodiments, R³ is a 5-membered heterocycle is selected tetrazole and oxadiazole. In some embodiments, R³ is a 5-membered heterocycle selected from isothiazol-3-ol, isoxazole-3-ol, squaric acid, thiazolidinedione, oxazolidinedione, tetrazole, 1,2,4-oxadiazol-5(4H)-one, 1,2,4-thiadiazol-5(4H)-one, 1,2,4-oxadiazole-5(4H)-thione, 2-methyl-2,4-dihydro-3H-1,2,4-triazole-3-thione, 3H-1,2,3,5-oxathiadiazole 2-oxide, 3-(methylsulfonyl)-4H-1,2,4-triazole, or 1,4-dihydro-5H-tetrazol-5-one, 3-hydroxyquinolin-2-one, and tetramic acid, any of which is optionally substituted with one or more R⁹. In some embodiments, R³ is a 5-membered heterocycle is selected from

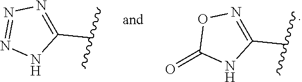 [SMILES: *c1nnn[nH]1 ; *c1noc(=O)[nH]1]

In some embodiments, R³ is selected from

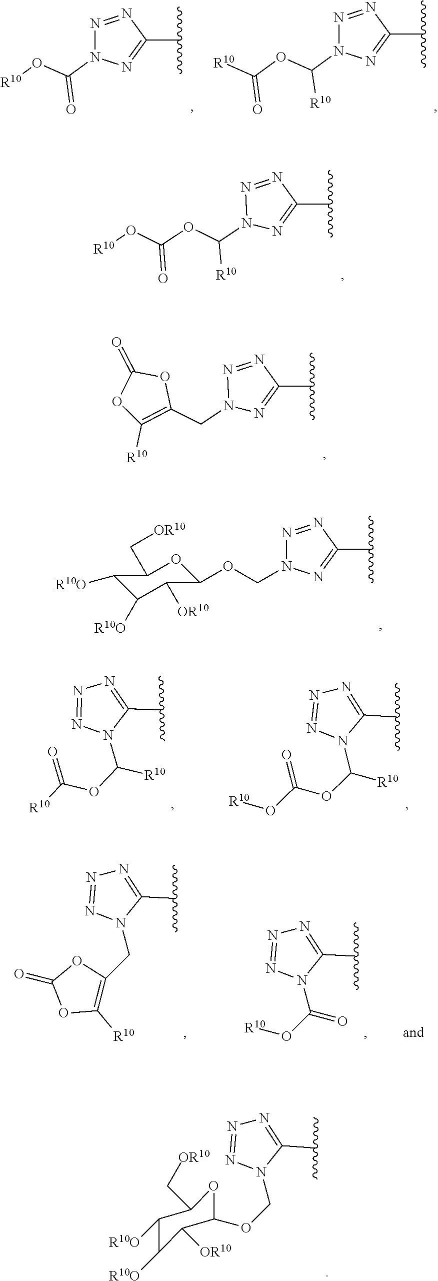

In some embodiments, Q is —O— and R³ is tetrazole.

In certain embodiments, for a compound or salt of Formula (II), Q is —W—; R³ is additionally selected from halogen, —OR¹⁰, —SR¹⁰, —N(R¹⁰)₂, —C(O)R¹⁰, —C(O)N(R¹⁰)₂, —N(R¹⁰)C(O)R¹⁰, —N(R¹⁰)C(O)N(R¹⁰)₂, —OC(O)N(R¹⁰)₂, —N(R¹⁰)C(O)OR¹⁰, —C(O)OR¹⁰, —OC(O)R¹⁰, —S(O)R¹⁰, —S(O)₂R¹⁰, —NO₂, —CN; and —W— is C₁₋₆ alkylene optionally substituted with one or more substituents independently selected from halogen, —CN, —OH, —SH, —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₃₋₁₀ carbocycle, and 3- to 10-membered heterocycle. In some embodiments, Q is —W—; and R³ is additionally selected from halogen, —CN, —OH, —SH, —NO₂, —NH₂, —O—C₁₋₆ alkyl, —S—C₁₋₆ alkyl, —N(C₁₋₆ alkyl)₂, and —NH(C₁₋₆ alkyl), and —W— is C₁₋₆ alkylene optionally substituted with one or more substituents independently selected from halogen, —CN, —OH, —SH, —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₃₋₁₀ carbocycle, and 3- to 10-membered heterocycle. In some embodiments, Q is —W—; and R³ is additionally selected from —OH, —SH, —NH₂, —O—C₁₋₆ alkyl, —S—C₁₋₆ alkyl, —N(C₁₋₆ alkyl)₂, and —NH(C₁₋₆ alkyl); and —W— is C₁ alkylene optionally substituted with one or more substituents independently selected from halogen, —CN, —OH, —SH, —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₃₋₁₀ carbocycle, and 3- to 10-membered heterocycle. In some embodiments, Q is —W—; and R³ is additionally selected from —OH, —SH, —NH₂, —O—C₁₋₆ alkyl, —S—C₁₋₆ alkyl, —N(C₁₋₆ alkyl)₂, and —NH(C₁₋₆ alkyl); and —W— is —C(═O)—.

In certain embodiments, for a compound or salt of Formula (II), halogen, —OR⁷, —SR⁷, —N(R⁷)₂, —C(O)R⁷, —C(O)N(R⁷)₂, —N(R⁷)C(O)R⁷, —N(R⁷)C(O)N(R⁷)₂, —OC(O)N(R⁷)₂, —N(R⁷)C(O)OR⁷, —C(O)OR⁷, —OC(O)R⁷, —S(O)R⁷, —S(O)₂R⁷, —NO₂, and —CN; C₁₋₆ alkyl, C₂₋₆ alkenyl, and C₂₋₆ alkynyl, each of which is optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —C(O)OR¹⁰, —OC(O)R¹⁰, —N(R¹⁰)C(O)N(R¹⁰)₂, —OC(O)N(R⁰)₂, —N(R¹⁰)C(O)OR¹⁰, —S(O)R¹⁰, —S(O)₂R¹⁰, —NO₂, ═O, ═S, ═N(R¹⁰), —CN, C₃₋₁₀ carbocycle and 3- to 10-membered heterocycle, wherein the C₃₋₁₀ carbocycle and 3- to 10-membered heterocycle are each optionally substituted with one or more R⁹; and C₃₋₁₀ carbocycle and 3- to 10-membered heterocycle, each of which is optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —N(R¹⁰)C(O)N(R¹⁰)₂, —OC(O)N(R¹⁰)₂, —N(R¹⁰)C(O)OR¹⁰, —C(O)OR¹⁰, —OC(O)R¹⁰, —S(O)R¹⁰, —S(O)₂R¹⁰, —NO₂, ═O, ═S, ═N(R¹⁰), —CN, C₁₋₆ alkyl, C₂₋₆ alkenyl, and C₂₋₆ alkynyl, wherein C₁₋₆ alkyl, C₂₋₆ alkenyl, and C₂₋₆ alkynyl are each optionally substituted with one or more R⁹. In some embodiments, each R⁴ is independently selected from: halogen, —OR⁷, —SR⁷, —N(R⁷)₂, —NO₂, and —CN; C₁₋₆ alkyl, C₂₋₆ alkenyl, and C₂₋₆ alkynyl, each of which is optionally substituted with one or more substituents independently selected from halogen, —CN, —OH, —SH, —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₃₋₁₀ carbocycle, 3- to 10-membered heterocycle; and C₃₋₆ carbocycle, and 3- to 6-membered heterocycle, each of which is optionally substituted with one or more substituents independently selected from halogen, —CN, —OH, —SH, —NO₂, —NH₂, ═O, ═S, —O—C₁₋₆ alkyl, —S—C₁₋₆ alkyl, —N(C₁₋₆ alkyl)₂, and —NH(C₁₋₆ alkyl). In some embodiments, R⁴ is independently selected from —OR⁷, —SR⁷, and —N(R⁷)₂. In some embodiments, m is 1 and R⁴ is —OR⁷.

In certain embodiments, for a compound or salt of Formula (II), each R⁷ is selected from: hydrogen; C₁₋₆ alkyl, C₂₋₆ alkenyl, and C₂₋₆ alkynyl, each of which is optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —C(O)OR¹⁰, —OC(O)R¹⁰, —N(R¹⁰)C(O)N(R¹⁰)₂, —OC(O)N(R¹⁰)₂, —N(R¹⁰)C(O)OR¹⁰, —S(O)R¹⁰, —S(O)₂R¹⁰, —NO₂, ═O, ═S, ═N(R¹⁰), —CN, C₃₋₁₀ carbocycle and 3- to 10-membered heterocycle, wherein the C₃₋₁₀ carbocycle and 3- to 10-membered heterocycle are each optionally substituted with one or more R⁹; and C₃₋₁₀ carbocycle and 3- to 10-membered heterocycle, each of which is optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —N(R¹⁰)C(O)N(R¹⁰)₂, —OC(O)N(R¹⁰)₂, —N(R¹⁰)C(O)OR¹⁰, —C(O)OR¹⁰, —OC(O)R¹⁰, —S(O)R¹⁰, —S(O)₂R¹⁰, —NO₂, ═O, ═S, ═N(R¹⁰), —CN, C₁₋₆ alkyl, C₂₋₆ alkenyl, and C₂₋₆ alkynyl, wherein C₁₋₆ alkyl, C₂₋₆ alkenyl, and C₂₋₆ alkynyl are each optionally substituted with one or more R⁹. In some embodiments, each R⁷ is selected from: C₁₋₆ alkyl, C₂₋₆ alkenyl, and C₂₋₆ alkynyl, each of which is optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —C(O)OR¹⁰, —OC(O)R¹⁰, —N(R¹⁰)C(O)N(R¹⁰)₂, —OC(O)N(R¹⁰)₂, —N(R¹⁰)C(O)OR¹⁰, —S(O)R¹⁰, —S(O)₂R¹⁰, —NO₂, ═O, ═S, ═N(R¹⁰), —CN, C₃₋₁₀ carbocycle and 3- to 10-membered heterocycle, wherein the C₃₋₁₀ carbocycle and 3- to 10-membered heterocycle are each optionally substituted with one or more R⁹; and C₃₋₁₀ carbocycle and 3- to 10-membered heterocycle, each of which is optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —N(R¹⁰)C(O)N(R¹⁰)₂, —OC(O)N(R¹⁰)₂, —N(R¹⁰)C(O)OR¹⁰, —C(O)OR¹⁰, —OC(O)R¹⁰, —S(O)R¹⁰, —S(O)₂R¹⁰, —NO₂, ═O, ═S, ═N(R¹⁰), —CN, C₁₋₆ alkyl, C₂₋₆ alkenyl, and C₂₋₆ alkynyl, wherein C₁₋₆ alkyl, C₂₋₆ alkenyl, and C₂₋₆ alkynyl are each optionally substituted with one or more R⁹. In some embodiments, each R⁷ is selected from: C₁₋₆ alkyl optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —C(O)OR¹⁰, —OC(O)R¹⁰, —N(R¹⁰)C(O)N(R¹⁰)₂, —OC(O)N(R¹⁰)₂, —N(R¹⁰)C(O)OR¹⁰, —S(O)R¹⁰, —S(O)₂R¹⁰, —NO₂, ═O, ═S, ═N(R¹⁰), —CN, C₃₋₁₀ carbocycle and 3- to 10-membered heterocycle, wherein the C₃₋₁₀ carbocycle and 3- to 10-membered heterocycle are each optionally substituted with one or more R⁹; and C₃₋₁₀ carbocycle and 3- to 10-membered heterocycle, each of which is optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —N(R¹⁰)C(O)N(R¹⁰)₂, —OC(O)N(R¹⁰)₂, —N(R¹⁰)C(O)OR¹⁰, —C(O)OR¹⁰, —OC(O)R¹⁰, —S(O)R¹⁰, —S(O)₂R¹⁰, —NO₂, ═O, ═S, ═N(R¹⁰), —CN, C₁₋₆ alkyl, C₂₋₆ alkenyl, and C₂₋₆ alkynyl, wherein C₁₋₆ alkyl, C₂₋₆ alkenyl, and C₂₋₆ alkynyl are each optionally substituted with one or more R⁹. In some embodiments, R⁷ is selected from: C₁₋₆ alkyl optionally substituted with one or more substituents independently selected from C₃₋₁₀ carbocycle and 3- to 10-membered heterocycle, wherein the C₃₋₁₀ carbocycle and 3- to 10-membered heterocycle are each optionally substituted with one or more R⁹; and C₃₋₁₀ carbocycle and 3- to 10-membered heterocycle, each of which is optionally substituted with one or more substituents independently selected from halogen, —OR¹⁰, —SR¹⁰, —N(R¹⁰)₂, —CN, and C₁₋₆ alkyl optionally substituted with one or more R⁹. In some embodiments, R⁷ is C₁₋₆ alkyl substituted with one or more substituents independently selected from C₃₋₁₀ carbocycle and 3- to 10-membered heterocycle, wherein the C₃₋₁₀ carbocycle and 3- to 10-membered heterocycle are each optionally substituted with one or more substituents independently selected from halogen, —CN, —OH, —SH, —NO₂, —NH₂, —O—C₁₋₆ alkyl, —S—C₁₋₆ alkyl, —N(C₁₋₆ alkyl)₂, and —NH(C₁₋₆ alkyl). In some embodiments, R⁷ is C₁₋₆ alkyl substituted with one or more substituents independently selected from C₃₋₆ carbocycle and 3- to 6-membered heterocycle, wherein the C₃₋₆ carbocycle and 3- to 6-membered heterocycle are each optionally substituted with one or more substituents independently selected from halogen, —CN, —OH, —SH, —NO₂, —NH₂, —O—C₁₋₆ alkyl, —S—C₁₋₆ alkyl, —N(C₁₋₆ alkyl)₂, and —NH(C₁₋₆ alkyl). R⁷ is selected from C₃₋₁₀ carbocycle and 3- to 10-membered heterocycle, each of which is optionally substituted with one or more substituents independently selected from halogen, —CN, —OH, —SH, —NO₂, —NH₂, —O—C₁₋₆ alkyl, —S—C₁₋₆ alkyl, —N(C₁₋₆ alkyl)₂, and —NH(C₁₋₆ alkyl).

In certain embodiments, for a compound or salt of Formula (II), R⁶ is selected from hydrogen, halogen, —OR¹⁰, —SR¹⁰, —N(R¹⁰)₂, —NO₂, and —CN; and C₁₋₆ alkyl optionally substituted with one or more substituents independently selected from halogen, —OR¹⁰, —SR¹⁰, —N(R¹⁰)₂, —NO₂, and —CN. In some embodiments, R⁶ is selected from hydrogen, halogen, —CN, —OH, —SH, —NO₂, —NH₂, —O—C₁₋₆ alkyl, —S—C₁₋₆ alkyl, —N(C₁₋₆ alkyl)₂, and —NH(C₁₋₆ alkyl). In some embodiments, R⁶ is selected from hydrogen and halogen.

In certain embodiments, for a compound or salt of Formula (II), Z is —NR⁸—. In some embodiments, R⁸ is selected from hydrogen; C₁₋₆ alkyl, C₂₋₆ alkenyl, and C₂₋₆ alkynyl, each of which is optionally substituted with one or more substituents independently selected from halogen, —SR¹⁰, —N(R¹⁰)₂, —C(O)R¹⁰, —C(O)N(R¹⁰)₂, —N(R¹⁰)C(O)R¹⁰, —C(O)OR¹⁰, —OC(O)R¹⁰, —N(R¹⁰)C(O)N(R¹⁰)₂, —OC(O)N(R¹⁰)₂, —N(R¹⁰)C(O)OR¹⁰, —S(O)R¹⁰, —S(O)₂R¹⁰, —NO₂, ═O, ═S, ═N(R¹⁰), —CN, C₃₋₁₀ carbocycle and 3- to 10-membered heterocycle, wherein the C₃₋₁₀ carbocycle and 3- to 10-membered heterocycle are each optionally substituted with one or more R⁹; and C₃₋₁₀ carbocycle and 3- to 10-membered heterocycle, each of which is optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —N(R¹⁰)C(O)N(R¹⁰)₂, —OC(O)N(R¹⁰)₂, —N(R¹⁰)C(O)OR¹⁰, —C(O)OR¹⁰, —OC(O)R¹⁰, —S(O)R¹⁰, —S(O)₂R¹⁰, —NO₂, ═O, ═S, ═N(R¹⁰), —CN, C₁₋₆ alkyl, C₂₋₆ alkenyl, and C₂₋₆ alkynyl, wherein C₁₋₆ alkyl, C₂₋₆ alkenyl, and C₂₋₆ alkynyl are each optionally substituted with one or more R⁹. In some embodiments, R⁸ is selected from hydrogen. In some embodiments, R⁸ is selected from C₁₋₆ alkyl, C₂₋₆ alkenyl, and C₂₋₆ alkynyl, each of which is optionally substituted with one or more substituents independently selected from halogen, —SR¹⁰, —N(R¹⁰)₂, —C(O)R¹⁰, —C(O)N(R¹⁰)₂, —N(R¹⁰)C(O)R¹⁰, —C(O)OR¹⁰, —OC(O)R¹⁰, —N(R¹⁰)C(O)N(R¹⁰)₂, —OC(O)N(R¹⁰)₂, —N(R¹⁰)C(O)OR¹⁰, —S(O)R¹⁰, —S(O)₂R¹⁰, —NO₂, ═O, ═S, ═N(R¹⁰), —CN, C₃₋₁₀ carbocycle and 3- to 10-membered heterocycle, wherein the C₃₋₁₀ carbocycle and 3- to 10-membered heterocycle are each optionally substituted with one or more R⁹. In some embodiments, R⁸ is selected from C₁₋₆ alkyl, C₂₋₆ alkenyl, and C₂₋₆ alkynyl, each of which is optionally substituted with one or more substituents independently selected from halogen, —CN, —SH, —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₃₋₁₀ carbocycle, 3- to 10-membered heterocycle. In some embodiments, R⁸ is selected from C₁₋₆ alkyl optionally substituted with one or more substituents independently selected from halogen, —CN, —SH, —NO₂, —NH₂, —O—C₁₋₆ alkyl, —S—C₁₋₆ alkyl, —N(C₁₋₆ alkyl)₂, and —NH(C₁₋₆ alkyl). In some embodiments, R⁸ is selected from C₁₋₆ alkyl. In some embodiments, R⁸ is selected from C₃₋₁₀ carbocycle optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —N(R¹⁰)C(O)N(R¹⁰)₂, —OC(O)N(R¹⁰)₂, —N(R¹⁰)C(O)OR¹⁰, —C(O)OR¹⁰, —OC(O)R¹⁰, —S(O)R¹⁰, —S(O)₂R¹⁰, —NO₂, ═O, ═S, ═N(R¹⁰), —CN, C₁₋₆ alkyl, C₂₋₆ alkenyl, and C₂₋₆ alkynyl, wherein C₁₋₆ alkyl, C₂₋₆ alkenyl, and C₂₋₆ alkynyl are each optionally substituted with one or more R⁹. In some embodiments, R⁸ is selected from C₃₋₁₀ carbocycle optionally substituted with one or more substituents independently selected from halogen, —CN, —OH, —SH, —NO₂, —NH₂, —O—C₁₋₆ alkyl, —S—C₁₋₆ alkyl, —N(C₁₋₆ alkyl)₂, and —NH(C₁₋₆ alkyl). In some embodiments, R⁸ is selected from 3- to 10-membered heterocycle optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —N(R¹⁰)C(O)N(R¹⁰)₂, —OC(O)N(R¹⁰)₂, —N(R¹⁰)C(O)OR¹⁰, —C(O)OR¹⁰, —OC(O)R¹⁰, —S(O)R¹⁰, —S(O)₂R¹⁰, —NO₂, ═O, ═S, ═N(R¹⁰), —CN, C₁₋₆ alkyl, C₂₋₆ alkenyl, and C₂₋₆ alkynyl, wherein C₁₋₆ alkyl, C₂₋₆ alkenyl, and C₂₋₆ alkynyl are each optionally substituted with one or more R⁹. In some embodiments, R⁸ is selected from 3- to 10-membered heterocycle optionally substituted with one or more substituents independently selected from halogen, —CN, —OH, —SH, —NO₂, —NH₂, —O—C₁₋₆ alkyl, —S—C₁₋₆ alkyl, —N(C₁₋₆ alkyl)₂, and —NH(C₁₋₆ alkyl). In some embodiments, R⁸ is hydrogen.

In certain embodiments, for a compound or salt of Formula (II), each R⁹ is selected from: halogen, —R¹⁰, —OR¹⁰, —SR¹⁰, —N(R¹⁰)₂, —C(O)R¹⁰, —C(O)N(R¹⁰)₂, —N(R¹⁰)C(O)R¹⁰, —N(R¹⁰)C(O)N(R¹⁰)₂, —OC(O)N(R¹⁰)₂, —N(R¹⁰)C(O)OR¹⁰, —C(O)OR¹⁰, —OC(O)R¹⁰, —OC(O)₂R¹⁰, —S(O)R¹⁰, —S(O)₂R¹⁰, —NO₂, ═O, ═S, ═N(R¹⁰), and —CN; C₁₋₃ alkyl, C₂₋₃ alkenyl, and C₂₋₃ alkynyl, each of which is optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —N(R¹⁰)C(O)N(R¹⁰)₂, —OC(O)N(R¹⁰)₂, —N(R¹⁰)C(O)OR¹⁰, —C(O)OR¹⁰, —OC(O)R¹⁰, —S(O)R¹⁰, —S(O)₂R¹⁰, —NO₂, ═O, ═S, ═N(R¹⁰), and —CN; C₃₋₁₀ carbocycle and 3- to 10-membered heterocycle, each of which is optionally substituted with one or more substituents independently selected from halogen, —R¹⁰, —OR¹⁰, —SR¹⁰, —N(R¹⁰)₂, —C(O)R¹⁰, —C(O)N(R¹⁰)₂, —N(R¹⁰)C(O)R¹⁰, —N(R¹⁰)C(O)N(R¹⁰)₂, —OC(O)N(R¹⁰)₂, —N(R¹⁰)C(O)OR¹⁰, —C(O)OR¹⁰, —OC(O)R¹⁰, —S(O)R¹⁰, —S(O)₂R¹⁰, —NO₂, ═O, ═S, ═N(R¹⁰), and —CN; and

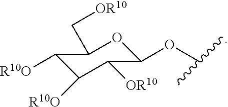

In some embodiments, each R⁹ is selected from: halogen, —CN, —OH, —SH, —NO₂, —NH₂, —O—C₁₋₆ alkyl, —S—C₁₋₆ alkyl, —N(C₁₋₆ alkyl)₂, and —NH(C₁₋₆ alkyl); and C₁₋₃ alkyl optionally substituted with one or more substituents independently selected from halogen, —CN, —OH, —SH, —NO₂, —NH₂, —O—C₁₋₆ alkyl, —S—C₁₋₆ alkyl, —N(C₁₋₆ alkyl)₂, and —NH(C₁₋₆ alkyl).

In certain embodiments, for a compound or salt of Formula (II), each R¹⁰ is hydrogen; C₁₋₆ alkyl, C₂₋₆ alkenyl, and C₂₋₆ alkynyl, each of which is optionally substituted with one or more substituents independently selected from halogen, —CN, —OH, —SH, —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₃₋₁₀ carbocycle, 3- to 10-membered heterocycle; and C₃₋₁₀ carbocycle, and 3- to 10-membered heterocycle, each of which is optionally substituted with one or more substituents independently selected from halogen, —CN, —OH, —SH, —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₁₋₆ alkyl, C₂₋₆ alkenyl, C₂₋₆ alkynyl, C₃₋₁₀ carbocycle, 3- to 10-membered heterocycle, and C₁₋₆ haloalkyl. In some embodiments, each R¹⁰ is hydrogen.

In certain embodiments, for a compound or salt of Formula (II), W is C₁₋₆ alkylene, C₂₋₆ alkenylene, and C₂₋₆ alkynylene, each of which is optionally substituted with one or more substituents independently selected from halogen, —CN, —OH, —SH, —NO₂, —NH₂, ═O, ═S, —O—C₁₋₆ alkyl, —S—C₁₋₆ alkyl, —N(C₁₋₆ alkyl)₂, and —NH(C₁₋₆ alkyl), C₃₋₁₀ carbocycle, and 3- to 10-membered heterocycle. In some embodiments, W is C₁₋₆ alkylene optionally substituted with one or more substituents independently selected from halogen, —CN, —OH, —SH, —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₃₋₁₀ carbocycle, and 3- to 10-membered heterocycle. In some embodiments, W is C₁₋₆ alkylene optionally substituted with one or more substituents independently selected from halogen, —CN, —OH, —SH, —NO₂, —NH₂, —O—C₁₋₆ alkyl, —S—C₁₋₆ alkyl, —N(C₁₋₆ alkyl)₂, —NH(C₁₋₆ alkyl), C₃₋₁₀ carbocycle, and 3- to 10-membered heterocycle. In some embodiments, W is C₁₋₆ alkylene optionally substituted with one or more substituents independently selected from halogen, —CN, —OH, —SH, —NO₂, —NH₂, —O—C₁₋₆ alkyl, —S—C₁₋₆ alkyl, —N(C₁₋₆ alkyl)₂, and —NH(C₁₋₆ alkyl). In some embodiments, W is C₁₋₆ alkylene optionally substituted with one or more substituents independently selected from C₃₋₁₀ carbocycle, and 3- to 10-membered heterocycle. In some embodiments, W is C₁₋₆ alkylene substituted with one or more substituents independently selected from C₃₋₆ carbocycle, and 3- to 5-membered heterocycle. In some embodiments, W is C₁₋₆ alkylene substituted with one or more substituents independently selected from C₃₋₆ carbocycle. In some embodiments, W is selected from

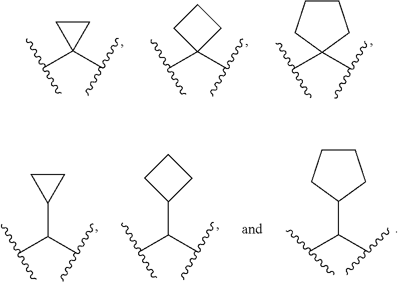 [SMILES: *C1(*)CC1 ; *C1(*)CCC1 ; *C1(*)CCCC1 ; *C(*)C1CC1 ; *C(*)C1CCC1 ; *C(*)C1CCCC1]

In some embodiments, W is C₁₋₆ alkylene.

Methods of administration of a compound or salt of Formula (I) or (II) discussed herein may be used for the treatment of cancer. In some embodiments, the treatment is an immunotherapy treatment. In some embodiments, the cancer is a solid tumor. In some embodiments, the sloid tumor is a sarcoma, carcinoma, or lymphoma. In some embodiments, the solid tumor is in the breast, brain, kidney, ovary, cervix, pancreas, thyroid, colon, bladder, prostate, liver, stomach, or lung.

In some embodiments, a compound or salt of Formula (I) or (II) discussed herein may be used to modulate G protein couple receptor 35 (GPR35). In some embodiments, the compounds disclosed herein inhibit GPR35 expresed in immune cells and promote anti-tumor immunity. In some embodiments, the compounds disclosed herein inhibit tumor cell expressed GPR35 and tumor cell growth. Such compounds are efficacious in the treatment of solid tumors alone or in combination with additional treatment agents.

### Pharmaceutical Formulations

The compositions and methods described herein may be considered useful as pharmaceutical compositions for administration to a subject in need thereof. Pharmaceutical compositions may comprise at least the a compound or salt of Formula (I) or (II) described herein and one or more pharmaceutically acceptable carriers, diluents, excipients, stabilizers, dispersing agents, suspending agents, and/or thickening agents.

Pharmaceutical compositions comprising a compound or salt of Formula (I) or (II) may be formulated using one or more physiologically-acceptable carriers comprising excipients and auxiliaries. Formulation may be modified depending upon the route of administration chosen. Pharmaceutical compositions comprising a compound, salt or conjugate may be manufactured, for example, by lyophilizing the compound, salt or conjugate, mixing, dissolving, emulsifying, encapsulating or entrapping the conjugate. The pharmaceutical compositions may also include the compounds, salts or conjugates in a free-base form or pharmaceutically-acceptable salt form.

Methods for formulation of a compound or salt of Formula (I) or (II) may include formulating any of the compounds, salts or conjugates with one or more inert, pharmaceutically-acceptable excipients or carriers to form a solid, semi-solid, or liquid composition. Solid compositions may include, for example, powders, tablets, dispersible granules and capsules, and in some aspects, the solid compositions further contain nontoxic, auxiliary substances, for example wetting or emulsifying agents, pH buffering agents, and other pharmaceutically-acceptable additives. Alternatively, the compounds, salts or conjugates may be lyophilized or in powder form for reconstitution with a suitable vehicle, e.g., sterile pyrogen-free water, before use.

Pharmaceutical compositions comprising a compound or salt of Formula (I) or (II) may comprise at least one active ingredient (e.g., a compound, salt or conjugate and other agents). The active ingredients may be entrapped in microcapsules prepared, for example, by coacervation techniques or by interfacial polymerization (e.g., hydroxymethylcellulose or gelatin microcapsules and poly-(methylmethacylate) microcapsules, respectively), in colloidal drug-delivery systems (e.g., liposomes, albumin microspheres, microemulsions, nano-particles and nanocapsules) or in macroemulsions.

The compositions and formulations may be sterilized. Sterilization may be accomplished by filtration through sterile filtration.

The compositions described herein are administered to a subject by appropriate administration routes, including but not limited to, oral, parenteral (e.g., intravenous, subcutaneous, intramuscular), intranasal, buccal, topical, rectal, or transdermal administration routes. The compositions described herein include, but are not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations, and mixed immediate and controlled release formulations.

The compositions comprising a compound or salt of Formula (I) or (II) may be formulated for administration as an injection. Non-limiting examples of formulations for injection may include a sterile suspension, solution or emulsion in oily or aqueous vehicles. Suitable oily vehicles may include, but are not limited to, lipophilic solvents or vehicles such as fatty oils or synthetic fatty acid esters, or liposomes. Aqueous injection suspensions may contain substances which increase the viscosity of the suspension. The suspension may also contain suitable stabilizers. Injections may be formulated for bolus injection or continuous infusion. Alternatively, the compositions may be lyophilized or in powder form for reconstitution with a suitable vehicle, e.g., sterile pyrogen-free water, before use.

For parenteral administration, a compound or salt of Formula (I) or (II) may be formulated in a unit dosage injectable form (e.g., solution, suspension, emulsion) in association with a pharmaceutically acceptable parenteral vehicle. Such vehicles may be inherently non-toxic, and non-therapeutic. Vehicles may be water, saline, Ringer's solution, dextrose solution, and 5% human serum albumin. Non-aqueous vehicles such as fixed oils and ethyl oleate may also be used. Liposomes may be used as carriers. The vehicle may contain minor amounts of additives such as substances that enhance isotonicity and chemical stability (e.g., buffers and preservatives).

In one embodiment the invention relates to methods and compositions of Formula (I) or (II) formulated for formulated into a pharmaceutical composition suitable for intramuscular, subcutaneous, or intravenous injection. In one aspect, formulations suitable for intramuscular, subcutaneous, or intravenous injection include physiologically acceptable sterile aqueous or non-aqueous solutions, dispersions, suspensions or emulsions, and sterile powders for reconstitution into sterile injectable solutions or dispersions. Examples of suitable aqueous and non-aqueous carriers, diluents, solvents, or vehicles include water, ethanol, polyols (propyleneglycol, polyethylene-glycol, glycerol, cremophor and the like), vegetable oils and organic esters, such as ethyl oleate. In some aspects of the disclosure, formulations suitable for subcutaneous injection contain additives such as preserving, wetting, emulsifying, and dispensing agents. Prolonged absorption of the injectable pharmaceutical form can be brought about by the use of agents delaying absorption, such as aluminum monostearate and gelatin.

For intravenous injections, compounds described herein are formulated in aqueous solutions, preferably in physiologically compatible buffers such as Hank's solution, Ringer's solution, or physiological saline buffer.

Parenteral injections may involve bolus injection or continuous infusion. Formulations for injection may be presented in unit dosage form, e.g., in ampoules or in multi-dose containers, with an added preservative. The pharmaceutical composition described herein may be in a form suitable for parenteral injection as a sterile suspensions, solutions or emulsions in oily or aqueous vehicles, and may contain formulatory agents such as suspending, stabilizing and/or dispersing agents. In one aspect, the active ingredient is in powder form for constitution with a suitable vehicle, e.g., sterile pyrogen-free water, before use.

In certain aspects of the disclosure, delivery systems for pharmaceutical compounds may be employed, such as, for example, liposomes and emulsions. In certain aspects of the disclosure, compositions provided herein can also include an mucoadhesive polymer, selected from among, for example, carboxymethylcellulose, carbomer (acrylic acid polymer), poly(methylmethacrylate), polyacrylamide, polycarbophil, acrylic acid/butyl acrylate copolymer, sodium alginate and dextran.

In one embodiment the invention relates to methods and compositions of Formula (I) or (II) formulated for oral delivery to a subject in need. In one embodiment a composition is formulated so as to deliver one or more pharmaceutically active agents to a subject through a mucosa layer in the mouth or esophagus. In another embodiment the composition is formulated to deliver one or more pharmaceutically active agents to a subject through a mucosa layer in the stomach and/or intestines.

In one embodiment compositions of Formula (I) or (II) are provided in modified release dosage forms. Suitable modified release dosage vehicles include, but are not limited to, hydrophilic or hydrophobic matrix devices, water-soluble separating layer coatings, enteric coatings, osmotic devices, multi-particulate devices, and combinations thereof. The compositions may also comprise non-release controlling excipients.

In another embodiment compositions of Formula (I) or (II) are provided in enteric coated dosage forms. These enteric coated dosage forms can also comprise non-release controlling excipients. In one embodiment the compositions are in the form of enteric-coated granules, as controlled-release capsules for oral administration. The compositions can further comprise cellulose, disodium hydrogen phosphate, hydroxypropyl cellulose, pyridazine, lactose, mannitol, or sodium lauryl sulfate. In another embodiment the compositions are in the form of enteric-coated pellets, as controlled-release capsules for oral administration. The compositions can further comprise glycerol monostearate 40-50, hydroxypropyl cellulose, pyridazine, magnesium stearate, methacrylic acid copolymer type C, polysorbate 80, sugar spheres, talc, or triethyl citrate.

In another embodiment the compositions of Formula (I) or (II) are enteric-coated controlled-release tablets for oral administration. The compositions can further comprise carnauba wax, crospovidone, diacetylated monoglycerides, ethylcellulose, hydroxypropyl cellulose, pyridazine phthalate, magnesium stearate, mannitol, sodium hydroxide, sodium stearyl fumarate, talc, titanium dioxide, or yellow ferric oxide.

Sustained-release preparations comprising a compound or salt of Formula (I) or (II) may be also be prepared. Examples of sustained-release preparations may include semipermeable matrices of solid hydrophobic polymers that may contain the compound, salt or conjugate, and these matrices may be in the form of shaped articles (e.g., films or microcapsules). Examples of sustained-release matrices may include polyesters, hydrogels (e.g., poly(2-hydroxyethyl-methacrylate), or poly(vinyl alcohol)), polylactides, copolymers of L-glutamic acid and γ ethyl-L-glutamate, non-degradable ethylene-vinyl acetate, degradable lactic acid-glycolic acid copolymers such as the LUPRON DEPO™ (i.e., injectable microspheres composed of lactic acid-glycolic acid copolymer and leuprolide acetate), and poly-D-(−)-3-hydroxybutyric acid.

Pharmaceutical formulations comprising a compound or salt of Formula Formula (I) or (II) may be prepared for storage by mixing a compound, salt or conjugate with a pharmaceutically acceptable carrier, excipient, and/or a stabilizer. This formulation may be a lyophilized formulation or an aqueous solution. Acceptable carriers, excipients, and/or stabilizers may be nontoxic to recipients at the dosages and concentrations used. Acceptable carriers, excipients, and/or stabilizers may include buffers such as phosphate, citrate, and other organic acids; antioxidants including ascorbic acid and methionine; preservatives, polypeptides; proteins, such as serum albumin or gelatin; hydrophilic polymers; amino acids; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes; and/or non-ionic surfactants or polyethylene glycol.

In another embodiment the compositions of Formula (I) or (II) can further comprise calcium stearate, crospovidone, hydroxypropyl methylcellulose, iron oxide, mannitol, methacrylic acid copolymer, polysorbate 80, povidone, propylene glycol, sodium carbonate, sodium lauryl sulfate, titanium dioxide, and triethyl citrate.

In another embodiment compositions of Formula (I) or (II) are provided in effervescent dosage forms. These effervescent dosage forms can also comprise non-release controlling excipients.

In another embodiment compositions of Formula (I) or (II) can be provided in a dosage form that has at least one component that can facilitate the immediate release of an active agent, and at least one component that can facilitate the controlled release of an active agent. In a further embodiment the dosage form can be capable of giving a discontinuous release of the compound in the form of at least two consecutive pulses separated in time from 0.1 up to 24 hours. The compositions can comprise one or more release controlling and non-release controlling excipients, such as those excipients suitable for a disruptable semi-permeable membrane and as swellable substances.

In another embodiment compositions Formula (I) or (II) are provided in a dosage form for oral administration to a subject, which comprise one or more pharmaceutically acceptable excipients or carriers, enclosed in an intermediate reactive layer comprising a gastric juice-resistant polymeric layered material partially neutralized with alkali and having cation exchange capacity and a gastric juice-resistant outer layer.

In some embodiments, the compositions of Formula (I) or (II) provided herein can be in unit-dosage forms or multiple-dosage forms. Unit-dosage forms, as used herein, refer to physically discrete units suitable for administration to human or non-human animal subjects and packaged individually. Each unit-dose can contain a predetermined quantity of an active ingredient(s) sufficient to produce the desired therapeutic effect, in association with the required pharmaceutical carriers or excipients. Examples of unit-dosage forms include, but are not limited to, ampoules, syringes, and individually packaged tablets and capsules. In some embodiments, unit-dosage forms may be administered in fractions or multiples thereof. A multiple-dosage form is a plurality of identical unit-dosage forms packaged in a single container, which can be administered in segregated unit-dosage form. Examples of multiple-dosage forms include, but are not limited to, vials, bottles of tablets or capsules, or bottles of pints or gallons. In another embodiment the multiple dosage forms comprise different pharmaceutically active agents.

In some embodiments, the compositions of Formula (I) or (II) may also be formulated as a modified release dosage form, including immediate-, delayed-, extended-, prolonged-, sustained-, pulsatile-, controlled-, extended, accelerated- and fast-, targeted-, programmed-release, and gastric retention dosage forms. These dosage forms can be prepared according to known methods and techniques (see, Remington: The Science and Practice of Pharmacy, supra; Modified-Release Drug Delivery Technology, Rathbone et al., Eds., Drugs and the Pharmaceutical Science, Marcel Dekker, Inc.: New York, N.Y., 2002; Vol. 126, which are herein incorporated by reference in their entirety).

### Combination Therapies

Also contemplated herein are combination therapies, for example, co-administering a disclosed compound and an additional active agent, as part of a specific treatment regimen intended to provide the beneficial effect from the co-action of these therapeutic agents. The beneficial effect of the combination includes, but is not limited to, pharmacokinetic or pharmacodynamic co-action resulting from the combination of therapeutic agents. Administration of these therapeutic agents in combination typically is carried out over a defined time period (usually hours, days, weeks, months or years depending upon the combination selected). Combination therapy is intended to embrace administration of multiple therapeutic agents in a sequential manner, that is, wherein each therapeutic agent is administered at a different time, as well as administration of these therapeutic agents, or at least two of the therapeutic agents, in a substantially simultaneous manner.

Substantially simultaneous administration is accomplished, for example, by administering to the subject a single formulation or composition, (e.g., a tablet or capsule having a fixed ratio of each therapeutic agent or in multiple, single formulations (e.g., capsules) for each of the therapeutic agents. Sequential or substantially simultaneous administration of each therapeutic agent is effected by any appropriate route including, but not limited to, oral routes, intravenous routes, intramuscular routes, and direct absorption through mucous membrane tissues. The therapeutic agents are administered by the same route or by different routes. For example, a first therapeutic agent of the combination selected is administered by intravenous injection while the other therapeutic agents of the combination are administered orally. Alternatively, for example, all therapeutic agents are administered orally or all therapeutic agents are administered by intravenous injection.

The components of the combination are administered to a patient simultaneously or sequentially. It will be appreciated that the components are present in the same pharmaceutically acceptable carrier and, therefore, are administered simultaneously. Alternatively, the active ingredients are present in separate pharmaceutical carriers, such as, conventional oral dosage forms, that are administered either simultaneously or sequentially.

### EXAMPLES

The invention now being generally described, it will be more readily understood by reference to the following examples which are included merely for purposes of illustration of certain aspects and embodiments of the present invention, and are not intended to limit the invention in any way.

In some embodiments, compound 5 described herein is prepared as described in Scheme A.

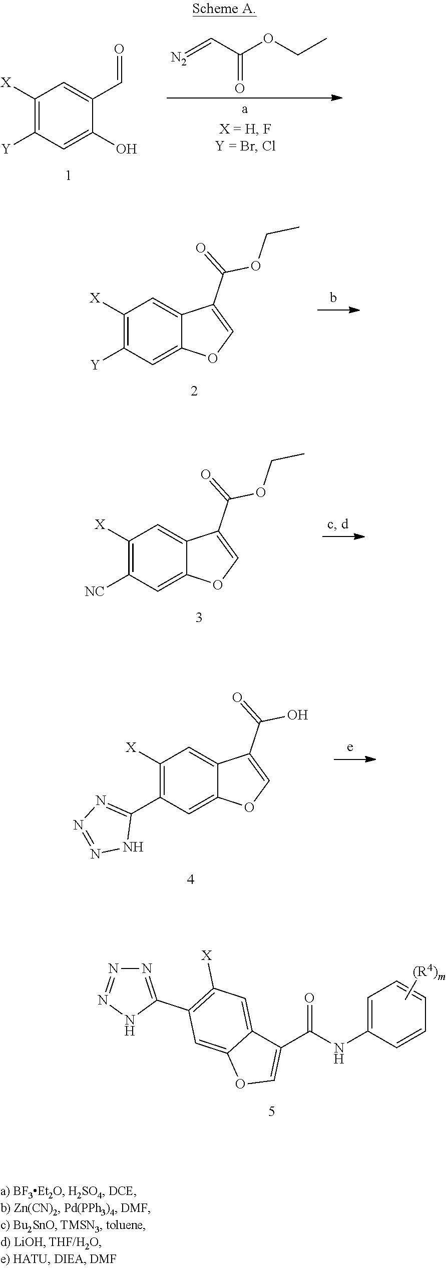

Compound 5 can be prepared via the synthetic route described in Scheme A. A convenient and selective one-pot reaction with commercially available 2-hydroxybenzaldehyde (1) and ethyl diazoacetate efficiently offers 3-ethoxycarbonylbenzofuran (2), which is converted to compound 3 by the palladium-catalyzed cyanation with cyanaide salts such as NaCN and Zn(CN)₂. The synthesis of 5-substituted 1H-tetrazole from the organic nitrile (3) can be accomplished by a method among the numerous catalysts and methodologies, and the hydrolysis of (1H-tetrazol-5-yl)benzofuran-3-carboxylate in a basic medium affords the key intermediate 4. Finally, compound 5 is obtained from the amide coupling reaction of the carboxylic acid (4) with corresponding substituted aromatic amines in the presence of coupling reagents such as HATU, EDCl, or CDI.

In some embodiments, compounds described herein are prepared as described in Scheme B.

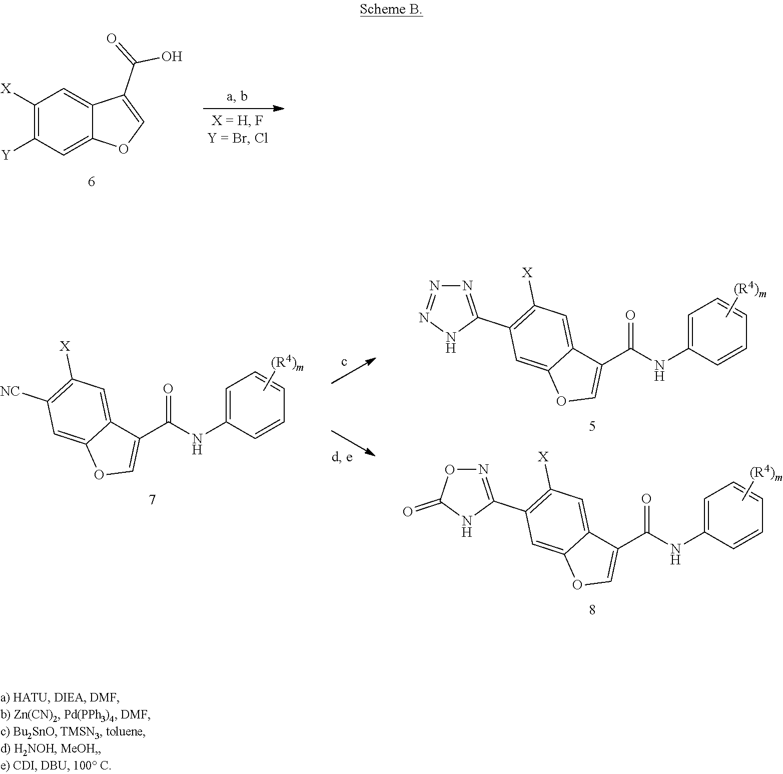

Compound 5 can be alternatively prepared via the synthetic route described in Scheme B. Compound 7 is acquired from a two-step sequence, the amide coupling and transition metal catalyzed cyanation, starting from the benzofuran acid (6). Then, the tetrazole of 5 can be introduced by using a similar manner as described in step c, Scheme A. The cyano group is also converted to other 5-membered heterocycles such as 1,2,4-oxadiazol-5(4H)-one (e.g., 8) via the N-hydroxy imidamide intermediate.

In some embodiments, compounds described herein are prepared as described in Scheme C.

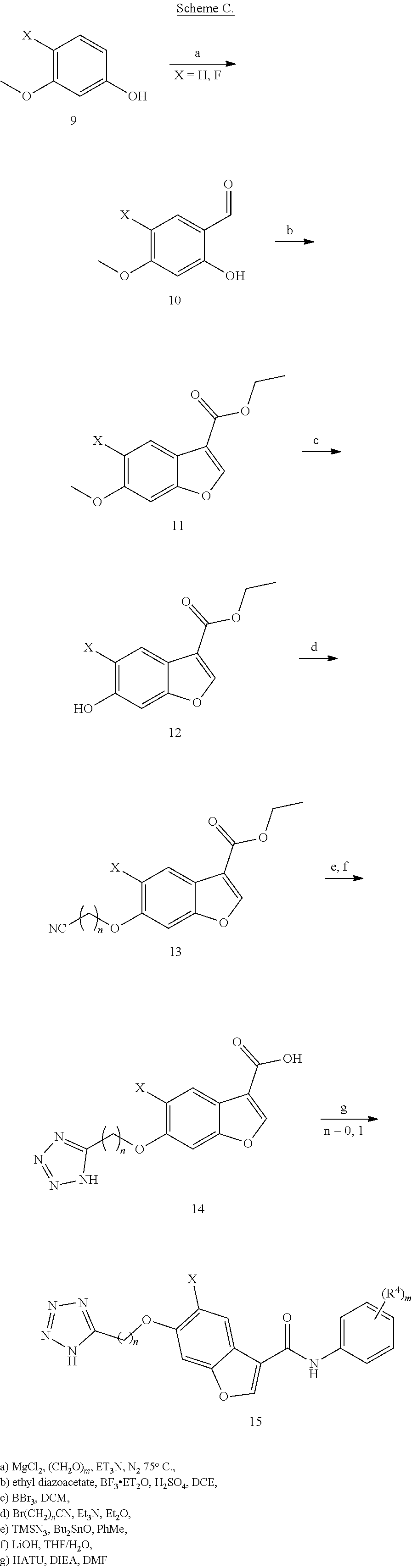

Compound 15 can be prepared via the synthetic route described in Scheme C. Commercially available 9 is converted to the hydroxybenzaldehyde (10), which is subsequently transformed to 3-ethoxycarbonylbenzofuran (11) under a similar condition described in step a, Scheme A. BBr₃-facilitated demethylation and then re-alkylation with Br—(CH₂)ₙCN produce compound 13. Starting from 13, the final compound (15) is synthesized by a similar manner as described in step c-e, Scheme A.

In some embodiments, compounds described herein are prepared as described in Scheme D.

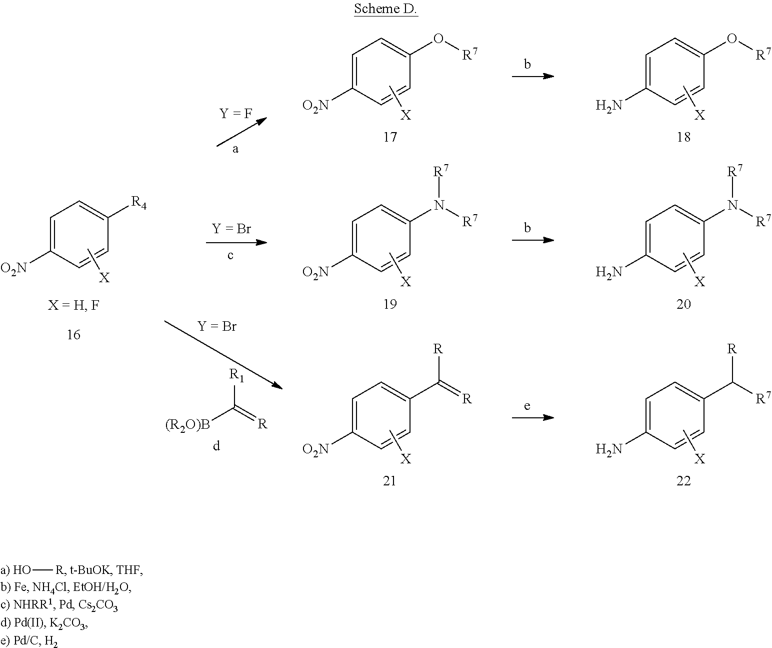

Substituted anilines (18, 20, 22) can be prepared via the synthetic route described in Scheme D. A nucleophilic aromatic substitution (S_{N}Ar) reaction of the nitrobenzene (16, Y═F) with the corresponding alcohols in the presence of a base affords compound 17 that is subsequently reduced to the oxyanilines (18). Compound 19 is acquired from the aryl bromide (16, Y═Br) via the palladium-catalyzed coupling reactions of the corresponding amines known as the Buchwald-Hartwig amination, and then reduced to the diaminobenzenes (20). Finally, (Cyclo)alkylation of the aryl bromide (16, Y═Br) to compound 22 is completed by a two-step reaction, the Suzuki-Miyaura cross-coupling with the corresponding alkenyl boronic acids/esters and the alkenyl reduction.

In some embodiments, compounds described herein are prepared as described in Scheme E.

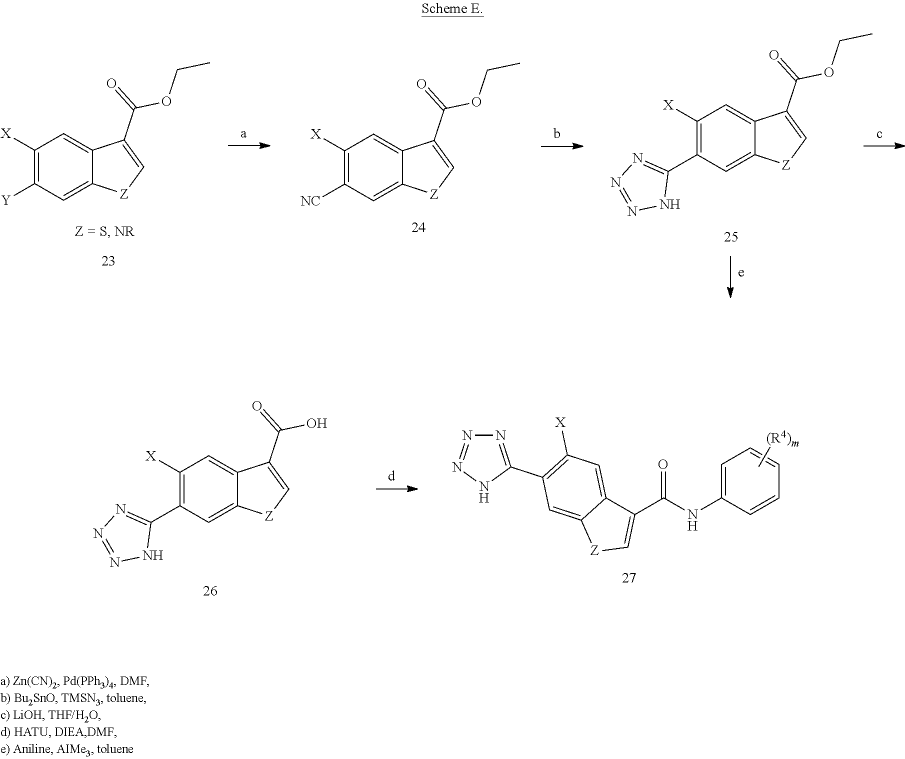

Compound 27 can be prepared by a similar manner as described in Scheme E. Compound 27 is acquired from a similar manner as described in step b to e, Example A. Alternatively, it can be directly obtained from compound 25 by AlMe₃-promoted amide formation with the corresponding anilines.

The compounds of Formula (I), or a pharmaceutically acceptable salt thereof, as well as combination therapies, are administered before, during or after the occurrence of a disease or condition, and the timing of administering the composition containing a compound varies.

Table 1 below describes the compounds disclosed herein.

TABLE 1

| Cmpd No. | Structure/Name |
|---|---|
| 1-1 | 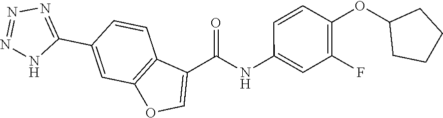 [SMILES: O=C(Nc1ccc(OC2CCCC2)c(F)c1)c1coc2cc(-c3nnn[nH]3)ccc12] |
| | N-(4-(cyclopentyloxy)-3-fluorophenyl)-6-(1H-tetrazol-5- |
| | yl)benzofuran-3-carboxamide |
| 1-2 | 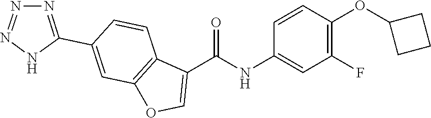 [SMILES: O=C(Nc1ccc(OC2CCC2)c(F)c1)c1coc2cc(-c3nnn[nH]3)ccc12] |
| | N-(4-cyclobutoxy-3-fluorophenyl)-6-(1H-tetrazol-5-yl)benzofuran-3- |
| | carboxamide |
| 1-3 | 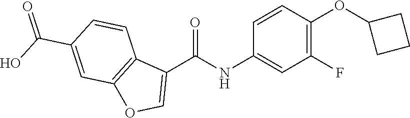 [SMILES: O=C(O)c1ccc2c(C(=O)Nc3ccc(OC4CCC4)c(F)c3)coc2c1] |
| | 3-((4-cyclobutoxy-3-fluorophenyl)carbamoyl)benzofuran-6- |
| | carboxylic acid |
| 1-4 | 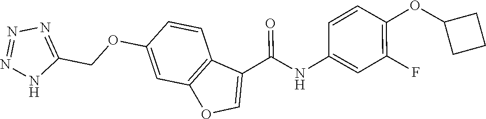 [SMILES: O=C(Nc1ccc(OC2CCC2)c(F)c1)c1coc2cc(OCc3nnn[nH]3)ccc12] |
| | 6-((1H-tetrazol-5-yl)methoxy)-N-(4-cyclobutoxy-3- |
| | fluorophenyl)benzofuran-3-carboxamide |
| 1-5 | 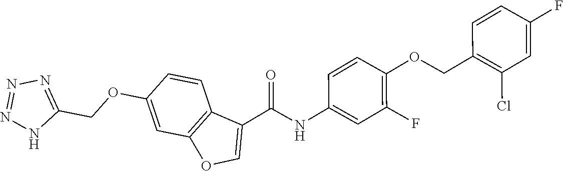 [SMILES: O=C(Nc1ccc(OCc2ccc(F)cc2Cl)c(F)c1)c1coc2cc(OCc3nnn[nH]3)ccc12] |
| | 6-((1H-tetrazol-5-yl)methoxy)-N-(4-((2-chloro-4-fluorobenzyl)oxy)- |
| | 3-fluorophenyl)benzofuran-3-carboxamide |
| 1-6 | 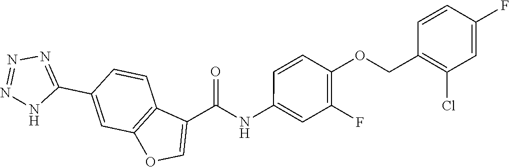 [SMILES: O=C(Nc1ccc(OCc2ccc(F)cc2Cl)c(F)c1)c1coc2cc(-c3nnn[nH]3)ccc12] |
| | N-(4-((2-chloro-4-fluorobenzyl)oxy)-3-fluorophenyl)-6-(1H-tetrazol- |
| | 5-yl)benzofuran-3-carboxamide |
| 1-7 | 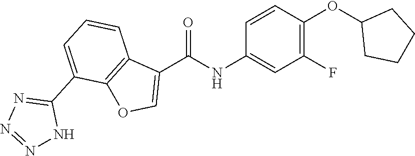 [SMILES: O=C(Nc1ccc(OC2CCCC2)c(F)c1)c1coc2c(-c3nnn[nH]3)cccc12] |
| | N-(4-(cyclopentyloxy)-3-fluorophenyl)-7-(1H-tetrazol-5- |
| | yl)benzofuran-3-carboxamide |
| 1-8 | 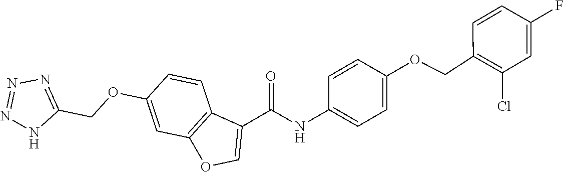 [SMILES: O=C(Nc1ccc(OCc2ccc(F)cc2Cl)cc1)c1coc2cc(OCc3nnn[nH]3)ccc12] |
| | 6-((1H-tetrazol-5-yl)methoxy)-N-(4-((2-chloro-4- |
| | fluorobenzyl)oxy)phenyl)benzofuran-3-carboxamide |
| 1-9 | 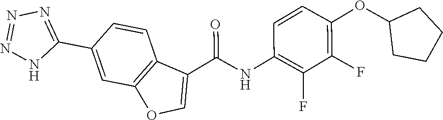 [SMILES: O=C(Nc1ccc(OC2CCCC2)c(F)c1F)c1coc2cc(-c3nnn[nH]3)ccc12] |
| | N-(4-(cyclopentyloxy)-2,3-difluorophenyl)-6-(1H-tetrazol-5- |
| | yl)benzofuran-3-carboxamide |
| 1-10 | 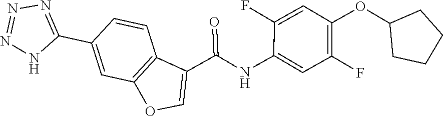 [SMILES: O=C(Nc1cc(F)c(OC2CCCC2)cc1F)c1coc2cc(-c3nnn[nH]3)ccc12] |
| | N-(4-(cyclopentyloxy)-2,5-difluorophenyl)-6-(1H-tetrazol-5- |
| | yl)benzofuran-3-carboxamide |
| 1-11 | 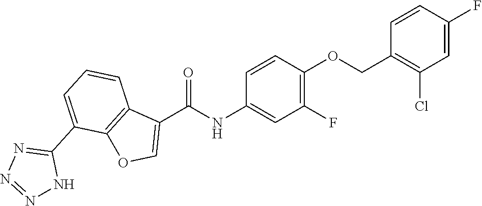 [SMILES: O=C(Nc1ccc(OCc2ccc(F)cc2Cl)c(F)c1)c1coc2c(-c3nnn[nH]3)cccc12] |
| | N-(4-((2-chloro-4-fluorobenzyl)oxy)-3-fluorophenyl)-7-(1H-tetrazol- |
| | 5-yl)benzofuran-3-carboxamide |
| 1-12 | 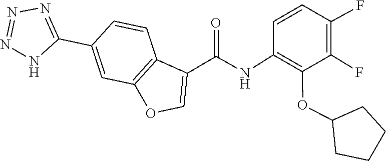 [SMILES: O=C(Nc1ccc(F)c(F)c1OC1CCCC1)c1coc2cc(-c3nnn[nH]3)ccc12] |
| | N-(2-(cyclopentyloxy)-3,4-difluorophenyl)-6-(1H-tetrazol-5- |
| | yl)benzofuran-3-carboxamide |
| 1-13 | 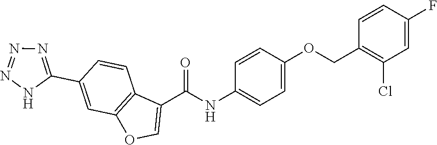 [SMILES: O=C(Nc1ccc(OCc2ccc(F)cc2Cl)cc1)c1coc2cc(-c3nnn[nH]3)ccc12] |
| | N-(4-((2-chloro-4-fluorobenzyl)oxy)phenyl)-6-(1H-tetrazol-5- |
| | yl)benzofuran-3-carboxamide |
| 1-14 | 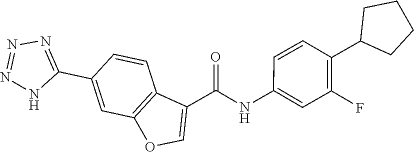 [SMILES: O=C(Nc1ccc(C2CCCC2)c(F)c1)c1coc2cc(-c3nnn[nH]3)ccc12] |
| | N-(4-cyclopentyl-3-fluorophenyl)-6-(1H-tetrazol-5-yl)benzofuran-3- |
| | carboxamide |
| 1-15 | 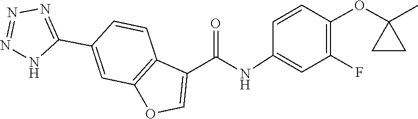 [SMILES: CC1(Oc2ccc(NC(=O)c3coc4cc(-c5nnn[nH]5)ccc34)cc2F)CC1] |
| | N-(3-fluoro-4-(1-methylcyclopropoxy)phenyl)-6-(1H-tetrazol-5- |
| | yl)benzofuran-3-carboxamide |
| 1-16 | 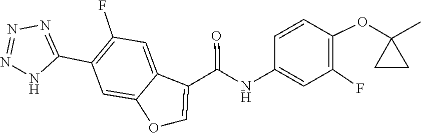 [SMILES: CC1(Oc2ccc(NC(=O)c3coc4cc(-c5nnn[nH]5)c(F)cc34)cc2F)CC1] |
| | 5-fluoro-N-(3-fluoro-4-(1-methylcyclopropoxy)phenyl)-6-(1H- |
| | tetrazol-5-yl)benzofuran-3-carboxamide |
| 1-17 | 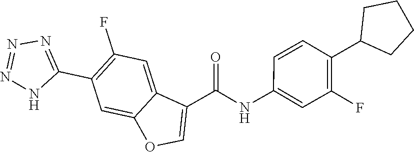 [SMILES: O=C(Nc1ccc(C2CCCC2)c(F)c1)c1coc2cc(-c3nnn[nH]3)c(F)cc12] |
| | N-(4-cyclopentyl-3-fluorophenyl)-5-fluoro-6-(1H-tetrazol-5- |
| | yl)benzofuran-3-carboxamide |
| 1-18 | 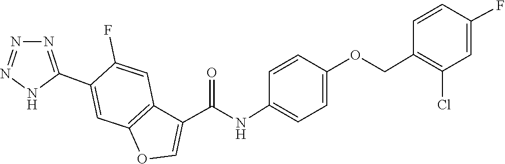 [SMILES: O=C(Nc1ccc(OCc2ccc(F)cc2Cl)cc1)c1coc2cc(-c3nnn[nH]3)c(F)cc12] |
| | N-(4-((2-chloro-4-fluorobenzyl)oxy)phenyl)-5-fluoro-6-(1H-tetrazol- |
| | 5-yl)benzofuran-3-carboxamide |
| 1-19 | 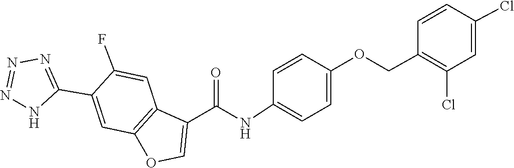 [SMILES: O=C(Nc1ccc(OCc2ccc(Cl)cc2Cl)cc1)c1coc2cc(-c3nnn[nH]3)c(F)cc12] |
| | N-(4-((2,4-dichlorobenzyl)oxy)phenyl)-5-fluoro-6-(1H-tetrazol-5- |
| | yl)benzofuran-3-carboxamide |
| 1-20 | 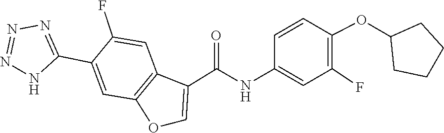 [SMILES: O=C(Nc1ccc(OC2CCCC2)c(F)c1)c1coc2cc(-c3nnn[nH]3)c(F)cc12] |
| | N-(4-(cyclopentyloxy)-3-fluorophenyl)-5-fluoro-6-(1H-tetrazol-5- |
| | yl)benzofuran-3-carboxamide |
| 1-21 | 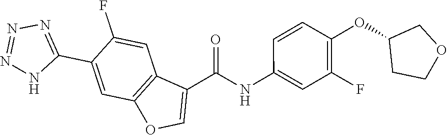 [SMILES: O=C(Nc1ccc(O[C@H]2CCOC2)c(F)c1)c1coc2cc(-c3nnn[nH]3)c(F)cc12] |
| | (S)-5-fluoro-N-(3-fluoro-4-((tetrahydrofuran-3-yl)oxy)phenyl)-6- |
| | (1H-tetrazol-5-yl)benzofuran-3-carboxamide |
| 1-22 | 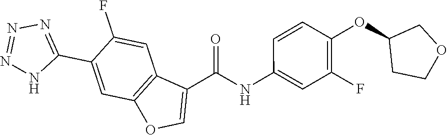 [SMILES: O=C(Nc1ccc(O[C@@H]2CCOC2)c(F)c1)c1coc2cc(-c3nnn[nH]3)c(F)cc12] |
| | (R)-5-fluoro-N-(3-fluoro-4-((tetrahydrofuran-3-yl)oxy)phenyl)-6- |
| | (1H-tetrazol-5-yl)benzofuran-3-carboxamide |
| 1-23 | 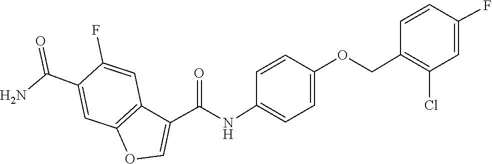 [SMILES: NC(=O)c1cc2occ(C(=O)Nc3ccc(OCc4ccc(F)cc4Cl)cc3)c2cc1F] |
| | N3-(4-((2-chloro-4-fluorobenzyl)oxy)phenyl)-5-fluorobenzofuran- |
| | 3,6-dicarboxamide |
| 1-24 | 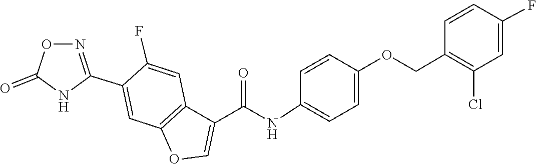 [SMILES: O=C(Nc1ccc(OCc2ccc(F)cc2Cl)cc1)c1coc2cc(-c3noc(=O)[nH]3)c(F)cc12] |
| | N-(4-((2-chloro-4-fluorobenzyl)oxy)phenyl)-5-fluoro-6-(5-oxo-4,5- |
| | dihydro-1,2,4-oxadiazol-3-yl)benzofuran-3-carboxamide |
| 1-25 | 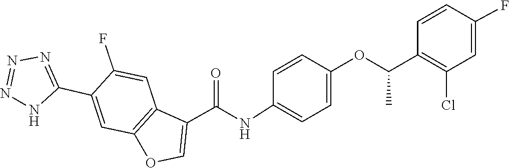 [SMILES: C[C@H](Oc1ccc(NC(=O)c2coc3cc(-c4nnn[nH]4)c(F)cc23)cc1)c1ccc(F)cc1Cl] |
| | (S)-N-(4-(1-(2-chloro-4-fluorophenyl)ethoxy)phenyl)-5-fluoro-6- |
| | (1H-tetrazol-5-yl)benzofuran-3-carboxamide |
| 1-26 | 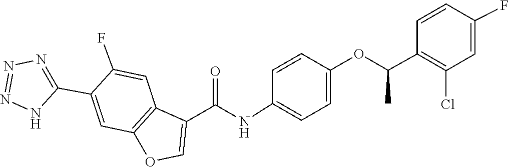 [SMILES: C[C@@H](Oc1ccc(NC(=O)c2coc3cc(-c4nnn[nH]4)c(F)cc23)cc1)c1ccc(F)cc1Cl] |
| | (R)-N-(4-(1-(2-chloro-4-fluorophenyl)ethoxy)phenyl)-5-fluoro-6- |
| | (1H-tetrazol-5-yl)benzofuran-3-carboxamide |
| 1-27 | 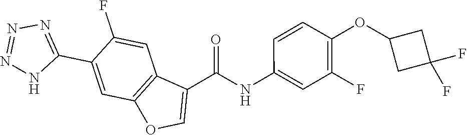 [SMILES: O=C(Nc1ccc(OC2CC(F)(F)C2)c(F)c1)c1coc2cc(-c3nnn[nH]3)c(F)cc12] |
| | N-(4-(3,3-difluorocyclobutoxy)-3-fluorophenyl)-5-fluoro-6-(1H- |
| | tetrazol-5-yl)benzofuran-3-carboxamide |
| 1-28 | 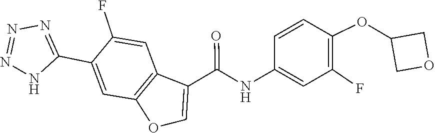 [SMILES: O=C(Nc1ccc(OC2COC2)c(F)c1)c1coc2cc(-c3nnn[nH]3)c(F)cc12] |
| | 5-fluoro-N-(3-fluoro-4-(oxetan-3-yloxy)phenyl)-6-(1H-tetrazol-5- |
| | yl)benzofuran-3-carboxamide |
| 1-29 | 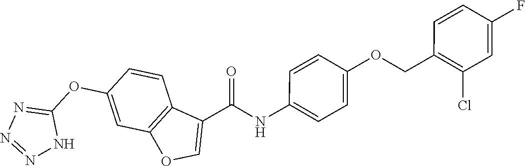 [SMILES: O=C(Nc1ccc(OCc2ccc(F)cc2Cl)cc1)c1coc2cc(Oc3nnn[nH]3)ccc12] |
| | 6-((1H-tetrazol-5-yl)oxy)-N-(4-((2-chloro-4- |
| | fluorobenzyl)oxy)phenyl)benzofuran-3-carboxamide |
| 1-30 | 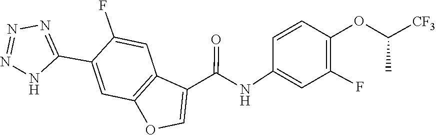 [SMILES: C[C@H](Oc1ccc(NC(=O)c2coc3cc(-c4nnn[nH]4)c(F)cc23)cc1F)C(F)(F)F] |
| | (S)-5-fluoro-N-(3-fluoro-4-((1,1,1-trifluoropropan-2-yl)oxy)phenyl)- |
| | 6-(1H-tetrazol-5-yl)benzofuran-3-carboxamide |
| 1-31 | 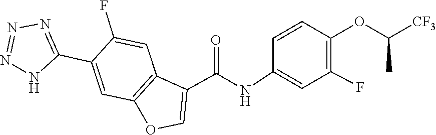 [SMILES: C[C@@H](Oc1ccc(NC(=O)c2coc3cc(-c4nnn[nH]4)c(F)cc23)cc1F)C(F)(F)F] |
| | (R)-5-fluoro-N-(3-fluoro-4-((1,1,1-trifluoropropan-2-yl)oxy)phenyl)- |
| | 6-(1H-tetrazol-5-yl)benzofuran-3-carboxamide |
| 1-32 | 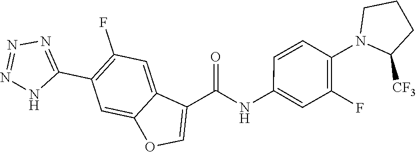 [SMILES: O=C(Nc1ccc(N2CCC[C@H]2C(F)(F)F)c(F)c1)c1coc2cc(-c3nnn[nH]3)c(F)cc12] |
| | (S)-5-fluoro-N-(3-fluoro-4-(2-(trifluoromethyl)pyrrolidin-1- |
| | yl)phenyl)-6-(1H-tetrazol-5-yl)benzofuran-3-carboxamide |
| 1-33 | 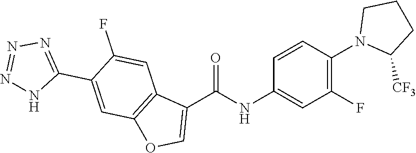 [SMILES: O=C(Nc1ccc(N2CCC[C@@H]2C(F)(F)F)c(F)c1)c1coc2cc(-c3nnn[nH]3)c(F)cc12] |
| | (R)-5-fluoro-N-(3-fluoro-4-(2-(trifluoromethyl)pyrrolidin-1- |
| | yl)phenyl)-6-(1H-tetrazol-5-yl)benzofuran-3-carboxamide |
| 1-34 | 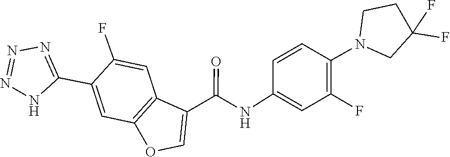 [SMILES: O=C(Nc1ccc(N2CCC(F)(F)C2)c(F)c1)c1coc2cc(-c3nnn[nH]3)c(F)cc12] |
| | N-(4-(3,3-difluoropyrrolidin-1-yl)-3-fluorophenyl)-5-fluoro-6-(1H- |
| | tetrazol-5-yl)benzofuran-3-carboxamide |
| 1-35 | 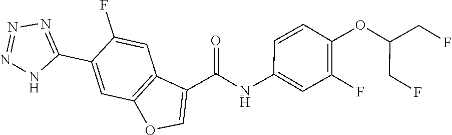 [SMILES: O=C(Nc1ccc(OC(CF)CF)c(F)c1)c1coc2cc(-c3nnn[nH]3)c(F)cc12] |
| | N-(4-((1,3-difluoropropan-2-yl)oxy)-3-fluorophenyl)-5-fluoro-6-(1H- |
| | tetrazol-5-yl)benzofuran-3-carboxamide |
| 1-36 | 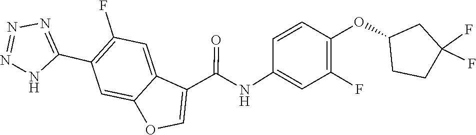 [SMILES: O=C(Nc1ccc(O[C@H]2CCC(F)(F)C2)c(F)c1)c1coc2cc(-c3nnn[nH]3)c(F)cc12] |
| | (S)-N-(4-((3,3-difluorocyclopentyl)oxy)-3-fluorophenyl)-5-fluoro-6- |
| | (1H-tetrazol-5-yl)benzofuran-3-carboxamide |
| 1-37 | 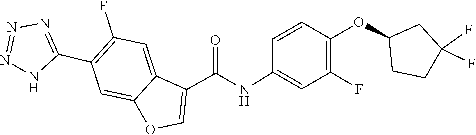 [SMILES: O=C(Nc1ccc(O[C@@H]2CCC(F)(F)C2)c(F)c1)c1coc2cc(-c3nnn[nH]3)c(F)cc12] |
| | (R)-N-(4-((3,3-difluorocyclopentyl)oxy)-3-fluorophenyl)-5-fluoro-6- |
| | (1H-tetrazol-5-yl)benzofuran-3-carboxamide |
| 1-38 | 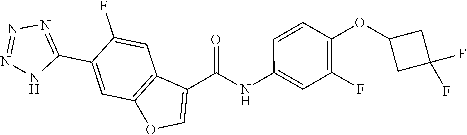 [SMILES: O=C(Nc1ccc(OC2CC(F)(F)C2)c(F)c1)c1coc2cc(-c3nnn[nH]3)c(F)cc12] |
| | 6-((1H-tetrazol-5-yl)oxy)-N-(4-(3,3-difluorocyclobutoxy)-3- |
| | fluorophenyl)-5-fluorobenzofuran-3-carboxamide |
| 1-39 | 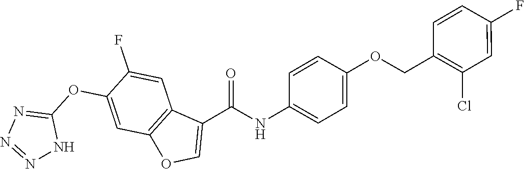 [SMILES: O=C(Nc1ccc(OCc2ccc(F)cc2Cl)cc1)c1coc2cc(Oc3nnn[nH]3)c(F)cc12] |
| | 6-((1H-tetrazol-5-yl)oxy)-N-(4-((2-chloro-4- |
| | fluorobenzyl)oxy)phenyl)-5-fluorobenzofuran-3-carboxamide |
| 1-40 | 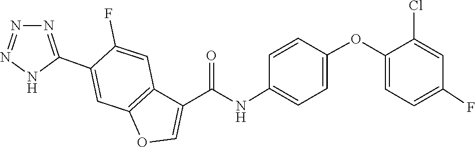 [SMILES: O=C(Nc1ccc(Oc2ccc(F)cc2Cl)cc1)c1coc2cc(-c3nnn[nH]3)c(F)cc12] |
| | N-(4-(2-chloro-4-fluorophenoxy)phenyl)-5-fluoro-6-(1H-tetrazol-5- |
| | yl)benzofuran-3-carboxamide |
| 1-41 | 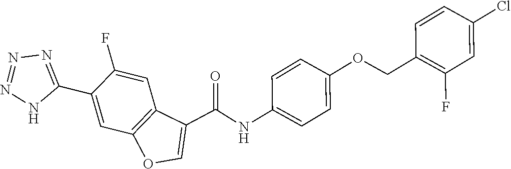 [SMILES: O=C(Nc1ccc(OCc2ccc(Cl)cc2F)cc1)c1coc2cc(-c3nnn[nH]3)c(F)cc12] |
| | N-(4-((4-chloro-2-fluorobenzyl)oxy)phenyl)-5-fluoro-6-(1H-tetrazol- |
| | 5-yl)benzofuran-3-carboxamide |
| 1-42 | 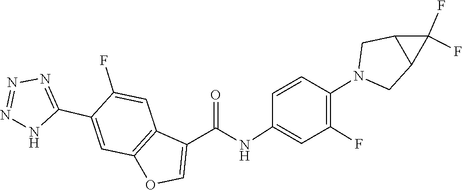 [SMILES: O=C(Nc1ccc(N2CC3C(C2)C3(F)F)c(F)c1)c1coc2cc(-c3nnn[nH]3)c(F)cc12] |
| | N-(4-(6,6-difluoro-3-azabicyclo[3.1.0]hexan-3-yl)-3-fluorophenyl)- |
| | 5-fluoro-6-(1H-tetrazol-5-yl)benzofuran-3-carboxamide |
| 1-43 | 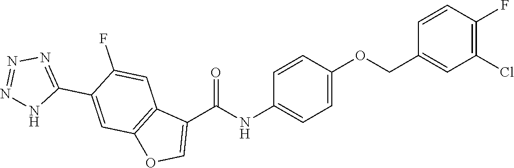 [SMILES: O=C(Nc1ccc(OCc2ccc(F)c(Cl)c2)cc1)c1coc2cc(-c3nnn[nH]3)c(F)cc12] |
| | N-(4-((3-chloro-4-fluorobenzyl)oxy)phenyl)-5-fluoro-6-(1H-tetrazol- |
| | 5-yl)benzofuran-3-carboxamide |
| 1-44 | 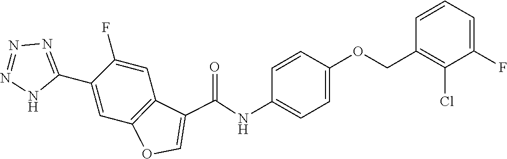 [SMILES: O=C(Nc1ccc(OCc2cccc(F)c2Cl)cc1)c1coc2cc(-c3nnn[nH]3)c(F)cc12] |
| | N-(4-((2-chloro-3-fluorobenzyl)oxy)phenyl)-5-fluoro-6-(1H-tetrazol- |
| | 5-yl)benzofuran-3-carboxamide |
| 1-45 | 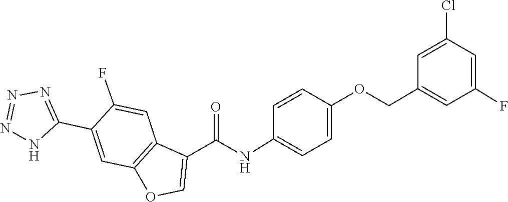 [SMILES: O=C(Nc1ccc(OCc2cc(F)cc(Cl)c2)cc1)c1coc2cc(-c3nnn[nH]3)c(F)cc12] |
| | N-(4-((3-chloro-5-fluorobenzyl)oxy)phenyl)-5-fluoro-6-(1H-tetrazol- |
| | 5-yl)benzofuran-3-carboxamide |
| 1-46 | 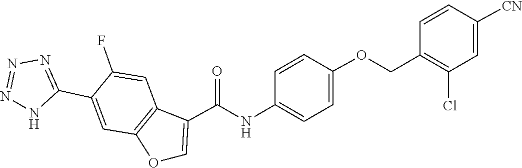 [SMILES: N#Cc1ccc(COc2ccc(NC(=O)c3coc4cc(-c5nnn[nH]5)c(F)cc34)cc2)c(Cl)c1] |
| | N-(4-((2-chloro-4-cyanobenzyl)oxy)phenyl)-5-fluoro-6-(1H-tetrazol- |
| | 5-yl)benzofuran-3-carboxamide |
| 1-47 | 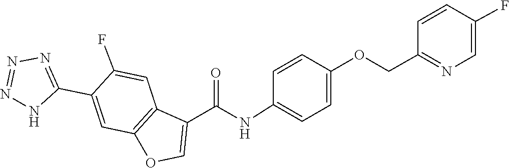 [SMILES: O=C(Nc1ccc(OCc2ccc(F)cn2)cc1)c1coc2cc(-c3nnn[nH]3)c(F)cc12] |
| | 5-fluoro-N-(4-((5-fluoropyridin-2-yl)methoxy)phenyl)-6-(1H- |
| | tetrazol-5-yl)benzofuran-3-carboxamide |
| 1-48 | 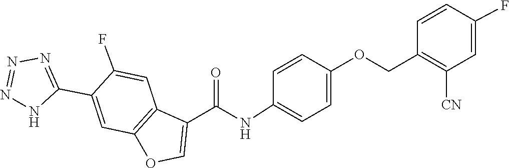 [SMILES: N#Cc1cc(F)ccc1COc1ccc(NC(=O)c2coc3cc(-c4nnn[nH]4)c(F)cc23)cc1] |
| | N-(4-((2-cyano-4-fluorobenzyl)oxy)phenyl)-5-fluoro-6-(1H-tetrazol- |
| | 5-yl)benzofuran-3-carboxamide |
| 1-49 | 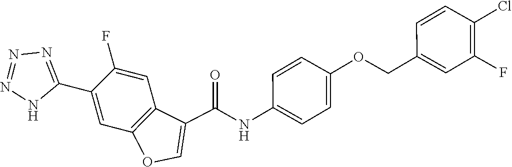 [SMILES: O=C(Nc1ccc(OCc2ccc(Cl)c(F)c2)cc1)c1coc2cc(-c3nnn[nH]3)c(F)cc12] |
| | N-(4-((4-chloro-3-fluorobenzyl)oxy)phenyl)-5-fluoro-6-(1H-tetrazol- |
| | 5-yl)benzofuran-3-carboxamide |
| 1-50 | 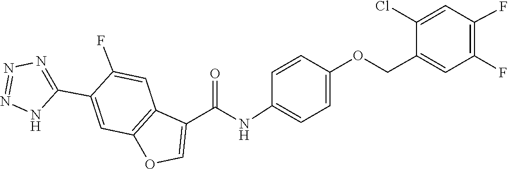 [SMILES: O=C(Nc1ccc(OCc2cc(F)c(F)cc2Cl)cc1)c1coc2cc(-c3nnn[nH]3)c(F)cc12] |
| | N-(4-((2-chloro-4,5-difluorobenzyl)oxy)phenyl)-5-fluoro-6-(1H- |
| | tetrazol-5-yl)benzofuran-3-carboxamide |
| 1-51 | 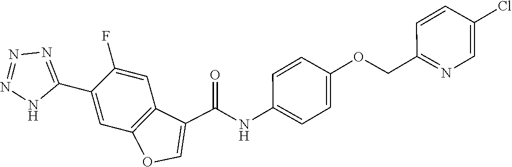 [SMILES: O=C(Nc1ccc(OCc2ccc(Cl)cn2)cc1)c1coc2cc(-c3nnn[nH]3)c(F)cc12] |
| | N-(4-((5-chloropyridin-2-yl)methoxy)phenyl)-5-fluoro-6-(1H- |
| | tetrazol-5-yl)benzofuran-3-carboxamide |
| 1-52 | 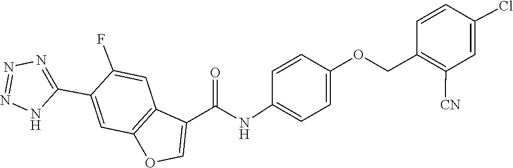 [SMILES: N#Cc1cc(Cl)ccc1COc1ccc(NC(=O)c2coc3cc(-c4nnn[nH]4)c(F)cc23)cc1] |
| | N-(4-((4-chloro-2-cyanobenzyl)oxy)phenyl)-5-fluoro-6-(1H-tetrazol- |
| | 5-yl)benzofuran-3-carboxamide |
| 1-53 | 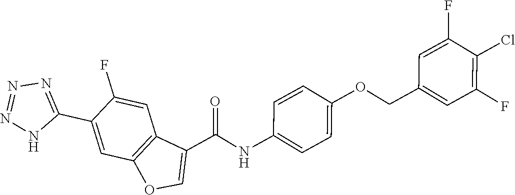 [SMILES: O=C(Nc1ccc(OCc2cc(F)c(Cl)c(F)c2)cc1)c1coc2cc(-c3nnn[nH]3)c(F)cc12] |
| | N-(4-((4-chloro-3,5-difluorobenzyl)oxy)phenyl)-5-fluoro-6-(1H- |
| | tetrazol-5-yl)benzofuran-3-carboxamide |
| 1-54 | 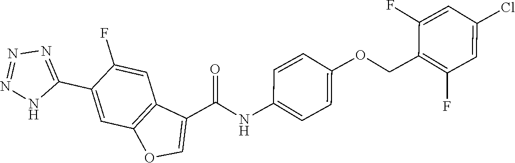 [SMILES: O=C(Nc1ccc(OCc2c(F)cc(Cl)cc2F)cc1)c1coc2cc(-c3nnn[nH]3)c(F)cc12] |
| | N-(4-((4-chloro-2,6-difluorobenzyl)oxy)phenyl)-5-fluoro-6-(1H- |
| | tetrazol-5-yl)benzofuran-3-carboxamide |
| 1-55 | 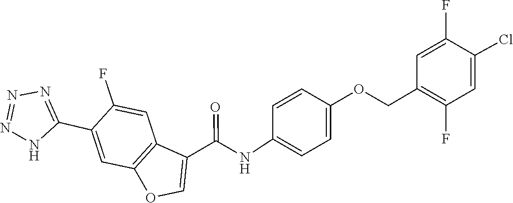 [SMILES: O=C(Nc1ccc(OCc2cc(F)c(Cl)cc2F)cc1)c1coc2cc(-c3nnn[nH]3)c(F)cc12] |
| | N-(4-((4-chloro-2,5-difluorobenzyl)oxy)phenyl)-5-fluoro-6-(1H- |
| | tetrazol-5-yl)benzofuran-3-carboxamide |
| 1-56 | 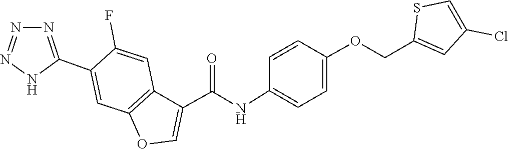 [SMILES: O=C(Nc1ccc(OCc2cc(Cl)cs2)cc1)c1coc2cc(-c3nnn[nH]3)c(F)cc12] |
| | N-(4-((4-chlorothiophen-2-yl)methoxy)phenyl)-5-fluoro-6-(1H- |
| | tetrazol-5-yl)benzofuran-3-carboxamide |
| 1-57 | 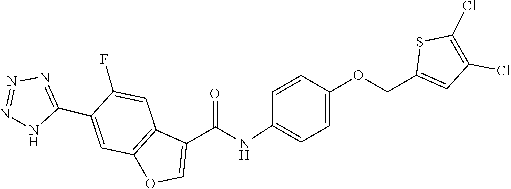 [SMILES: O=C(Nc1ccc(OCc2cc(Cl)c(Cl)s2)cc1)c1coc2cc(-c3nnn[nH]3)c(F)cc12] |
| | N-(4-((4,5-dichlorothiophen-2-yl)methoxy)phenyl)-5-fluoro-6-(1H- |
| | tetrazol-5-yl)benzofuran-3-carboxamide |
| 1-58 | 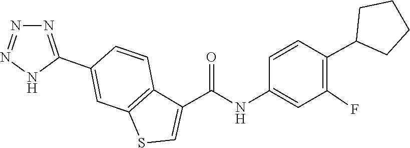 [SMILES: O=C(Nc1ccc(C2CCCC2)c(F)c1)c1csc2cc(-c3nnn[nH]3)ccc12] |
| | N-(4-cyclopentyl-3-fluorophenyl)-6-(1H-tetrazol-5- |
| | yl)benzo[b]thiophene-3-carboxamide |
| 1-59 | 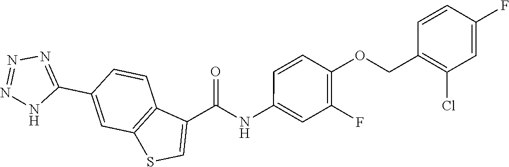 [SMILES: O=C(Nc1ccc(OCc2ccc(F)cc2Cl)c(F)c1)c1csc2cc(-c3nnn[nH]3)ccc12] |
| | N-(4-((2-chloro-4-fluorobenzyl)oxy)-3-fluorophenyl)-6-(1H-tetrazol- |
| | 5-yl)benzo[b]thiophene-3-carboxamide |
| 1-60 | 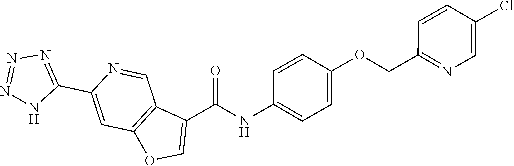 [SMILES: O=C(Nc1ccc(OCc2ccc(Cl)cn2)cc1)c1coc2cc(-c3nnn[nH]3)ncc12] |
| | N-(4-((5-chloropyridin-2-yl)methoxy)phenyl)-6-(1H-tetrazol-5- |
| | yl)furo[3,2-c]pyridine-3-carboxamide |
| 1-61 | 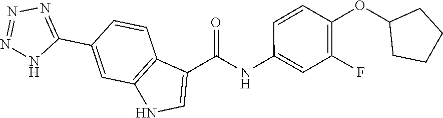 [SMILES: O=C(Nc1ccc(OC2CCCC2)c(F)c1)c1c[nH]c2cc(-c3nnn[nH]3)ccc12] |
| | N-(4-(cyclopentyloxy)-3-fluorophenyl)-6-(1H-tetrazol-5-yl)-1H- |
| | indole-3-carboxamide |
| 1-62 | 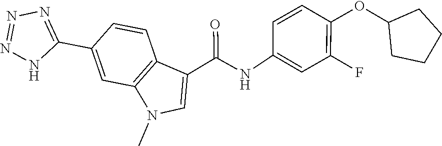 [SMILES: Cn1cc(C(=O)Nc2ccc(OC3CCCC3)c(F)c2)c2ccc(-c3nnn[nH]3)cc21] |
| | N-(4-(cyclopentyloxy)-3-fluorophenyl)-1-methyl-6-(1H-tetrazol-5- |
| | yl)-1H-indole-3-carboxamide |
| 1-63 | 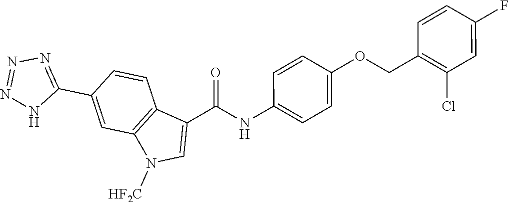 |
| | N-(4-((2-chloro-4-fluorobenzyl)oxy)phenyl)-1-(difluoromethyl)-6- |
| | (1H-tetrazol-5-yl)-1H-indole-3-carboxamide |
| 1-64 | 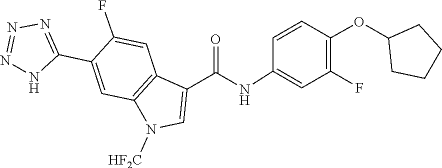 |
| | N-(4-(cyclopentyloxy)-3-fluorophenyl)-1-(difluoromethyl)-5-fluoro- |
| | 6-(1H-tetrazol-5-yl)-1H-indole-3-carboxamide |
| 1-65 | 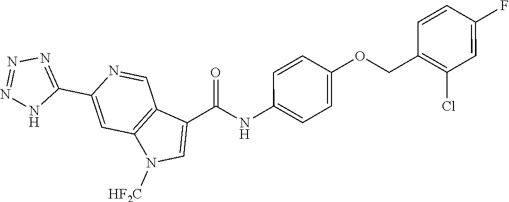 |
| | N-(4-((2-chloro-4-fluorobenzyl)oxy)phenyl)-1-(difluoromethyl)-6- |
| | (1H-tetrazol-5-yl)-1H-pyrrolo[3,2-c]pyridine-3-carboxamide |

Note that for compounds disclosed in the Table 1 above, the depicted stereochemistry for compounds 1-25, 1-26, 1-30, 1-31, 1-36, and 1-37, was arbitrarily assigned as R or S.

### Example 1: N-(4-cyclobutoxy-3-fluorophenyl)-6-(1H-tetrazol-5-yl)benzofuran-3-carboxamide

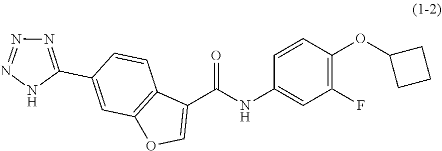 [SMILES: O=C(Nc1ccc(OC2CCC2)c(F)c1)c1coc2cc(-c3nnn[nH]3)ccc12]

### Step 1: Preparation of 1-Cyclobutoxy-2-Fluoro-4-Nitrobenzene

To a solution of cyclobutanol (3.399 g, 47.14 mmol) in THE (100 mL) was added KO^{t}Bu (5.290 g, 47.14 mmol, 1.5 eq.) at 0° C. The resulting mixture was stirred at 0° C. for 1 hr, and then treated with 1,2-difluoro-4-nitrobenzene (5 g, 31.43 mmol, 1 eq.). The mixture was stirred at 0° C. and slowly warmed to room temperature over a period of 4 hrs. It was quenched with water (50 mL) and extracted with ethyl acetate (50 mL×3). The combined organic extracts were washed with brine, dried over anhydrous Na₂SO₄, filtered and concentrated to dryness under reduced pressure to provide the crude product, which was purified by silica gel chromatography to afford the title compound (4.5 g, 50% yield). LCMS (M+H)⁺: 212.1.

### Step 2: Preparation of 4-Cyclobutoxy-3-Fluoroaniline

To a solution of 1-cyclobutoxy-2-fluoro-4-nitrobenzene (4.5 g, 21 mmol) in MeOH (100 mL) was added Pd/C (4.5 g, 1.0 eq.) at room temperature under an inert atmosphere. The reaction was stirred at room temperature under H₂ atmosphere for 2 hrs. Then it was filtered and concentrated. The residue was purified by silica gel chromatography to afford the title compound as yellow solid (3.4 g, 85% yield). LCMS (M+H)⁺: 182.0.

### Step 3: Preparation of 6-Bromo-N-(4-Cyclobutoxy-3-Fluorophenyl)Benzofuran-3-Carboxamide

To a solution of 6-bromobenzofuran-3-carboxylic acid (300 mg, 1.24 mmol) and 4-cyclobutoxy-3-fluoroaniline (226 mg, 1.24 mmol, 1 eq.) and HATU (568 mg, 1.49 mmol, 1.2 eq.) in DMF (10 mL) was added DIEA (483 mg, 3.73 mmol, 3 eq.) at room temperature. The reaction was stirred at room temperature for 2 hrs and quenched with water. Then the resulting mixture was extracted with ethyl acetate (3×50 mL). The combined organic layers were washed with brine, dried over anhydrous Na₂SO₄, filtered and concentrated. The residue was purified by silica gel chromatography (15%-50% ethyl acetate/petroleum ether) to afford the title compound as a brown solid (440 mg, 68.9%). LCMS (M+H)⁺: 404.1.

### Step 4: Preparation of 6-Cyano-N-(4-Cyclobutoxy-3-Fluorophenyl)Benzofuran-3-Carboxamide

The solution of 6-bromo-N-(4-cyclobutoxy-3-fluorophenyl)benzofuran-3-carboxamide (440 mg, 1.09 mmol) and Zn(CN)₂ (153 mg, 1.31 mmol) and Zn (14.2 mg, 218 μmol) and Pd₂(dba)₃ (99.6 mg, 109 μmol) and dppf (121 mg, 218 μmol) in DMA (10 mL) was stirred at 100° C. for 2 hrs under N₂. The reaction was cooled to room temperature and quenched with water. The resulting mixture was extracted with ethyl acetate (3×50 mL). The combined organic layers were washed with brine, dried over anhydrous Na₂SO₄, filtered and concentrated. The residue was purified by silica gel chromatography (15%-50% ethyl acetate/petroleum ether) to afford the title compound as a yellow solid (220 mg, 41.9%). LCMS (M+H)⁺: 351.3.

### Step 5: Preparation of N-(4-Cyclobutoxy-3-Fluorophenyl)-6-(1H-Tetrazol-5-Yl)Benzofuran-3-Carboxamide

To a solution of 6-cyano-N-(4-cyclobutoxy-3-fluorophenyl)benzofuran-3-carboxamide (90 mg, 0.26 mmol) and BU₂SnO (32 mg,0.13 mmol) in toluene (5 mL) was drop wisely added TMSN₃ (88.8 mg, 0.77 mmol). The mixture was stirred at 100° C. for 2 hrs under N₂. The reaction was cooled to room temperature and concentrated. The residue was purified by XBridge Prep OBD C18 Column, 30×150 mm 5 μm column (eluent: 18% to 48% (v/v) Acetonitrile and Water with 10 MMNOL/L NH₄HCO₃) to afford the title compound as a white solid (37.6 mg, 37% Yield). LCMS (M+H)⁺: 394.0. ¹H NMR (300 MHz, DMSO-d₆) δ 10.24 (s, 1H), 8.77 (s, 1H), 8.20 (d, J=1.2 Hz, 1H), 8.16-8.03 (m, 2H), 7.73 (dd, J=13.6, 2.5 Hz, 1H), 7.46-7.36 (m, 1H), 7.04 (t, J=9.2 Hz, 2H), 4.80-4.65 (m, 1H), 2.48-2.37 (m, 2H), 2.18-2.00 (m, 2H), 1.88-1.73 (m, 1H), 1.73-1.56 (m, 1H).

The following compounds were synthesized following Example 1:

| Cmpd | | MS | |
|---|---|---|---|
| No. | Structure/Name | (M + H)⁺ | ¹H NMR |
| 1-1 | 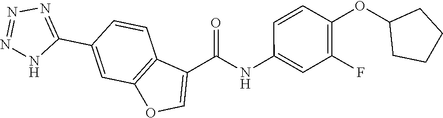 [SMILES: O=C(Nc1ccc(OC2CCCC2)c(F)c1)c1coc2cc(-c3nnn[nH]3)ccc12] N-(4-(cyclopentyloxy)-3-fluorophenyl)-6- (1H-tetrazol-5-yl)benzofuran-3-carboxamide | 408.1 | (300 MHz, DMSO-d₆) δ 10.29 (s, 1H), 8.88 (s, 1H), 8.36 − 8.23 (m, 2H), 8.07 (dd, J = 8.2, 1.4 Hz, 1H), 7.70 (dd, J = 13.6, 2.5 Hz, 1H), 7.40 (d, J = 9.0, 1H), 7.15 (t, J = 9.4 Hz, 1H), 4.88 − 4.77 (m, 1H), 1.93 − 1.80 (m, 2H), 1.80 − 1.67 (m, 4H), 1.72 − 1.53 (m, |
| | | | 2H). NH was not observed. |
| 1-6 | 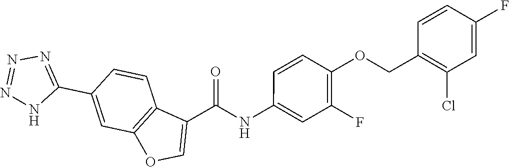 [SMILES: O=C(Nc1ccc(OCc2ccc(F)cc2Cl)c(F)c1)c1coc2cc(-c3nnn[nH]3)ccc12] | 482.0 | (400 MHz, DMSO-d₆) δ 10.25 (s, 1H), 8.73 (s, 1H), 8.16 (d, J = 1.1 Hz, 1H), 8.13 − 8.00 (m, 2H), 7.75 (dd, J = 13.6, 2.5 Hz, 1H), 7.71 − 7.63 (m, 1H), 7.53 (dd, J = 8.9, 2.6 Hz, 1H), 7.48 − 7.40 (m, 1H), 7.34 − 7.24 (m, 2H), 7.09 (s, 1H), 5.18 (s, 2H). |
| | N-(4-((2-chloro-4-fluorobenzyl)oxy)-3- | | |
| | fluorophenyl)-6-(1H-tetrazol-5- | | |
| | yl)benzofuran-3-carboxamide | | |
| 1-9 | 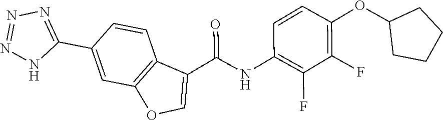 [SMILES: O=C(Nc1ccc(OC2CCCC2)c(F)c1F)c1coc2cc(-c3nnn[nH]3)ccc12] N-(4-(cyclopentyloxy)-2,3-difluorophenyl)-6- (1H-tetrazol-5-yl)benzofuran-3-carboxamide | 426.1 | (400 MHz, DMSO-d₆) δ 10.18 (s, 1H), 8.87 (s, 1H), 8.29 (d, J = 8.0 Hz, 1H), 8.18 − 8.20 (m, 1H), 8.06 − 8.08 (m, 1H), 7.30 − 7.34 (m, 1H), 7.03 − 7.08 (m, 1H), 4.93 − 4.96 (m, 1H), 1.93 − 1.99 (m, 2H), 1.60 − 1.79 (m, 6H), NH was not observed. |
| 1-10 | 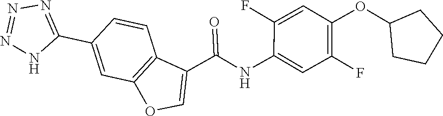 [SMILES: O=C(Nc1cc(F)c(OC2CCCC2)cc1F)c1coc2cc(-c3nnn[nH]3)ccc12] N-(4-(cyclopentyloxy)-2,5-difluorophenyl)-6- (1H-tetrazol-5-yl)benzofuran-3-carboxamide | 426.1 | (400 MHz, DMSO-d₆) δ 10.01 (s, 1H), 8.78 (s, 1H), 8.18 (d, J = 8.0 Hz, 1H), 8.03 − 8.09 (m, 2H), 7.55 − 7.59 (m, 1H), 7.22 −7.27 (m, 1H), 7.09 (s, 1H), 4.90 − 4.94 (m, 1H), 1.89 − 1.98 (m, 2H), 1.70 − 1.80 (m, 4H), 1.50 − 1.61 (s, 2H). |
| 1-12 | 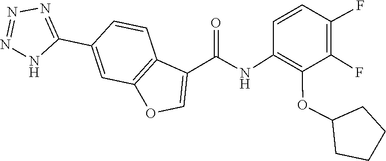 [SMILES: O=C(Nc1ccc(F)c(F)c1OC1CCCC1)c1coc2cc(-c3nnn[nH]3)ccc12] | 426.1 | (400 MHz, DMSO-d₆) δ 9.89 (s, 1H), 8.94 (d, J = 8.0 Hz, 1H), 8.32 (s, 1H), 8.23 − 8.25 (m, 1H), 8.06 − 8.09 (m, 1H), 7.43 − 7.48 (m, 1H), 7.18 − 7.25 (m, 1H), 4.81 (s, 1H), 1.82 (s, 2H), 1.68 (s, 4H), 1.49 (s, 2H), NH was not observed. |
| | N-(2-(cyclopentyloxy)-3,4-difluorophenyl)-6- | | |
| | (1H-tetrazol-5-yl)benzofuran-3-carboxamide | | |
| 1-14 | 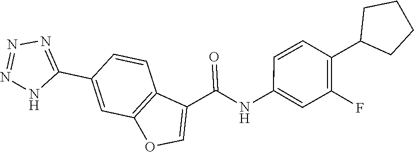 [SMILES: O=C(Nc1ccc(C2CCCC2)c(F)c1)c1coc2cc(-c3nnn[nH]3)ccc12] N-(4-cyclopentyl-3-fluorophenyl)-6-(1H- tetrazol-5-yl)benzofuran-3-carboxamide | 392.0 | (300 MHz, DMSO-d₆) δ 10.37 (s, 1H), 8.82 (s, 1H), 8.22 (s, 1H), 8.15 (d, J = 8.2 Hz, 1H), 8.08 (d, J = 8.2 Hz, 1H), 7.69 (dd, J = 13.0, 2.1 Hz, 1H), 7.45 (dd, J = 8.4, 2.1 Hz, 1H), 7.33 (t, J = 8.6 Hz, 1H), 3.25 − 3.07 (m, 1H), 2.08 − 1.91 (m, 2H), 1.87 − 1.43 (m, 6H), NH was not observed. |
| 1-15 | 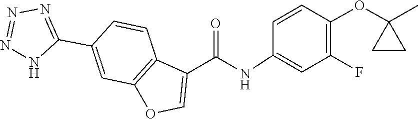 [SMILES: CC1(Oc2ccc(NC(=O)c3coc4cc(-c5nnn[nH]5)ccc34)cc2F)CC1] N-(3-fluoro-4-(1- methylcyclopropoxy)phenyl)-6-(1H-tetrazol- 5-yl)benzofuran-3-carboxamide | 394.0 | (300 MHz, DMSO-d₆) δ 10.27 (s, 1H), 8.79 (s, 1H), 8.22 (s, 1H), 8.15 (d, J = 8.2 Hz, 1H), 8.07 (d, J = 8.3 Hz, 1H), 7.75 (dd, J = 13.5, 2.5 Hz, 1H), 7.50 − 7.40 (m, 1H), 7.34 (t, J = 9.1 Hz, 1H), 7.14 (brs, 1H), 1.52 (s, 3H), 1.02 − 0.87 (m, 2H), 0.87 − 0.72 (m, 2H). |

### Example 2: 3-((4-cyclobutoxy-3-fluorophenyl)carbamoyl)benzofuran-6-carboxylic acid

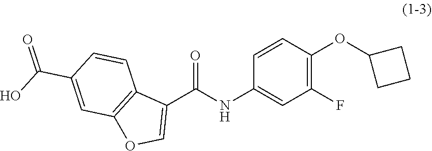 [SMILES: O=C(O)c1ccc2c(C(=O)Nc3ccc(OC4CCC4)c(F)c3)coc2c1]

### Step 1: Preparation of Ethyl 3-((4-Cyclobutoxy-3-Fluorophenyl)Carbamoyl)Benzofuran-6-Carboxylate

To a solution of 6-cyano-N-(4-cyclobutoxy-3-fluorophenyl)benzofuran-3-carboxamide (100 mg, 285 μmol) in EtOH (5 mL) was added SOCl₂ (1.02 g, 8.56 mmol, 30 eq.) at 0° C. The mixture was stirred at 80° C. for 16 hrs. The reaction was cooled to room temperature, quenched with water (50 mL) and extracted with ethyl acetate (50 mL×3). The combined organic extracts were washed with brine, dried over anhydrous Na₂SO₄, filtered and concentrated. The crude product was purified by silica gel chromatography to afford the title compound as a white solid (52 mg, 46% yield). LCMS (M+H)⁺: 398.0.

### Step 2: Preparation of 3-((4-Cyclobutoxy-3-Fluorophenyl)Carbamoyl)Benzofuran-6-Carboxylic Acid

To a solution of ethyl 3-((4-cyclobutoxy-3-fluorophenyl)carbamoyl)benzofuran-6-carboxylate (52 mg, 0.13 mmol) in THF/MeOH/H₂O (3/1/1, 5 mL) was added lithium hydroxide (4.8 mg, 0.20 mmol, 2 eq.) at 0° C. The mixture was stirred at 0° C. for 2 hrs and then adjusted the pH to 5-6 by adding 2 M HCl. The reaction was concentrated, and the residue was purified further by preparative HPLC using a XBridge Prep OBD C18 150 mm×30 mm×5 m column (eluent: 15% to 45% (v/v) CH₃CN and H₂O with 10 MMOL/L NH₄HCO₃) to give the title compound as a white solid (19.6 mg, 40% yield). LCMS (M+H)⁺: 370.0. ¹H NMR (400 MHz, DMSO-d₆) δ δ 10.30 (s, 1H), 8.88 (s, 1H), 8.19-8.10 (m, 2H), 7.97 (d, J=8.2 Hz, 1H), 7.69 (d, J=13.5 Hz, 1H), 7.38 (d, J=8.1 Hz, 2H), 7.01 (t, J=9.3 Hz, 1H), 4.75-4.61 (m, 1H), 2.45-2.35 (m, 2H), 2.13-1.94 (m, 2H), 1.83-1.71 (m, 1H), 1.68-1.53 (m, 1H).

### Example 3: N-(4-((2-chloro-4-fluorobenzyl)oxy)phenyl)-6-(1H-tetrazol-5-yl)benzofuran-3-carboxamide

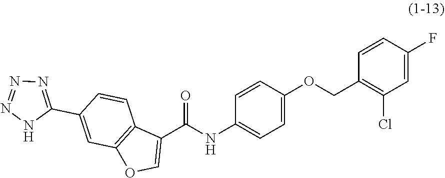 [SMILES: O=C(Nc1ccc(OCc2ccc(F)cc2Cl)cc1)c1coc2cc(-c3nnn[nH]3)ccc12]

### Step 1: Preparation of 2-Chloro-4-Fluoro-1-((4-Nitrophenoxy)Methyl)Benzene

To a solution of 1-fluoro-4-nitrobenzene (2 g, 0.01 mmol) in THF (50 mL) was added (2-chloro-4-fluorophenyl)methanol (2 g, 0.01 mmol, 1 eq.). The resulting mixture was cooled to 0° C., and then treated with KO^{t}Bu (2 g, 0.02 mmol, 1.5 eq.). The reaction was stirred at room temperature for 4 hrs, quenched with water (50 mL) and extracted with ethyl acetate (50 mL×3). The combined organic extracts were washed with brine, dried over anhydrous Na₂SO₄, filtered and concentrated. The crude product was recrystallized by DCM/PE solvent to afford the title compound as a white solid (4 g, 60% yield).

### Step 2: Preparation of 4-((2-Chloro-4-Fluorobenzyl)Oxy)Aniline

To a solution of 2-chloro-4-fluoro-1-((4-nitrophenoxy)methyl)benzene (2 g, 0.02 mol) in EtOH/H₂O (6:1, 70 mL) was added Fe powder (2 g, 5 eq.) and NH₄Cl (4 g, 10 eq.) at room temperature. The mixture was stirred at 85° C. for 2 hrs, cooled to room temperature and filtered. The filtrate was extracted with ethyl acetate (50 mL×3). The combined organic extracts were washed with brine, dried over anhydrous Na₂SO₄, filtered and concentrated. The residue was purified by silica gel chromatography to afford the product as a brown solid (1.5 g, 70% yield). CMS (M+H)⁺: 251.9.

### Step 3: Preparation of Ethyl 6-(1H-Tetrazol-5-Yl)Benzofuran-3-Carboxylate

To a solution of ethyl 6-cyanobenzofuran-3-carboxylate (1.6 g, 7.4 mmol) and Bu₂SnO (1.1 g, 4.5 mmol) in toluene (30 mL) was drop wisely added TMSN₃ (2.6 g, 22 mmol). The mixture was stirred at 100° C. for 2 hrs under N₂. The reaction was cooled to room temperature, quenched with water (50 mL) and extracted with ethyl acetate (50 mL×3). The combined organic extracts were washed with brine, dried over anhydrous Na₂SO₄, filtered and concentrated. The residue was purified by silica gel chromatography (17% MeOH/DCM) to afford the title compound (1.6 g, 83%) was obtained as a yellow solid. LCMS (M+H)⁺: 259.1.

### Step 4: Preparation of 6-(1H-Tetrazol-5-Yl)Benzofuran-3-Carboxylic Acid

To a solution of ethyl 6-(1H-tetrazol-5-yl)benzofuran-3-carboxylate (250 mg, 968 μmol) in THF/MeOH/H₂O (10 mL, v/v/v=3/1/1) was added LiOH (46.4 mg, 1.94 mmol) at 0° C. Then the reaction was slowly warm to room temperature and stirred for 2 hrs. The pH of the reaction was adjusted to ˜7 by adding 1 M HCl. After removal of the volatile solvent, the residue was purified by C18 column (eluent: 10% to 30% (v/v) CH₃CN and H₂O with 10 MMOL/L NH₄HCO₃) to afford the title compound as a yellow solid (205 mg, 85%). LCMS (M+H)⁺: 231.0.

### Step 5: Preparation of 3-((4-Cyclobutoxy-3-Fluorophenyl)Carbamoyl)Benzofuran-6-Carboxylic Acid

To a solution of 6-(1H-tetrazol-5-yl)benzofuran-3-carboxylic acid (150 mg, 652 μmol), 4-((2-chloro-4-fluorobenzyl)oxy)aniline (164 mg, 652 μmol, 1 eq.) and HATU (297 mg, 782 μmol, 1.2 eq.) in DMF (10 mL) was added DIEA (253 mg, 1.95 mmol, 3 eq.) at room temperature. The resulting mixture was stirred at room temperature for 2 hrs, quenched with water and extracted with ethyl acetate (3×50 mL). The combined organic layers were washed with brine, dried over anhydrous Na₂SO₄, filtered and concentrated. The residue was purified by Xselect CSH OBD Column, 30×150 mm 5 μm column (eluent: 44% to 74% (v/v) Acetonitrile and Water with 0.1% FA) to afford the title compound as a yellow solid (40.3 mg, 13.2% Yield). LCMS (M+H)⁺: 463.9. ¹H NMR (400 MHz, DMSO-d₆) δ 10.22 (s, 1H), 8.90 (s, 1H), 8.37-8.27 (m, 2H), 8.09 (dd, J=8.2, 1.4 Hz, 1H), 7.74-7.64 (m, 3H), 7.53 (dd, J=8.9, 2.7 Hz, 1H), 7.34-7.24 (m, 1H), 7.15-7.03 (m, 2H), 5.13 (s, 2H), —NH was not observed.

### Example 4: 6-((1H-tetrazol-5-yl)methoxy)-N-(4-((2-chloro-4-fluorobenzyl)oxy)phenyl)benzofuran-3-carboxamide

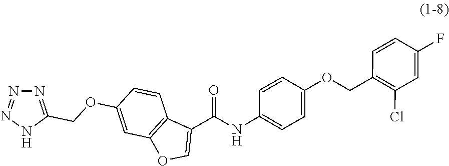 [SMILES: O=C(Nc1ccc(OCc2ccc(F)cc2Cl)cc1)c1coc2cc(OCc3nnn[nH]3)ccc12]

### Step 1: Preparation of Methyl 6-(Cyanomethoxy)Benzofuran-3-Carboxylate

To a solution of methyl 6-hydroxybenzofuran-3-carboxylate (1.5 g, 7.8 mmol) and 2-bromoacetonitrile (1.1 g, 9.4 mmol, 1.2 eq.) in Acetone (15 mL) was added K₂CO₃ (2.2 g, 16 mmol, 2 eq.) at room temperature. The reaction mixture was stirred at 60° C. for 6 hrs, cooled to room temperature, quenched with water (50 mL) and extracted with ethyl acetate (50 mL×3). The combined organic extracts were washed with brine, dried over anhydrous Na₂SO₄, filtered and concentrated. The crude product was purified by silica gel chromatography to afford the title compound as a white solid (1.08 g, 53% yield).

### Step 2: Preparation of Methyl 6-((1H-Tetrazol-5-Yl)Methoxy)Benzofuran-3-Carboxylate

To a solution of methyl 6-(cyanomethoxy)benzofuran-3-carboxylate (lg, 4 mmol) in toluene (20 mL) was added TMSN₃ (1 g, 0.01 mmol, 3 equiv.) and Bu₂SnO (0.5 g, 2 mmol, 0.5 equiv.). After removal of air from the reaction flask, it was backfilled with Nitrogen. The reaction was stirred at 100° C. for 2 hrs under N₂, cooled to room temperature, quenched with water (50 mL) and extracted with ethyl acetate (50 mL×3). The combined organic extracts were washed with brine, dried over anhydrous Na₂SO₄, filtered and concentrated. The residue was purified by silica gel chromatography to afford the title compound as a white solid (1 g, 80% yield). LCMS (M+H)⁺: 275.0.

### Step 3: Preparation of 6-((1H-Tetrazol-5-Yl)Methoxy)Benzofuran-3-Carboxylic Acid

To a solution of methyl 6-((1H-tetrazol-5-yl)methoxy)benzofuran-3-carboxylate (900 mg, 3.28 mmol) in THF/MeOH/H₂O (3/1/1, 5 mL) was added lithium hydroxide (157 mg, 6.56 mmol, 2 eq.) at 0° C. The mixture was stirred at room temperature for 2 hrs and adjusted the pH to 5-6 by adding 2 M HCl. The reaction was concentrated, and the residue was purified further by silica gel column (eluent: 15% to 25% (v/v) DCM and MeOH) to afford the title compound as a white solid (650 mg, 74.1% yield). LCMS (M+H)⁺: 261.0.

### Step 4: Preparation of 6-((1H-Tetrazol-5-Yl)Methoxy)-N-(4-((2-Chloro-4-Fluorobenzyl)Oxy)Phenyl)Benzofuran-3-Carboxamide

To a solution of 6-((1H-tetrazol-5-yl)methoxy)benzofuran-3-carboxylic acid (150 mg, 576 μmol), 4-((2-chloro-4-fluorobenzyl)oxy)aniline (145 mg, 576 mol, 1 eq.) and HATU (263 mg, 692 μmol, 1.2 eq.) in DMF (5 mL) was added DIEA (224 mg, 1.73 mmol, 3 eq.) at room temperature. The reaction was stirred at room temperature for 2 hrs. After the reaction completed, it was quenched with water and extracted with ethyl acetate (3×50 mL). The combined organic layers were washed with brine, dried over anhydrous Na₂SO₄, filtered and concentrated. The residue was purified by silica gel chromatography (150%-40% methanol/dichloromthane) to afford the crude product. Then crude product was further purified by XBridge Prep OBD C18 Column, 30×150 mm 5 μm column (eluent: 25% to 55% (v/v) Acetonitrile and Water with 10 MMNOL/L, NH₄HCO₃) to give the title compound as a white solid (24.0 mg, 8.33 00 Yield). LCMS (M+H)⁺: 494.0. ¹H NMR (400 MHz, DMSO-d₆) δ 10.05 (s, 1H), 8.62 (s, 1H), 7.91 (d, J=8.7 Hz, 1H), 7.72-7.62 (m, 3H), 7.58-7.48 (m, 2H), 7.34-7.24 (m, 1H), 7.11 (s, 1H), 7.09-6.99 (m, 3H), 5.21 (s, 2H), 5.13 (s, 2H).

The following compounds were synthesized following Example 4:

| Cmpd | | MS | |
|---|---|---|---|
| No. | Structure/Name | (M + H)⁺ | ¹H NMR |
| 1-4 | 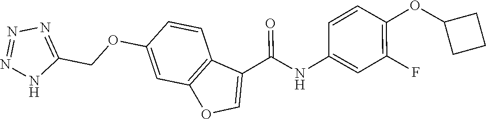 [SMILES: O=C(Nc1ccc(OC2CCC2)c(F)c1)c1coc2cc(OCc3nnn[nH]3)ccc12] | 424.1 | (300 MHz, Methanol- d₄) δ 8.40 (s, 1H), 7.96 (d, J = 8.7 Hz, 1H), 7.62 (dd, J = 13.2, 2.5 Hz, 1H), 7.39 − 7.27 (m, 2H), 7.09 (dd, J = 8.7, 2.3 Hz, 1H), 6.95 (t, J = 9.1 Hz, 1H), |
| | 6-((1H-tetrazol-5-yl)methoxy)-N-(4-cyclobutoxy-3- | | 5.37 (s, 2H), 4.79 − |
| | fluorophenyl)benzofuran-3-carboxamide | | 4.64 (m, 1H), 2.56 − |
| | | | 2.38 (m, 2H), 2.28 − |
| | | | 2.08 (m, 2H), 1.99 − |
| | | | 1.61 (m, 2H). |
| 1-5 | 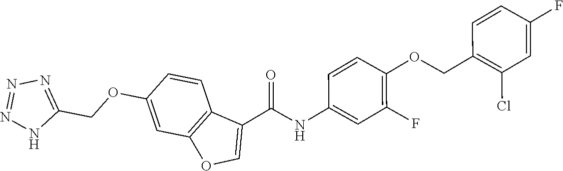 [SMILES: O=C(Nc1ccc(OCc2ccc(F)cc2Cl)c(F)c1)c1coc2cc(OCc3nnn[nH]3)ccc12] | 512.0 | (300 MHz, Methanol- d₄) δ 8.39 (s, 1H), 7.97 (d, J = 8.7 Hz, 1H), 7.73 − 7.59 (m, 2H), 7.43 − 7.27 (m, 3H), 7.22 − 7.06 (m, 3H), 5.37 (s, 2H), 5.22 (s, 2H). |
| | 6-((1H-tetrazol-5-yl)methoxy)-N-(4-((2-chloro-4- | | |
| | fluorobenzyl)oxy)-3-fluorophenyl)benzofuran-3- | | |
| | carboxamide | | |

### Example 5: 6-((1H-tetrazol-5-yl)oxy)-N-(4-((2-chloro-4-fluorobenzyl)oxy)phenyl)benzofuran-3-carboxamide

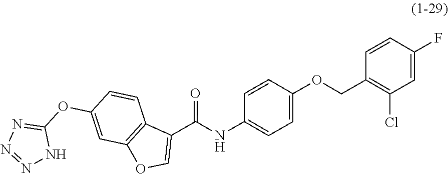 [SMILES: O=C(Nc1ccc(OCc2ccc(F)cc2Cl)cc1)c1coc2cc(Oc3nnn[nH]3)ccc12]

### Step 1: Preparation of Ethyl 6-Cyanatobenzofuran-3-Carboxylate

To a solution of ethyl 6-hydroxybenzofuran-3-carboxylate (1000 mg, 4.803 mmol) and BrCN (610.5 mg, 5.763 mmol, 1.2 eq.) in Et₂O (10 mL) was added Et₃N (972 mg, 9.606 mmol, 2 eq.) at 0° C. The mixture was stirred at 0° C. for 3 hrs, quenched with water (50 mL) and extracted with ethyl acetate (50 mL×3). The combined organic extracts were washed with brine, dried over anhydrous Na₂SO₄, filtered and concentrated. The residue was purified by silica gel chromatography to afford the product as a white solid (309 mg, 54.7% yield).

### Step 2: Preparation of Ethyl 6-((1H-Tetrazol-5-Yl)Oxy)Benzofuran-3-Carboxylate

To a solution of ethyl 6-cyanatobenzofuran-3-carboxylate (800 mg,1.30 mmol) in toluene (1 mL) was added TMSN₃ (448 mg, 3.89 mmol, 3 equiv.) and Bu₂SnO (162 mg, 0.649 mmol, 0.5 equiv.). After removal of air from the reaction flask, it was backfilled with Nitrogen. The reaction was stirred at 100° C. for 3 hrs under N₂, cooled to room temperature, quenched with water (50 mL) and extracted with ethyl acetate (50 mL×3). The combined organic extracts were washed with brine, dried over anhydrous Na₂SO₄, filtered and concentrated. The crude product was purified by silica gel chromatography to afford the title compound as a white solid (261 mg, 73.3% yield).

### Step 3: Preparation of 6-((1H-Tetrazol-5-Yl)Methoxy)-N-(4-((2-Chloro-4-Fluorobenzyl)Oxy)Phenyl)Benzofuran-3-Carboxamide

To a solution of ethyl 6-(1H-tetrazol-5-yl)benzofuran-3-carboxylate (299 mg, 1.16 mmol) and 4-((2-chloro-4-fluorobenzyl)oxy)aniline (350 mg, 1.39 mmol, 1.2 eq.) in toluene (10 mL) was added AlMe₃ (100 mg, 1.39 mmol, 1.2 eq.) at 0° C. The resulting mixture was stirred at 80° C. for 2 hrs under N₂. After the reaction completed, it was cooled to room temperature, quenched with water and concentrated. The residue was purified by C18 column (eluent: 35% to 55% (v/v) CH₃CN and H₂O with 10 MMOL/L NH₄HCO₃) to afford the title compound as a light yellow solid (158 mg, 29.4% Yield). LCMS (M+H)⁺: 480.0. ¹H NMR (300 MHz, DMSO-d₆) δ 10.24 (s, 1H), 8.93 (s, 1H), 8.38-8.26 (m, 2H), 8.09 (dd, J=8.2, 1.4 Hz, 1H), 7.74-7.61 (m, 3H), 7.51 (dd, J=8.9, 2.6 Hz, 1H), 7.34-7.21 (m, 1H), 7.19-6.94 (m, 2H), 5.12 (s, 2H), —NH was not observed.

### Example 6: 6-((1H-tetrazol-5-yl)oxy)-N-(4-(3,3-difluorocyclobutoxy)-3-fluorophenyl)-5-fluorobenzofuran-3-carboxamide

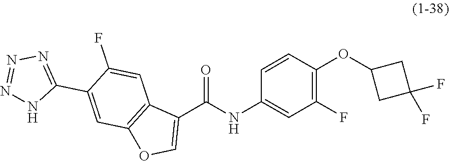 [SMILES: O=C(Nc1ccc(OC2CC(F)(F)C2)c(F)c1)c1coc2cc(-c3nnn[nH]3)c(F)cc12]

### Step 1: Preparation of 4-(3,3-Difluorocyclobutoxy)-3-Fluoroaniline

The title compound was prepared following step 1 and step 2 of Example 1. LCMS (M−H)⁻: 216.0.

### Step 2: Preparation of 5-Fluoro-2-Hydroxy-4-Methoxybenzaldehyde

To an ice-cold solution of 4-fluoro-3-methoxyphenol (1.42 g, 10.0 mmol) in MeCN (50 mL) under N₂ was added anhydrous magnesium chloride (1.90 g, 20.0 mmol) portion wise. After adding solid paraformaldehyde (2.10 g, 69.9 mmol) in the reaction mixture, triethylamine (5.60 mL, 40.0 mmol) was added dropwise. The reaction flask was sealed, and the mixture was stirred at 75° C. for 2 hrs. The reaction was cooled to room temperature, diluted with EtOAc, and washed sequentially with 1 M HCl, water and brine. The organic layer was dried over anhydrous Na₂SO₄ and concentrated to afford the title compound (1.54 g, 91%) as an off-white solid. This material was used in the next step without further purification. LCMS (M+H)⁺: 171.2.

### Step 3: Preparation of Ethyl 5-Fluoro-6-Methoxybenzofuran-3-Carboxylate

To a solution of 5-fluoro-2-hydroxy-4-methoxybenzaldehyde (3.5 g, 21 mmol) in DCM (10 mL) was added BF₄·OEt₂ (0.33 g, 2.1 mmol, 0.1 eq.) and ethyl 2-diazoacetate (4.7 g, 41 mmol, 2 eq.) at room temperature. After stirring the reaction for 30 min, it was treated with H₂SO₄ (2.0 g, 21 mmol, 1 eq.) and stirred at room temperature for an additional 2 hrs. The reaction was quenched with water and extracted with DCM (3×100 mL). The combined organic layers were washed with brine, dried over anhydrous Na₂SO₄, filtered and concentrated. The residue was purified by silica gel column to afford the title compound (3.9 g, 80%).

### Step 4: Preparation of Ethyl 5-Fluoro-6-Hydroxybenzofuran-3-Carboxylate

To a solution of ethyl 5-fluoro-6-methoxybenzofuran-3-carboxylate (3.9 g, 16 mmol) was added 3,3-difluorocyclobutan-1-ol (4.1 g, 16 mmol). The reaction mixture was cooled to 0° C. and stirred at 0° C. for 2 hrs. The reaction was quenched with water (50 mL) and extracted with ethyl acetate (50 mL×3). The combined organic extracts were washed with brine, dried over anhydrous Na₂SO₄, filtered and concentrated. The crude product was purified by silica gel chromatography to afford the title compound as a white solid (2.9 g, 79% yield). LCMS (M+H)⁺: 225.0.

### Step 5: Preparation of Ethyl 6-Cyanato-5-Fluorobenzofuran-3-Carboxylate

To a solution of ethyl 5-fluoro-6-hydroxybenzofuran-3-carboxylate (600 mg, 2.66 mmol) and BrCN (338 mg, 3.19 mmol, 1.2 eq.) in Et₂O (10 mL) was added Et₃N (537 mg, 5.32 mmol, 2 eq.) at 0° C. The mixture was stirred at 0° C. for 3 hrs, quenched with water (50 mL) and extracted with ethyl acetate (50 mL×3). The combined organic extracts were washed with brine, dried over anhydrous Na₂SO₄, filtered and concentrated. The crude product was purified by silica gel chromatography to afford the title compound as a white solid (200 mg, 30% yield).

### Step 6: Preparation of Ethyl 6-((1H-Tetrazol-5-Yl)Oxy)-5-Fluorobenzofuran-3-Carboxylate

To a solution of ethyl 6-cyanato-5-fluorobenzofuran-3-carboxylate (350 mg, 1.39 mmol) and Bu₂SnO (173 mg, 697 μmol) in toluene (10 mL) was drop wisely added TMSN₃ (482 mg, 4.18 mmol). The mixture was stirred at 100° C. for 2 hrs under N₂. After the reaction completed, it was cooled to room temperature, quenched with water and extracted with ethyl acetate (3×50 mL). The combined organic layers were washed with brine, dried over anhydrous Na₂SO₄, filtered and concentrated. Then the residue was purified directly by C18 column (eluent: 35% to 55% (v/v) Acetonitrile and Water with 10 MMOL/L NH₄HCO₃) to give the title compound as a yellow solid (250 mg, 62% Yield). LCMS (M+H)⁺: 293.0.

### Step 7: Preparation of 6-((1H-Tetrazol-5-Yl)Oxy)-5-Fluorobenzofuran-3-Carboxylic Acid

To a solution of ethyl 6-((1H-tetrazol-5-yl)oxy)-5-fluorobenzofuran-3-carboxylate (230 mg, 0.787 mmol) in THF/MeOH/H₂O (10 mL, v/v/v=3/1/1) was added LiOH (94 mg, 3.94 mmol, 5 eq.) at 0° C. The reaction was slowly warmed to room temperature and stirred for 2 hrs. After adjusting the pH to ˜7, the reaction was concentrated and purified directly by C18 column (eluent: 10% to 30% (v/v) CH₃CN and H₂O with 10 MMOL/L NH₄HCO₃) to afford the title compound (150 mg, 72%). LCMS (M+H)⁺: 264.9.

### Step 8: Preparation of 6-((1H-Tetrazol-5-Yl)Oxy)-N-(4-(3,3-Difluorocyclobutoxy)-3-Fluorophenyl)-5-Fluorobenzofuran-3-Carboxamide

To a solution of 6-((1H-tetrazol-5-yl)oxy)-5-fluorobenzofuran-3-carboxylic acid (130 mg, 492 μmol) and 4-(3,3-difluorocyclobutoxy)-3-fluoroaniline (107 mg, 492 μmol, 1 eq.) in pyridine (10 mL) was added EDCI (113 mg, 591 μmol, 1.2 eq.). The reaction was stirred at room temperature for 2 hrs, quenched with water and extracted with ethyl acetate (3×50 mL). The combined organic layers were washed with brine, dried over anhydrous Na₂SO₄, filtered and concentrated. The residue was purified directly by C18 column (eluent: 35% to 55% (v/v) Acetonitrile and Water with 10 MMOL/L NH₄HCO₃) to afford the title compound as a yellow solid (15.1 mg, 6.52% Yield). LCMS (M+H)⁺: 464.1. ¹H NMR (300 MHz, DMSO-d₆) δ 10.34 (s, 1H), 8.86 (s, 1H), 7.93-8.07 (m, 2H), 7.76 (dd, J=13.6, 2.5 Hz, 1H), 7.43-7.56 (m, 1H), 7.09-7.19 (m, 2H), 4.82 (s, 1H), 3.12-3.32 (m, 2H), 2.81-2.93 (m, 2H).

The following compounds were synthesized following Example 6:

| Cmpd | | MS | |
|---|---|---|---|
| No. | Structure/Name | (M + H)⁺ | ¹H NMR |
| 1-39 | 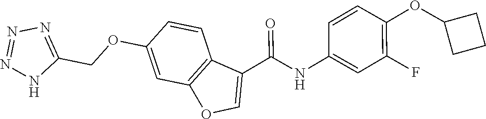 [SMILES: O=C(Nc1ccc(OC2CCC2)c(F)c1)c1coc2cc(OCc3nnn[nH]3)ccc12] | 498.1 | (400 MHz, DMSO-d₆) δ 8.74 (s, 1H), 7.86 (d, J = 10.6 Hz, 1H), 7.61-7.72 (m, 4H), 7.51-7.60 (m, 1H), 7.18-7.28 (m, 1H), 7.07 (d, J = 10.6 Hz, 2H), 5.10 (s, 2H). 2 NH |
| | | | were not observed. |
| | 6-((1H-tetrazol-5-yl)oxy)-N-(4-((2-chloro-4- | | |
| | fluorobenzyl)oxy)phenyl)-5-fluorobenzofuran-3-carboxamide | | |

### Example 7: 5-fluoro-N-(3-fluoro-4-(1-methylcyclopropoxy)phenyl)-6-(1H-tetrazol-5-yl)benzofuran-3-carboxamide

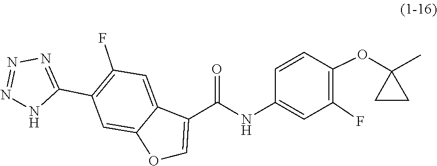 [SMILES: CC1(Oc2ccc(NC(=O)c3coc4cc(-c5nnn[nH]5)c(F)cc34)cc2F)CC1]

### Step 1: Preparation of 4-(3,3-Difluorocyclobutoxy)-3-Fluoroaniline

The title compound was prepared following step 1 and step 2 of Example 1. LCMS (M−H)⁺: 182.0.

### Step 2: Preparation of 5-Fluoro-2-Hydroxy-4-Methoxybenzaldehyde

To a solution of 4-chloro-5-fluoro-2-hydroxybenzaldehyde (5 g, 0.03 mol) in DCM (50 mL) was added 2-chloro-6-methyl-2-ethoxy-2-oxoethane-1-diazonium (0.01 kg, 0.1 mol, 4 eq.). After removal of the air in the reaction flask, it was backfilled with N₂. The reaction mixture was treated with BF₄·OEt₂ (0.5 g, 3 mmol, 0.1 eq.) at 0° C. and stirred for 2 hrs under N₂. After removal of the volatile solvent, the residue was treated with H₂SO₄. The resulting mixture was stirred at 0° C. for 2 hrs, carefully quenched with water (50 mL) and extracted with DCM (100 mL×3). The combined organic extracts were washed with brine, dried over anhydrous Na₂SO₄, filtered and concentrated. The crude product was purified by silica gel chromatography to afford the title compound as a yellow solid (3.5 g, 50% yield).

### Step 3: Preparation of Ethyl 5-Fluoro-6-Methoxybenzofuran-3-Carboxylate

The reaction mixture of ethyl 6-chloro-5-fluorobenzofuran-3-carboxylate (1 g, 4 mmol) and Zn(CN)₂ (0.6 g, 5 mmol) and Zn (0.05 g, 0.8 mmol) and Pd₂(dba)₃ (0.4 g, 0.4 mmol) and dppf (0.5 g, 0.8 mmol) in DMF (30 mL) was degassed by bubbling with N₂ for 5 min and stirred at 100° C. for 2 hrs under N₂. After the reaction completed, it was cooled to room temperature, quenched with water and extracted with ethyl acetate (3×50 mL). The combined organic layers were washed with brine, dried over anhydrous Na₂SO₄, filtered and concentrated. Then the residue was purified by silica gel column to afford the title compound as a yellow solid (720 mg, 70% Yield).

### Step 4: Preparation of Ethyl 5-Fluoro-6-Hydroxybenzofuran-3-Carboxylate

To a solution of ethyl 6-cyano-5-fluorobenzofuran-3-carboxylate (900 mg, 3.86 mmol) and Bu₂SnO (480 mg, 1.93 mmol) in toluene (20 mL) was drop wisely added TMSN₃ (1.33 g, 11.6 mmol). The mixture was stirred at 100° C. for 2 hrs under N₂. After the reaction completed, it was cooled to room temperature, quenched with water and extracted with ethyl acetate (3×20 mL). The combined organic layers were washed with brine, dried over anhydrous Na₂SO₄, filtered and concentrated. The residue was purified by silica gel chromatography (5%-20% methanol/DCM) to afford the title compound as a yellow solid (810 mg, 71% yield). LCMS (M+H)⁺: 277.1.

### Step 5: Preparation of Ethyl 6-Cyanato-5-Fluorobenzofuran-3-Carboxylate

To a solution of ethyl 5-fluoro-6-(1H-tetrazol-5-yl)benzofuran-3-carboxylate (770 mg, 2.79 mmol) in THF/H₂O (10 mL, v/v=3/1) was added LiOH (134 mg, 5.58 mmol) at 0° C. The reaction was slowly warmed to room temperature and stirred for 2 hrs. After the reaction completed, its pH was adjusted to ˜7. The reaction was concentrated to afford the title compound (700 mg, 79% yield) which was used directly without further purification. LCMS (M+H)⁺: 249.0.

### Step 6: Preparation of Ethyl 6-((1H-Tetrazol-5-Yl)Oxy)-5-Fluorobenzofuran-3-Carboxylate

To a solution of 5-fluoro-6-(1H-tetrazol-5-yl)benzofuran-3-carboxylic acid (200 mg, 806 μmol) and 3-fluoro-4-(1-methylcyclopropoxy)aniline (146 mg, 806 μmol, 1 eq.) and HATU (367 mg, 967 μmol, 1.2 eq.) in DMF (10 mL) was added DIEA (312 mg, 2.42 mmol, 3 eq.) at room temperature. The reaction was stirred at room temperature for 2 hrs. After the reaction completed, it was quenched with water and extracted with ethyl acetate (3×50 mL). The combined organic layers were washed with brine, dried over anhydrous Na₂SO₄, filtered and concentrated. The residue was purified by C18 column (eluent: 30% to 50% (v/v) Acetonitrile and Water with 10 MMOL/L NH₄HCO₃) to afford the crude product. The crude product was purified further by Xselect CSH OBD C18 Column, 30×150 mm 5 μm column (eluent: 40% to 66% (v/v) Acetonitrile and Water with 0.05% TFA) to afford the title compound as a light yellow solid (45.6 mg, 13.7% Yield). LCMS (M+H)⁺: 412.0. ¹H NMR (300 MHz, DMSO-d₆) δ 10.38 (s, 1H), 8.98 (s, 1H), 8.40 (d, J=5.4 Hz, 1H), 8.06 (d, J=10.3 Hz, 1H), 7.74 (dd, J=13.6, 2.4 Hz, 1H), 7.50-7.23 (m, 2H), 1.52 (s, 3H), 1.01-0.92 (m, 2H), 0.92-0.72 (m, 2H), —NH was not observed.

The following compounds were synthesized following Example 7:

| Cmpd | | MS | |
|---|---|---|---|
| No. | Structure/Name | (M + H)⁺ | ¹H NMR |
| 1-20 | 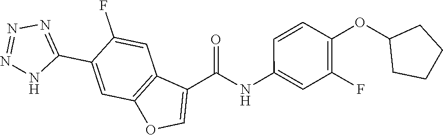 [SMILES: O=C(Nc1ccc(OC2CCCC2)c(F)c1)c1coc2cc(-c3nnn[nH]3)c(F)cc12] N-(4-(cyclopentyloxy)-3-fluorophenyl)-5- fluoro-6-(1H-tetrazol-5-yl)benzofuran-3-carboxamide | 426.1 | (300 MHz, DMSO-d₆) δ 8.89 (s, 1H), 8.31 (d, J = 5.4, 1H), 7.97 (d, J = 10.2, 1H), 7.69 (dd, J = 13.5, 2.5 Hz, 1H), 7.46- 7.36 (m, 1H), 7.17 (t, J = 9.3, 1H), 4.90 − 4.80 (m, 1H), 2.01 − 1.84 (m, 2H), 1.88 − 1.64 (m, 4H), 1.64 − 1.53 (m, 2H), 2 NH were not observed. |
| 1-21 | 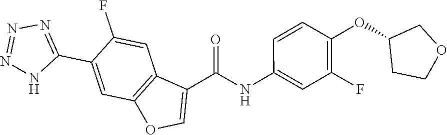 [SMILES: O=C(Nc1ccc(O[C@H]2CCOC2)c(F)c1)c1coc2cc(-c3nnn[nH]3)c(F)cc12] (S)-5-fluoro-N-(3-fluoro-4- ((tetrahydrofuran-3-yl)oxy)phenyl)-6-(1H- tetrazol-5-yl)benzofuran-3-carboxamide | 428.0 | (300 MHz, DMSO-d₆) δ 10.37 (s, 1H), 8.96 (s, 1H), 8.36 (d, J = 5.4 Hz, 1H), 8.02 (d, J = 10.3 Hz, 1H), 7.75 (dd, J = 13.6, 2.5 Hz, 1H), 7.43 (d, J = 9.0 Hz, 1H), 7.20 (t, J = 9.2 Hz, 1H), 5.12 − 5.01 (m, 1H), 4.04 − 3.68 (m, 4H), 2.31 − 2.13 (m, 1H), 2.09 − 1.94 (m, 1H), NH was not observed. |
| 1-22 | 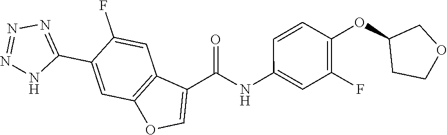 [SMILES: O=C(Nc1ccc(O[C@@H]2CCOC2)c(F)c1)c1coc2cc(-c3nnn[nH]3)c(F)cc12] (R)-5-fluoro-N-(3-fluoro-4- ((tetrahydrofuran-3-yl)oxy)phenyl)-6-(1H- tetrazol-5-yl)benzofuran-3-carboxamide | 428.0 | (300 MHz, DMSO-d₆) δ 10.36 (s, 1H), 8.95 (s, 1H), 8.35 (d, J = 5.5 Hz, 1H), 8.01 (d, J = 10.3 Hz, 1H), 7.75 (dd, J = 13.6, 2.5 Hz, 1H), 7.43 (d, J = 9.0 Hz, 1H), 7.20 (t, J = 9.2 Hz, 1H), 5.12 − 5.01 (m, 1H), 3.94 − 3.73 (m, 4H), 2.29 − 2.13 (m, 1H), 2.08 − 1.94 (m, 1H), NH was not observed. |
| 1-27 | 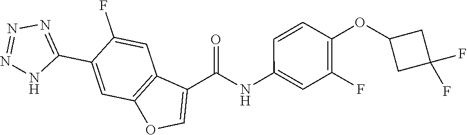 [SMILES: O=C(Nc1ccc(OC2CC(F)(F)C2)c(F)c1)c1coc2cc(-c3nnn[nH]3)c(F)cc12] N-(4-(3,3-difluorocyclobutoxy)-3- fluorophenyl)-5-fluoro-6-(1H-tetrazol-5- | 448.1 | (300 MHz, DMSO-d₆) δ 10.47 (s, 1H), 9.03 (s, 1H), 8.35 (d, J = 5.4 Hz, 1H), 8.01 (d, J = 10.2 Hz, 1H), 7.78 (d, J = 13.6 Hz, 1H), 7.46 (d, J = 8.8 Hz, 1H), 7.12 (t, J = 9.2 Hz, 1H), 4.88 − 4.73 (m, 1H), 3.32 − 3.07 (m, 2H), 2.87 − 2.65 (m, 2H), NH was not observed. |
| | yl)benzofuran-3-carboxamide | | |
| 1-28 | 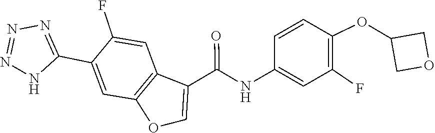 [SMILES: O=C(Nc1ccc(OC2COC2)c(F)c1)c1coc2cc(-c3nnn[nH]3)c(F)cc12] 5-fluoro-N-(3-fluoro-4-(oxetan-3-yloxy)phenyl)-6-(1H-tetrazol-5- yl)benzofuran-3-carboxamide | 414.0 | (300 MHz, DMSO-d₆) δ 10.40 (s, 1H), 8.95 (s, 1H), 8.32 (d, J = 5.4 Hz, 1H), 7.98 (d, J = 10.3 Hz, 1H), 7.77 (dd, J = 13.6, 2.5 Hz, 1H), 7.45 − 7.34 (m, 1H), 6.88 (t, J = 9.2 Hz, 1H), 5.38 − 5.24 (m, 1H), 4.99 − 4.88 (m, 2H), 4.65 − 4.55 (m, 2H), NH was not observed. |
| 1-35 | 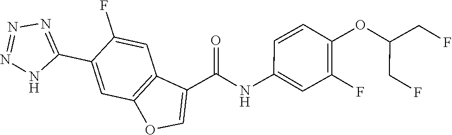 [SMILES: O=C(Nc1ccc(OC(CF)CF)c(F)c1)c1coc2cc(-c3nnn[nH]3)c(F)cc12] N-(4-((1,3-difluoropropan-2-yl)oxy)-3- | 436.0 | (300 MHz, DMSO-d₆) δ 10.48 (s, 1H), 8.98 (s, 1H), 8.25 (d, J = 5.5 Hz, 1H), 7.91 (d, J = 10.3 Hz, 1H), 7.80 (dd, J = 13.5, 2.5 Hz, 1H), 7.53 − 7.44 (m, 1H), 7.32 (t, J = 9.2 Hz, 1H), 4.97 − 4.73 (m, 3H), 4.73 − 4.58 (m, 2H), NH was not observed. |
| | fluorophenyl)-5-fluoro-6-(1H-tetrazol-5- | | |
| | yl)benzofuran-3-carboxamide | | |

### Example 8: N-(4-((2-chloro-4-fluorobenzyl)oxy)phenyl)-5-fluoro-6-(1H-tetrazol-5-yl)benzofuran-3-carboxamide

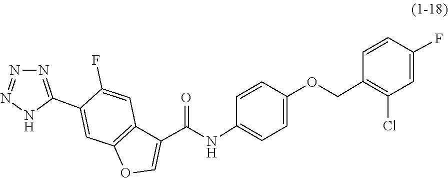 [SMILES: O=C(Nc1ccc(OCc2ccc(F)cc2Cl)cc1)c1coc2cc(-c3nnn[nH]3)c(F)cc12]

### Step 1: Preparation of N-(4-((2-Chloro-4-Fluorobenzyl)Oxy)Phenyl)-5-Fluoro-6-(1H-Tetrazol-5-Yl)Benzofuran-3-Carboxamide

To a solution of 5-fluoro-6-(1H-tetrazol-5-yl)benzofuran-3-carboxylic acid (200 mg, 806 μmol) and 4-((2-chloro-4-fluorobenzyl)oxy)aniline (203 mg, 806 μmol, 1 eq.) in pyridine (10 mL) was added EDCI (185 mg, 967 μmol, 1.2 eq.) at room temperature. The reaction was stirred at room temperature for 2 hrs, quenched with water and extracted with ethyl acetate (3×50 mL). The combined organic layers were washed with brine, dried over anhydrous Na₂SO₄, filtered and concentrated. Then the residue was directly purified by C18 column (eluent: 35% to 55% (v/v) Acetonitrile and Water with 10 MMOL/L NH₄HCO₃) to afford the title compound as a yellow solid (108.3 mg, 27.4% Yield). LCMS (M+H)⁺: 482.0. ¹H NMR (300 MHz, DMSO-d₆) δ 10.28 (s, 1H), 9.01 (s, 1H), 8.38 (d, J=5.4 Hz, 1H), 8.05 (d, J=10.3 Hz, 1H), 7.75-7.62 (m, 3H), 7.53 (dd, J=8.9, 2.6 Hz, 1H), 7.35-7.22 (m, 1H), 7.18-7.01 (m, 2H), 5.13 (s, 2H), —NH was not observed.

### Example 9: N-(4-((4-chlorothiophen-2-yl)methoxy)phenyl)-5-fluoro-6-(1H-tetrazol-5-yl)benzofuran-3-carboxamide

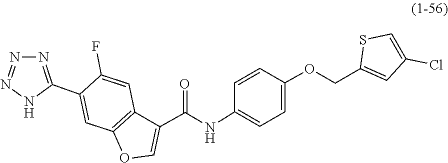 [SMILES: O=C(Nc1ccc(OCc2cc(Cl)cs2)cc1)c1coc2cc(-c3nnn[nH]3)c(F)cc12]

### Step 1: Preparation of 4-Chloro-2-((4-Nitrophenoxy)Methyl)Thiophene

To a solution of (4-chlorothiophen-2-yl)methanol (500 mg, 3.36 mmol) and 1-fluoro-4-nitrobenzene (712 mg, 5.05 μmol, 1.5 eq.) in THF (10 mL) was added t-BuOK (566 mg, 5.05 mmol, 1.5 equiv.) at 0° C. The reaction mixture was warmed to room temperature and stirred for 2 hrs. The reaction was quenched with water (50 mL) and extracted with ethyl acetate (50 mL×3). The combined organic extracts were washed with brine, dried over anhydrous Na₂SO₄, filtered and concentrated. The residue was purified by silica gel chromatography to afford the title compound as a white solid (800 mg, 88.2% yield).

### Step 2: Preparation of 4-((4-Chlorothiophen-2-Yl)Methoxy)Aniline

To a solution of 4-chloro-2-((4-nitrophenoxy)methyl)thiophene (750 mg, 2.78 mmol) in EtOH/H₂O (4:1, 12.5 mL) was added NH₄Cl (1.5 g, 27.8 mmol, 10 eq.) and Fe (0.78 g, 13.9 mmol, 5 eq.). After removal of the air in the reaction flask, it was backfilled with N₂. The reaction was stirred at 85° C. for 2 hrs under N₂. It was quenched with water (50 mL) and extracted with ethyl acetate (50 mL×3). The combined organic extracts were washed with brine, dried over anhydrous Na₂SO₄, filtered and concentrated. The crude product was purified by silica gel chromatography to afford the title compound as a white solid (600 mg, 90% yield). LCMS (M+H)⁺: 240.0.

### Step 3: Preparation of N-(4-((4-Chlorothiophen-2-Yl)Methoxy)Phenyl)-5-Fluoro-6-(1H-Tetrazol-5-Yl)Benzofuran-3-Carboxamide

To a solution of 5-fluoro-6-(1H-tetrazol-5-yl)benzofuran-3-carboxylic acid (150 mg, 0.604 mmol) in DMF (5 mL) was added 4-((4-chlorothiophen-2-yl)methoxy)aniline (174 mg, 0.725 mmol, 1.2 eq.), HATU (275 mg, 0.725 mmol, 1.2 eq.) and DIEA (234 mg, 1.81 mmol, 3 eq.). The reaction was stirred at room temperature for 1 hr, quenched with water (50 mL) and extracted with ethyl acetate (50 mL×3). The combined organic extracts were washed with brine, dried over anhydrous Na₂SO₄, filtered and concentrated. The crude product was further purified by XBridge Prep C18 OBD Column (eluent: 22% to 47% (v/v) CH₃CN and H₂O with 10 MMOL/L NH₄HCO₃) to afford the title compound as a white solid (20.1 mg, 6.93% yield). LCMS (M+H)⁺: 470.1. ¹H NMR (300 MHz, DMSO-d₆) δ 10.14 (s, 1H), 8.78 (s, 1H), 8.11 (d, J=5.6 Hz, 1H), 7.80 (d, J=10.3 Hz, 1H), 7.68-7.60 (m, 2H), 7.57 (d, J=1.6 Hz, 1H), 7.24-7.17 (m, 1H), 7.18-7.08 (brs, 1H), 7.09-6.97 (m, 2H), 5.25 (s, 2H).

### Example 10: N-(4-((4-chloro-3-fluorobenzyl)oxy)phenyl)-5-fluoro-6-(1H-tetrazol-5-yl)benzofuran-3-carboxamide

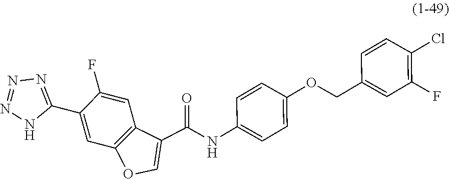 [SMILES: O=C(Nc1ccc(OCc2ccc(Cl)c(F)c2)cc1)c1coc2cc(-c3nnn[nH]3)c(F)cc12]

### Step 1: Preparation of 4-((4-Chloro-3-Fluorobenzyl)Oxy)Aniline

The title compound was prepared following step 1 and step 2 of Example 1. LCMS (M−H)⁺: 251.9.

### Step 2: Preparation of N-(4-((4-Chloro-3-Fluorobenzyl)Oxy)Phenyl)-5-Fluoro-6-(1H-Tetrazol-5-Yl)Benzofuran-3-Carboxamide

To a solution of 4-((4-chloro-3-fluorobenzyl)oxy)aniline (50 mg, 0.2 mmol), 5-fluoro-6-(1H-tetrazol-5-yl)benzofuran-3-carboxylic acid (49 mg, 0.2 mmol, 1 eq.) and HATU (91 mg, 0.24 mmol, 1.2 eq.) in DMF (5 mL) was added DIEA ‘(77 mg, 0.6 mmol, 3 eq.) at room temperature. The reaction was stirred at room temperature for 2 hrs. After the reaction completed, it was quenched with water and extracted with ethyl acetate (3×50 mL). The combined organic layers were washed with brine, dried over anhydrous Na₂SO₄, filtered and concentrated. Then the residue was purified by XBridge Prep C18 OBD Column, 30*150 mm, 5 m; Mobile Phase A: Water(10 mmol/L NH4HCO3), Mobile Phase B: Acetonitrile; Flow rate: 60 mL/min; Gradient: 30% B to 60% B in 7 min to afford the title compound as a white solid (11.9 mg, 12.0% Yield). LCMS (M+H)⁺: 482.0. ¹H NMR (300 MHz, DMSO-d₆) δ 10.14 (s, 1H), 8.81 (s, 1H), 8.12 (d, J=5.6 Hz, 1H), 7.81 (d, J=10.3 Hz, 1H), 7.72-7.57 (m, 3H), 7.52 (dd, J=10.3, 1.9 Hz, 1H), 7.34 (d, J=8.3 Hz, 1H), 7.16 (s, 1H), 7.10-6.99 (m, 2H), 5.13 (s, 2H).

### Example 11: N-(4-((5-chloropyridin-2-yl)methoxy)phenyl)-5-fluoro-6-(1H-tetrazol-5-yl)benzofuran-3-carboxamide

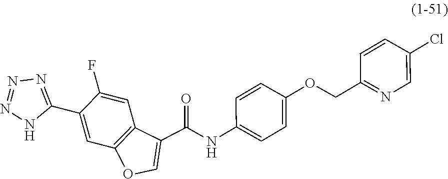 [SMILES: O=C(Nc1ccc(OCc2ccc(Cl)cn2)cc1)c1coc2cc(-c3nnn[nH]3)c(F)cc12]

### Step 1: Preparation of 4-((5-Chloropyridin-2-Yl)Methoxy)Aniline

The title compound was prepared following step 1 and step 2 of Example 1. LCMS (M−H)⁺: 235.2.

### Step 2: Preparation of N-(4-((5-Chloropyridin-2-Yl)Methoxy)Phenyl)-5-Fluoro-6-(1H-Tetrazol-5-Yl)Benzofuran-3-Carboxamide

To a solution of 4-((5-chloropyridin-2-yl)methoxy)aniline (50 mg, 0.21 mmol), 5-fluoro-6-(1H-tetrazol-5-yl)benzofuran-3-carboxylic acid (53 mg, 0.21 mmol, 1 eq.) and HATU (97 mg, 0.26 mmol, 1.2 eq.) in DMF (5 mL) was added DIEA (82 mg, 0.64 mmol, 3 eq.) at room temperature. The reaction was stirred at room temperature for 2 hrs. After the reaction completed, it was quenched with water and extracted with ethyl acetate (3×50 mL). The combined organic layers were washed with brine, dried over anhydrous Na₂SO₄, filtered and concentrated. The residue was purified by C18 reverse column (eluent: 30% to 50% (v/v) Acetonitrile and Water with 10 MMOL/L NH₄HCO₃) to give the crude product. Then the crude product was further purified by XBridge Prep OBD C18 Column, 19*250 mm, 5 m; Mobile Phase A: Water(10 mmol/L NH₄HCO₃), Mobile Phase B: MeOH—HPLC; Flow rate: 25 mL/min; Gradient: 45% B to 65% B in 10 min to afford the title compound as a white solid (13.9 mg, 14% Yield). LCMS (M+H)⁺: 465.0. ¹H NMR (300 MHz, DMSO-d₆) δ 10.16 (s, 1H), 8.83 (s, 1H), 8.65 (d, J=2.5 Hz, 1H), 8.16 (d, J=5.5 Hz, 1H), 7.99 (dd, J=8.4, 2.5 Hz, 1H), 7.85 (d, J=10.3 Hz, 1H), 7.71-7.62 (m, 2H), 7.58 (d, J=8.4 Hz, 1H), 7.18-6.96 (m, 3H), 5.19 (s, 2H).

### Example 12: N-(4-((4-chloro-3,5-difluorobenzyl)oxy)phenyl)-5-fluoro-6-(1H-tetrazol-5-yl)benzofuran-3-carboxamide

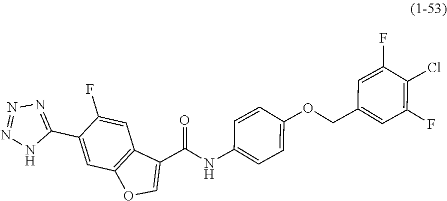 [SMILES: O=C(Nc1ccc(OCc2cc(F)c(Cl)c(F)c2)cc1)c1coc2cc(-c3nnn[nH]3)c(F)cc12]

### Step 1: Preparation of 4-((4-Chloro-3,5-Difluorobenzyl)Oxy)Aniline

The title compound was prepared following step 1 and step 2 of Example 1. LCMS (M−H)⁺: 270.1.

### Step 2: Preparation of N-(4-((4-Chloro-3,5-Difluorobenzyl)Oxy)Phenyl)-5-Fluoro-6-(1H-Tetrazol-5-Yl)Benzofuran-3-Carboxamide

To a solution of 4-((4-chloro-3,5-difluorobenzyl)oxy)aniline (50 mg, 0.19 mmol), 5-fluoro-6-(1H-tetrazol-5-yl)benzofuran-3-carboxylic acid (46 mg, 0.19 mmol, 1 eq.) and HATU (85 mg, 0.22 mmol, 1.2 eq.) in DMF (5 mL) was added DIEA (72 mg, 0.56 mmol, 3 eq.) at room temperature. The reaction was stirred at room temperature for 2 hrs, quenched with water and extracted with ethyl acetate (3×50 mL). The combined organic layers were washed with brine, dried over anhydrous Na₂SO₄, filtered and concentrated. The residue was purified by C18 reverse column (eluent: 30% to 50% (v/v) Acetonitrile and Water with 10 MMOL/L NH₄HCO₃) to give the crude product. Then the crude product was further purified by XBridge Prep C18 OBD Column, 30*100 mm, 5 μm; Mobile Phase A: Water(10 mmol/L NH₄HCO₃), Mobile Phase B: Acetonitrile; Flow rate: 60 mL/min; Gradient: 30% B to 53% B in 9 min to afford the title compound as a white solid (14.2 mg, 15 Yield). LCMS (M+H): 500.0. ¹H NMR (300 MHz, DMSO-d₆) δ 10.16 (s, 1H), 8.83 (s, 1H), 8.16 (d, J=5.6 Hz, 1H), 7.84 (d, J=10.3 Hz, 1H), 7.73-7.61 (m, 2H), 7.50-7.39 (m, 2H), 7.19-6.94 (m, 3H), 5.15 (s, 2H).

The following compounds were synthesized following Example 12:

| Cmpd | | MS | |
|---|---|---|---|
| No. | Structure/Name | (M + H)⁺ | ¹H NMR |
| 1-19 | 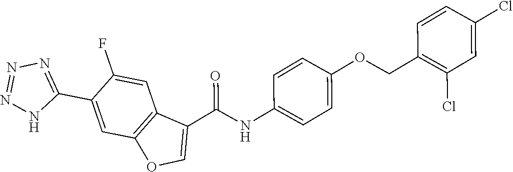 [SMILES: O=C(Nc1ccc(OCc2ccc(Cl)cc2Cl)cc1)c1coc2cc(-c3nnn[nH]3)c(F)cc12] | 498.0 | (300 MHz, DMSO-d₆) δ 10.21 (s, 1H), 8.90 (s, 1H), 8.26 (d, J = 5.5 Hz, 1H), 7.93 (d, J = 10.3 Hz, 1H), 7.75 − 7.60 (m, 4H), 7.51 (dd, J = 8.3, 2.2 Hz, 1H), 7.07 (m, 3H), 5.16 (s, 2H). |
| | N-(4-((2,4-dichlorobenzyl)oxy)phenyl)-5- | | |
| | fluoro-6-(1H-tetrazol-5-yl)benzofuran-3- | | |
| | carboxamide | | |
| 1-41 | 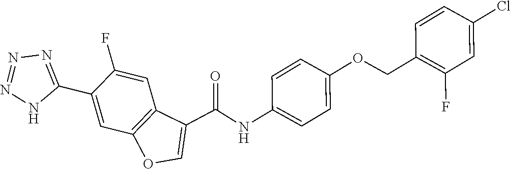 [SMILES: O=C(Nc1ccc(OCc2ccc(Cl)cc2F)cc1)c1coc2cc(-c3nnn[nH]3)c(F)cc12] | 482.0 | (300 MHz, DMSO-d₆) δ 8.85 (s, 1H), 8.24 (d, J = 5.5 Hz, 1H), 7.92 (d, J = 10.3 Hz, 1H), 7.72 − 7.55 (m, 3H), 7.50 (dd, J = 10.0, 2.1 Hz, 1H), 7.35 (dd, J = 8.2, 2.2 Hz, 1H), 7.12 − 7.01 (m, 2H), 5.13 (s, 2H), 2 NH were not observed. |
| | N-(4-((4-chloro-2-fluorobenzyl)oxy)phenyl)-5- | | |
| | fluoro-6-(1H-tetrazol-5-yl)benzofuran-3- | | |
| | carboxamide | | |
| 1-43 | 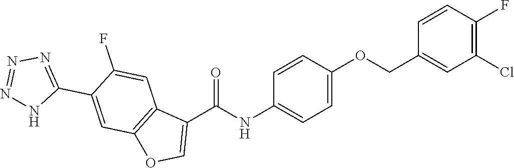 [SMILES: O=C(Nc1ccc(OCc2ccc(F)c(Cl)c2)cc1)c1coc2cc(-c3nnn[nH]3)c(F)cc12] | 482.0 | (300 MHz, DMSO-d₆) δ 10.25 (s, 1H), 8.98 (s, 1H), 8.40 (d, J = 5.2 Hz, 1H), 8.06 (d, J = 10.2 Hz, 1H), 7.77 − 7.64 (m, 3H), 7.58 − 7.41 (m, 2H), 7.13 − 7.03 (m, 2H), 5.13 (s, 2H), NH was not observed. |
| | N-(4-((3-chloro-4-fluorobenzyl)oxy)phenyl)-5- | | |
| | fluoro-6-(1H-tetrazol-5-yl)benzofuran-3- | | |
| | carboxamide | | |
| 1-44 | 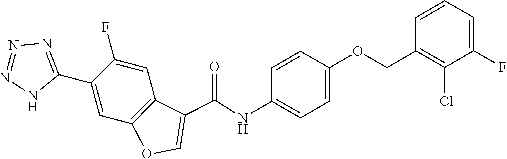 [SMILES: O=C(Nc1ccc(OCc2cccc(F)c2Cl)cc1)c1coc2cc(-c3nnn[nH]3)c(F)cc12] | 482.0 | (300 MHz, DMSO-d₆) δ 10.15 (s, 1H), 8.82 (s, 1H), 8.15 (d, J = 5.5 Hz, 1H), 7.83 (d, J = 10.3 Hz, 1H), 7.72- 7.64(m, 2H), 7.53 − 7.37 (m, 3H), 7.14 − 7.02 (m, 3H), 5.21 (s, 2H). |
| | N-(4-((2-chloro-3-fluorobenzyl)oxy)phenyl)-5- | | |
| | fluoro-6-(1H-tetrazol-5-yl)benzofuran-3- | | |
| | carboxamide | | |
| 1-45 | 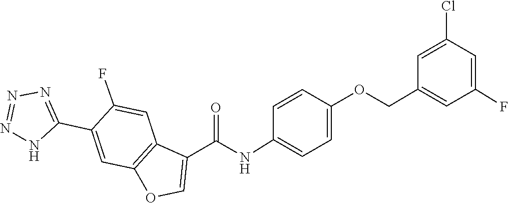 [SMILES: O=C(Nc1ccc(OCc2cc(F)cc(Cl)c2)cc1)c1coc2cc(-c3nnn[nH]3)c(F)cc12] | 482.0 | (300 MHz, DMSO-d₆) δ 10.14 (s, 1H), 8.81 (s, 1H), 8.15 (d, J = 5.9 Hz, 1H), 7.82 (dd, J = 11.0, 4.9 Hz, 1H), 7.72 − 7.61 (m, 2H), 7.47 − 7.27 (m, 3H), 7.22 − 6.96 (m, 3H), 5.15 (s, 2H). |
| | N-(4-((3-chloro-5-fluorobenzyl)oxy)phenyl)-5- | | |
| | fluoro-6-(1H-tetrazol-5-yl)benzofuran-3- | | |
| | carboxamide | | |
| 1-46 | 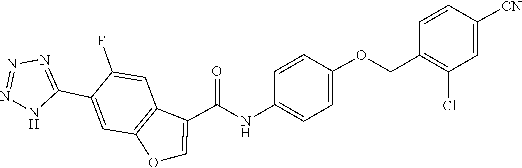 [SMILES: N#Cc1ccc(COc2ccc(NC(=O)c3coc4cc(-c5nnn[nH]5)c(F)cc34)cc2)c(Cl)c1] | 489.0 | (300 MHz, DMSO-d₆) δ 10.18 (s, 1H), 8.87 (s, 1H), 8.25 − 8.12 (m, 2H), 7.94 − 7.86 (m, 2H), 7.80 (d, J = 8.2 Hz, 1H), 7.73 − 7.64 (m, 2H), 7.13 − 7.04 (m, 2H), 5.25 (s, 2H), NH was not observed. |
| | N-(4-((2-chloro-4-cyanobenzyl)oxy)phenyl)-5- | | |
| | fluoro-6-(1H-tetrazol-5-yl)benzofuran-3- | | |
| | carboxamide | | |
| 1-47 | 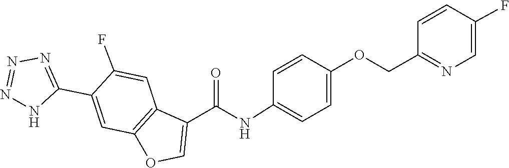 [SMILES: O=C(Nc1ccc(OCc2ccc(F)cn2)cc1)c1coc2cc(-c3nnn[nH]3)c(F)cc12] | 449.0 | (300 MHz, DMSO-d₆) δ 10.18 (s, 1H), 8.88 (s, 1H), 8.60 (d, J = 2.9 Hz, 1H), 8.24 (d, J = 5.5 Hz, 1H), 7.95 − 7.88 (m, 1H), 7.86 − 7.73 (m, 1H), 7.72 − 7.58 (m, 3H), 7.12 − 7.00 (m, 2H), 5.18 (s, 2H), NH was not observed. |
| | 5-fluoro-N-(4-((5-fluoropyridin-2- | | |
| | yl)methoxy)phenyl)-6-(1H-tetrazol-5- | | |
| | yl)benzofuran-3-carboxamide | | |
| 1-48 | 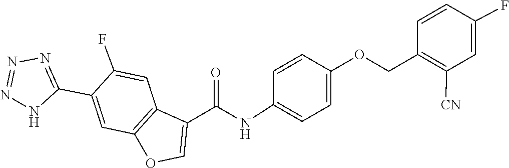 [SMILES: N#Cc1cc(F)ccc1COc1ccc(NC(=O)c2coc3cc(-c4nnn[nH]4)c(F)cc23)cc1] | 473.1 | (300 MHz, DMSO-d₆) δ 10.17 (s, 1H), 8.82 (s, 1H), 8.13 (d, J = 5.6 Hz, 1H), 7.95 (dd, J = 8.6, 2.7 Hz, 1H), 7.87 − 7.76 (m, 2H), 7.74 − 7.60 (m, 3H), 7.21 − 7.00 (m, 3H), 5.23 (s, 2H). |
| | N-(4-((2-cyano-4-fluorobenzyl)oxy)phenyl)-5- | | |
| | fluoro-6-(1H-tetrazol-5-yl)benzofuran-3- | | |
| | carboxamide | | |
| 1-50 | 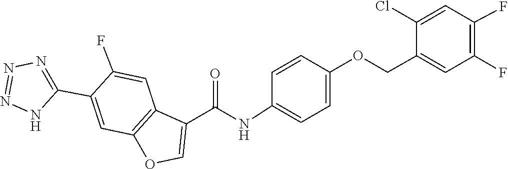 [SMILES: O=C(Nc1ccc(OCc2cc(F)c(F)cc2Cl)cc1)c1coc2cc(-c3nnn[nH]3)c(F)cc12] | 500.0 | (300 MHz, DMSO-d₆) δ 10.19 (s, 1H), 8.86 (s, 1H), 8.20 (d, J = 5.6 Hz, 1H), 7.93 − 7.65 (m, 5H), 7.16 − 7.01 (m, 3H), 5.12 (s, 2H). |
| | N-(4-((2-chloro-4,5- | | |
| | difluorobenzyl)oxy)phenyl)-5-fluoro-6-(1H- | | |
| | tetrazol-5-yl)benzofuran-3-carboxamide | | |
| 1-52 | 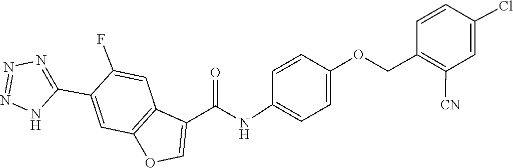 [SMILES: N#Cc1cc(Cl)ccc1COc1ccc(NC(=O)c2coc3cc(-c4nnn[nH]4)c(F)cc23)cc1] | 489.1 | (300 MHz, DMSO-d₆) δ 10.15 (s, 1H), 8.81 (s, 1H), 8.16 − 8.07 (m, 2H), 7.90 − 7.73 (m, 3H), 7.73 − 7.63 (m, 2H), 7.14 − 6.95 (m, 3H), 5.25 (s, 2H). |
| | N-(4-((4-chloro-2-cyanobenzyl)oxy)phenyl)-5- | | |
| | fluoro-6-(1H-tetrazol-5-yl)benzofuran-3- | | |
| | carboxamide | | |
| 1-54 | 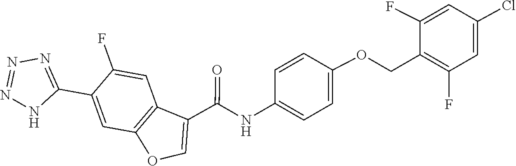 [SMILES: O=C(Nc1ccc(OCc2c(F)cc(Cl)cc2F)cc1)c1coc2cc(-c3nnn[nH]3)c(F)cc12] | 500.1 | (300 MHz, DMSO-d₆) δ 10.22 (s, 1H), 8.91 (s, 1H), 8.29 (d, J = 5.5 Hz, 1H), 7.96 (d, J = 10.3 Hz, 1H), 7.73 − 7.63 (m, 2H), 7.55 − 7.42 (m, 2H), 7.12 − 7.00 (m, 2H), 5.10 (s, 2H), NH was not observed. |
| | N-(4-((4-chloro-2,6- | | |
| | difluorobenzyl)oxy)phenyl)-5-fluoro-6-(1H- | | |
| | tetrazol-5-yl)benzofuran-3-carboxamide | | |
| 1-55 | 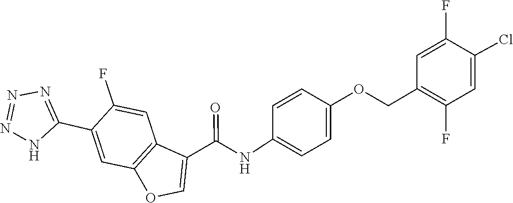 [SMILES: O=C(Nc1ccc(OCc2cc(F)c(Cl)cc2F)cc1)c1coc2cc(-c3nnn[nH]3)c(F)cc12] | 500.0 | (300 MHz, DMSO-d₆) δ 10.18 (s, 1H), 8.86 (s, 1H), 8.21 (d, J = 5.5 Hz, 1H), 7.89 (d, J = 10.3 Hz, 1H), 7.79 − 7.62 (m, 4H), 7.13 − 7.03 (m, 2H), 5.13 (s, 2H), NH was not observed. |
| | N-(4-((4-chloro-2,5- | | |
| | difluorobenzyl)oxy)phenyl)-5-fluoro-6-(1H- | | |
| | tetrazol-5-yl)benzofuran-3-carboxamide | | |
| 1-57 | 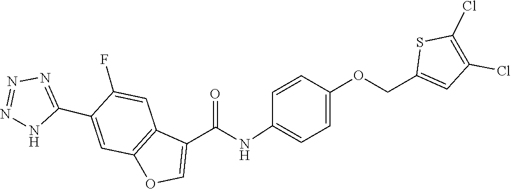 [SMILES: O=C(Nc1ccc(OCc2cc(Cl)c(Cl)s2)cc1)c1coc2cc(-c3nnn[nH]3)c(F)cc12] | 503.9 | (300 MHz, DMSO-d₆) δ 10.17 (s, 1H), 8.84 (s, 1H), 8.18 (d, J = 5.4 Hz, 1H), 7.86 (d, J = 10.3 Hz, 1H), 7.67 (d, J = 8.6 Hz, 2H), 7.29 (s, 1H), 7.05 (d, J = 8.8 Hz, 2H), 5.25 (s, 2H), NH was not observed. |
| | N-(4-((4,5-dichlorothiophen-2- | | |
| | yl)methoxy)phenyl)-5-fluoro-6-(1H-tetrazol-5- | | |
| | yl)benzofuran-3-carboxamide | | |

### Example 13: N-(4-cyclopentyl-3-fluorophenyl)-5-fluoro-6-(1H-tetrazol-5-yl)benzofuran-3-carboxamide

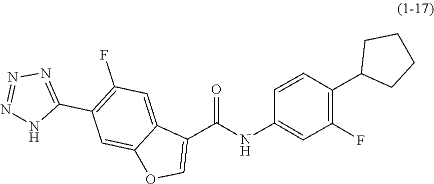 [SMILES: O=C(Nc1ccc(C2CCCC2)c(F)c1)c1coc2cc(-c3nnn[nH]3)c(F)cc12]

### Step 1: Preparation of 1-(Cyclopent-1-En-1-Yl)-2-Fluoro-4-Nitrobenzene

To a solution of 1-bromo-2-fluoro-4-nitrobenzene (1 g, 5 mmol) in 1,4-Doixane/H₂O (3:1, 12 mL) was added 2-(cyclopent-1-en-1-yl)-4,4,5,5-tetramethyl-1,3,2-dioxaborolane (0.9 g, 5 mmol, 1 eq.), Pd(dppf)Cl₂ (0.3 g, 0.5 mmol, 0.1 eq.), and K₂CO₃ (1 g, 9 mmol, 2 eq.). After removing the air in the reaction flask, it was backfilled with N₂. The reaction was stirred at 110° C. for 30 min under N₂. After the reaction completed, it was cooled to room temperature, quenched with water (50 mL) and extracted with ethyl acetate (50 mL×3). The combined organic extracts were washed with brine, dried over anhydrous Na₂SO₄, filtered and concentrated. The crude product was purified by silica gel chromatography to afford the product as a white solid (800 mg, 60% yield).

### Step 2: Preparation of 4-Cyclopentyl-3-Fluoroaniline

To a solution of 1-(cyclopent-1-en-1-yl)-2-fluoro-4-nitrobenzene (800 mg, 3.86 mmol) in MeOH (10 mL) was added wet Pd/C (400 mg, 0.5 eq.) at room temperature. After removing the air in the reaction flask, it was backfilled with N₂. After repeating the above process three times, the reaction flask was backfilled with H₂. The reaction was stirred at room temperature for 2 hrs. After the reaction completed, it was carefully filtered through a pad of Celite. The filtrate was concentrated to afford the crude product (650 mg, 88% yield), which was used directly in next step. LCMS (M+H)⁺: 180.1.

### Step 3: Preparation of N-(4-Cyclopentyl-3-Fluorophenyl)-5-Fluoro-6-(1H-Tetrazol-5-Yl)Benzofuran-3-Carboxamide

To a solution of 5-fluoro-6-(1H-tetrazol-5-yl)benzofuran-3-carboxylic acid (200 mg, 806 μmol) and 4-cyclopentyl-3-fluoroaniline (144 mg, 806 μmol, 1 eq.) and HATU (367 mg, 967 μmol, 1.2 eq.) in DMF (10 mL) was added DIEA (312 mg, 2.42 mmol, 3 eq.) at room temperature. The reaction was stirred at room temperature for 2 hrs. After the reaction completed, it was quenched with water and extracted with ethyl acetate (3×50 mL). The combined organic layers were washed with brine, dried over anhydrous sodium sulfate, filtered and concentrated. Then the residue was purified directly by C18 column (eluent: 35% to 55% (v/v) Acetonitrile and Water with 10 MMOL/L NH₄HCO₃) to afford the title compound as a yellow solid (40.1 mg, 11.9% Yield). LCMS (M+H)⁺: 410.0. ¹H NMR (300 MHz, DMSO-d₆) δ 10.36 (s, 1H), 8.86 (s, 1H), 8.16 (d, J=5.6 Hz, 1H), 7.84 (d, J=10.2 Hz, 1H), 7.68 (dd, J=13.0, 2.1 Hz, 1H), 7.45 (dd, J=8.5, 2.1 Hz, 1H), 7.34 (t, J=8.5 Hz, 1H), 7.17 (brs, 1H), 3.25-3.08 (m, 1H), 2.07-1.92 (m, 2H), 1.84-1.49 (m, 6H).

### Example 14: (S)-5-fluoro-N-(3-fluoro-4-(2-(trifluoromethyl)pyrrolidin-1-yl)phenyl)-6-(1H-tetrazol-5-yl)benzofuran-3-carboxamide

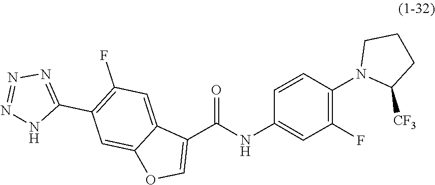 [SMILES: O=C(Nc1ccc(N2CCC[C@H]2C(F)(F)F)c(F)c1)c1coc2cc(-c3nnn[nH]3)c(F)cc12]

### Step 1: Preparation of(S)-1-(2-Fluoro-4-Nitrophenyl)-2-(Trifluoromethyl)Pyrrolidine

To a solution of 1-bromo-2-fluoro-4-nitrobenzene (1 g, 5 mmol) in 1,4-dioxane (40 mL) was added (S)-2-(trifluoromethyl)pyrrolidine (0.6 g, 5 mmol), XPhos Pd G3 (0.4 g, 0.5 mmol), XPhos (0.2 g, 0.5 mmol) and Cs₂CO₃ (3 g, 9 mmol). The reaction was stirred at 90° C. for 2 hrs. After the reaction completed, it was cooled to room temperature and filtered through a pad of Celite. The filtrate was concentrated, and the residue was purified by silica gel chromatography to afford the title compound as an off-white solid (1 g, 80% yield). LCMS (M+H)⁺: 279.2.

### Step 2: Preparation of (S)-3-Fluoro-4-(2-(Trifluoromethyl)Pyrrolidin-1-Yl)Aniline

The title compound was prepared following step 2 of Example 1. LCMS (M−H)⁺: 249.1.

### Step 3: Preparation of (S)-5-Fluoro-N-(3-Fluoro-4-(2-(Trifluoromethyl)Pyrrolidin-1-Yl)Phenyl)-6-(1H-Tetrazol-5-Yl)Benzofuran-3-Carboxamide

To a solution of ethyl 5-fluoro-6-(1H-tetrazol-5-yl)benzofuran-3-carboxylate (200 mg, 742 μmol) and (S)-3-fluoro-4-(2-(trifluoromethyl)pyrrolidin-1-yl)aniline (216 mg, 869 μmol, 1.5 eq.) in toluene (10 mL) was added AlMe₃ (104 mg, 1.45 mmol, 2 eq.) at 0° C. The reaction was stirred at 80° C. for 2 hrs under N². After the reaction completed, it was cooled to room temperature, quenched with water and extracted with ethyl acetate (3×50 mL). The combined organic layers were washed with brine, dried over anhydrous Na₂SO₄, filtered and concentrated. Then the residue was purified directly by C18 column (eluent: 35% to 55% (v/v) Acetonitrile and Water with 10 MMOL/L NH₄HCO₃) to afford the title compound as a pink solid (34.7 mg, 9.85% Yield). LCMS (M+H)⁺: 479.0. ¹H NMR (300 MHz, DMSO-d₆) δ 10.27 (s, 1H), 8.88 (s, 1H), 8.26 (d, J=5.4 Hz, 1H), 7.94 (d, J=10.5 Hz, 1H), 7.66 (dd, J=15.6, 2.1 Hz, 1H), 7.36 (m, 1H), 7.15-7.21 (m, 1H), 4.75 (m, 1H), 3.62 (m, 1H), 3.15 (m, 1H), 2.22 (m, 1H), 1.92-2.16 (m, 3H), —NH was not observed.

The following compounds were synthesized following Example 14:

| Cmpd | | MS | |
|---|---|---|---|
| No. | Structure | (M + H)⁺ | ¹H NMR |
| 1-33 | 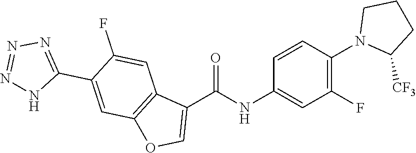 [SMILES: O=C(Nc1ccc(N2CCC[C@@H]2C(F)(F)F)c(F)c1)c1coc2cc(-c3nnn[nH]3)c(F)cc12] | 479.1 | (300 MHz, DMSO-d₆) δ 10.29 (s, 1H), 8.90 (s, 1H), 8.28 (d, J = 5.5 Hz, 1H), 7.95 (d, J = 10.3 Hz, 1H), 7.64 (dd, J = 15.6, 2.4 Hz, 1H), 7.38 (m, 1H), 7.10 − 7.21 (m, 2H), 4.74 (m, 1 H), 3.68 (m, 1H), 3.15 (m, 1H), 2.22 (m, 1H), 1.90 − 2.09 (m, 3H). |
| | (R)-5-fluoro-N-(3-fluoro-4-(2- | | |
| | (trifluoromethyl)pyrrolidin-1-yl)phenyl)-6- | | |
| | (1H-tetrazol-5-yl)benzofuran-3- | | |
| | carboxamide | | |
| 1-34 | 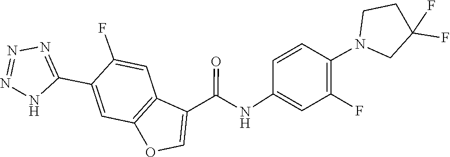 [SMILES: O=C(Nc1ccc(N2CCC(F)(F)C2)c(F)c1)c1coc2cc(-c3nnn[nH]3)c(F)cc12] N-(4-(3,3-difluoropyrrolidin-1-yl)-3- | 447.0 | (400 MHz, DMSO-d₆) δ 10.29 (s, 1H), 8.89 (s, 1H), 8.26 (d, J = 5.4 Hz, 1H), 7.93 (d, J = 10.2 Hz, 1H), 7.69 (dd, J = 15.6, 2.3 Hz, 1H), 7.39 (dd, J = 8.9, 2.4 Hz, 1H), 7.21- 6.95 (m, 1H), 6.90 (t, J = 9.5 Hz, 1H), 3.72 (t, J = 13.5 Hz, 2H), 3.50 (m, 2H), 2.42 (m, 2H). |
| | fluorophenyl)-5-fluoro-6-(1H-tetrazol-5- | | |
| | yl)benzofuran-3-carboxamide | | |
| 1-42 | 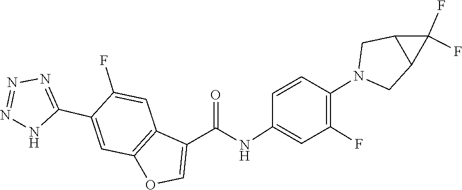 [SMILES: O=C(Nc1ccc(N2CC3C(C2)C3(F)F)c(F)c1)c1coc2cc(-c3nnn[nH]3)c(F)cc12] | 459.0 | (300 MHz, DMSO-d₆) δ 8.78 (s, 1H), 8.14 (d, J = 5.5 Hz, 1H), 7.82 (d, J = 10.2 Hz, 1H), 7.62 (dd, J = 16.2, 2.4 Hz, 1H), 7.35 (dd, J = 9.0, 2.4 Hz, 1H), 6.78 (t, J = 9.6 Hz, 1H), 3.79 − 3.70 (m, 2H), 3.67 − 3.58 (m, 2H), 2.67 − 2.56 (m, 2H), 2 NH were not observed. |
| | N-(4-(6,6-difluoro-3-azabicyclo[3.1.0]hexan-3-yl)-3- | | |
| | fluorophenyl)-5-fluoro-6-(1H-tetrazol-5- | | |
| | yl)benzofuran-3-carboxamide | | |

### Example 15: N-(4-((2-chloro-4-fluorobenzyl)oxy)phenyl)-5-fluoro-6-(5-oxo-4,5-dihydro-1,2,4-oxadiazol-3-yl benzofuran-3-carboxamide

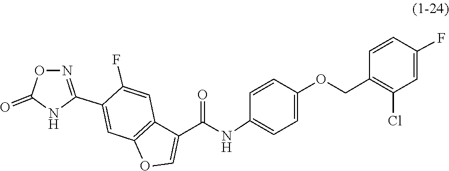 [SMILES: O=C(Nc1ccc(OCc2ccc(F)cc2Cl)cc1)c1coc2cc(-c3noc(=O)[nH]3)c(F)cc12]

### Step 1: Preparation of N-(4-((2-Chloro-4-Fluorobenzyl)Oxy)Phenyl)-6-Cyano-5-Fluorobenzofuran-3-Carboxamide

To a solution of ethyl 6-cyano-5-fluorobenzofuran-3-carboxylate (150 mg, 640 μmol) and 4-((2-chloro-4-fluorobenzyl)oxy)aniline (165 mg, 640 μmol, 1 eq.) in toluene (10 mL) was added AlMe₃ (57 mg, 788 μmol, 1.2 eq.) at 0° C. The reaction was stirred at 80° C. for 2 hrs under N₂. After the reaction completed, it was cooled to room temperature and quenched with water. Then the resulting mixture was extracted with ethyl acetate (3×50 mL). The combined organic layers were washed with brine, dried over anhydrous Na₂SO₄, filtered and concentrated. The residue was purified directly by C18 column (eluent: 35% to 55% (v/v) Acetonitrile and Water with 10 MMOL/L NH₄HCO₃) to give the title compound (84 mg, 30%). LCMS (M+H)⁺: 439.0.

### Step 2: Preparation of (Z)—N-(4-((2-Chloro-4-Fluorobenzyl)Oxy)Phenyl)-5-Fluoro-6-(N′-Hydroxycarbamimidoyl)Benzofuran-3-Carboxamide

To a solution of H₂NOH—HCl (79 mg, 1.14 mmol, 2.5 eq.) and K₂CO₃ (94.5 mg, 684 μmol, 1.5 eq.) in MeOH (10 mL) was added N-(4-((2-chloro-4-fluorobenzyl)oxy)phenyl)-6-cyano-5-fluorobenzofuran-3-carboxamide (200 mg, 456 μmol, 1 eq.) at room temperature. The reaction was stirred at 70° C. for 4 hrs. After the reaction completed, it was cooled to room temperature, quenched with water and extracted with ethyl acetate (3×20 mL). The combined organic layers were washed with brine, dried over anhydrous Na₂SO₄, filtered, and concentrated. The residue was purified by silica gel chromatography to afford the title compound as an off-white solid (200 mg, 65% Yield). LCMS (M+H)⁺: 472.0.

### Step 3: Preparation of N-(4-((2-Chloro-4-Fluorobenzyl)Oxy)Phenyl)-5-Fluoro-6-(5-Oxo-4,5-Dihydro-1,2,4-Oxadiazol-3-Yl)Benzofuran-3-Carboxamide

To a solution of (Z)—N-(4-((2-chloro-4-fluorobenzyl)oxy)phenyl)-5-fluoro-6-(N′-hydroxycarbamimidoyl)benzofuran-3-carboxamide (200 mg, 424 μmol) and di(1H-imidazol-1-yl)methanone (82.5 mg, 509 μmol, 1.2 eq.) in dioxane (10 mL) was added DBU (71 mg, 466 μmol, 1.1 eq.). The reaction was stirred at 100° C. for 3 hrs and cooled to room temperature under N₂. After the reaction completed, its pH was adjusted to ˜2 with 2 M HCl. The resulting mixture was extracted with ethyl acetate (3×50 mL). The combined organic layers were washed with brine, dried over anhydrous Na₂SO₄, filtered, and concentrated. Then the residue was purified by C18 reverse column to afford the title compound as an off-white solid (37.7 mg, 17.5% Yield). LCMS (M−H)⁻: 495.9. ¹H NMR (300 MHz, DMSO-d₆) δ 10.17 (s, 1H), 8.87 (s, 1H), 8.05 (d, J=5.4 Hz, 1H), 7.81 (d, J=10.5 Hz, 1H), 7.74-7.61 (m, 3H), 7.54 (dd, J=8.8, 2.6 Hz, 1H), 7.36-7.23 (m, 1H), 7.16-7.01 (m, 2H), 5.14 (s, 2H), —NH was not observed.

### Example 16: N³-(4-((2-chloro-4-fluorobenzyl)oxy)phenyl)-5-fluorobenzofuran-3,6-dicarboxamide

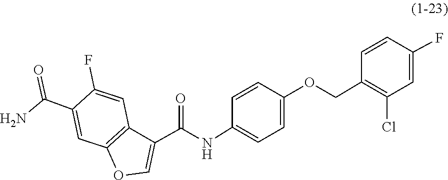 [SMILES: NC(=O)c1cc2occ(C(=O)Nc3ccc(OCc4ccc(F)cc4Cl)cc3)c2cc1F]

### Step 1: Preparation of N³-(4-((2-Chloro-4-Fluorobenzyl)Oxy)Phenyl)-5-Fluorobenzofuran-3,6-Dicarboxamide

To a solution of N-(4-((2-chloro-4-fluorobenzyl)oxy)phenyl)-6-cyano-5-fluorobenzofuran-3-carboxamide (120 mg, 273 μmol) in toluene (10 mL) was added (E)-acetaldehyde oxime (16.2 mg, 273 μmol, 1 eq.) and Pd(AcO)₂ (6.14 mg, 27.3 μmol, 0.1 eq.) and PPh₃ (71.7 mg, 273 μmol, 1 eq.). The resulting mixture was stirred at 80° C. for 2 hrs under N₂. After the reaction completed, it was cooled to room temperature, quenched with water and extracted with ethyl acetate (3×50 mL). The combined organic layers were washed with brine, dried over anhydrous Na₂SO₄, filtered, and concentrated. The residue was purified by silica gel column to afford the crude product. The crude product was further purified by XBridge Prep OBD C18 Column, 30×150 mm 5 μm column (eluent: 43% to 68% (v/v) Acetonitrile and Water with 10 MMOL/L NH₄HCO₃) to afford the title compound as a light yellow solid (32.4 mg, 25.7% Yield). LCMS (M+H)⁺: 457.0. ¹H NMR (300 MHz, DMSO-d₆) δ 10.21 (s, 1H), 8.92 (s, 1H), 7.98 (d, J=5.6 Hz, 1H), 7.88-7.63 (m, 6H), 7.55 (dd, J=8.9, 2.6 Hz, 1H), 7.36-7.23 (m, 1H), 7.12-7.01 (m, 2H), 5.14 (s, 2H).

### Example 17: (S)—N-(4-((3,3-difluorocyclopentyl)oxy)-3-fluorophenyl)-5-fluoro-6-(1H-tetrazol-5-yl)benzofuran-3-carboxamide & (R)—N-(4-((3,3-difluorocyclopentyl)oxy)-3-fluorophenyl)-5-fluoro-6-(1H-tetrazol-5-yl)benzofuran-3-carboxamide

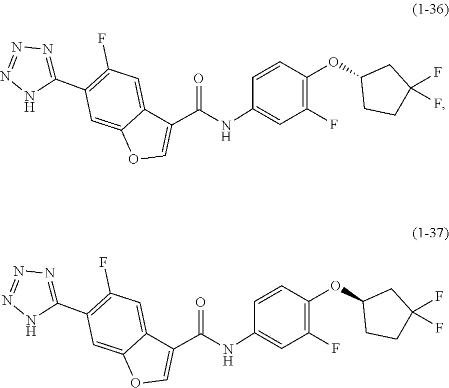 [SMILES: O=C(Nc1ccc(O[C@H]2CCC(F)(F)C2)c(F)c1)c1coc2cc(-c3nnn[nH]3)c(F)cc12 ; O=C(Nc1ccc(O[C@@H]2CCC(F)(F)C2)c(F)c1)c1coc2cc(-c3nnn[nH]3)c(F)cc12]

### Step 1: Preparation of 4-((3,3-Difluorocyclopentyl)Oxy)-3-Fluoroaniline

The title compound was prepared following step 1 and step 2 of Example 1. LCMS (M−H)⁺: 232.2.

### Step 2: Preparation of (Rac)-N-(4-((3,3-Difluorocyclopentyl)Oxy)-3-Fluorophenyl)-5-Fluoro-6-(1H-Tetrazol-5-Yl)Benzofuran-3-Carboxamide

To a solution of 5-fluoro-6-(1H-tetrazol-5-yl)benzofuran-3-carboxylic acid (280 mg, 1.13 mmol), 4-((3,3-difluorocyclopentyl)oxy)-3-fluoroaniline (287 mg, 1.24 mmol, 1.1 eq.) and HATU (514 mg, 1.35 mmol, 1.2 eq.) in DMF (10 mL) was added DIEA (437 mg, 3.38 mmol, 3 equiv) at room temperature. The reaction was stirred at room temperature for 2 hrs. After the reaction completed, it was quenched with water and extracted with ethyl acetate (3×50 mL). The combined organic layers were washed with brine, dried over anhydrous Na₂SO₄, filtered, and concentrated. The residue was purified directly by C18 column (eluent: 25% to 55% (v/v) Acetonitrile and Water with 10 MMOL/L NH₄HCO₃) to afford the title compound as a yellow solid (300 mg, 57.3% Yield). LCMS (M+H)⁺: 462.2.

### Step 3: Preparation of (S)-5-Fluoro-N-(3-Fluoro-4-(2-(Trifluoromethyl)Pyrrolidin-1-Yl)Phenyl)-6-(1H-Tetrazol-5-Yl)Benzofuran-3-Carboxamide & (R)-5-Fluoro-N-(3-Fluoro-4-(2-(Trifluoromethyl)Pyrrolidin-1-Yl)Phenyl)-6-(1H-Tetrazol-5-Yl)Benzofuran-3-Carboxamide

A sample of (Rac)-N-(4-((3,3-difluorocyclopentyl)oxy)-3-fluorophenyl)-5-fluoro-6-(1H-tetrazol-5-yl)benzofuran-3-carboxamide (300 mg, 650 μmol) was purified by chiral-HPLC using a CHIRALPAK IF, 2*25 cm, 5 μm column (eluent: Hex(0.1% TFA):EtOH=70:30) to afford (S)—N-(4-((3,3-difluorocyclopentyl)oxy)-3-fluorophenyl)-5-fluoro-6-(1H-tetrazol-5-yl)benzofuran-3-carboxamide as an off-white solid (31.9 mg, 99.511% purity, 99.7% ee) (2nd peak) and (R)-isomer (1st Peak). The stereochemistry was arbitrary assigned. (S)-isomer: LCMS (M+H)⁺: 462.1. ¹H NMR (300 MHz, DMSO-d6) δ 8.95 (s, 1H), 8.39 (d, J=4.8 Hz, 1H), 8.04 (d, J=10.0 Hz, 1H), 7.74 (dd, J=13.5, 2.5 Hz, 1H), 7.43 (d, J=8.9 Hz, 1H), 7.20 (t, J=9.2 Hz, 1H), 4.97 (s, 1H), 2.79-2.60 (m, 1H), 2.40-2.10 (m, 4H), 2.08-1.90 (m, 1H), 2 —NH were not observed. (R)-isomer: LCMS (M+H)⁺: 462.0. 1H NMR (300 MHz, DMSO-d₆) δ 8.96 (s, 1H), 8.39 (d, J=5.4 Hz, 1H), 8.04 (d, J=10.3 Hz, 1H), 7.74 (dd, J=13.5, 2.5 Hz, 1H), 7.43 (d, J=8.9 Hz, 1H), 7.20 (t, J=9.2 Hz, 1H), 4.98 (s, 1H), 2.77-2.60 (m, 1H), 2.40-2.09 (m, 4H), 2.04-1.93 (m, 1H), 2 —NH were not observed.

The following compounds were synthesized following Example 17:

| Cmpd | | MS | |
|---|---|---|---|
| No. | Structure/Name | (M + H)⁺ | ¹H NMR |
| 1-25 | 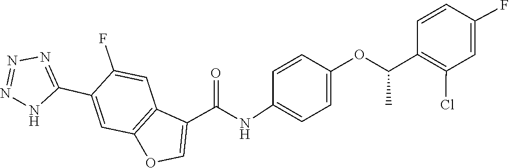 [SMILES: C[C@H](Oc1ccc(NC(=O)c2coc3cc(-c4nnn[nH]4)c(F)cc23)cc1)c1ccc(F)cc1Cl] | 496.0 | (300 MHz, DMSO-d₆) δ 8.76 (s, 1H), 8.26 (d, J = 5.4 Hz, 1H), 7.91 (d, J = 10.2 Hz, 1H), 7.56 − 7.45 (m, 3H), 7.38 (dd, J = 8.8, 2.6 Hz, 1H), 7.23 − 7.10 (m, 1H), 6.82 (d, J = 9.1 Hz, 2H), 5.71 − 5.58 (m, 1H), 1.53 (d, J = 6.3 Hz, 3H), 2 NH were not observed. |
| | (S)-N-(4-(1-(2-chloro-4- | | |
| | fluorophenyl)ethoxy)phenyl)-5-fluoro-6-(1H- | | |
| | tetrazol-5-yl)benzofuran-3-carboxamide | | |
| 1-26 | 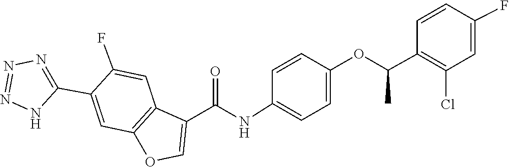 [SMILES: C[C@@H](Oc1ccc(NC(=O)c2coc3cc(-c4nnn[nH]4)c(F)cc23)cc1)c1ccc(F)cc1Cl] | 496.0 | (300 MHz, DMSO-d₆) δ 8.77 (s, 1H), 8.26 (d, J = 5.4 Hz, 1H), 7.92 (d, J = 10.3 Hz, 1H), 7.55 − 7.44 (m, 3H), 7.37 (dd, J = 8.8, 2.6 Hz, 1H), 7.2− 7.09 (m, 1H), 6.82 (d, J = 8.9 Hz, 2H), 5.69 − 5.57 (m, 1H), 1.52 (d, J = 6.2 Hz, 3H), 2 NH were not observed. |
| | (R)-N-(4-(1-(2-chloro-4- | | |
| | fluorophenyl)ethoxy)phenyl)-5-fluoro-6-(1H- | | |
| | tetrazol-5-yl)benzofuran-3-carboxamide | | |
| 1-30 | 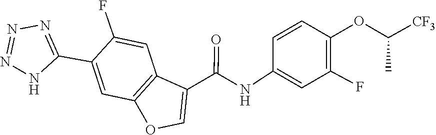 [SMILES: C[C@H](Oc1ccc(NC(=O)c2coc3cc(-c4nnn[nH]4)c(F)cc23)cc1F)C(F)(F)F] (S)-5-fluoro-N-(3-fluoro-4-((1,1,1- | 454.0 | (400 MHz, DMSO-d₆) δ 9.02 (s, 1H), 8.38 (d, J = 5.6 Hz, 1H), 8.03 (d, J = 10.2 Hz, 1H), 7.76 (dd, J = 13.2, 2.5 Hz, 1H), 7.42 − 7.51 (m, 1H), 7.35 − 7.41 (m, 1H), 5.01 − 5.12 (m, 1H), 1.46 (d, J = 6.4 Hz, 3H), 2 NH were not observed. |
| | trifluoropropan-2-yl)oxy)phenyl)-6-(1H- | | |
| | tetrazol-5-yl)benzofuran-3-carboxamide | | |
| 1-31 | 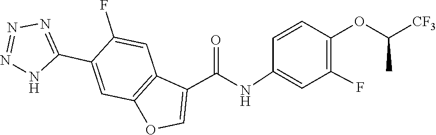 [SMILES: C[C@@H](Oc1ccc(NC(=O)c2coc3cc(-c4nnn[nH]4)c(F)cc23)cc1F)C(F)(F)F] (R)-5-fluoro-N-(3-fluoro-4-((1,1,1- | 454.0 | (400 MHz, DMSO-d₆) δ 8.93 (s, 1H), 8.38 (d, J = 5.4 Hz, 1H), 8.03 (d, J = 10.2 Hz, 1H), 7.76 (dd, J = 13.2, 2.5 Hz, 1H), 7.42 − 7.51 (m, 1H), 7.35 − 7.41 (m, 1H), 5.01 − 5.12 (m, 1H), 1.46 (d, J = 6.8 Hz, 3H), 2 NH were not observed. |
| | trifluoropropan-2-yl)oxy)phenyl)-6-(1H- | | |
| | tetrazol-5-yl)benzofuran-3-carboxamide | | |

### Example 18: N-(4-(2-chloro-4-fluorophenoxy)phenyl)-5-fluoro-6-(1H-tetrazol-5-yl)benzofuran-3-carboxamide

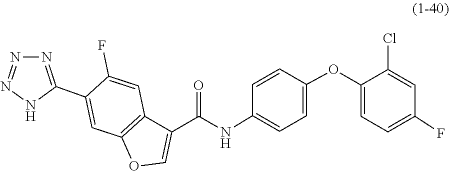 [SMILES: O=C(Nc1ccc(Oc2ccc(F)cc2Cl)cc1)c1coc2cc(-c3nnn[nH]3)c(F)cc12]

### Step 1: Preparation of 4-(2-Chloro-4-Fluorophenoxy)Aniline

The title compound was prepared following step 1 and step 2 of Example 1. LCMS (M−H)⁺: 238.1.

### Step 2: Preparation of N-(4-(2-Chloro-4-Fluorophenoxy)Phenyl)-5-Fluoro-6-(1H-Tetrazol-5-Yl)Benzofuran-3-Carboxamide

To a solution of ethyl 5-fluoro-6-(1H-tetrazol-5-yl)benzofuran-3-carboxylate (232 mg, 842 mol) and 4-(2-chloro-4-fluorophenoxy)aniline (200 mg, 842 mol, 1 eq.) in toluene (10 mL) was added AlMe₃ (72.8 mg, 1.01 mmol, 1.2 eq.) at 0° C. The reaction was stirred at 80° C. for 2 hrs under N₂. After the reaction completed, it was cooled to room temperature, quenched with water and extracted with EtOAc (3×20 mL). The combined organic layers were washed with brine, dried over anhydrous Na₂SO₄, filtered, and concentrated. The residue was purified by C18 reverse column to afford the title compound as a white solid (42.3 mg, 10.7% Yield). LCMS (M+H)⁺: 468.0. ¹H NMR (300 MHz, DMSO-d₆) δ 8.87 (s, 1H), 8.21 (d, J=5.5 Hz, 1H), 7.88 (d, J=10.3 Hz, 1H), 7.81-7.70 (m, 2H), 7.63 (dd, J=8.4, 3.0 Hz, 1H), 7.32-7.15 (m, 2H), 7.05-6.94 (m, 2H), 2 —NH was not observed.

### Example 19: N-(4-(cyclopentyloxy)-3-fluorophenyl)-7-(1H-tetrazol-5-yl)benzofuran-3-carboxamide

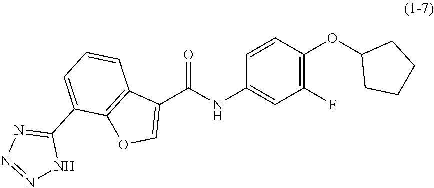 [SMILES: O=C(Nc1ccc(OC2CCCC2)c(F)c1)c1coc2c(-c3nnn[nH]3)cccc12]

### Step 1: Preparation of 4-(Cyclopentyloxy)-3-Fluoroaniline

The title compound was prepared following step 1 and step 2 of Example 1. LCMS (M−H)⁺: 195.9.

### Step 2: Preparation of Ethyl 7-(1H-Tetrazol-5-Yl)Benzofuran-3-Carboxylate

To a solution of ethyl 7-cyanobenzofuran-3-carboxylate (1 g, 4 mmol) in toluene (20 mL) was added TMSN₃ (2 g, 0.01 mmol, 3 eq.) and Bu₂SnO (0.5 g, 2 mmol, 0.5 eq.). After removing the air in the reaction flask under vacuum, it was backfilled with Nitrogen. The reaction was stirred at 100° C. for 2 hrs. After the reaction completed, it was cooled to room temperature, quenched with water (50 mL) and extracted with ethyl acetate (50 mL×3). The combined organic extracts were washed with brine, dried over anhydrous Na₂SO₄, filtered and concentrated. The crude product was purified by silica gel chromatography to afford the title compound as a white solid (1.05 g, 80% yield). LCMS (M+H)⁺: 259.0.

### Step 3: Preparation of 7-(1H-Tetrazol-5-Yl)Benzofuran-3-Carboxylic Acid

To a solution of ethyl 7-(1H-tetrazol-5-yl)benzofuran-3-carboxylate (980 mg, 3.79 mmol) in THF/MeOH/H₂O (10 mL, v/v/v=3/1/1) was added LiOH (182 mg, 7.59 mmol) at 0° C. Then the solution was slowly warmed to room temperature and stirred for 2 hrs. After the reaction completed, its pH was adjusted to ˜7. The crude product was precipitated out during the pH adjustment. The solid product was filtered, collected, and further purified by C18 column (eluent: 25% to 45% (v/v) CH₃CN and H₂O with 10 MMOL/L NH₄HCO₃) to afford the title compound (1.0 g, 90%). LCMS (M+H)⁺: 231.0.

### Step 4: Preparation of N-(4-(Cyclopentyloxy)-3-Fluorophenyl)-7-(1H-Tetrazol-5-Yl)Benzofuran-3-Carboxamide

To a solution of 7-(1H-tetrazol-5-yl)benzofuran-3-carboxylic acid (300 mg, 1.3 mmol), 4-(cyclopentyloxy)-3-fluoroaniline (254 mg, 1.3 mmol, 1 eq.) and HATU (594 mg, 1.56 mmol, 1.2 eq.) in DMF (10 mL) was added DIEA (505 mg, 3.91 mmol, 3 eq.) at room temperature. The reaction was stirred at room temperature for 2 hrs. After the reaction completed, it was quenched with water and extracted with ethyl acetate (3×50 mL). The combined organic extracts were washed with brine, dried over anhydrous Na₂SO₄, filtered and concentrated. Then the residue was purified by silica gel chromatography (15%-40% methanol/DCM) to give the crude product. Then the crude product was further purified by XBridge Prep OBD C18 Column, 30×150 mm 5 μm column (eluent: 20% to 50% (v/v) Acetonitrile and Water with 10 MMOL/L NH4HCO3) to afford the title compound as a white solid (97.7 mg, 18.2% Yield). LCMS (M+H)⁺: 408.1. ¹H NMR (400 MHz, DMSO-d₆) δ 10.30 (s, 1H), 8.90 (s, 1H), 8.22 (dd, J=7.9, 1.3 Hz, 1H), 8.03 (dd, J=7.6, 1.3 Hz, 1H), 7.74 (dd, J=13.6, 2.5 Hz, 1H), 7.54 (t, J=7.7 Hz, 1H), 7.47-7.39 (m, 1H), 7.18 (t, J=9.3 Hz, 1H), 4.90-4.81 (m, 1H), 1.97-1.83 (m, 2H), 1.81-1.68 (m, 4H), 1.68-1.53 (m, 2H), NH was not observed.

The following compounds were synthesized following Example 19:

| Cmpd | | MS | |
|---|---|---|---|
| No. | Structure | (M + H)⁺ | ¹H NMR |
| 1-11 | 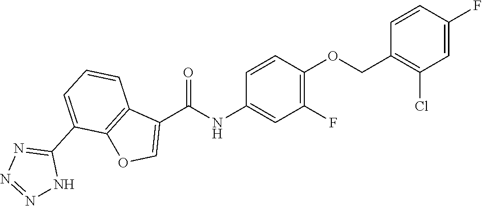 [SMILES: O=C(Nc1ccc(OCc2ccc(F)cc2Cl)c(F)c1)c1coc2c(-c3nnn[nH]3)cccc12] | 482.0 | (400 MHz, DMSO-d₆) δ 10.31 (s, 1H), 8.87 (s, 1H), 8.11 (dd, J = 7.9, 1.3 Hz, 1H), 8.00 (dd, J = 7.6, 1.3 Hz, 1H), 7.79 (dd, J = 13.6, 2.5 Hz, 1H), 7.69 (dd, J = 8.6, 6.2 Hz, 1H), 7.55 (dd, J = 8.9, 2.6 Hz, 1H), 7.51 − 7.42 (m, 2H), 7.36 − 7.26 (m, 2H), 7.17 (s, 1H), 5.21 (s, 2H). |
| | N-(4-((2-chloro-4-fluorobenzyl)oxy)-3- | | |
| | fluorophenyl)-7-(1H-tetrazol-5- | | |
| | yl)benzofuran-3-carboxamide | | |

### Example 20: N-(4-cyclopentyl-3-fluorophenyl)-6-(1H-tetrazol-5-yl)benzo[b]thiophene-3-carboxamide

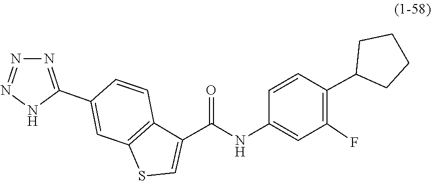 [SMILES: O=C(Nc1ccc(C2CCCC2)c(F)c1)c1csc2cc(-c3nnn[nH]3)ccc12]

### Step 1: Preparation of Ethyl 6-Cyanobenzo[b]Thiophene-3-Carboxylate

To a solution of ethyl 6-bromobenzo[b]thiophene-3-carboxylate (800 mg, 2.81 mmol) in DMA (10 mL) was added Zn(CN)₂ (394 mg, 3.37 mmol), Zn (36 mg, 561 μmol), Pd₂(dba)₃ (257 mg, 281 μmol) and dppf (311 mg, 561 μmol) under an inert atmosphere. The resulting mixture was stirred at 100° C. for 2 hrs under N₂. After the reaction was completed, it was cooled to room temperature, quenched with water, and extracted with ethyl acetate (3×50 mL). The combined organic layers were washed with brine, dried over anhydrous Na₂SO₄, filtered and concentrated. The residue was purified by silica gel chromatography (10%-55% ethyl acetate/petroleum ether) to afford the title compound as a yellow solid (500 mg, 73.2% yield).

### Step 2: Preparation of Ethyl 6-(1H-Tetrazol-5-Yl)Benzo[b]Thiophene-3-Carboxylate

To a solution of ethyl 6-cyanobenzo[b]thiophene-3-carboxylate (490 mg, 2.12 mmol) and Bu₂SnO (264 mg, 1.06 mmol) in toluene (10 mL) was drop wisely added TMSN₃ (732 mg, 6.36 mmol). The resulting mixture was stirred at 100° C. for 2 hrs under N₂. After the reaction was completed, it was cooled to room temperature and concentrated. The residue was directly purified by silica gel chromatography (5%-20% methanol/DCM) to afford the title compound as a white solid (490 mg, 83.5% Yield). LCMS (M+H)⁺: 275.9.

### Step 3: Preparation of N-(4-Cyclopentyl-3-Fluorophenyl)-6-(1H-Tetrazol-5-Yl)Benzo[b]Thiophene-3-Carboxamide

To a solution of ethyl 6-(1H-tetrazol-5-yl)benzo[b]thiophene-3-carboxylate (220 mg, 802 μmol) and 4-cyclopentyl-3-fluoroaniline (144 mg, 802 μmol, 1 eq.) in toluene (10 mL) was added AlMe₃ (69.4 mg, 962 μmol, 1.2 eq.) at 0° C. The resulting mixture was stirred at 80° C. for 2 hrs under N₂. The reaction was cooled to room temperature, quenched with water and extracted with ethyl acetate (3×50 mL). The combined organic layers were washed with brine, dried over anhydrous Na₂SO₄, filtered and concentrated. The residue was purified by C18 column (eluent: 35% to 55% (v/v) CH₃CN and H₂O with 10 MMOL/L NH₄HCO₃) to afford the crude product. The crude product was further purified by XBridge Prep OBD C18 Column, 30×150 mm 5 μm column (eluent: 38% to 58% (v/v) Acetonitrile and Water with 10 MMOL/L NH₄HCO₃) to give the title compound as a white solid (35.4 mg, 10.8% Yield). LCMS (M+H)⁺: 408.1. ¹H NMR (300 MHz, DMSO-d₆) δ 10.54 (s, 1H), 8.79 (d, J=1.5 Hz, 1H), 8.75 (s, 1H), 8.60 (d, J=8.6 Hz, 1H), 8.14 (dd, J=8.5, 1.6 Hz, 1H), 7.71 (dd, J=13.0, 2.1 Hz, 1H), 7.50 (dd, J=8.5, 2.1 Hz, 1H), 7.34 (t, J=8.6 Hz, 1H), 3.26-3.11 (m, 1H), 2.06-1.92 (m, 2H), 1.89-1.47 (m, 6H), —NH was not observed.

### Example 21: N-(4-((2-chloro-4-fluorobenzyl)oxy)-3-fluorophenyl)-6-(1H-tetrazol-5-yl)benzo[b]thiophene-3-carboxamide

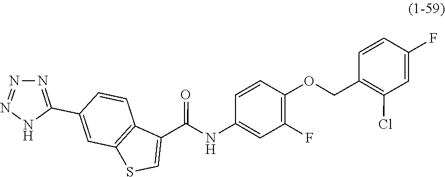 [SMILES: O=C(Nc1ccc(OCc2ccc(F)cc2Cl)c(F)c1)c1csc2cc(-c3nnn[nH]3)ccc12]

### Step 1: Preparation of N-(4-((2-Chloro-4-Fluorobenzyl)Oxy)-3-Fluorophenyl)-6-(1H-Tetrazol-5-Yl)Benzo[b]Thiophene-3-Carboxamide

To a solution of ethyl 6-(1H-tetrazol-5-yl)benzo[b]thiophene-3-carboxylate (200 mg, 729 μmol) and 4-((2-chloro-4-fluorobenzyl)oxy)-3-fluoroaniline (197 mg, 729 μmol, 1 eq.) in toluene (10 mL) was added AlMe₃ (63.1 mg, 875 μmol, 1.2 eq.) at 0° C. Then the reaction was stirred at 80° C. for 2 hrs under N₂. After the reaction completed, it was cooled to room temperature and quenched with water. The resulting mixture was extracted with ethyl acetate (3×50 mL). The combined organic layers were washed with brine, dried over anhydrous Na₂SO₄, filtered and concentrated. The residue was purified by silica gel chromatography (20%-50% methanol/DCM) to give the crude product. The crude product was further purified by C18 column (eluent: 25% to 60% (v/v) Acetonitrile and Water with 10 MMOL/L NH₄HCO₃) to afford the title compound as a white solid (49.9 mg, 13.6% Yield). LCMS (M+H)⁺: 497.9. ¹H NMR (400 MHz, DMSO-d₆) δ 10.46 (s, 1H), 8.70 (s, 1H), 8.62 (s, 1H), 8.50 (dd, J=8.6, 0.6 Hz, 1H), 8.13 (dd, J=8.5, 1.5 Hz, 1H), 7.80 (dd, J=13.6, 2.5 Hz, 1H), 7.69 (dd, J=8.6, 6.3 Hz, 1H), 7.62-7.47 (m, 2H), 7.38-7.27 (m, 2H), 7.11 (brs, 1H), 5.21 (s, 2H).

### Example 22: Luciferase Reporter Assay

HEK293 cells stably transfected with human GPR35 were maintained in Dulbecco's Modified Eagle Medium (DMEM) supplemented with 10% FBS and 300 μg/mL Zeocin and 200 μg/mL Hygromycin at 37° C., 10% CO₂. On Day 1, cells were plated in poly-D-lysine coated 96-well tissue culture plate in regular growth medium (DMEM/10% FBS/300 μg/mL Zeocin/200 μg/mL Hygromycin. On the next day, luciferase reporter plasmid DNA with YAP responsive element was transfected into cells using lipofectamine 2000 (Invitrogen) following manufacture's protocol. After transfection for 6 hr., cells were treated with testing compounds in fasting DMEM medium (phenol red free, FBS free) at varying compound concentrations ranging from 10 μM to 169 μM in a 3-fold serial diluted fashion. After an overnight incubation, the luciferase activities were measured with SteadyLite Plus Kit (PerkinElmer) according to manufacturer's protocol. IC50 was determined by fitting the dose-response data to a four-parameter logistic curve using GraphPad Prism.

Luciferase IC₅₀ data are provided in Table 2. The symbol “+” indicates IC₅₀ is greater than 1 mM. “++” indicates IC₅₀>100 nM but ≤1 mM. “+++” indicates IC₅₀≤100 nM.

TABLE 2

| | Luciferase |
|---|---|
| Cpd | Reporter |
| No. | (IC₅₀) |
| 1-1 | +++ |
| 1-2 | +++ |
| 1-3 | + |
| 1-4 | + |
| 1-5 | + |
| 1-6 | ++ |
| 1-7 | + |
| 1-8 | + |
| 1-9 | + |
| 1-10 | +++ (partial) |
| 1-11 | + |
| 1-12 | + |
| 1-13 | +++ |
| 1-14 | +++ |
| 1-15 | +++ |
| 1-16 | +++ |
| 1-17 | +++ |
| 1-18 | +++ |
| 1-19 | ++ |
| 1-20 | +++ |
| 1-21 | ++ (partial) |
| 1-22 | + |
| 1-23 | + |
| 1-24 | +++ |
| 1-25 | + |
| 1-26 | + |
| 1-27 | ++ |
| 1-28 | ++ |
| 1-29 | + |
| 1-30 | +++ |
| 1-31 | ++ |
| 1-32 | ++ |
| 1-33 | ++ |
| 1-34 | +++ |
| 1-35 | ++ |
| 1-36 | +++ |
| 1-37 | +++ |
| 1-38 | +++ |
| 1-39 | ++ |
| 1-40 | ++ |
| 1-41 | +++ |
| 1-42 | ++ |
| 1-43 | +++ |
| 1-44 | ++ |
| 1-45 | ++ |
| 1-46 | ++ |
| 1-47 | ++ (partial) |
| 1-48 | ++ (partial) |
| 1-49 | +++ |
| 1-50 | ++ |
| 1-51 | +++ |
| 1-52 | ++ |
| 1-53 | +++ |
| 1-54 | ++ |
| 1-55 | ++ |
| 1-56 | +++ |
| 1-57 | ++ |
| 1-58 | +++ |

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### ADDITIONAL NUMBERED EMBODIMENTS

1. A compound represented by Formula (I):

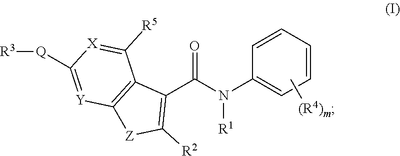

or a pharmaceutically acceptable salt thereof, wherein:
X is selected from CR⁶ and N;
Y is selected from CR¹² and N;
Z is selected from —O—, —S—, and —NR⁸—;
Q is absent or selected from:
   —W—, —O—, —O—W—, —W—O—, —S—, —S—W—, —W—S—, —NR¹⁰—W—, —W—NR¹⁰—, —C(O)—, —C(O)N(R¹⁰)—, —N(R¹⁰)C(O)—, —C(O)O—, —OC(O)—, —N(R¹⁰)C(O)N(R¹⁰)—, —OC(O)N(R¹⁰)—, —N(R¹⁰)C(O)O—, —S(O)—, —S(O)₂—, and —P(O)₂O—;
m is selected from 1, 2, 3, 4, and 5;
R¹ is selected from:
   hydrogen;
   C₁₋₆ alkyl, C₂₋₆ alkenyl, and C₂₋₆ alkynyl, each of which is optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —C(O)OR¹⁰, —OC(O)R¹⁰, —N(R¹⁰)C(O)N(R¹⁰)₂, —OC(O)N(R¹⁰)₂, —N(R¹⁰)C(O)OR¹⁰, —S(O)R¹⁰, —S(O)₂R¹⁰, —NO₂, ═O, ═S, ═N(R¹⁰), —CN, C₃₋₁₀ carbocycle and 3- to 10-membered heterocycle, wherein the C₃₋₁₀ carbocycle and 3- to 10-membered heterocycle are each optionally substituted with one or more R⁹; and
   C₃₋₁₀ carbocycle and 3- to 10-membered heterocycle, each of which is optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —N(R¹⁰)C(O)N(R¹⁰)₂, —OC(O)N(R¹⁰)₂, —N(R¹⁰)C(O)OR¹⁰, —C(O)OR¹⁰, —OC(O)R¹⁰, —S(O)R¹⁰, —S(O)₂R¹⁰, —NO₂, ═O, ═S, ═N(R¹⁰), —CN, C₁₋₆ alkyl, C₂₋₆ alkenyl, and C₂₋₆ alkynyl, wherein C₁₋₆ alkyl, C₂₋₆ alkenyl, and C₂₋₆ alkynyl are each optionally substituted with one or more R⁹;
R² is selected from:
   hydrogen;
   halogen, —OR¹⁰, —SR¹⁰, —N(R¹⁰)₂, —C(O)R¹⁰, —C(O)N(R¹⁰)₂, —N(R¹⁰)C(O)R¹⁰, —N(R¹⁰)C(O)N(R¹⁰)₂, —OC(O)N(R¹⁰)₂, —N(R¹⁰)C(O)OR¹⁰, —C(O)OR¹⁰, —OC(O)R¹⁰, —S(O)R¹⁰, —S(O)₂R¹⁰, —NO₂, and —CN;
   C₁₋₆ alkyl, C₂₋₆ alkenyl, and C₂₋₆ alkynyl, each of which is optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —C(O)OR¹⁰, —OC(O)R¹⁰, —N(R¹⁰)C(O)N(R¹⁰)₂, —OC(O)N(R¹⁰)₂, —N(R¹⁰)C(O)OR¹⁰, —S(O)R¹⁰, —S(O)₂R¹⁰, —NO₂, ═O, ═S, ═N(R¹⁰), —CN, C₃₋₁₀ carbocycle and 3- to 10-membered heterocycle, wherein the C₃₋₁₀ carbocycle and 3- to 10-membered heterocycle are each optionally substituted with one or more R⁹; and
   C₃₋₆ carbocycle and 3- to 6-membered heterocycle, each of which is optionally substituted with one or more substituents independently selected from halogen, —OH, —SR¹⁰, —N(R¹⁰)₂, —C(O)R¹⁰, —C(O)N(R¹⁰)₂, —N(R¹⁰)C(O)R¹⁰, —N(R¹⁰)C(O)N(R¹⁰)₂, —OC(O)N(R¹⁰)₂, —N(R¹⁰)C(O)OR¹⁰, —C(O)OR¹⁰, —OC(O)R¹⁰, —S(O)R¹⁰, —S(O)₂R¹⁰, —NO₂, ═O, ═S, ═N(R¹⁰), —CN, C₁₋₆ alkyl, C₂₋₆ alkenyl, and C₂₋₆ alkynyl, wherein C₁₋₆ alkyl, C₂₋₆ alkenyl, and C₂₋₆ alkynyl are each optionally substituted with one or more R⁹;
R³ is selected from:
   C₃₋₆ carbocycle and 3- to 6-membered heterocycle, each of which is optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —N(R¹⁰)C(O)N(R¹⁰)₂, —OC(O)N(R¹⁰)₂, —N(R¹⁰)C(O)OR¹⁰, —C(O)OR¹⁰, —OC(O)R¹⁰, —S(O)R¹⁰, —S(O)₂R¹⁰, —NO₂, ═O, ═S, ═N(R¹⁰), —CN, C₁₋₆ alkyl, C₂₋₆ alkenyl, and C₂₋₆ alkynyl, wherein C₁₋₆ alkyl, C₂₋₆ alkenyl, and C₂₋₆ alkynyl are each optionally substituted with one or more R⁹; and
   wherein if Q is —W—, then R³ is additionally selected from halogen, —OR¹⁰, —SR¹⁰, —N(R¹⁰)₂, —C(O)R¹⁰, —C(O)N(R¹⁰)₂, —N(R¹⁰)C(O)R¹⁰, —N(R¹⁰)C(O)N(R¹⁰)₂, —OC(O)N(R¹⁰)₂, —N(R¹⁰)C(O)OR¹⁰, —C(O)OR¹⁰, —OC(O)R¹⁰, —S(O)R¹⁰, —S(O)₂R¹⁰, —NO₂, and —CN; and
each R⁴ is independently selected from:
   halogen, —OR⁷, —SR⁷, —N(R⁷)₂, —C(O)R⁷, —C(O)N(R⁷)₂, —N(R⁷)C(O)R⁷, —N(R⁷)C(O)N(R⁷)₂, —OC(O)N(R⁷)₂, —N(R⁷)C(O)OR⁷, —C(O)OR⁷, —OC(O)R⁷, —S(O)R⁷, —S(O)₂R⁷, —NO₂, and —CN;
   C₁₋₆ alkyl, C₂₋₆ alkenyl, and C₂₋₆ alkynyl, each of which is optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —C(O)OR¹⁰, —OC(O)R¹⁰, —N(R¹⁰)C(O)N(R¹⁰)₂, —OC(O)N(R¹⁰)₂, —N(R¹⁰)C(O)OR¹⁰, —S(O)R¹⁰, —S(O)₂R¹⁰, —NO₂, ═O, ═S, ═N(R¹⁰), —CN, C₃₋₁₀ carbocycle and 3- to 10-membered heterocycle, wherein the C₃₋₁₀ carbocycle and 3- to 10-membered heterocycle are each optionally substituted with one or more R⁹; and
   C₃₋₁₀ carbocycle and 3- to 10-membered heterocycle, each of which is optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —N(R¹⁰)C(O)N(R¹⁰)₂, —OC(O)N(R¹⁰)₂, —N(R¹⁰)C(O)OR¹⁰, —C(O)OR¹⁰, —OC(O)R¹⁰, —S(O)R¹⁰, —S(O)₂R¹⁰, —NO₂, ═O, ═S, ═N(R¹⁰), —CN, C₁₋₆ alkyl, C₂₋₆ alkenyl, and C₂₋₆ alkynyl, wherein C₁₋₆ alkyl, C₂₋₆ alkenyl, and C₂₋₆ alkynyl are each optionally substituted with one or more R⁹;
R⁵ is selected from:
   hydrogen, halogen, —OR¹⁰, —SR¹⁰, —N(R¹⁰)₂, —NO₂, and —CN; and
   C₁₋₆ alkyl optionally substituted with one or more substituents independently selected from halogen, —OR¹⁰, —SR¹⁰, —N(R¹⁰)₂, —NO₂, and —CN;
R⁶ is selected from:
   hydrogen, halogen, —OR¹⁰, —SR¹⁰, —N(R¹⁰)₂, —NO₂, and —CN; and
   C₁₋₆ alkyl optionally substituted with one or more substituents independently selected from halogen, —OR¹⁰, —SR¹⁰, —N(R¹⁰)₂, —NO₂, and —CN;
each R⁷ is independently selected from:
   hydrogen;
   C₁₋₆ alkyl, C₂₋₆ alkenyl, and C₂₋₆ alkynyl, each of which is optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —C(O)OR¹⁰, —OC(O)R¹⁰, —N(R¹⁰)C(O)N(R¹⁰)₂, —OC(O)N(R¹⁰)₂, —N(R¹⁰)C(O)OR¹⁰, —S(O)R¹⁰, —S(O)₂R¹⁰, —NO₂, ═O, ═S, ═N(R¹⁰), —CN, C₃₋₁₀ carbocycle and 3- to 10-membered heterocycle, wherein the C₃₋₁₀ carbocycle and 3- to 10-membered heterocycle are each optionally substituted with one or more R⁹; and
   C₃₋₁₀ carbocycle and 3- to 10-membered heterocycle, each of which is optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —N(R¹⁰)C(O)N(R¹⁰)₂, —OC(O)N(R¹⁰)₂, —N(R¹⁰)C(O)OR¹⁰, —C(O)OR¹⁰, —OC(O)R¹⁰, —S(O)R¹⁰, —S(O)₂R¹⁰, —NO₂, ═O, ═S, ═N(R¹⁰), —CN, C₁₋₆ alkyl, C₂₋₆ alkenyl, and C₂₋₆ alkynyl, wherein C₁₋₆ alkyl, C₂₋₆ alkenyl, and C₂₋₆ alkynyl are each optionally substituted with one or more R⁹;
R⁸ is selected from:
   hydrogen;
   C₁₋₆ alkyl, C₂₋₆ alkenyl, and C₂₋₆ alkynyl, each of which is optionally substituted with one or more substituents independently selected from halogen, —SR¹⁰, —N(R¹⁰)₂, —C(O)R¹⁰, —C(O)N(R¹⁰)₂, —N(R¹⁰)C(O)R¹⁰, —C(O)OR¹⁰, —OC(O)R¹⁰, —N(R¹⁰)C(O)N(R¹⁰)₂, —OC(O)N(R¹⁰)₂, —N(R¹⁰)C(O)OR¹⁰, —S(O)R¹⁰, —S(O)₂R¹⁰, —NO₂, ═O, ═S, ═N(R¹⁰), —CN, C₃₋₁₀ carbocycle and 3- to 10-membered heterocycle, wherein the C₃₋₁₀ carbocycle and 3- to 10-membered heterocycle are each optionally substituted with one or more R⁹; and
   C₃₋₁₀ carbocycle and 3- to 10-membered heterocycle, each of which is optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —N(R¹⁰)C(O)N(R¹⁰)₂, —OC(O)N(R¹⁰)₂, —N(R¹⁰)C(O)OR¹⁰, —C(O)OR¹⁰, —OC(O)R¹⁰, —S(O)R¹⁰, —S(O)₂R¹⁰, —NO₂, ═O, ═S, ═N(R¹⁰), —CN, C₁₋₆ alkyl, C₂₋₆ alkenyl, and C₂₋₆ alkynyl, wherein C₁₋₆ alkyl, C₂₋₆ alkenyl, and C₂₋₆ alkynyl are each optionally substituted with one or more R⁹;
each R⁹ is independently selected from:
   halogen, —R¹⁰, —OR¹⁰, —SR¹⁰, —N(R¹⁰)₂, —C(O)R¹⁰, —C(O)N(R¹⁰)₂, —N(R¹⁰)C(O)R¹⁰, —N(R¹⁰)C(O)N(R¹⁰)₂, —OC(O)N(R¹⁰)₂, —N(R¹⁰)C(O)OR¹⁰, —C(O)OR¹⁰, —OC(O)R¹⁰, —OC(O)₂R¹⁰, —S(O)R¹⁰, —S(O)₂R¹⁰, —NO₂, ═O, ═S, ═N(R¹⁰), and —CN; and
   C₁₋₃ alkyl, C₂₋₃ alkenyl, and C₂₋₃ alkynyl, each of which is optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —N(R¹⁰)C(O)N(R¹⁰)₂, —OC(O)N(R¹⁰)₂, —N(R¹⁰)C(O)OR¹⁰, —C(O)OR¹⁰, —OC(O)R¹⁰, —S(O)R¹⁰, —S(O)₂R¹⁰, —NO₂, ═O, ═S, ═N(R¹⁰), and —CN; and
   C₃₋₁₀ carbocycle and 3- to 10-membered heterocycle, each of which is optionally substituted with one or more substituents independently selected from halogen, —R¹⁰, —OR¹⁰, —SR¹⁰, —N(R¹⁰)₂, —C(O)R¹⁰, —C(O)N(R¹⁰)₂, —N(R¹⁰)C(O)R¹⁰, —N(R¹⁰)C(O)N(R¹⁰)₂, —OC(O)N(R¹⁰)₂, —N(R¹⁰)C(O)OR¹⁰, —C(O)OR¹⁰, —OC(O)R¹⁰, —S(O)R¹⁰, —S(O)₂R¹⁰, —NO₂, ═O, ═S, ═N(R¹⁰), and —CN; and

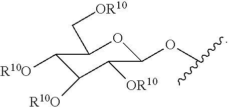

each R¹⁰ is independently selected from:
   hydrogen; and
   C₁₋₆ alkyl, C₂₋₆ alkenyl, and C₂₋₆ alkynyl, each of which is optionally substituted with one or more substituents independently selected from halogen, —CN, —OH, —SH, —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₃₋₁₀ carbocycle, 3- to 10-membered heterocycle; and
   C₃₋₁₀ carbocycle, and 3- to 10-membered heterocycle, each of which is optionally substituted with one or more substituents independently selected from halogen, —CN, —OH, —SH, —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₁₋₆ alkyl, C₂₋₆ alkenyl, C₂₋₆ alkynyl, C₃₋₁₀ carbocycle, 3- to 10-membered heterocycle, and C₁₋₆ haloalkyl;
W is selected from:
   C₁₋₆ alkylene, C₂₋₆ alkenylene, and C₂₋₆ alkynylene, each of which is optionally substituted with one or more substituents independently selected from halogen, —CN, —OH, —SH, —NO₂, —NH₂, =0, ═S, —O—C₁₋₆ alkyl, —S—C₁₋₆ alkyl, —N(C₁₋₆ alkyl)₂, —NH(C₁₋₆ alkyl), C₃₋₁₀ carbocycle, and 3- to 10-membered heterocycle;
R¹² is selected from:
   hydrogen, halogen, —OR¹⁰, —SR¹⁰, —N(R¹⁰)₂, —NO₂, and —CN; and
   C₁₋₆ alkyl optionally substituted with one or more substituents independently selected from halogen, —OR¹⁰, —SR¹⁰, —N(R¹⁰)₂, —NO₂, and —CN;
wherein if Z is NH then R⁴ is not NH₂, and
wherein if Z is O or S, R⁴ is —OR³, and R⁵ is H, then W—R³ is additionally selected from C₁₋₆ alkyl, C₂₋₆ alkenyl, and C₂₋₆ alkynyl.
   2. The compound or salt of embodiment 1, wherein Z is selected from —O— and —S—.

3. The compound or salt of embodiment 1 or 2, wherein Z is —O—.

4. The compound or salt of any one of embodiments 1 to 3, wherein X is CR⁶.

5. The compound or salt of any one of embodiments 1 to 4, wherein Y is CR¹².

6. The compound or salt of any one of embodiments 1 to 5, wherein Q is absent or selected from —W—, —O—, —OW—, and —WO—.

7. The compound or salt of any one of embodiments 1 to 6, wherein Q is absent or —O—.

8. The compound or salt of any one of embodiments 1 to 7, wherein Q is absent.

9. The compound or salt of any one of embodiments 1 to 8, wherein m is selected from 1 and 2.

10. The compound or salt of any one of embodiments 1 to 9, wherein m is 1.

11. The compound or salt of any one of embodiments 1 to 10, wherein R¹ is selected from:
hydrogen;
C₁₋₆ alkyl, C₂₋₆ alkenyl, and C₂₋₆ alkynyl, each of which is optionally substituted with one or more substituents independently selected from halogen, —CN, —OH, —SH, —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₃₋₁₀ carbocycle, 3- to 10-membered heterocycle; and
C₃₋₆ carbocycle, and 3- to 6-membered heterocycle, each of which is optionally substituted with one or more substituents independently selected from halogen, —CN, —OH, —SH, —NO₂, —NH₂, ═O, ═S, —O—C₁₋₆ alkyl, —S—C₁₋₆ alkyl, —N(C₁₋₆ alkyl)₂, and —NH(C₁₋₆ alkyl).

12. The compound or salt of any one of embodiments 1 to 11, wherein R¹ is selected from hydrogen and C₁₋₃ alkyl.

13. The compound or salt of any one of embodiments 1 to 12, wherein R² is selected from:
hydrogen;
halogen, —CN, —OH, —SH, —NO₂, —NH₂, ═O, ═S, —O—C₁₋₆ alkyl, —S—C₁₋₆ alkyl, —N(C₁₋₆ alkyl)₂, —NH(C₁₋₆ alkyl);
C₁₋₆ alkyl, C₂₋₆ alkenyl, and C₂₋₆ alkynyl, each of which is optionally substituted with one or more substituents independently selected from halogen, —CN, —OH, —SH, —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₃₋₁₀ carbocycle, 3- to 10-membered heterocycle; and
C₃₋₆ carbocycle, and 3- to 6-membered heterocycle, each of which is optionally substituted with one or more substituents independently selected from halogen, —CN, —OH, —SH, —NO₂, —NH₂, ═O, ═S, —S—C₁₋₆ alkyl, —N(C₁₋₆ alkyl)₂, and —NH(C₁₋₆ alkyl).

14. The compound or salt of any one of embodiments 1 to 13, wherein R² is selected from:
hydrogen;
halogen, —CN, —OH, —SH, —NO₂, —NH₂, ═O, ═S, —O—C₁₋₆ alkyl, —S—C₁₋₆ alkyl, —N(C₁₋₆ alkyl)₂, —NH(C₁₋₆ alkyl);
C₁₋₆ alkyl optionally substituted with one or more substituents independently selected from halogen, —CN, —OH, —SH, —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₃₋₁₀ carbocycle, 3- to 10-membered heterocycle; and
C₆ carbocycle, and 5- to 6-membered heterocycle, each of which is optionally substituted with one or more substituents independently selected from halogen.

15. The compound or salt of any one of embodiments 1 to 14, wherein R² is selected from:
hydrogen;
halogen, —CN, —OH, —SH, —NO₂, —NH₂, ═O, ═S, —O—C₁₋₆ alkyl, —S—C₁₋₆ alkyl, —N(C₁₋₆ alkyl)₂, —NH(C₁₋₆ alkyl);
C₁₋₆ alkyl optionally substituted with one or more substituents independently selected from halogen, —CN, —OH, —SH, —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₃₋₁₀ carbocycle, 3- to 10-membered heterocycle; and

16. The compound or salt of any one of embodiments 1 to 15, wherein R² is selected from hydrogen, halogen, and C₁₋₃ alkyl.

17. The compound or salt of any one of embodiments 1 to 16, wherein R³ is a C₃₋₆ carbocycle optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —N(R¹⁰)C(O)N(R¹⁰)₂, —OC(O)N(R¹⁰)₂, —N(R¹⁰)C(O)OR¹⁰, —C(O)OR¹⁰, —OC(O)R¹⁰, —S(O)R¹⁰, —S(O)₂R¹⁰, —NO₂, ═O, ═S, ═N(R¹⁰), —CN, C₁₋₆ alkyl, C₂₋₆ alkenyl, and C₂₋₆ alkynyl, wherein C₁₋₆ alkyl, C₂₋₆ alkenyl, and C₂₋₆ alkynyl are each optionally substituted with one or more R⁹.

18. The compound or salt of any one of embodiments 1 to 16, wherein R³ is a C₆ carbocycle optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —N(R¹⁰)C(O)N(R¹⁰)₂, —OC(O)N(R¹⁰)₂, —N(R¹⁰)C(O)OR¹⁰, —C(O)OR¹⁰, —OC(O)R¹⁰, —S(O)R¹⁰, —S(O)₂R¹⁰, —NO₂, ═O, ═S, ═N(R¹⁰), —CN, C₁₋₆ alkyl, C₂₋₆ alkenyl, and C₂₋₆ alkynyl, wherein C₁₋₆ alkyl, C₂₋₆ alkenyl, and C₂₋₆ alkynyl are each optionally substituted with one or more R⁹.

19. The compound or salt of any one of embodiments 1 to 16, wherein R³ is phenyl optionally substituted with one or more substituents independently selected from halogen, —CN, —OH, —SH, —NO₂, —NH₂, —O—C₁₋₆ alkyl, —S—C₁₋₆ alkyl, —N(C₁₋₆ alkyl)₂, and —NH(C₁₋₆ alkyl).

20. The compound or salt of any one of embodiments 1 to 16, wherein R³ is a 3- to 6-membered heterocycle optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —N(R¹⁰)C(O)N(R¹⁰)₂, —OC(O)N(R¹⁰)₂, —N(R¹⁰)C(O)OR¹⁰, —C(O)OR¹⁰, —OC(O)R¹⁰, —S(O)R¹⁰, —S(O)₂R¹⁰, —NO₂, ═O, ═S, ═N(R¹⁰), —CN, C₁₋₆ alkyl, C₂₋₆ alkenyl, and C₂₋₆ alkynyl, wherein C₁₋₆ alkyl, C₂₋₆ alkenyl, and C₂₋₆ alkynyl are each optionally substituted with one or more R⁹.

21. The compound or salt of any one of embodiments 1 to 16, wherein R³ is a 5- to 6-membered heterocycle optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —N(R¹⁰)C(O)N(R¹⁰)₂, —OC(O)N(R¹⁰)₂, —N(R¹⁰)C(O)OR¹⁰, —C(O)OR¹⁰, —OC(O)R¹⁰, —S(O)R¹⁰, —S(O)₂R¹⁰, —NO₂, ═O, ═S, ═N(R¹⁰), —CN, C₁₋₆ alkyl, C₂₋₆ alkenyl, and C₂₋₆ alkynyl, wherein C₁₋₆ alkyl, C₂₋₆ alkenyl, and C₂₋₆ alkynyl are each optionally substituted with one or more R⁹.

22. The compound or salt of any one of embodiments 1 to 16, wherein R³ is a 6-membered heterocycle optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —N(R¹⁰)C(O)N(R¹⁰)₂, —OC(O)N(R¹⁰)₂, —N(R¹⁰)C(O)OR¹⁰, —C(O)OR¹⁰, —OC(O)R¹⁰, —S(O)R¹⁰, —S(O)₂R¹⁰, —NO₂, ═O, ═S, ═N(R¹⁰), —CN, C₁₋₆ alkyl, C₂₋₆ alkenyl, and C₂₋₆ alkynyl, wherein C₁₋₆ alkyl, C₂₋₆ alkenyl, and C₂₋₆ alkynyl are each optionally substituted with one or more R⁹.

23. The compound or salt of any one of embodiments 1 to 16, wherein R³ is 6-membered heterocycle optionally substituted with one or more substituents independently selected from halogen, —CN, —OH, —SH, —NO₂, —NH₂, —O—C₁₋₆ alkyl, —S—C₁₋₆ alkyl, —N(C₁₋₆ alkyl)₂, and —NH(C₁₋₆ alkyl).

24. The compound or salt of any one of embodiments 1 to 16, wherein R³ is a 5-membered heterocycle optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —N(R¹⁰)C(O)N(R¹⁰)₂, —OC(O)N(R¹⁰)₂, —N(R¹⁰)C(O)OR¹⁰, —C(O)OR¹⁰, —OC(O)R¹⁰, —S(O)R¹⁰, —S(O)₂R¹⁰, —NO₂, ═O, ═S, ═N(R¹⁰), —CN, C₁₋₆ alkyl, C₂₋₆ alkenyl, and C₂₋₆ alkynyl, wherein C₁₋₆ alkyl, C₂₋₆ alkenyl, and C₂₋₆ alkynyl are each optionally substituted with one or more R⁹.

25. The compound or salt of any one of embodiments 1 to 16, wherein R³ is a 5-membered heterocycle optionally substituted with one or more substituents independently selected from halogen, —CN, —OH, ═O, ═S, ═NH, —SH, —NO₂, —NH₂, —O—C₁₋₆ alkyl, —S—C₁₋₆ alkyl, —N(C₁₋₆ alkyl)₂, —NH(C₁₋₆ alkyl), and C₁₋₆ alkyl.

26. The compound or salt of any one of embodiments 1 to 16, wherein R³ is a 5-membered heterocycle is selected from isoxazole, oxazole, thiadiazole, oxadiazole, pyrazole, tetrazole, and thiazole, any of which is optionally substituted with one or more R⁹.

27. The compound or salt of any one of embodiments 1 to 16, wherein R³ is a 5-membered heterocycle is selected tetrazole and oxadiazole.

28. The compound or salt of any one of embodiments 1 to 16, wherein R³ is a 5-membered heterocycle selected from isothiazol-3-ol, isoxazole-3-ol, squaric acid, thiazolidinedione, oxazolidinedione, tetrazole, 1,2,4-oxadiazol-5(4H)-one, 1,2,4-thiadiazol-5(4H)-one, 1,2,4-oxadiazole-5(4H)-thione, 2-methyl-2,4-dihydro-3H-1,2,4-triazole-3-thione, 3H-1,2,3,5-oxathiadiazole 2-oxide, 3-(methylsulfonyl)-4H-1,2,4-triazole, or 1,4-dihydro-5H-tetrazol-5-one, 3-hydroxyquinolin-2-one, and tetramic acid, any of which is optionally substituted with one or more R⁹.

29. The compound or salt of any one of embodiments 1 to 16, wherein R³ is a 5-membered heterocycle is selected from

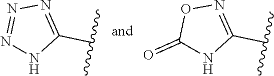 [SMILES: *c1nnn[nH]1 ; *c1noc(=O)[nH]1]

optionally substituted with one or more R⁹.

30. The compound or salt of any one of embodiments 1 to 16, wherein R³ is selected from

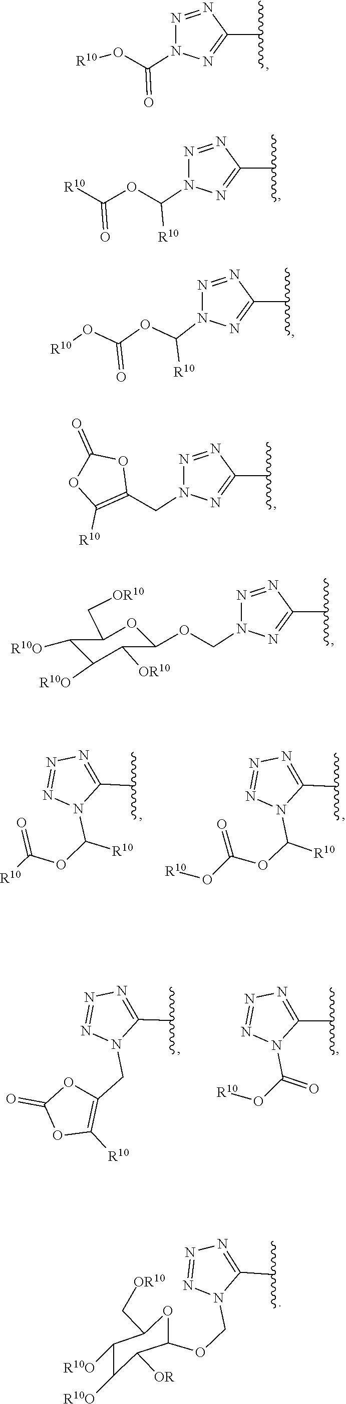

31. The compound or salt of any one of embodiments 1 to 16, wherein
Q is —W—;
R³ is additionally selected from halogen, —OR¹⁰, —SR¹⁰, —N(R¹⁰)₂, —C(O)R¹⁰, —C(O)N(R¹⁰)₂, —N(R¹⁰)C(O)R¹⁰, —N(R¹⁰)C(O)N(R¹⁰)₂, —OC(O)N(R¹⁰)₂, —N(R¹⁰)C(O)OR¹⁰, —C(O)OR¹⁰, —OC(O)R¹⁰, —S(O)R¹⁰, —S(O)₂R¹⁰, —NO₂, —CN; and
—W— is C₁₋₆ alkylene optionally substituted with one or more substituents independently selected from halogen, —CN, —OH, —SH, —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₃₋₁₀ carbocycle, and 3- to 10-membered heterocycle.

32. The compound or salt of any one of embodiments 1 to 16, wherein
Q is —W—; and
R³ is additionally selected from halogen, —CN, —OH, —SH, —NO₂, —NH₂, —O—C₁₋₆ alkyl, —S—C₁₋₆ alkyl, —N(C₁₋₆ alkyl)₂, and —NH(C₁₋₆ alkyl), and
—W— is C₁₋₆ alkylene optionally substituted with one or more substituents independently selected from halogen, —CN, —OH, —SH, —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₃₋₁₀ carbocycle, and 3- to 10-membered heterocycle.

33. The compound or salt of any one of embodiments 1 to 16, wherein
Q is —W—; and
R³ is additionally selected from —OH, —SH, —NH₂, —O—C₁₋₆ alkyl, —S—C₁₋₆ alkyl, —N(C₁₋₆ alkyl)₂, and —NH(C₁₋₆ alkyl); and
—W— is C₁ alkylene optionally substituted with one or more substituents independently selected from halogen, —CN, —OH, —SH, —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₃₋₁₀ carbocycle, and 3- to 10-membered heterocycle.

34. The compound or salt of any one of embodiments 1 to 16, wherein
Q is —W—; and
R³ is additionally selected from —OH, —SH, —NH₂, —O—C₁₋₆ alkyl, —S—C₁₋₆ alkyl, —N(C₁₋₆ alkyl)₂, and —NH(C₁₋₆ alkyl); and
—W— is —C(═O)—.

35. The compound or salt of any one of embodiments 1 to 34, wherein each R⁴ is independently selected from:
halogen, —OR⁷, —SR⁷, —N(R⁷)₂, —NO₂, and —CN;
C₁₋₆ alkyl, C₂₋₆ alkenyl, and C₂₋₆ alkynyl, each of which is optionally substituted with one or more substituents independently selected from halogen, —CN, —OH, —SH, —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₃₋₁₀ carbocycle, 3- to 10-membered heterocycle; and
C₃₋₆ carbocycle, and 3- to 6-membered heterocycle, each of which is optionally substituted with one or more substituents independently selected from halogen, —CN, —OH, —SH, —NO₂, —NH₂, =0, ═S, —O—C₁₋₆ alkyl, —S—C₁₋₆ alkyl, —N(C₁₋₆ alkyl)₂, and —NH(C₁₋₆ alkyl);

36. The compound or salt of any one of embodiments 1 to 35, wherein R⁴ is independently selected from —OR⁷, —SR⁷, and —N(R⁷)₂.

37. The compound or salt of any one of embodiments 1 to 36, wherein m is 1 and R⁴ is —OR⁷.

38. The compound or salt of any one of embodiments 1 to 37, wherein each R⁷ is selected from:
C₁₋₆ alkyl, C₂₋₆ alkenyl, and C₂₋₆ alkynyl, each of which is optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —C(O)OR¹⁰, —OC(O)R¹⁰, —N(R¹⁰)C(O)N(R¹⁰)₂, —OC(O)N(R¹⁰)₂, —N(R¹⁰)C(O)OR¹⁰, —S(O)R¹⁰, —S(O)₂R¹⁰, —NO₂, ═O, ═S, ═N(R¹⁰), —CN, C₃₋₁₀ carbocycle and 3- to 10-membered heterocycle, wherein the C₃₋₁₀ carbocycle and 3- to 10-membered heterocycle are each optionally substituted with one or more R⁹; and
C₃₋₁₀ carbocycle and 3- to 10-membered heterocycle, each of which is optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —N(R¹⁰)C(O)N(R¹⁰)₂, —OC(O)N(R¹⁰)₂, —N(R¹⁰)C(O)OR¹⁰, —C(O)OR¹⁰, —OC(O)R¹⁰, —S(O)R¹⁰, —S(O)₂R¹⁰, —NO₂, ═O, ═S, ═N(R¹⁰), —CN, C₁₋₆ alkyl, C₂₋₆ alkenyl, and C₂₋₆ alkynyl, wherein C₁₋₆ alkyl, C₂₋₆ alkenyl, and C₂₋₆ alkynyl are each optionally substituted with one or more R⁹.

39. The compound or salt of any one of embodiments 1 to 38, wherein each R⁷ is selected from:
C₁₋₆ alkyl optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —C(O)OR¹⁰, —OC(O)R¹⁰, —N(R¹⁰)C(O)N(R¹⁰)₂, —OC(O)N(R¹⁰)₂, —N(R¹⁰)C(O)OR¹⁰, —S(O)R¹⁰, —S(O)₂R¹⁰, —NO₂, ═O, ═S, ═N(R¹⁰), —CN, C₃₋₁₀ carbocycle and 3- to 10-membered heterocycle, wherein the C₃₋₁₀ carbocycle and 3- to 10-membered heterocycle are each optionally substituted with one or more R⁹; and
C₃₋₁₀ carbocycle and 3- to 10-membered heterocycle, each of which is optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —N(R¹⁰)C(O)N(R¹⁰)₂, —OC(O)N(R¹⁰)₂, —N(R¹⁰)C(O)OR¹⁰, —C(O)OR¹⁰, —OC(O)R¹⁰, —S(O)R¹⁰, —S(O)₂R¹⁰, —NO₂, ═O, ═S, ═N(R¹⁰), —CN, C₁₋₆ alkyl, C₂₋₆ alkenyl, and C₂₋₆ alkynyl, wherein C₁₋₆ alkyl, C₂₋₆ alkenyl, and C₂₋₆ alkynyl are each optionally substituted with one or more R⁹.

40. The compound or salt of any one of embodiments 1 to 39, wherein each R⁷ is selected from:
C₁₋₆ alkyl optionally substituted with one or more substituents independently selected from C₃₋₁₀ carbocycle and 3- to 10-membered heterocycle, wherein the C₃₋₁₀ carbocycle and 3- to 10-membered heterocycle are each optionally substituted with one or more R⁹; and
C₃₋₁₀ carbocycle and 3- to 10-membered heterocycle, each of which is optionally substituted with one or more substituents independently selected from halogen, —OR¹⁰, —SR¹⁰, —N(R¹⁰)₂, —CN, and C₁₋₆ alkyl optionally substituted with one or more R⁹.

41. The compound or salt of any one of embodiments 1 to 40, wherein R⁷ is C₁₋₆ alkyl substituted with one or more substituents independently selected from C₃₋₁₀ carbocycle and 3- to 10-membered heterocycle, wherein the C₃₋₁₀ carbocycle and 3- to 10-membered heterocycle are each optionally substituted with one or more substituents independently selected from halogen, —CN, —OH, —SH, —NO₂, —NH₂, —O—C₁₋₆ alkyl, —S—C₁₋₆ alkyl, —N(C₁₋₆ alkyl)₂, and —NH(C₁₋₆ alkyl).

42. The compound or salt of any one of embodiments 1 to 41, wherein R⁷ is C₁₋₆ alkyl substituted with one or more substituents independently selected from C₃₋₆ carbocycle and 3- to 6-membered heterocycle, wherein the C₃₋₆ carbocycle and 3- to 6-membered heterocycle are each optionally substituted with one or more substituents independently selected from halogen, —CN, —OH, —SH, —NO₂, —NH₂, —O—C₁₋₆ alkyl, —S—C₁₋₆ alkyl, —N(C₁₋₆ alkyl)₂, and —NH(C₁₋₆ alkyl).

43. The compound or salt of any one of embodiments 1 to 40, wherein R⁷ is selected from C₃₋₁₀ carbocycle and 3- to 10-membered heterocycle, each of which is optionally substituted with one or more substituents independently selected from halogen, —CN, —OH, —SH, —NO₂, —NH₂, —O—C₁₋₆ alkyl, —S—C₁₋₆ alkyl, —N(C₁₋₆ alkyl)₂, and —NH(C₁₋₆ alkyl).

44. The compound or salt of any one of embodiments 1 to 43, wherein R⁵ is selected from hydrogen, halogen, —CN, —OH, —SH, —NO₂, —NH₂, —O—C₁₋₆ alkyl, —S—C₁₋₆ alkyl, —N(C₁₋₆ alkyl)₂, and —NH(C₁₋₆ alkyl).

45. The compound or salt of any one of embodiments 1 to 44, wherein R⁵ is hydrogen.

46. The compound or salt of any one of embodiments 1 to 45, wherein R⁶ is selected from hydrogen, halogen, —CN, —OH, —SH, —NO₂, —NH₂, —O—C₁₋₆ alkyl, —S—C₁₋₆ alkyl, —N(C₁₋₆ alkyl)₂, and —NH(C₁₋₆ alkyl).

47. The compound or salt of any one of embodiments 1 to 46, wherein R⁶ is selected from hydrogen and halogen.

48. The compound or salt of any one of embodiments 1 to 47, wherein Z is —NR⁸—.

49. The compound or salt of any one of embodiments 1 to 48, wherein R⁸ is selected from hydrogen.

50. The compound or salt of any one of embodiments 1 to 48, wherein R⁸ is selected from C₁₋₆ alkyl, C₂₋₆ alkenyl, and C₂₋₆ alkynyl, each of which is optionally substituted with one or more substituents independently selected from halogen, —SR¹⁰, —N(R¹⁰)₂, —C(O)R¹⁰, —C(O)N(R¹⁰)₂, —N(R¹⁰)C(O)R¹⁰, —C(O)OR¹⁰, —OC(O)R¹⁰, —N(R¹⁰)C(O)N(R¹⁰)₂, —OC(O)N(R¹⁰)₂, —N(R¹⁰)C(O)OR¹⁰, —S(O)R¹⁰, —S(O)₂R¹⁰, —NO₂, ═O, ═S, ═N(R¹⁰), —CN, C₃₋₁₀ carbocycle and 3- to 10-membered heterocycle, wherein the C₃₋₁₀ carbocycle and 3- to 10-membered heterocycle are each optionally substituted with one or more R⁹.

51. The compound or salt of any one of embodiments 1 to 48, wherein R⁸ is selected from C₁₋₆ alkyl, C₂₋₆ alkenyl, and C₂₋₆ alkynyl, each of which is optionally substituted with one or more substituents independently selected from halogen, —CN, —SH, —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₃₋₁₀ carbocycle, 3- to 10-membered heterocycle.

52. The compound or salt of any one of embodiments 1 to 48, wherein R⁸ is selected from C₁₋₆ alkyl optionally substituted with one or more substituents independently selected from halogen, —CN, —SH, —NO₂, —NH₂, —O—C₁₋₆ alkyl, —S—C₁₋₆ alkyl, —N(C₁₋₆ alkyl)₂, and —NH(C₁₋₆ alkyl).

53. The compound or salt of any one of embodiments 1 to 48, wherein R⁸ is selected from C₁₋₆ alkyl.

54. The compound or salt of any one of embodiments 1 to 48, wherein R⁸ is selected from C₃₋₁₀ optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —N(R¹⁰)C(O)N(R¹⁰)₂, —OC(O)N(R¹⁰)₂, —N(R¹⁰)C(O)OR¹⁰, —C(O)OR¹⁰, —OC(O)R¹⁰, —S(O)R¹⁰, —S(O)₂R¹⁰, —NO₂, ═O, ═S, ═N(R¹⁰), —CN, C₁₋₆ alkyl, C₂₋₆ alkenyl, and C₂₋₆ alkynyl, wherein C₁₋₆ alkyl, C₂₋₆ alkenyl, and C₂₋₆ alkynyl are each optionally substituted with one or more R⁹.

55. The compound or salt of any one of embodiments 1 to 48, wherein R⁸ is selected from C₃₋₁₀ carbocycle optionally substituted with one or more substituents independently selected from halogen, —CN, —OH, —SH, —NO₂, —NH₂, —O—C₁₋₆ alkyl, —S—C₁₋₆ alkyl, —N(C₁₋₆ alkyl)₂, and —NH(C₁₋₆ alkyl).

56. The compound or salt of any one of embodiments 1 to 48, wherein R⁸ is selected from 3- to 10-membered heterocycle optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —N(R¹⁰)C(O)N(R¹⁰)₂, —OC(O)N(R¹⁰)₂, —N(R¹⁰)C(O)OR¹⁰, —C(O)OR¹⁰, —OC(O)R¹⁰, —S(O)R¹⁰, —S(O)₂R¹⁰, —NO₂, ═O, ═S, ═N(R¹⁰), —CN, C₁₋₆ alkyl, C₂₋₆ alkenyl, and C₂₋₆ alkynyl, wherein C₁₋₆ alkyl, C₂₋₆ alkenyl, and C₂₋₆ alkynyl are each optionally substituted with one or more R⁹.

57. The compound or salt of any one of embodiments 1 to 48, wherein R⁸ is selected from 3- to 10-membered heterocycle optionally substituted with one or more substituents independently selected from halogen, —CN, —OH, —SH, —NO₂, —NH₂, —O—C₁₋₆ alkyl, —S—C₁₋₆ alkyl, —N(C₁₋₆ alkyl)₂, and —NH(C₁₋₆ alkyl).

58. The compound or salt of any one of embodiments 1 to 57, wherein R⁸ is hydrogen.

59. The compound or salt of any one of embodiments 1 to 58, wherein each R⁹ is selected from:
halogen, —CN, —OH, —SH, —NO₂, —NH₂, —O—C₁₋₆ alkyl, —S—C₁₋₆ alkyl, —N(C₁₋₆ alkyl)₂, and —NH(C₁₋₆ alkyl); and
C₁₋₃ alkyl optionally substituted with one or more substituents independently selected from halogen, —CN, —OH, —SH, —NO₂, —NH₂, —O—C₁₋₆ alkyl, —S—C₁₋₆ alkyl, —N(C₁₋₆ alkyl)₂, and —NH(C₁₋₆ alkyl).

60. The compound or salt of any one of embodiments 1 to 59, wherein each R¹⁰ is hydrogen.

61. The compound or salt of any one of embodiments 1 to 60, wherein W is C₁₋₆ alkylene optionally substituted with one or more substituents independently selected from halogen, —CN, —OH, —SH, —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₃₋₁₀ carbocycle, and 3- to 10-membered heterocycle.

62. The compound or salt of any one of embodiments 1 to 60, wherein W is C₁₋₆ alkylene optionally substituted with one or more substituents independently selected from halogen, —CN, —OH, —SH, —NO₂, —NH₂, —O—C₁₋₆ alkyl, —S—C₁₋₆ alkyl, —N(C₁₋₆ alkyl)₂, —NH(C₁₋₆ alkyl), C₃₋₁₀ carbocycle, and 3- to 10-membered heterocycle.

63. The compound or salt of any one of embodiments 1 to 60, wherein W is C₁₋₆ alkylene optionally substituted with one or more substituents independently selected from halogen, —CN, —OH, —SH, —NO₂, —NH₂, —O—C₁₋₆ alkyl, —S—C₁₋₆ alkyl, —N(C₁₋₆ alkyl)₂, and —NH(C₁₋₆ alkyl).

64. The compound or salt of any one of embodiments 1 to 60, wherein W is C₁₋₆ alkylene optionally substituted with one or more substituents independently selected from C₃₋₁₀ carbocycle, and 3- to 10-membered heterocycle.

65. The compound or salt of any one of embodiments 1 to 60, wherein W is C₁₋₆ alkylene substituted with one or more substituents independently selected from C₃₋₆ carbocycle, and 3- to 5-membered heterocycle.

66. The compound or salt of any one of embodiments 1 to 60, wherein W is C₁₋₆ alkylene substituted with one or more substituents independently selected from C₃₋₆ carbocycle.

67. The compound or salt of any one of embodiments 1 to 60, wherein W is selected from

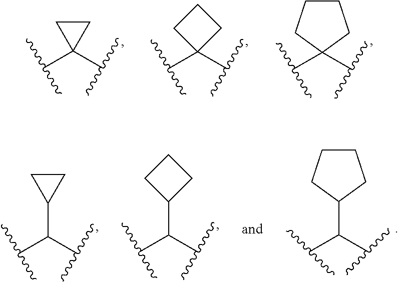 [SMILES: *C1(*)CC1 ; *C1(*)CCC1 ; *C1(*)CCCC1 ; *C(*)C1CC1 ; *C(*)C1CCC1 ; *C(*)C1CCCC1]

68. The compound or salt ofany one of embodiments 1 to 60, wherein W is C₁₋₆ alkylene.

69. The compound or salt of any one of embodiments 1 to 68, wherein R¹² is selected from hydrogen and halogen.

70. The compound or salt of embodiment 1, selected from:

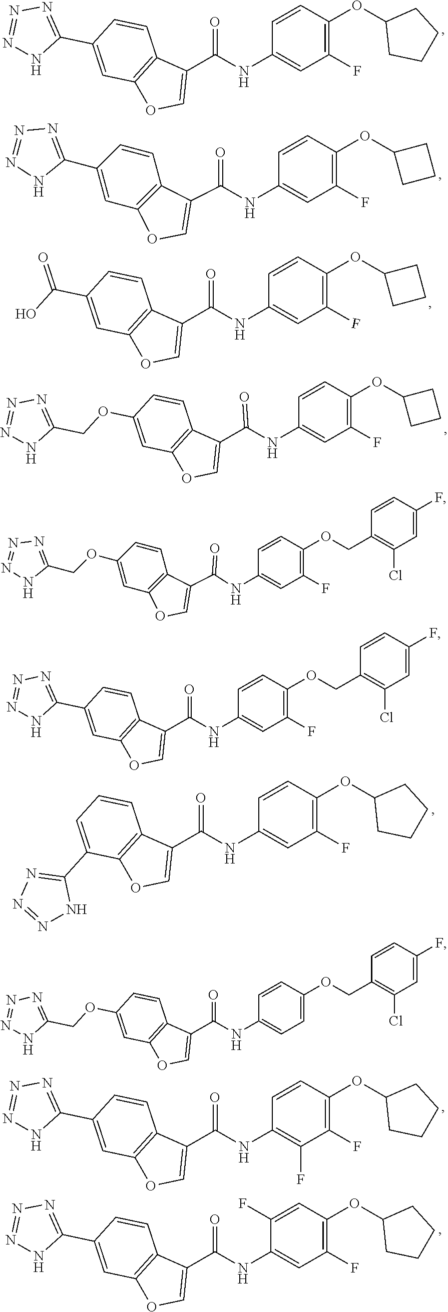

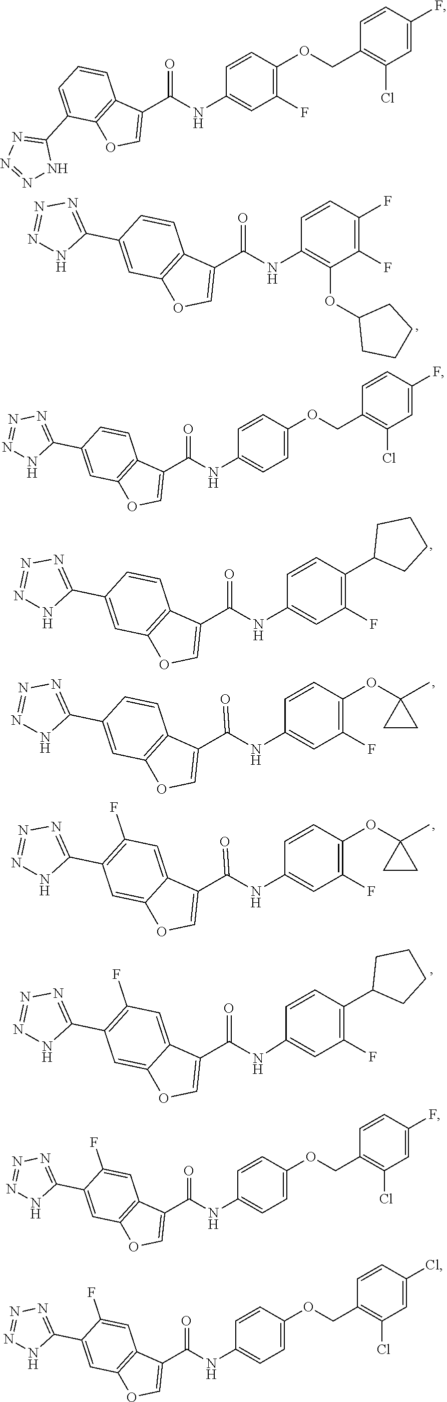

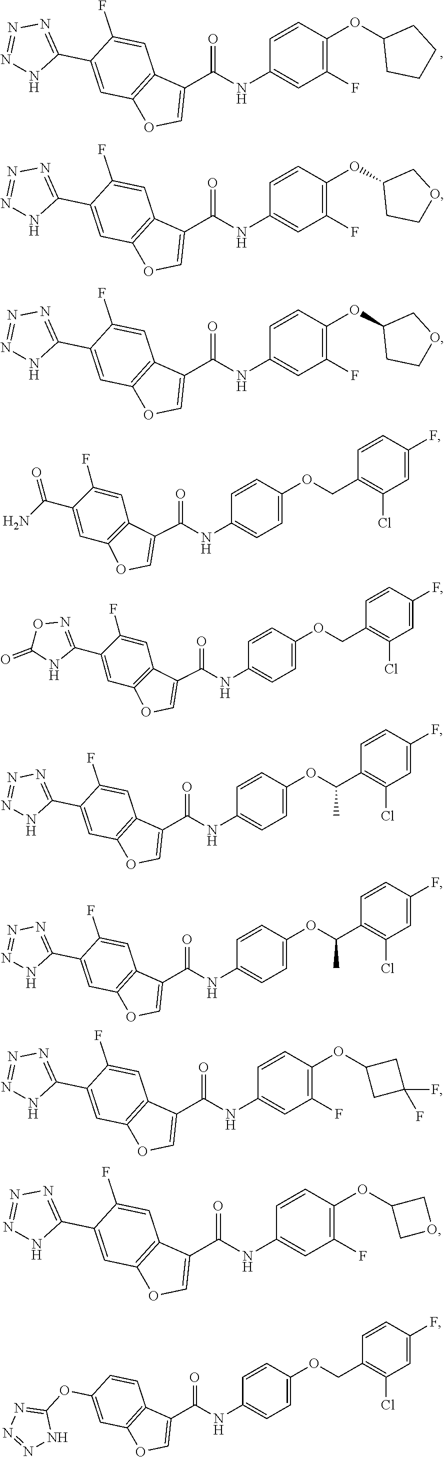

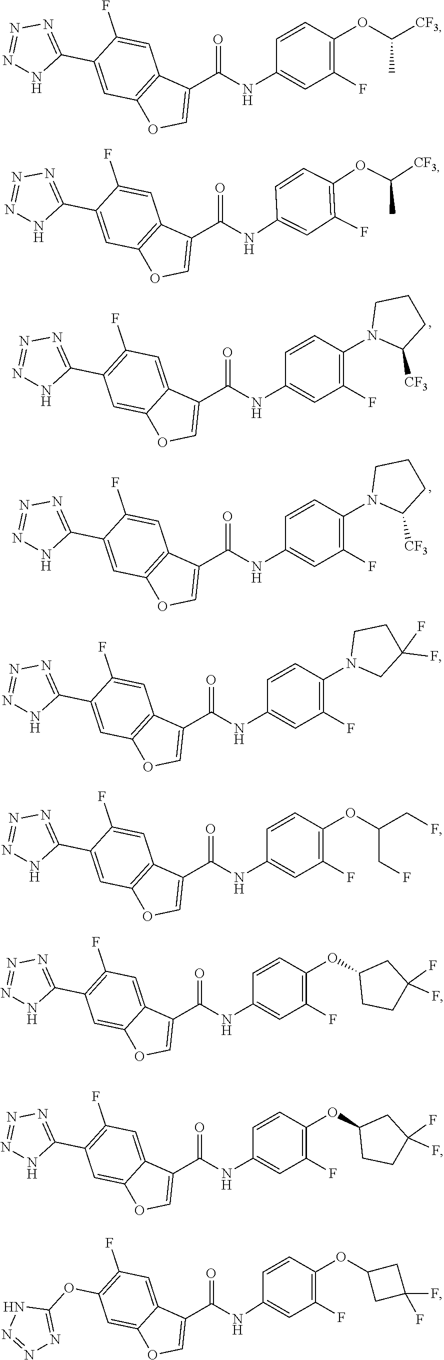

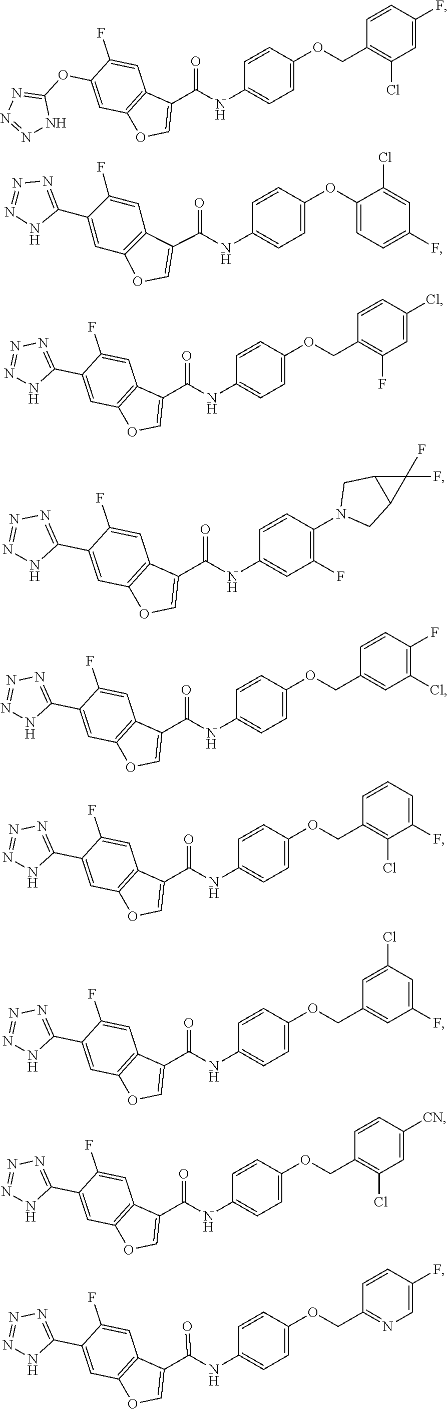

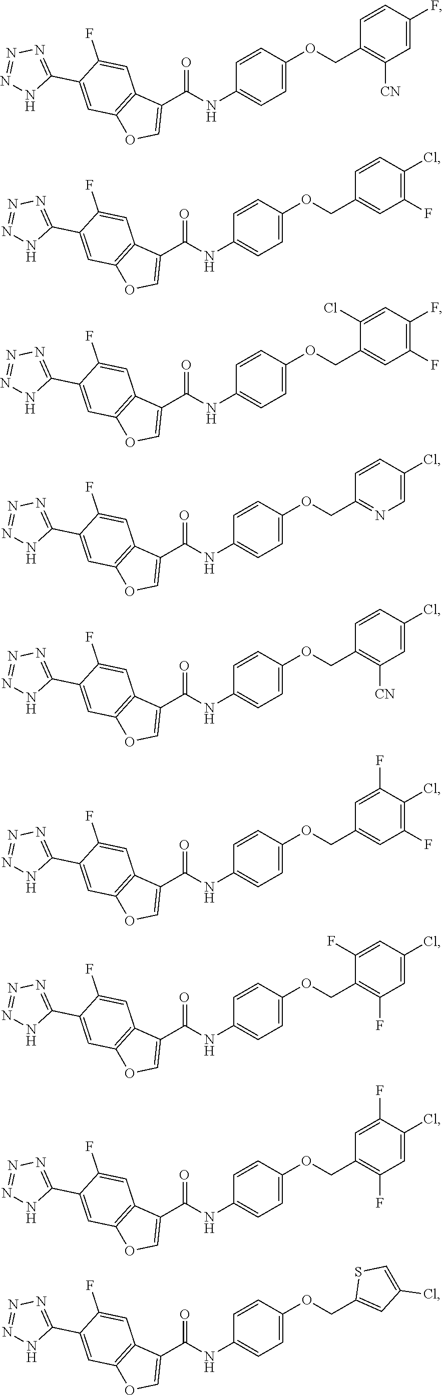

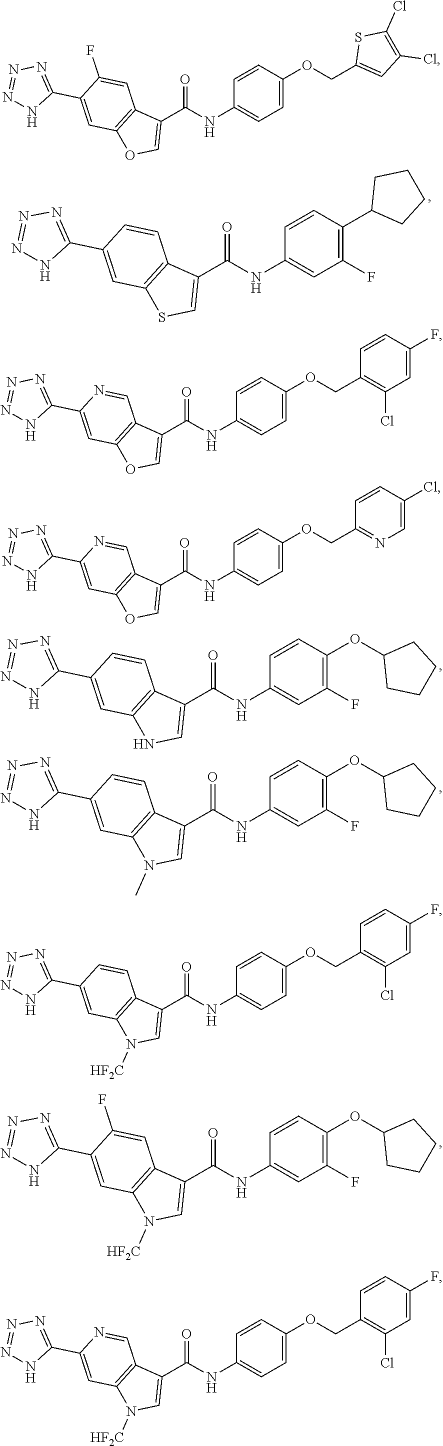

and combinations thereof.

71. A pharmaceutical composition comprising a compound or salt of any one of embodiments 1 to 70 and a pharmaceutically acceptable excipient.

72. A method of treating cancer, comprising administering to a subject in need thereof a compound or salt of any one of embodiments 1 to 70.

73. A method of treating cancer, comprising administering to a subject in need thereof a compound represented by Formula (II):

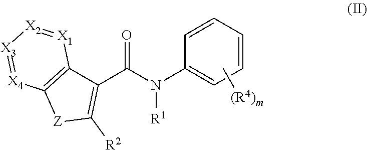

or a pharmaceutically acceptable salt, wherein:
X₁, X₂, X₃, and X₄ are each independently selected from C-Q-R³, CR⁶ and N;
Z is selected from —O—, —S—, and —NR⁸—;
Q is absent or selected from:
   —W—, —O—, —OW—, —WO—, —S—, —SW—, —WS—, —NR¹⁰—W—, —W—NR₁₀—, C(O)—, —C(O)N(R¹⁰)—, —N(R¹⁰)C(O)—, —C(O)O—, —OC(O)—, —N(R¹⁰)C(O)N(R¹⁰)0-, —OC(O)N(R¹⁰)—, —N(R¹⁰)C(O)O—, —S(O)—, —S(O)₂—, and —P(O)₂O—;
m is selected from 1, 2, 3, 4, and 5;
R¹ is selected from:
   hydrogen;
   C₁₋₆ alkyl, C₂₋₆ alkenyl, and C₂₋₆ alkynyl, each of which is optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —C(O)OR¹⁰, —OC(O)R¹⁰, —N(R¹⁰)C(O)N(R¹⁰)₂, —OC(O)N(R¹⁰)₂, —N(R¹⁰)C(O)OR¹⁰, —S(O)R¹⁰, —S(O)₂R¹⁰, —NO₂, ═O, ═S, ═N(R¹⁰), —CN, C₃₋₁₀ carbocycle and 3- to 10-membered heterocycle, wherein the C₃₋₁₀ carbocycle and 3- to 10-membered heterocycle are each optionally substituted with one or more R⁹; and
   C₃₋₁₀ carbocycle and 3- to 10-membered heterocycle, each of which is optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —N(R¹⁰)C(O)N(R¹⁰)₂, —OC(O)N(R¹⁰)₂, —N(R¹⁰)C(O)OR¹⁰, —C(O)OR¹⁰, —OC(O)R¹⁰, —S(O)R¹⁰, —S(O)₂R¹⁰, —NO₂, ═O, ═S, ═N(R¹⁰), —CN, C₁₋₆ alkyl, C₂₋₆ alkenyl, and C₂₋₆ alkynyl, wherein C₁₋₆ alkyl, C₂₋₆ alkenyl, and C₂₋₆ alkynyl are each optionally substituted with one or more R⁹;
R² is selected from:
   hydrogen;
   halogen, —OR¹⁰, —SR¹⁰, —N(R¹⁰)₂, —C(O)R¹⁰, —C(O)N(R¹⁰)₂, —N(R¹⁰)C(O)R¹⁰, —N(R¹⁰)C(O)N(R¹⁰)₂, —OC(O)N(R¹⁰)₂, —N(R¹⁰)C(O)OR¹⁰, —C(O)OR¹⁰, —OC(O)R¹⁰, —S(O)R¹⁰, —S(O)₂R¹⁰, —NO₂, ═O, ═S, ═N(R¹⁰), and —CN;
   C₁₋₆ alkyl, C₂₋₆ alkenyl, and C₂₋₆ alkynyl, each of which is optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —C(O)OR¹⁰, —OC(O)R¹⁰, —N(R¹⁰)C(O)N(R¹⁰)₂, —OC(O)N(R¹⁰)₂, —N(R¹⁰)C(O)OR¹⁰, —S(O)R¹⁰, —S(O)₂R¹⁰, —NO₂, ═O, ═S, ═N(R¹⁰), —CN, C₃₋₁₀ carbocycle and 3- to 10-membered heterocycle, wherein the C₃₋₁₀ carbocycle and 3- to 10-membered heterocycle are each optionally substituted with one or more R⁹; and
   C₃₋₁₀ carbocycle and 3- to 10-membered heterocycle, each of which is optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —N(R¹⁰)C(O)N(R¹⁰)₂, —OC(O)N(R¹⁰)₂, —N(R¹⁰)C(O)OR¹⁰, —C(O)OR¹⁰, —OC(O)R¹⁰, —S(O)R¹⁰, —S(O)₂R¹⁰, —NO₂, ═O, ═S, ═N(R¹⁰), —CN, C₁₋₆ alkyl, C₂₋₆ alkenyl, and C₂₋₆ alkynyl, wherein C₁₋₆ alkyl, C₂₋₆ alkenyl, and C₂₋₆ alkynyl are each optionally substituted with one or more R⁹;
R³ is selected from:
   hydrogen;
   halogen, —OR¹⁰, —SR¹⁰, —N(R¹⁰)₂, —C(O)R¹⁰, —C(O)N(R¹⁰)₂, —N(R¹⁰)C(O)R¹⁰, —N(R¹⁰)C(O)N(R¹⁰)₂, —OC(O)N(R¹⁰)₂, —N(R¹⁰)C(O)OR¹⁰, —C(O)OR¹⁰, —OC(O)R¹⁰, —S(O)R¹⁰, —S(O)₂R¹⁰, —NO₂, ═O, ═S, ═N(R¹⁰), and —CN;
   C₁₋₆ alkyl, C₂₋₆ alkenyl, and C₂₋₆ alkynyl, each of which is optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —C(O)OR¹⁰, —OC(O)R¹⁰, —N(R¹⁰)C(O)N(R¹⁰)₂, —OC(O)N(R¹⁰)₂, —N(R¹⁰)C(O)OR¹⁰, —S(O)R¹⁰, —S(O)₂R¹⁰, —NO₂, ═O, ═S, ═N(R¹⁰), —CN, C₃₋₁₀ carbocycle and 3- to 10-membered heterocycle, wherein the C₃₋₁₀ carbocycle and 3- to 10-membered heterocycle are each optionally substituted with one or more R⁹; and
   C₃₋₁₀ carbocycle and 3- to 10-membered heterocycle, each of which is optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —N(R¹⁰)C(O)N(R¹⁰)₂, —OC(O)N(R¹⁰)₂, —N(R¹⁰)C(O)OR¹⁰, —C(O)OR¹⁰, —OC(O)R¹⁰, —S(O)R¹⁰, —S(O)₂R¹⁰, —NO₂, ═O, ═S, ═N(R¹⁰), —CN, C₁₋₆ alkyl, C₂₋₆ alkenyl, and C₂₋₆ alkynyl, wherein C₁₋₆ alkyl, C₂₋₆ alkenyl, and C₂₋₆ alkynyl are each optionally substituted with one or more R⁹;
each R⁴ is independently selected from:
   halogen, —OR⁷, —SR⁷, —N(R⁷)₂, —C(O)R⁷, —C(O)N(R⁷)₂, —N(R⁷)C(O)R⁷, —N(R⁷)C(O)N(R⁷)₂, —OC(O)N(R⁷)₂, —N(R⁷)C(O)OR⁷, —C(O)OR⁷, —OC(O)R⁷, —S(O)R⁷, —S(O)₂R⁷, —NO₂, and —CN;
   C₁₋₆ alkyl, C₂₋₆ alkenyl, and C₂₋₆ alkynyl, each of which is optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —C(O)OR¹⁰, —OC(O)R¹⁰, —N(R¹⁰)C(O)N(R¹⁰)₂, —OC(O)N(R¹⁰)₂, —N(R¹⁰)C(O)OR¹⁰, —S(O)R¹⁰, —S(O)₂R¹⁰, —NO₂, ═O, ═S, ═N(R¹⁰), —CN, C₃₋₁₀ carbocycle and 3- to 10-membered heterocycle, wherein the C₃₋₁₀ carbocycle and 3- to 10-membered heterocycle are each optionally substituted with one or more R⁹; and
   C₃₋₁₀ carbocycle and 3- to 10-membered heterocycle, each of which is optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —N(R¹⁰)C(O)N(R¹⁰)₂, —OC(O)N(R¹⁰)₂, —N(R¹⁰)C(O)OR¹⁰, —C(O)OR¹⁰, —OC(O)R¹⁰, —S(O)R¹⁰, —S(O)₂R¹⁰, —NO₂, ═O, ═S, ═N(R¹⁰), —CN, C₁₋₆ alkyl, C₂₋₆ alkenyl, and C₂₋₆ alkynyl, wherein C₁₋₆ alkyl, C₂₋₆ alkenyl, and C₂₋₆ alkynyl are each optionally substituted with one or more R⁹;
each R⁶ is independently selected from:
   hydrogen, halogen, —OR¹⁰, —SR¹⁰, —N(R¹⁰)₂, —NO₂, and —CN; and
   C₁₋₆ alkyl optionally substituted with one or more substituents independently selected from halogen, —OR¹⁰, —SR¹⁰, —N(R¹⁰)₂, —NO₂, and —CN;
each R⁷ is independently selected from:
   hydrogen;
   C₁₋₆ alkyl, C₂₋₆ alkenyl, and C₂₋₆ alkynyl, each of which is optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —C(O)OR¹⁰, —OC(O)R¹⁰, —N(R¹⁰)C(O)N(R¹⁰)₂, —OC(O)N(R¹⁰)₂, —N(R¹⁰)C(O)OR¹⁰, —S(O)R¹⁰, —S(O)₂R¹⁰, —NO₂, ═O, ═S, ═N(R¹⁰), —CN, C₃₋₁₀ carbocycle and 3- to 10-membered heterocycle, wherein the C₃₋₁₀ carbocycle and 3- to 10-membered heterocycle are each optionally substituted with one or more R⁹; and
   C₃₋₁₀ carbocycle and 3- to 10-membered heterocycle, each of which is optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —N(R¹⁰)C(O)N(R¹⁰)₂, —OC(O)N(R¹⁰)₂, —N(R¹⁰)C(O)OR¹⁰, —C(O)OR¹⁰, —OC(O)R¹⁰, —S(O)R¹⁰, —S(O)₂R¹⁰, —NO₂, ═O, ═S, ═N(R¹⁰), —CN, C₁₋₆ alkyl, C₂₋₆ alkenyl, and C₂₋₆ alkynyl, wherein C₁₋₆ alkyl, C₂₋₆ alkenyl, and C₂₋₆ alkynyl are each optionally substituted with one or more R⁹;
R⁸ is selected from:
   hydrogen;
   C₁₋₆ alkyl, C₂₋₆ alkenyl, and C₂₋₆ alkynyl, each of which is optionally substituted with one or more substituents independently selected from halogen, —SR¹⁰, —N(R¹⁰)₂, —C(O)R¹⁰, —C(O)N(R¹⁰)₂, —N(R¹⁰)C(O)R¹⁰, —C(O)OR¹⁰, —OC(O)R¹⁰, —N(R¹⁰)C(O)N(R¹⁰)₂, —OC(O)N(R¹⁰)₂, —N(R¹⁰)C(O)OR¹⁰, —S(O)R¹⁰, —S(O)₂R¹⁰, —NO₂, ═O, ═S, ═N(R¹⁰), —CN, C₃₋₁₀ carbocycle and 3- to 10-membered heterocycle, wherein the C₃₋₁₀ carbocycle and 3- to 10-membered heterocycle are each optionally substituted with one or more R⁹; and
   C₃₋₁₀ carbocycle and 3- to 10-membered heterocycle, each of which is optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —N(R¹⁰)C(O)N(R¹⁰)₂, —OC(O)N(R¹⁰)₂, —N(R¹⁰)C(O)OR¹⁰, —C(O)OR¹⁰, —OC(O)R¹⁰, —S(O)R¹⁰, —S(O)₂R¹⁰, —NO₂, ═O, ═S, ═N(R¹⁰), —CN, C₁₋₆ alkyl, C₂₋₆ alkenyl, and C₂₋₆ alkynyl, wherein C₁₋₆ alkyl, C₂₋₆ alkenyl, and C₂₋₆ alkynyl are each optionally substituted with one or more R⁹;
each R⁹ is independently selected from:
   halogen, —R¹⁰, —OR¹⁰, —SR¹⁰, —N(R¹⁰)₂, —C(O)R¹⁰, —C(O)N(R¹⁰)₂, —N(R¹⁰)C(O)R¹⁰, —N(R¹⁰)C(O)N(R¹⁰)₂, —OC(O)N(R¹⁰)₂, —N(R¹⁰)C(O)OR¹⁰, —C(O)OR¹⁰, —OC(O)R¹⁰, —OC(O)₂R¹⁰, —S(O)R¹⁰, —S(O)₂R¹⁰, —NO₂, ═O, ═S, ═N(R¹⁰), and —CN; and
   C₁₋₃ alkyl, C₂₋₃ alkenyl, and C₂₋₃ alkynyl, each of which is optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —N(R¹⁰)C(O)N(R¹⁰)₂, —OC(O)N(R¹⁰)₂, —N(R¹⁰)C(O)OR¹⁰, —C(O)OR¹⁰, —OC(O)R¹⁰, —S(O)R¹⁰, —S(O)₂R¹⁰, —NO₂, ═O, ═S, ═N(R¹⁰), and —CN; and
   C₃₋₁₀ carbocycle and 3- to 10-membered heterocycle, each of which is optionally substituted with one or more substituents independently selected from halogen, —R¹⁰, —OR¹⁰, —SR¹⁰, —N(R¹⁰)₂, —C(O)R¹⁰, —C(O)N(R¹⁰)₂, —N(R¹⁰)C(O)R¹⁰, —N(R¹⁰)C(O)N(R¹⁰)₂, —OC(O)N(R¹⁰)₂, —N(R¹⁰)C(O)OR¹⁰, —C(O)OR¹⁰, —OC(O)R¹⁰, —S(O)R¹⁰, —S(O)₂R¹⁰, —NO₂, ═O, ═S, ═N(R¹⁰), and —CN; and

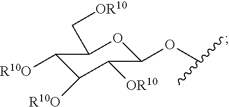

each R¹⁰ is independently selected from:
   hydrogen; and
   C₁₋₆ alkyl, C₂₋₆ alkenyl, and C₂₋₆ alkynyl, each of which is optionally substituted with one or more substituents independently selected from halogen, —CN, —OH, —SH, —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₃₋₁₀ carbocycle, 3- to 10-membered heterocycle; and
   C₃₋₁₀ carbocycle, and 3- to 10-membered heterocycle, each of which is optionally substituted with one or more substituents independently selected from halogen, —CN, —OH, —SH, —NO₂, —NH₂, =0, ═S, —O—C₁₋₆ alkyl, —S—C₁₋₆ alkyl, —N(C₁₋₆ alkyl)₂, —NH(C₁₋₆ alkyl), C₁₋₆ alkyl, C₂₋₆ alkenyl, C₂₋₆ alkynyl, C₃₋₁₀ carbocycle, 3- to 10-membered heterocycle, and C₁₋₆ haloalkyl;
W is selected from:
   C₁₋₆ alkylene, C₂₋₆ alkenylene, and C₂₋₆ alkynylene, each of which is optionally substituted with one or more substituents independently selected from halogen, —CN, —OH, —SH, —NO₂, —NH₂, ═O, ═S, —O—C₁₋₆ alkyl, —S—C₁₋₆ alkyl, —N(C₁₋₆ alkyl)₂, and —NH(C₁₋₆ alkyl), C₃₋₁₀ carbocycle, and 3- to 10-membered heterocycle.

74. The method of embodiment 73, wherein if Z is NH, then R⁴ is not NH₂.

75. The method of embodiment 73, wherein if Z is O or S, R⁴ is —OR³, and R⁵ is H, then W—R₃ is additionally selected from C₁₋₆ alkyl, C₂₋₆ alkenyl, and C₂₋₆ alkynyl.

76. The method of embodiment 73 or 74, wherein X₁, X₂, and X₃ are each independently CR⁶, and X₄ is C-Q-R³.

77. The method of embodiment 73, wherein Z is selected from —O— and —S—.

78. The method of embodiment 73 or 76, wherein Z is —O—.

79. The method of any one of embodiments 73 to 78, wherein X is CR⁶.

80. The method of any one of embodiments 73 to 79, wherein Y is CR¹².

81. The method of any one of embodiments 73 to 80, wherein Q is absent or selected from —W—, —O—, —OW—, and —WO—.

82. The method of any one of embodiments 73 to 81, wherein Q is absent or —O—.

83. The method of any one of embodiments 73 to 82, wherein Q is absent.

84. The method of any one of embodiments 73 to 83, wherein m is selected from 1 and 2.

85. The method of any one of embodiments 73 to 84, wherein m is 1.

86. The method of any one of embodiments 73 to 85, wherein R¹ is selected from:
hydrogen;
C₁₋₆ alkyl, C₂₋₆ alkenyl, and C₂₋₆ alkynyl, each of which is optionally substituted with one or more substituents independently selected from halogen, —CN, —OH, —SH, —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₃₋₁₀ carbocycle, 3- to 10-membered heterocycle; and
   C₃₋₆ carbocycle, and 3- to 6-membered heterocycle, each of which is optionally substituted with one or more substituents independently selected from halogen, —CN, —OH, —SH, —NO₂, —NH₂, ═O, ═S, —O—C₁₋₆ alkyl, —S—C₁₋₆ alkyl, —N(C₁₋₆ alkyl)₂, and —NH(C₁₋₆ alkyl).

87. The method of any one of embodiments 73 to 84, wherein R¹ is selected from hydrogen and C₁₋₃ alkyl.

88. The method of any one of embodiments 73 to 86, wherein R² is selected from:
hydrogen;
halogen, —CN, —OH, —SH, —NO₂, —NH₂, ═O, ═S, —O—C₁₋₆ alkyl, —S—C₁₋₆ alkyl, —N(C₁₋₆ alkyl)₂, —NH(C₁₋₆ alkyl);
C₁₋₆ alkyl, C₂₋₆ alkenyl, and C₂₋₆ alkynyl, each of which is optionally substituted with one or more substituents independently selected from halogen, —CN, —OH, —SH, —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₃₋₁₀ carbocycle, 3- to 10-membered heterocycle; and
C₃₋₆ carbocycle, and 3- to 6-membered heterocycle, each of which is optionally substituted with one or more substituents independently selected from halogen, —CN, —OH, —SH, —NO₂, —NH₂, ═O, ═S, —S—C₁₋₆ alkyl, —N(C₁₋₆ alkyl)₂, and —NH(C₁₋₆ alkyl).

89. The method of any one of embodiments 73 to 85, wherein R² is selected from:
hydrogen;
halogen, —CN, —OH, —SH, —NO₂, —NH₂, ═O, ═S, —O—C₁₋₆ alkyl, —S—C₁₋₆ alkyl, —N(C₁₋₆ alkyl)₂, —NH(C₁₋₆ alkyl);
C₁₋₆ alkyl optionally substituted with one or more substituents independently selected from halogen, —CN, —OH, —SH, —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₃₋₁₀ carbocycle, 3- to 10-membered heterocycle; and
C₆ carbocycle, and 5- to 6-membered heterocycle, each of which is optionally substituted with one or more substituents independently selected from halogen.

90. The method of any one of embodiments 73 to 86, wherein R² is selected from:
hydrogen;
halogen, —CN, —OH, —SH, —NO₂, —NH₂, ═O, ═S, —O—C₁₋₆ alkyl, —S—C₁₋₆ alkyl, —N(C₁₋₆ alkyl)₂, —NH(C₁₋₆ alkyl);
C₁₋₆ alkyl optionally substituted with one or more substituents independently selected from halogen, —CN, —OH, —SH, —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₃₋₁₀ carbocycle, and 3- to 10-membered heterocycle.

91. The method of any one of embodiments 73 to 86, wherein R² is selected from hydrogen, halogen, and C₁₋₃ alkyl.

92. The method of any one of embodiments 73 to 91, wherein R³ is a C₃₋₆ carbocycle optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —N(R¹⁰)C(O)N(R¹⁰)₂, —OC(O)N(R¹⁰)₂, —N(R¹⁰)C(O)OR¹⁰, —C(O)OR¹⁰, —OC(O)R¹⁰, —S(O)R¹⁰, —S(O)₂R¹⁰, —NO₂, ═O, ═S, ═N(R¹⁰), —CN, C₁₋₆ alkyl, C₂₋₆ alkenyl, and C₂₋₆ alkynyl, wherein C₁₋₆ alkyl, C₂₋₆ alkenyl, and C₂₋₆ alkynyl are each optionally substituted with one or more R⁹.

93. The method of any one of embodiments 73 to 91, wherein R³ is a C₆ carbocycle optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —N(R¹⁰)C(O)N(R¹⁰)₂, —OC(O)N(R¹⁰)₂, —N(R¹⁰)C(O)OR¹⁰, —C(O)OR¹⁰, —OC(O)R¹⁰, —S(O)R¹⁰, —S(O)₂R¹⁰, —NO₂, ═O, ═S, ═N(R¹⁰), —CN, C₁₋₆ alkyl, C₂₋₆ alkenyl, and C₂₋₆ alkynyl, wherein C₁₋₆ alkyl, C₂₋₆ alkenyl, and C₂₋₆ alkynyl are each optionally substituted with one or more R⁹.

94. The method of any one of embodiments 73 to 91, wherein R³ is phenyl optionally substituted with one or more substituents independently selected from halogen, —CN, —OH, —SH, —NO₂, —NH₂, —O—C₁₋₆ alkyl, —S—C₁₋₆ alkyl, —N(C₁₋₆ alkyl)₂, and —NH(C₁₋₆ alkyl).

95. The method of any one of embodiments 73 to 91, wherein R³ is a 3- to 6-membered heterocycle optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —N(R¹⁰)C(O)N(R¹⁰)₂, —OC(O)N(R¹⁰)₂, —N(R¹⁰)C(O)OR¹⁰, —C(O)OR¹⁰, —OC(O)R¹⁰, —S(O)R¹⁰, —S(O)₂R¹⁰, —NO₂, ═O, ═S, ═N(R¹⁰), —CN, C₁₋₆ alkyl, C₂₋₆ alkenyl, and C₂₋₆ alkynyl, wherein C₁₋₆ alkyl, C₂₋₆ alkenyl, and C₂₋₆ alkynyl are each optionally substituted with one or more R⁹.

96. The method of any one of embodiments 73 to 91, wherein R³ is a 5- to 6-membered heterocycle optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —N(R¹⁰)C(O)N(R¹⁰)₂, —OC(O)N(R¹⁰)₂, —N(R¹⁰)C(O)OR¹⁰, —C(O)OR¹⁰, —OC(O)R¹⁰, —S(O)R¹⁰, —S(O)₂R¹⁰, —NO₂, ═O, ═S, ═N(R¹⁰), —CN, C₁₋₆ alkyl, C₂₋₆ alkenyl, and C₂₋₆ alkynyl, wherein C₁₋₆ alkyl, C₂₋₆ alkenyl, and C₂₋₆ alkynyl are each optionally substituted with one or more R⁹.

97. The method of any one of embodiments 73 to 91, wherein R³ is a 6-membered heterocycle optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —N(R¹⁰)C(O)N(R¹⁰)₂, —OC(O)N(R¹⁰)₂, —N(R¹⁰)C(O)OR¹⁰, —C(O)OR¹⁰, —OC(O)R¹⁰, —S(O)R¹⁰, —S(O)₂R¹⁰, —NO₂, ═O, ═S, ═N(R¹⁰), —CN, C₁₋₆ alkyl, C₂₋₆ alkenyl, and C₂₋₆ alkynyl, wherein C₁₋₆ alkyl, C₂₋₆ alkenyl, and C₂₋₆ alkynyl are each optionally substituted with one or more R⁹.

98. The method of any one of embodiments 73 to 91, wherein R³ is 6-membered heterocycle optionally substituted with one or more substituents independently selected from halogen, —CN, —OH, —SH, —NO₂, —NH₂, —O—C₁₋₆ alkyl, —S—C₁₋₆ alkyl, —N(C₁₋₆ alkyl)₂, and —NH(C₁₋₆ alkyl).

99. The method of any one of embodiments 73 to 91, wherein R³ is a 5-membered heterocycle optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —N(R¹⁰)C(O)N(R¹⁰)₂, —OC(O)N(R¹⁰)₂, —N(R¹⁰)C(O)OR¹⁰, —C(O)OR¹⁰, —OC(O)R¹⁰, —S(O)R¹⁰, —S(O)₂R¹⁰, —NO₂, ═O, ═S, ═N(R¹⁰), —CN, C₁₋₆ alkyl, C₂₋₆ alkenyl, and C₂₋₆ alkynyl, wherein C₁₋₆ alkyl, C₂₋₆ alkenyl, and C₂₋₆ alkynyl are each optionally substituted with one or more R⁹.

100. The method of any one of embodiments 73 to 91, wherein R³ is a 5-membered heterocycle optionally substituted with one or more substituents independently selected from halogen, —CN, —OH, ═O, ═S, ═NH, —SH, —NO₂, —NH₂, —O—C₁₋₆ alkyl, —S—C₁₋₆ alkyl, —N(C₁₋₆ alkyl)₂, —NH(C₁₋₆ alkyl), and C₁₋₆ alkyl.

101. The method of any one of embodiments 73 to 91, wherein R³ is a 5-membered heterocycle is selected from isoxazole, oxazole, thiadiazole, oxadiazole, pyrazole, tetrazole, and thiazole, any of which is optionally substituted with one or more R⁹.

102. The method of any one of embodiments 73 to 91, wherein R³ is a 5-membered heterocycle is selected tetrazole and oxadiazole.

103. The method of any one of embodiments 73 to 91, wherein R³ is a 5-membered heterocycle selected from isothiazol-3-ol, isoxazole-3-ol, squaric acid, thiazolidinedione, oxazolidinedione, tetrazole, 1,2,4-oxadiazol-5(4H)-one, 1,2,4-thiadiazol-5(4H)-one, 1,2,4-oxadiazole-5(4H)-thione, 2-methyl-2,4-dihydro-3H-1,2,4-triazole-3-thione, 3H-1,2,3,5-oxathiadiazole 2-oxide, 3-(methylsulfonyl)-4H-1,2,4-triazole, or 1,4-dihydro-5H-tetrazol-5-one, 3-hydroxyquinolin-2-one, and tetramic acid, any of which is optionally substituted with one or more R⁹.

104. The method of any one of embodiments 73 to 91, wherein R³ is a 5-membered heterocycle is selected from

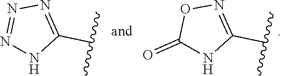 [SMILES: *c1nnn[nH]1 ; *c1noc(=O)[nH]1]

105. The compound or salt of any one of embodiments 73 to 91, wherein R³ is selected from,

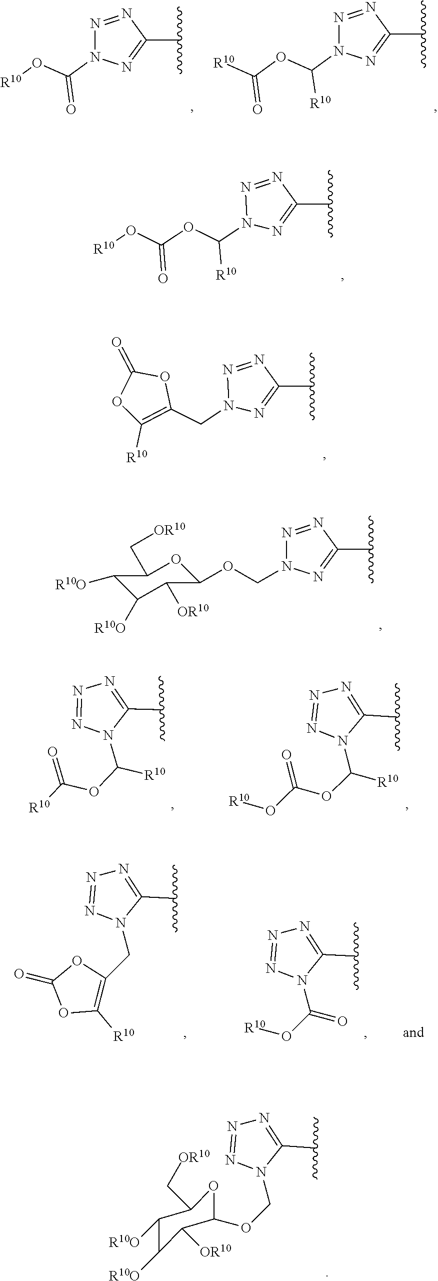

106. The method of any one of embodiments 73 to 91, wherein Q is —O— and R³ is tetrazole.

107. The method of any one of embodiments 73 to 91, wherein
Q is —W—;
R³ is additionally selected from halogen, —OR¹⁰, —SR¹⁰, —N(R¹⁰)₂, —C(O)R¹⁰, —C(O)N(R¹⁰)₂, —N(R¹⁰)C(O)R¹⁰, —N(R¹⁰)C(O)N(R¹⁰)₂, —OC(O)N(R¹⁰)₂, —N(R¹⁰)C(O)OR¹⁰, —C(O)OR¹⁰, —OC(O)R¹⁰, —S(O)R¹⁰, —S(O)₂R¹⁰, —NO₂, —CN; and
—W— is C₁₋₆ alkylene optionally substituted with one or more substituents independently selected from halogen, —CN, —OH, —SH, —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₃₋₁₀ carbocycle, and 3- to 10-membered heterocycle.

108. The method of any one of embodiments 73 to 91, wherein
Q is —W—; and
R³ is additionally selected from halogen, —CN, —OH, —SH, —NO₂, —NH₂, —O—C₁₋₆ alkyl, —S—C₁₋₆ alkyl, —N(C₁₋₆ alkyl)₂, and —NH(C₁₋₆ alkyl), and
—W— is C₁₋₆ alkylene optionally substituted with one or more substituents independently selected from halogen, —CN, —OH, —SH, —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₃₋₁₀ carbocycle, and 3- to 10-membered heterocycle.

109. The method of any one of embodiments 73 to 91, wherein
Q is —W—; and
R³ is additionally selected from —OH, —SH, —NH₂, —O—C₁₋₆ alkyl, —S—C₁₋₆ alkyl, —N(C₁₋₆ alkyl)₂, and —NH(C₁₋₆ alkyl); and
—W— is C₁ alkylene optionally substituted with one or more substituents independently selected from halogen, —CN, —OH, —SH, —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₃₋₁₀ carbocycle, and 3- to 10-membered heterocycle.

110. The method of any one of embodiments 73 to 91, wherein
Q is —W—; and
R³ is additionally selected from —OH, —SH, —NH₂, —O—C₁₋₆ alkyl, —S—C₁₋₆ alkyl, —N(C₁₋₆ alkyl)₂, and —NH(C₁₋₆ alkyl); and
—W— is —C(═O)—.

111. The method of any one of embodiments 73 to 110, wherein each R⁴ is independently selected from:
halogen, —OR⁷, —SR⁷, —N(R⁷)₂, —NO₂, and —CN;
C₁₋₆ alkyl, C₂₋₆ alkenyl, and C₂₋₆ alkynyl, each of which is optionally substituted with one or more substituents independently selected from halogen, —CN, —OH, —SH, —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₃₋₁₀ carbocycle, 3- to 10-membered heterocycle; and
C₃₋₆ carbocycle, and 3- to 6-membered heterocycle, each of which is optionally substituted with one or more substituents independently selected from halogen, —CN, —OH, —SH, —NO₂, —NH₂, ═O, ═S, —O—C₁₋₆ alkyl, —S—C₁₋₆ alkyl, —N(C₁₋₆ alkyl)₂, and —NH(C₁₋₆ alkyl);

112. The method of any one of embodiments 73 to 110, wherein R⁴ is independently selected from —OR⁷, —SR⁷, and —N(R⁷)₂.

113. The method of any one of embodiments 73 to 110, wherein m is 1 and R⁴ is —OR⁷.

114. The method of any one of embodiments 73 to 113, wherein each R⁷ is selected from:
C₁₋₆ alkyl, C₂₋₆ alkenyl, and C₂₋₆ alkynyl, each of which is optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —C(O)OR¹⁰, —OC(O)R¹⁰, —N(R¹⁰)C(O)N(R¹⁰)₂, —OC(O)N(R¹⁰)₂, —N(R¹⁰)C(O)OR¹⁰, —S(O)R¹⁰, —S(O)₂R¹⁰, —NO₂, ═O, ═S, ═N(R¹⁰), —CN, C₃₋₁₀ carbocycle and 3- to 10-membered heterocycle, wherein the C₃₋₁₀ carbocycle and 3- to 10-membered heterocycle are each optionally substituted with one or more R⁹; and
C₃₋₁₀ carbocycle and 3- to 10-membered heterocycle, each of which is optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —N(R¹⁰)C(O)N(R¹⁰)₂, —OC(O)N(R¹⁰)₂, —N(R¹⁰)C(O)OR¹⁰, —C(O)OR¹⁰, —OC(O)R¹⁰, —S(O)R¹⁰, —S(O)₂R¹⁰, —NO₂, ═O, ═S, ═N(R¹⁰), —CN, C₁₋₆ alkyl, C₂₋₆ alkenyl, and C₂₋₆ alkynyl, wherein C₁₋₆ alkyl, C₂₋₆ alkenyl, and C₂₋₆ alkynyl are each optionally substituted with one or more R⁹.

115. The method of any one of embodiments 73 to 113, wherein each R⁷ is selected from:
C₁₋₆ alkyl optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —C(O)OR¹⁰, —OC(O)R¹⁰, —N(R¹⁰)C(O)N(R¹⁰)₂, —OC(O)N(R¹⁰)₂, —N(R¹⁰)C(O)OR¹⁰, —S(O)R¹⁰, —S(O)₂R¹⁰, —NO₂, ═O, ═S, ═N(R¹⁰), —CN, C₃₋₁₀ carbocycle and 3- to 10-membered heterocycle, wherein the C₃₋₁₀ carbocycle and 3- to 10-membered heterocycle are each optionally substituted with one or more R⁹; and
C₃₋₁₀ carbocycle and 3- to 10-membered heterocycle, each of which is optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —N(R¹⁰)C(O)N(R¹⁰)₂, —OC(O)N(R¹⁰)₂, —N(R¹⁰)C(O)OR¹⁰, —C(O)OR¹⁰, —OC(O)R¹⁰, —S(O)R¹⁰, —S(O)₂R¹⁰, —NO₂, ═O, ═S, ═N(R¹⁰), —CN, C₁₋₆ alkyl, C₂₋₆ alkenyl, and C₂₋₆ alkynyl, wherein C₁₋₆ alkyl, C₂₋₆ alkenyl, and C₂₋₆ alkynyl are each optionally substituted with one or more R⁹.

116. The method of any one of embodiments 73 to 113, wherein each R⁷ is selected from:
C₁₋₆ alkyl optionally substituted with one or more substituents independently selected from C₃₋₁₀ carbocycle and 3- to 10-membered heterocycle, wherein the C₃₋₁₀ carbocycle and 3- to 10-membered heterocycle are each optionally substituted with one or more R⁹; and
C₃₋₁₀ carbocycle and 3- to 10-membered heterocycle, each of which is optionally substituted with one or more substituents independently selected from halogen, —OR¹⁰, —SR¹⁰, —N(R¹⁰)₂, —CN, and C₁₋₆ alkyl optionally substituted with one or more R⁹.

117. The method of any one of embodiments 73 to 113, wherein R⁷ is C₁₋₆ alkyl substituted with one or more substituents independently selected from C₃₋₁₀ carbocycle and 3- to 10-membered heterocycle, wherein the C₃₋₁₀ carbocycle and 3- to 10-membered heterocycle are each optionally substituted with one or more substituents independently selected from halogen, —CN, —OH, —SH, —NO₂, —NH₂, —O—C₁₋₆ alkyl, —S—C₁₋₆ alkyl, —N(C₁₋₆ alkyl)₂, and —NH(C₁₋₆ alkyl).

118. The method of any one of embodiments 73 to 113, wherein R⁷ is C₁₋₆ alkyl substituted with one or more substituents independently selected from C₃₋₆ carbocycle and 3- to 6-membered heterocycle, wherein the C₃₋₆ carbocycle and 3- to 6-membered heterocycle are each optionally substituted with one or more substituents independently selected from halogen, —CN, —OH, —SH, —NO₂, —NH₂, —O—C₁₋₆ alkyl, —S—C₁₋₆ alkyl, —N(C₁₋₆ alkyl)₂, and —NH(C₁₋₆ alkyl).

119. The method of any one of embodiments 73 to 113, wherein R⁷ is selected from C₃₋₁₀ carbocycle and 3- to 10-membered heterocycle, each of which is optionally substituted with one or more substituents independently selected from halogen, —CN, —OH, —SH, —NO₂, —NH₂, —O—C₁₋₆ alkyl, —S—C₁₋₆ alkyl, —N(C₁₋₆ alkyl)₂, and —NH(C₁₋₆ alkyl).

120. The method of any one of embodiments 73 to 45, wherein R⁶ is selected from hydrogen, halogen, —CN, —OH, —SH, —NO₂, —NH₂, —O—C₁₋₆ alkyl, —S—C₁₋₆ alkyl, —N(C₁₋₆ alkyl)₂, and —NH(C₁₋₆ alkyl).

121. The method of any one of embodiments 73 to 120, wherein R⁶ is selected from hydrogen and halogen.

122. The method of any one of embodiments 73 to 121, wherein Z is —NR⁸—.

123. The method of any one of embodiments 73 to 121, wherein R⁸ is selected from hydrogen.

124. The method of any one of embodiments 73 to 121, wherein R⁸ is selected from C₁₋₆ alkyl, C₂₋₆ alkenyl, and C₂₋₆ alkynyl, each of which is optionally substituted with one or more substituents independently selected from halogen, —SR¹⁰, —N(R¹⁰)₂, —C(O)R¹⁰, —C(O)N(R¹⁰)₂, —N(R¹⁰)C(O)R¹⁰, —C(O)OR¹⁰, —OC(O)R¹⁰, —N(R¹⁰)C(O)N(R¹⁰)₂, —OC(O)N(R¹⁰)₂, —N(R¹⁰)C(O)OR¹⁰, —S(O)R¹⁰, —S(O)₂R¹⁰, —NO₂, ═O, ═S, ═N(R¹⁰), —CN, C₃₋₁₀ carbocycle and 3- to 10-membered heterocycle, wherein the C₃₋₁₀ carbocycle and 3- to 10-membered heterocycle are each optionally substituted with one or more R⁹.

125. The method of any one of embodiments 73 to 121, wherein R⁸ is selected from C₁₋₆ alkyl, C₂₋₆ alkenyl, and C₂₋₆ alkynyl, each of which is optionally substituted with one or more substituents independently selected from halogen, —CN, —SH, —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₃₋₁₀ carbocycle, 3- to 10-membered heterocycle.

126. The method of any one of embodiments 73 to 121, wherein R⁸ is selected from C₁₋₆ alkyl optionally substituted with one or more substituents independently selected from halogen, —CN, —SH, —NO₂, —NH₂, —O—C₁₋₆ alkyl, —S—C₁₋₆ alkyl, —N(C₁₋₆ alkyl)₂, and —NH(C₁₋₆ alkyl).

127. The method of any one of embodiments 73 to 121, wherein R⁸ is selected from C₁₋₆ alkyl.

128. The method of any one of embodiments 73 to 121, wherein R⁸ is selected from C₃₋₁₀ optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —N(R¹⁰)C(O)N(R¹⁰)₂, —OC(O)N(R¹⁰)₂, —N(R¹⁰)C(O)OR¹⁰, —C(O)OR¹⁰, —OC(O)R¹⁰, —S(O)R¹⁰, —S(O)₂R¹⁰, —NO₂, ═O, ═S, ═N(R¹⁰), —CN, C₁₋₆ alkyl, C₂₋₆ alkenyl, and C₂₋₆ alkynyl, wherein C₁₋₆ alkyl, C₂₋₆ alkenyl, and C₂₋₆ alkynyl are each optionally substituted with one or more R⁹.

129. The method of any one of embodiments 73 to 121, wherein R⁸ is selected from C₃₋₁₀ carbocycle optionally substituted with one or more substituents independently selected from halogen, —CN, —OH, —SH, —NO₂, —NH₂, —O—C₁₋₆ alkyl, —S—C₁₋₆ alkyl, —N(C₁₋₆ alkyl)₂, and —NH(C₁₋₆ alkyl).

130. The method of any one of embodiments 73 to 121, wherein R⁸ is selected from 3- to 10-membered heterocycle optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —N(R¹⁰)C(O)N(R¹⁰)₂, —OC(O)N(R¹⁰)₂, —N(R¹⁰)C(O)OR¹⁰, —C(O)OR¹⁰, —OC(O)R¹⁰, —S(O)R¹⁰, —S(O)₂R¹⁰, —NO₂, ═O, ═S, ═N(R¹⁰), —CN, C₁₋₆ alkyl, C₂₋₆ alkenyl, and C₂₋₆ alkynyl, wherein C₁₋₆ alkyl, C₂₋₆ alkenyl, and C₂₋₆ alkynyl are each optionally substituted with one or more R⁹.

131. The method of any one of embodiments 73 to 121, wherein R⁸ is selected from 3- to 10-membered heterocycle optionally substituted with one or more substituents independently selected from halogen, —CN, —OH, —SH, —NO₂, —NH₂, —O—C₁₋₆ alkyl, —S—C₁₋₆ alkyl, —N(C₁₋₆ alkyl)₂, and —NH(C₁₋₆ alkyl).

132. The method of any one of embodiments 73 to 121, wherein R⁸ is hydrogen.

133. The method of any one of embodiments 73 to 132, wherein each R⁹ is selected from:
halogen, —CN, —OH, —SH, —NO₂, —NH₂, —O—C₁₋₆ alkyl, —S—C₁₋₆ alkyl, —N(C₁₋₆ alkyl)₂, and —NH(C₁₋₆ alkyl); and
C₁₋₃ alkyl optionally substituted with one or more substituents independently selected from halogen, —CN, —OH, —SH, —NO₂, —NH₂, —O—C₁₋₆ alkyl, —S—C₁₋₆ alkyl, —N(C₁₋₆ alkyl)₂, and —NH(C₁₋₆ alkyl).

134. The method of any one of embodiments 73 to 133, wherein each R¹⁰ is hydrogen.

135. The method of any one of embodiments 73 to 134, wherein W is C₁₋₆ alkylene optionally substituted with one or more substituents independently selected from halogen, —CN, —OH, —SH, —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₃₋₁₀ carbocycle, and 3- to 10-membered heterocycle.

136. The method of any one of embodiments 73 to 134, wherein W is C₁₋₆ alkylene optionally substituted with one or more substituents independently selected from halogen, —CN, —OH, —SH, —NO₂, —NH₂, —O—C₁₋₆ alkyl, —S—C₁₋₆ alkyl, —N(C₁₋₆ alkyl)₂, —NH(C₁₋₆ alkyl), C₃₋₁₀ carbocycle, and 3- to 10-membered heterocycle.

137. The method of any one of embodiments 73 to 134, wherein W is C₁₋₆ alkylene optionally substituted with one or more substituents independently selected from halogen, —CN, —OH, —SH, —NO₂, —NH₂, —O—C₁₋₆ alkyl, —S—C₁₋₆ alkyl, —N(C₁₋₆ alkyl)₂, and —NH(C₁₋₆ alkyl).

138. The method of any one of embodiments 73 to 134, wherein W is C₁₋₆ alkylene optionally substituted with one or more substituents independently selected from C₃₋₁₀ carbocycle, and 3- to 10-membered heterocycle.

139. The method of any one of embodiments 73 to 134, wherein W is C₁₋₆ alkylene substituted with one or more substituents independently selected from C₃₋₆ carbocycle, and 3- to 5-membered heterocycle.

140. The method of any one of embodiments 73 to 134, wherein W is C₁₋₆ alkylene substituted with one or more substituents independently selected from C₃₋₆ carbocycle.

141. The method of any one of embodiments 73 to 134, wherein W is selected from

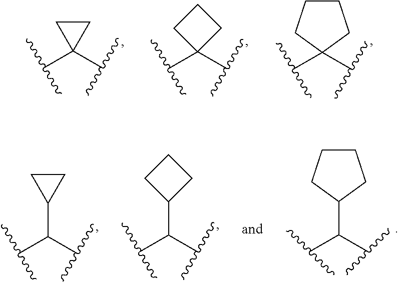 [SMILES: *C1(*)CC1 ; *C1(*)CCC1 ; *C1(*)CCCC1 ; *C(*)C1CC1 ; *C(*)C1CCC1 ; *C(*)C1CCCC1]

142. The method of any one of embodiments 73 to 141, wherein R¹² is selected from hydrogen and halogen.

143. The method of any one of embodiments 73 to 141, wherein the method in treating cancer is a cancer immunotherapy.

144. The method of any one of embodiments 73 to 143, wherein the cancer is a solid tumor.

145. The method of embodiment 143, wherein the solid tumor is a sarcoma, carcinoma, or lymphoma.

146. The method of embodiment 143, wherein the solid tumor is in the breast, brain, kidney, ovary, cervix, pancreas, thyroid, colon, bladder, prostate, liver, stomach, or lung.

1.-146. (canceled)

147. A compound represented by Formula (I): 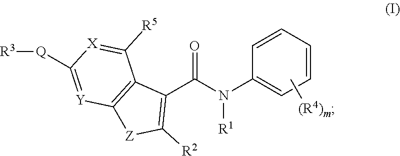
or a pharmaceutically acceptable salt thereof, wherein:
X is selected from CR⁶ and N;
Y is CH;
Z is selected from —O—, —S—, and —NR⁸—;
Q is absent or selected from: —O—, —W—, and —O—W—;
m is selected from 1, 2, 3, 4, and 5;
R¹ is hydrogen or C₁₋₆ alkyl;
R² is hydrogen;
R³ is a 5-membered heterocycle selected from 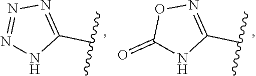
and wherein Q is —W—, then R³ is additionally selected from halogen, —OR¹⁰, —SR¹⁰, —N(R¹⁰)₂, —C(O)R¹⁰, —C(O)N(R¹⁰)₂, —N(R¹⁰)C(O)R¹⁰, —N(R¹⁰)C(O)N(R¹⁰)₂, —OC(O)N(R¹⁰)₂, —N(R¹⁰)C(O)OR¹⁰, —C(O)OR¹⁰, —OC(O)R¹⁰, —S(O)R¹⁰, —S(O)₂R¹⁰, —NO₂, —CN;
each R⁴ is independently selected from:
halogen;
—OR⁷;
C₁₋₆ alkyl optionally substituted with one or more substituents independently selected from halogen, C₃₋₁₀ carbocycle, and 3- to 10-membered heterocycle, wherein the C₃₋₁₀ carbocycle and 3- to 10-membered heterocycle are each optionally substituted with 1 to 3 R⁹; and
C₃₋₁₀ carbocycle and 3- to 10-membered heterocycle, each of which is optionally substituted with 1 to 3 substituents independently selected from halogen, —CN, and C₁₋₆ alkyl optionally substituted with 1 to 3 R⁹;
R⁵ is hydrogen;
R⁶ is hydrogen or halogen;
each R⁷ is independently selected from:
C₁₋₆ alkyl optionally substituted with one or more substituents independently selected from halogen, C₃₋₁₀ carbocycle and 3- to 10-membered heterocycle, wherein the C₃₋₁₀ carbocycle and 3- to 10-membered heterocycle are each optionally substituted with 1 to 3 R⁹; and
C₃₋₁₀ carbocycle and 3- to 10-membered heterocycle, each of which is optionally substituted with 1 to 3 substituents independently selected from halogen, CN, and C₁₋₆ alkyl optionally substituted with one or more R⁹;
R⁸ is hydrogen or C₁₋₆ alkyl optionally substituted with 1 to 3 halogen;
each R⁹ is independently selected from: halogen, —CN, and C₁₋₃ alkyl optionally substituted with 1 to 3 substituents independently selected from halogen and —CN;
each R¹⁰ is independently selected from: hydrogen or C₁₋₆ alkyl optionally substituted with one or more substituents independently selected from —CN, —OH, —SH, —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₃₋₁₀ carbocycle, and 3- to 10-membered heterocycle; and
W is C₁₋₆ alkylene or —C(O)—.

148. The compound or salt of claim 147, wherein Z is selected from —O— and —S—.

149. The compound or salt of claim 148, wherein Z is —O—.

150. The compound or salt of claim 147, wherein X is CR⁶.

151. The compound or salt of claim 147, wherein Q is absent or —O—.

152. The compound or salt of claim 147, wherein Q is absent.

153. The compound or salt of claim 147, wherein m is selected from 1 and 2.

154. The compound or salt of claim 147, wherein m is 1.

155. The compound or salt of claim 147, wherein R¹ is hydrogen.

156. The compound or salt of claim 147, wherein R¹ is C₁₋₆ alkyl.

157. The compound or salt of claim 147, wherein R³ is H

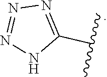 [SMILES: *c1nnn[nH]1]

158. The compound or salt of claim 147, wherein R³ is H

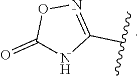 [SMILES: *c1noc(=O)[nH]1]

159. The compound or salt of claim 147, wherein m is 1 or 2 and one R⁴ is halogen.

160. The compound or salt of claim 147, wherein m is 1 or 2 and one R⁴ is —OR⁷.

161. The compound or salt of claim 147, wherein m is 1 or 2 and one R⁴ is C₁₋₆ alkyl optionally substituted with one or more substituents independently selected from halogen, C₃₋₁₀ carbocycle, and 3- to 10-membered heterocycle, wherein the C₃₋₁₀ carbocycle and 3- to 10-membered heterocycle are each optionally substituted with 1 to 3 R⁹.

162. The compound or salt of claim 147, wherein R⁶ is hydrogen.

163. The compound or salt of claim 147, wherein R⁶ is halogen.

164. The compound or salt of claim 147, selected from:

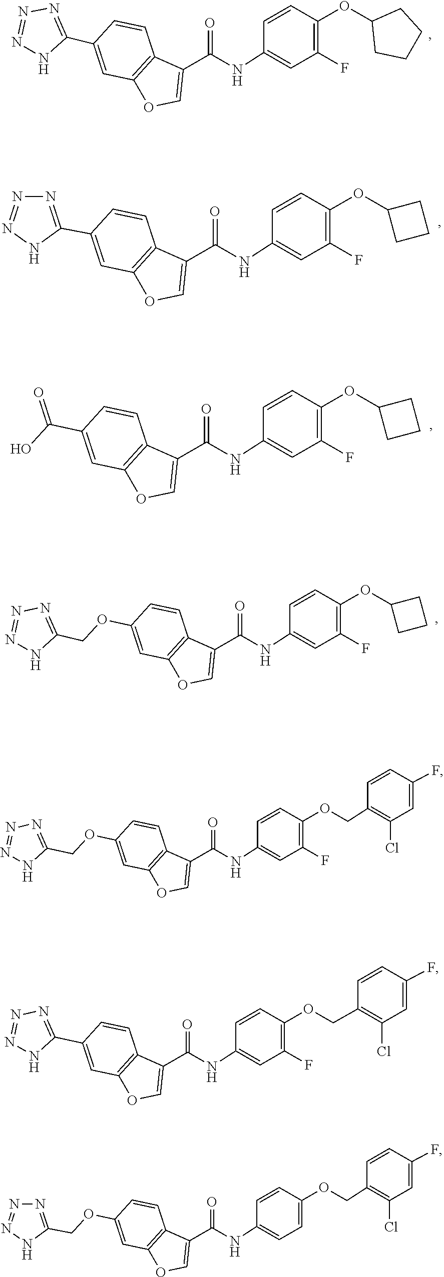

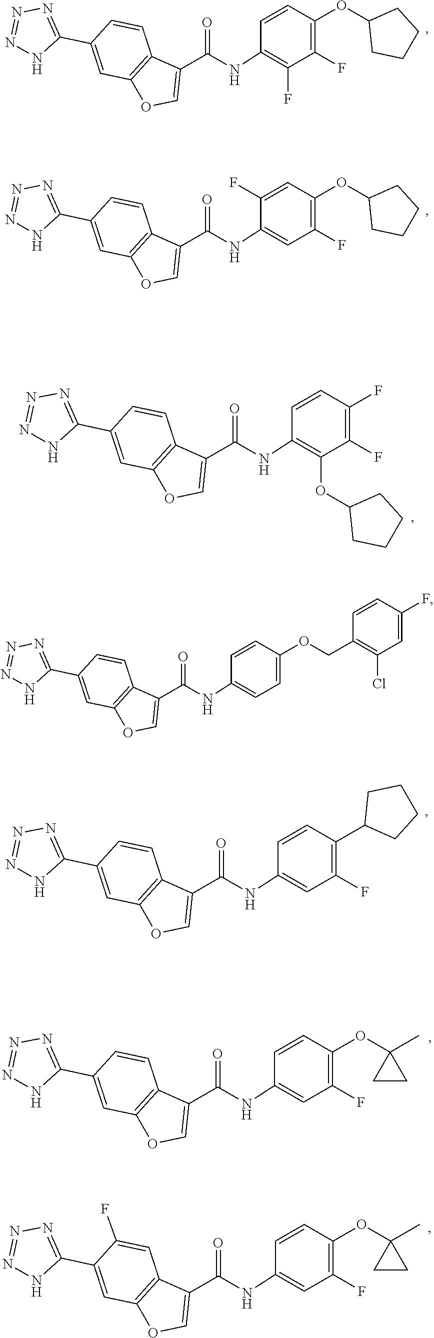

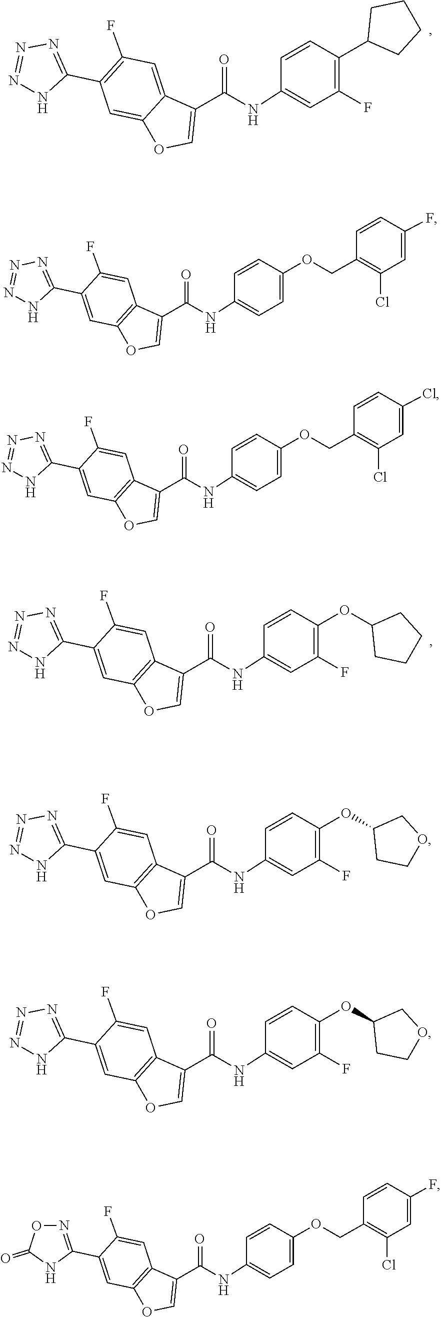

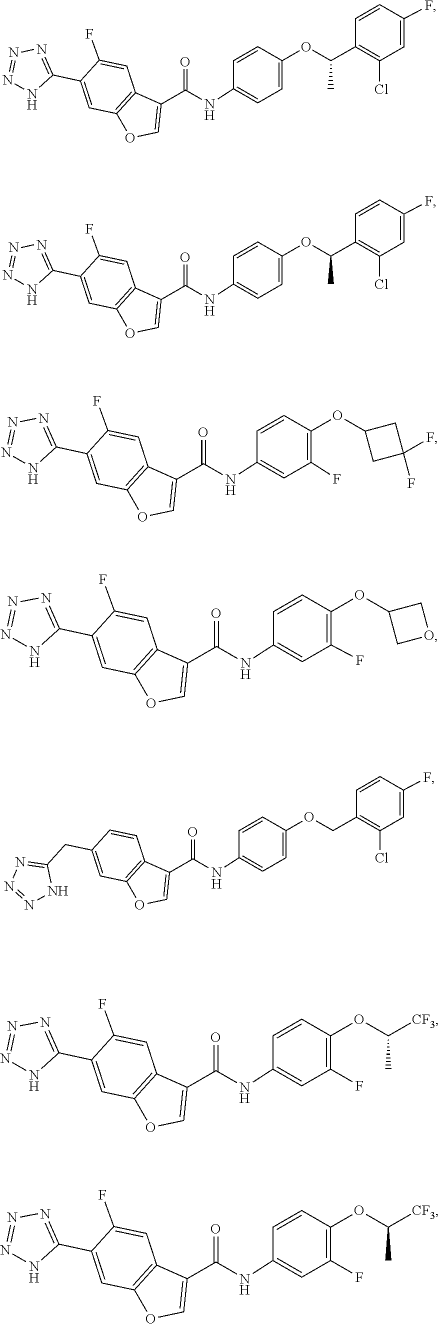

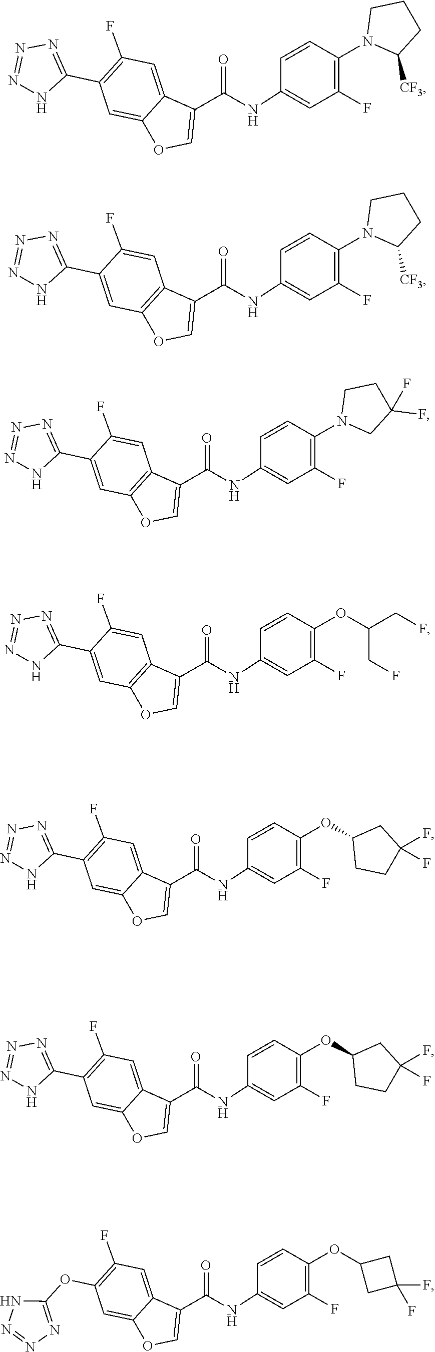

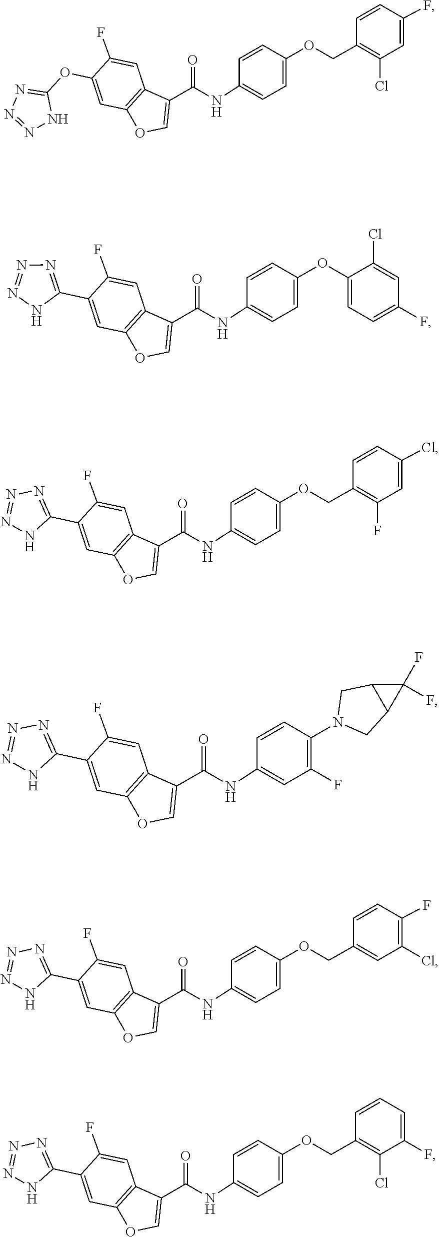 [SMILES: O=C(Nc1ccc(OCc2ccc(F)cc2Cl)cc1)c1coc2cc(Oc3nnn[nH]3)c(F)cc12 ; O=C(Nc1ccc(Oc2ccc(F)cc2Cl)cc1)c1coc2cc(-c3nnn[nH]3)c(F)cc12 ; O=C(Nc1ccc(OCc2ccc(Cl)cc2F)cc1)c1coc2cc(-c3nnn[nH]3)c(F)cc12 ; O=C(Nc1ccc(N2CC3C(C2)C3(F)F)c(F)c1)c1coc2cc(-c3nnn[nH]3)c(F)cc12 ; O=C(Nc1ccc(OCc2ccc(F)c(Cl)c2)cc1)c1coc2cc(-c3nnn[nH]3)c(F)cc12 ; O=C(Nc1ccc(OCc2cccc(F)c2Cl)cc1)c1coc2cc(-c3nnn[nH]3)c(F)cc12]

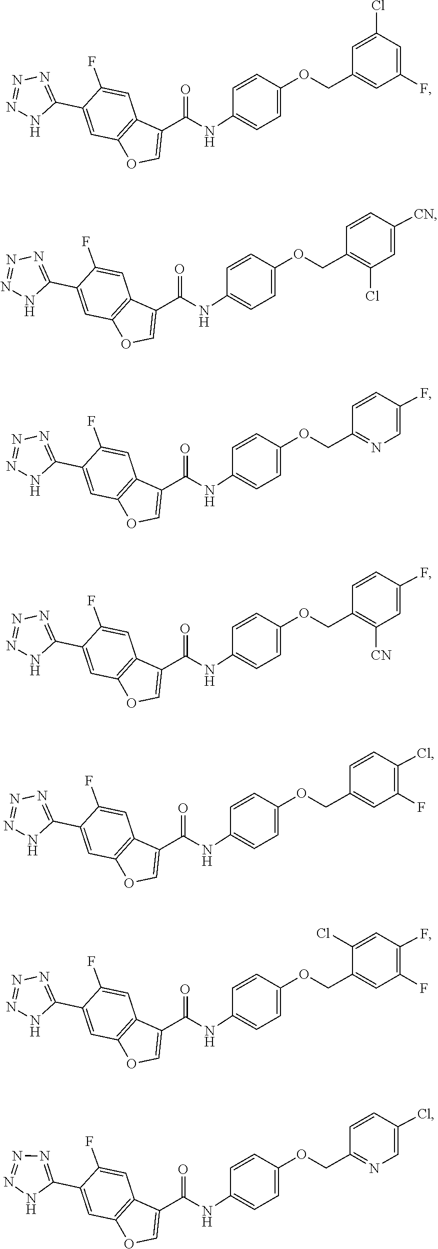

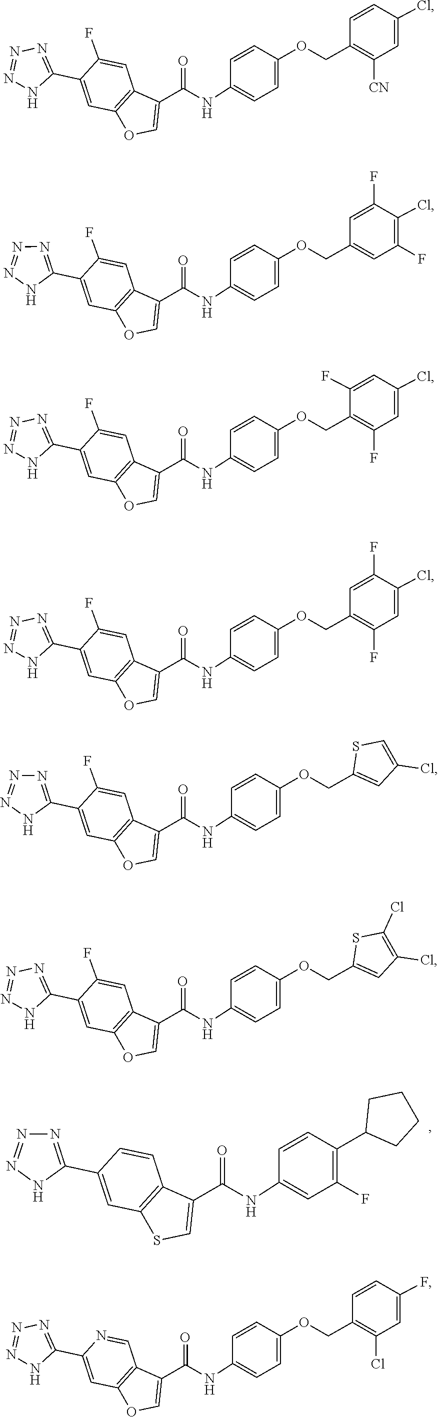

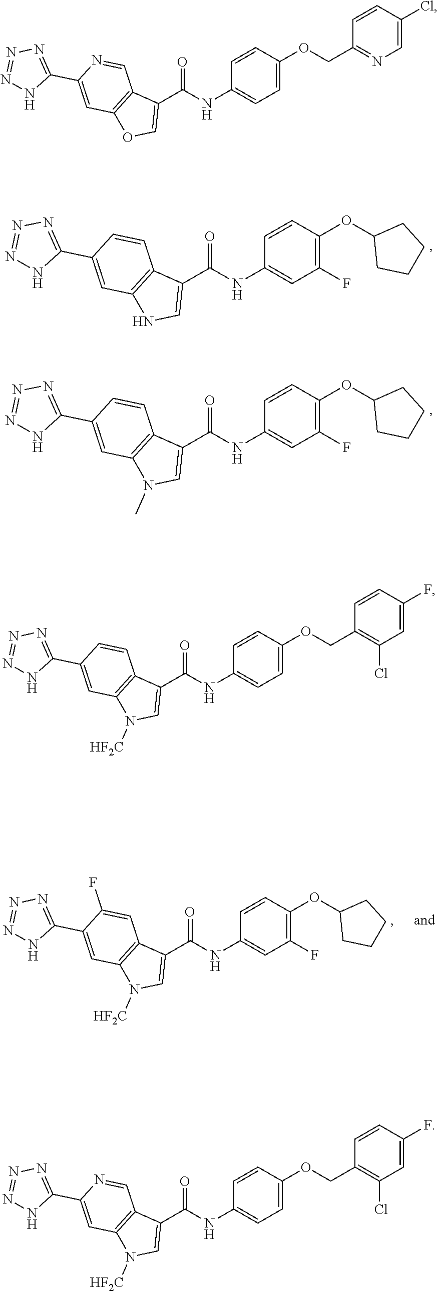

165. A pharmaceutical composition comprising a compound of claim 147 and a pharmaceutically acceptable excipient.

166. A method of treating cancer, comprising administering to a subject in need thereof a compound of claim 147 or a pharmaceutically acceptable salt thereof.

167. The method of claim 166, wherein the method in treating cancer is a cancer immunotherapy.

168. The method of claim 166, wherein the cancer is a solid tumor.

169. The method of claim 168, wherein the solid tumor is a sarcoma, carcinoma, or lymphoma.

170. The method of claim 169, wherein the solid tumor is in the breast, brain, kidney, ovary, cervix, pancreas, thyroid, colon, bladder, prostate, liver, stomach, or lung.
